(19) United States
(12) Patent Application Publication
(10) Pub. No.: US 2011/0245242 A1
(43) Pub. Date: Oct. 6, 2011

(54) Heterocyclically Substituted Anilinopyrimides

(75) Inventors: Jörg Nico Greul, Leverkusen (DE); Oliver Gaertzen, Koln (DE); Hendrik Helmke, Liederbach (DE); Stefan Hille, Neuss (DE); Kerstin Ilgö, Köln (DE); Amos Mattes, Langenfeld (DE); Pierre Wasnaire, Düsseldorf (DE); Carl Friedrich Nising, Langenfeld (DE); Ulrike Wachendorff-Neumann, Neuwied (DE); Arnd Voerste, Koln (DE); Peter Dahmen, Neuss (DE); Rurth Meissner, Leverkusen (DE); Christoph Andreas Braun, Düsseldorf (DE); Martin Kaubmann, Koln (DE); Hiroyuki Hadano, Tochigi (JP)

(73) Assignee: Bayer CropScience AG, Monheim (DE)

(21) Appl. No.: 13/061,885

(22) PCT Filed: Aug. 21, 2009
(86) PCT No.: PCT/EP09/06055

§ 371 Date: May 27, 2011

(30) Foreign Application Priority Data
Sep. 3, 2008 (EP) 08163621.9

(51) Int. Cl. A01N 43/54; C07D 403/12; C07D 413/12; A01N 43/76; C07D 401/12; A01N 43/84; C07D 239/42; C07D 239/48; A01P 3/00

(52) U.S. Cl. 514/230.8; 544/323; 514/275; 544/122; 544/326; 544/332; 544/330

(57) ABSTRACT

Heterocyclically substituted anilinopyrimidines of the formula (I)

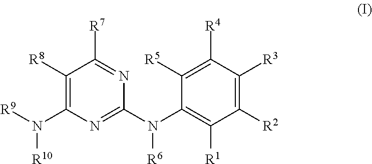

in which R¹ to R¹⁰ and L₁, L₂, E1, E2, E3, Y and Z have the meanings given in the description, and agrochemically active salts thereof, their use and also methods and compositions for controlling phytopathogenic harmful fungi in and/or on plants or in and/or on seed of plants, processes for preparing such compositions and treated seed and also their use for controlling phytopathogenic harmful fungi in agriculture, horticulture and forestry, in the protection of materials and in the domestic and hygiene field. The present invention furthermore relates to a process for preparing heterocyclically substituted anilinopyrimidinenes of the formula (I).

The invention relates to diaminopyrimidines and their agrochemically active salts, to their use and to methods and compositions for controlling phytopathogenic fungi in and/or on plants or in and/or on seed of plants, to processes for preparing such compositions and treated seed and also to the use for controlling phytopathogenic harmful fungi in agriculture, horticulture and forestry, in animal health, in the protection of materials and in the domestic and hygiene field. The present invention further more relates to a process for preparing heterocyclically substituted anilinopyrimidines.

It is already known that certain alkynyl-substituted diaminopyrimidines can be used as fungicidal crop protection angents (see DE 4029650 A1). However, in particular at low application rates, the fungicidal activity of these compounds is not always sufficient.

Since the ecological and economic demands made on modern fungicides are increasing constantly, for example with respect to activity spectrum, toxicity, selectivity, application rate, formation of residues and favourable manufacture, and there can furthermore be problems, for example, with resistance, there is a constant need to develop novel crop protection agents which, at least in some areas, have advantages over the known crop protection agents.

Surprisingly, it has now been found that the present heterocyclyl-substituted anilinopyrimidines solve at least some aspects of the objects mentioned and are suitable for the use as crop protection agents, in particular as fungicides.

Some of these substituted diaminopyrimidines are already known as pharmaceutically active components (see, for example, WO 07/140,957, WO 06/021544, WO 07/072,158, WO 07/003,596, WO 05/016893, WO 05/013996, WO 04/056807, WO 04/014382, WO 03/030909), but not their surprising fungicidal activity.

The invention provides compounds of the formula (I)

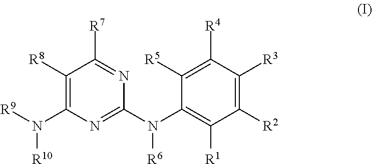

in which one or more of the symbols have one of the meanings below:
R¹ to R⁵ independently of one another represent hydrogen, OH, halogen, cyano, C₁-C₄-alkoxy, C₁-C₄-haloalkyl, NMe₂, SCH₃ or C₁-C₂-haloalkoxy,
   where exactly one of the radicals R² and R³ represents a group of the formula E1, E2 or E3,

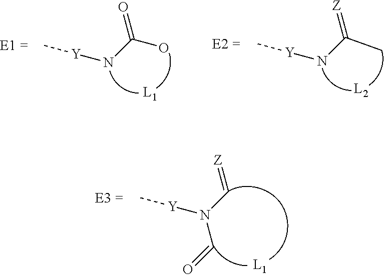

in which one or more of the symbols have one of the meanings below:
Y represents a direct bond, C═O or a C₁-C₃-alkylene chain substituted by straight-chain or branched C₁-C₄-alkyl, C₁-C₄-haloalkyl or C₁-C₄-alkoxyalkyl,
Z represents sulphur or oxygen,
L₁ represents an unsubstituted or substituted C₁- to C₄-alkylene chain or a C₂- to C₄-alkenyl chain,
   where double bonds are not cumulated and
   where the individual carbon atoms may carry one or more substituents, independently of one another selected from the list below:
   hydrogen, hydroxyl, oxo, straight-chain or branched C₁-C₄-alkyl, straight-chain or branched C₁-C₄-alkoxyalkyl, CH₂OH, straight-chain or branched C₁-C₄-alkoxycarbonyl, straight-chain or branched C₁-C₄-alkoxy-C₁-C₄-alkyl, straight-chain or branched phenyl or benzyl,
   or
   two substituents attached to two adjacent carbon atoms of the alkylene chain together with these two carbon atoms form
   a 5- or 6-membered unsubstituted or substituted saturated carbocycle,
   where the substituents independently of one another are selected from the list below:
   hydrogen, fluorine, optionally branched C₁-C₄-alkyl, optionally branched C₁-C₄-alkoxy or optionally branched C₁-C₄-haloalkyl,
   or
   a 5- or 6-membered unsubstituted or substituted saturated heterocycle which contains an oxygen atom or a sulphur atom,
   where the substituents independently of one another are selected from the list below:
   hydrogen, fluorine, optionally branched C₁-C₄-alkyl, optionally branched C₁-C₄-alkoxy or optionally branched C₁-C₄-haloalkyl,
   or
   an unsubstituted or substituted phenyl ring,
   where the substituents independently of one another are selected from the list below:
   hydrogen, halogen, CN, SCH₃, NO₂, optionally branched C₁-C₄-alkyl, optionally branched C₁-C₄-alkoxy, optionally branched C₁-C₄-alkylcarbonyl, optionally branched C₁-C₄-haloalkyl, optionally branched C₁-C₄-haloalkoxy,
L₂ is an unsubstituted or substituted C₁- to C₄-alkylene chain, a C₂- to C₄-alkenyl chain or a 1,3-linked cyclopentyl (3-oxo-2-azabicyclo[2.2.1]hept-2-yl) or a C₁- to C₄-alkylene chain interrupted by a heteroatom selected from the group consisting of oxygen, sulphur and nitrogen,
   where double bonds are not cumulated and
   where the individual carbon atoms may carry one or more substituents independently of one another selected from the list below:
   hydrogen, hydroxyl, CH₂OH, cyano, halogen, straight-chain or branched C₁-C₄-alkyl, straight-chain or branched C₁-C₄-haloalkyl, straight-chain or branched C₁-C₄-alkoxyalkyl, straight-chain or branched C₁-C₄-alkoxycarbonyl, straight-chain or substituted phenyl or benzyl, an optionally alkyl-substituted C₂-C₅-alkyl chain which may contain up to one oxygen, an optionally alkyl-substituted C₃-C₅-alkenyl chain,
R⁶ represents hydrogen, Me, C₁-C₄-alkylcarbonyl, CHO, C₁-C₄-alkoxy-C₁-C₄-alkylcarbonyl, C₁-C₄-alkoxy-C₁-C₄-alkyl, straight-chain or branched C₁-C₄-alkoxycarbonyl, COOBn, C₁-C₄-haloalkylcarbonyl, C₂-C₃-alkenyl, C₂-C₃-alkynyl, C₁-C₄-alkylsulphinyl, C₁-C₄-alkylsulphonyl, unsubstituted or substituted benzyl, C₁-C₄-trialkylsilyl, trialkylsilylethyl or C₁-C₄-dialkylmonophenylsilyl,
   where the substituents independently of one another are selected from the group consisting of hydrogen, halogen, nitro, C₁-C₄-alkyl, C₁-C₄-alkoxy, hydroxyl, C₁-C₄-haloalkyl and cyano,
R⁷ represents hydrogen, cyano, C₁-C₃-alkyl or C₁-C₃-haloalkyl,
R⁸ represents halogen, cyano, C₁-C₂-haloalkyl, methyl, SMe, SOMe or SO₂Me,
R⁹ represents hydrogen, straight-chain or branched C₁-C₃-alkyl, 2-methoxyethan-1-yl, prop-2-en-1-yl, C₁-C₄-alkoxy(C₁-C₄)alkyl, straight-chain or branched (C₁-C₄-alkyl)carbonyl, (C₁-C₄-haloalkyl)carbonyl, unsubstituted or substituted benzyl, C₁-C₆-trialkylsilyl, C₁-C₄-trialkylsilylethyl, C₁-C₄-dialkylmonophenylsilyl, (C₁-C₄-alkoxy)carbonyl, C₁-C₆-alkylsulphinyl, C₁-C₆-alkylsulphonyl, C₁-C₆-haloalkylsulphinyl or C₁-C₆-haloalkylsulphonyl,
   where the substituents independently of one another are selected from the group consisting of hydrogen, halogen, nitro, C₁-C₄-alkyl, C₁-C₄-alkoxy, hydroxyl, C₁-C₄-haloalkyl and cyano,
R¹⁰ represents straight-chain or branched unsubstituted or substituted C₁-C₇-alkyl, straight-chain or branched unsubstituted or substituted C₂-C₇-haloalkyl, unsubstituted or substituted C₃-C₇-cycloalkyl, straight-chain or branched unsubstituted or substituted C₃-C₇-cycloalkyl(C₁-C₃)alkyl, straight-chain or branched unsubstituted or substituted C₃-C₇-alkenyl, straight-chain or branched unsubstituted or substituted C₃-C₇-alkynyl, straight-chain or branched unsubstituted or substituted C₁-C₄-alkoxy(C₁-C₄)alkyl, straight-chain or branched unsubstituted or substituted C₁-C₄-haloalkoxy(C₁-C₄)alkyl, 2-methyl-1-(methylsulphanyl)propan-2-yl or oxetan-3-yl,
   or
R⁹ and R¹⁰ together with the nitrogen atom to which they are attached form an unsubstituted or substituted 3- to 7-membered saturated cycle which may contain up to one further heteroatom selected from the group consisting of oxygen, sulphur and nitrogen,
   where the substituents R¹⁰ independently of one another are selected from the group consisting of methyl, ethyl, isopropyl, cyclopropyl, fluorine, chlorine and/or bromine atoms, methoxy, ethoxy, methylmercapto, ethylmercapto, cyano, hydroxyl and CF₃,
   and also agrochemically active salts thereof.

The diaminopyrimidines of the formula (I) according to the invention and their agrochemically active salts are highly suitable for controlling phytopathogenic harmful fungi. The compounds according to the invention mentioned above have in particular strong fungicidal activity and can be used both in crop protection, in the domestic and hygiene field and in the protection of materials.

The compounds of the formula (I) can be present both in pure form and as mixtures of various possible isomeric forms, in particular of stereoisomers, such as E and Z, threo and erythro, and also optical isomers, such as R and S isomers or atropisomers, or else also of tautomers. What is claimed are both the E and the Z isomers, and the threo and erythro, and also the optical isomers, any mixtures of these isomers, and also the possible tautomeric forms.

Preference is given to compounds of the formula (I) in which one or more of the symbols have one of the meanings below:
R¹ to R⁵ independently of one another represent hydrogen, OH, Cl, F, Br, CH₃, CF₃, ethyl, OCH₃, SCH₃, OCF₂H or OCF₃,
   where exactly one of the radicals R² and R³ represents a group of the formula E1, E2 or E3

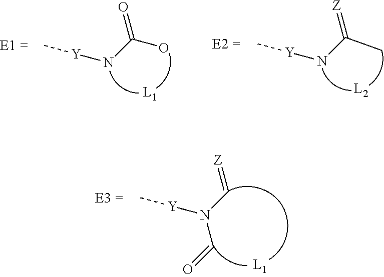

in which one or more of the symbols have one of the meanings below:
Y represents a direct bond or —CH₂—, —CH₂CH₂—, —CH(CH₃)CH₂—, —CH₂CH(CH₃)—, —CHMe-, —CHEt-, —CHOMe-, —CHCF₃— or C═O,
Z represents sulphur or oxygen,
L₁ represents an unsubstituted or substituted C₁- to C₄-alkylene chain or a C₂- to C₄-alkenyl chain,
   where the double bonds are not cumulated and
   where the individual carbon atoms may carry one or more substituents independently of one another selected from the list below:
   hydrogen, methyl, ethyl, hydroxyl, oxo, methoxy, OCH₂CH₃, OC(CH₃)₃, OCH(CH₃)₂, O-propyl, O-butyl, COOCH₃, COOCH₂CH₃, COOC(CH₃)₃, COOPr, COOCH(CH₃)₂, CH₂OH, CH₂OCH₃, CH₂OCH₂CH₃, CH₂CH(CH₃)₂, CH₂C(CH₃)₃, phenyl or benzyl,
   or
   two substituents attached to two adjacent carbon atoms of the alkylene chain together with these two carbon atoms form
   a 5- to 8-membered unsubstituted or substituted saturated carbocycle,
   where the substituents independently of one another are selected from the list below:
   hydrogen, fluorine, methyl, ethyl, propyl, isopropyl, tert-butyl, methoxy, ethoxy, propoxy, CF₃ or CHF₂,
   or
   a 5- or 6-membered unsubstituted or substituted saturated heterocycle which contains one oxygen atom or one sulphur atom,
   where the substituents independently of one another are selected from the list below:
   hydrogen, fluorine, methyl, ethyl, propyl, isopropyl, tert-butyl, methoxy, ethoxy, propoxy, CF₃ or CHF₂,
   or
   an unsubstituted or substituted phenyl ring,
   where the substituents independently of one another are selected from the list below:
   hydrogen, chlorine, fluorine, CN, NO₂, methyl, ethyl, propyl, isopropyl, tert-butyl, methoxy, ethoxy, propoxy, CF₃, CHF₂, OCF₃, OCHF₂, CO—CH₃ or COCH₂CH₃,
L₂ is an unsubstituted or substituted C₁- to C₄-alkylene chain, a C₂- to C₄-alkenyl chain or a 1,3-linked cyclopentyl (3-oxo-2-azabicyclo[2.2.1]hept-2-yl) or a C₁- to C₄-alkylene chain which is interrupted by a heteroatom selected from the group consisting of oxygen, sulphur and nitrogen,
   where double bonds are not cumulated and
   where the individual carbon atoms may carry one or more substituents independently of one another selected from the list below:
   hydrogen, fluorine, chlorine, methyl, ethyl, propyl, isopropyl, tert-butyl, cyano, CF₃, hydroxyl, methoxy, O-propyl, O-isopropyl, O-butyl, O-tert-butyl, COOCH₃, COOCH₂CH₃, COOC(CH₃)₃, COOCH(CH₃)₂, COOPr, COOBu, OCH₂CH₃, CH₂OH, CH₂OMe, CH₂OEt, CH₂C(CH₃)₃, CH₂CH(CH₃)₂, phenyl, benzyl, —CH₂OCH₂CH₂—, —CH(CH₃)OCH₂CH₂—, —CH₂OCH(CH₃)CH₂—, —CH₂OCH₂CH(CH₃)—, —CHC(OCH₃)CH₂—, —C(CH₂CH₃)C(CH₃)CH₂—, —C(CH₂CH₃)C(CH₂CH₃)CH₂—, C(CH₃)C(CH₃)CH₂—, —CH═CH═CH═CH— or —C(CH₃)═CH═CH═CH—,
R⁶ represents hydrogen, Me, COMe, CHO, COCH₂OCH₃, CH₂OCH₃, COOMe, COOEt, COOtertBu, COOBn, COCF₃, CH₂CH═CH₂, CH₂C≡CH, SOCH₃, SO₂CH₃ or benzyl,
R⁷ represents hydrogen, cyano, methyl, CF₃ or CFH₂,
R⁸ represents chlorine, bromine, fluorine, iodine, cyano, CF₃, CFH₂, CF₂H, CCl₃, methyl, SMe, SOMe or SO₂Me,
R⁹ represents hydrogen, methyl, ethyl, propyl, propan-2-yl, 2-methoxyethan-1-yl, prop-2-en-1-yl, CH₂OCH₃, COMe, COOMe, COOEt, COOtertBu, COCF₃ or benzyl,
R¹⁰ represents straight-chain or branched unsubstituted or substituted C₁-C₆-alkyl, straight-chain or branched unsubstituted or substituted C₃-C₆-cycloalkyl(C₁-C₂)alkyl, unsubstituted or substituted C₃-C₆-cycloalkyl, straight-chain or branched unsubstituted or substituted C₃-C₄-alkenyl, straight-chain or branched unsubstituted or substituted C₃-C₄-alkynyl, straight-chain or branched unsubstituted or substituted C₂-C₄-haloalkyl, straight-chain or branched unsubstituted or substituted C₁-C₂-alkoxy(C₁-C₄)alkyl, straight-chain or branched unsubstituted or substituted C₁-C₂-alkylmercapto-(C₁-C₄)alkyl or oxetan-3-yl,
   where the substituents in R¹⁰ independently of one another are selected from the group consisting of methyl, ethyl, isopropyl, cyclopropyl, fluorine, chlorine and/or bromine atoms, methoxy, ethoxy, methylmercapto, ethylmercapto, cyano, hydroxyl and CF₃,
   or
R⁹ and R¹⁰ together with the nitrogen atom to which they are attached form an azetidinyl, pyrrolidinyl, piperidinyl, morpholinyl, azepanyl, 4-methylpiperazin-1-yl, 2-methylpiperidin-1-yl, 2-methylpyrrolidin-1-yl, 2-methylazetidin-1-yl or thiomorpholinyl ring,
   and also agrochemically active salts thereof.

Particular preference is given to compounds of the formula (I) in which one or more of the symbols have one of the meanings below:

R¹ to R⁵ independently of one another represent hydrogen, OH, Cl, F, CH₃, CF₃, ethyl, OCH₃ or OCF₃,
where exactly one of the radicals R² and R³ represents a group of the formula E1, E2 or E3,

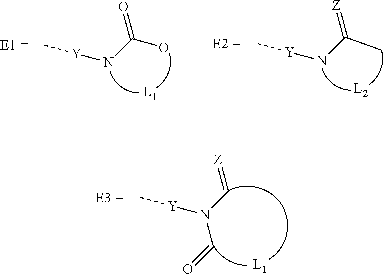

in which one or more of the symbols have one of the meanings below:
Y represents a direct bond or —CH₂—, —CH₂CH₂—, —CHMe-, —CHEt-, —CHOMe-, —CHCF₃— or C═O,
Z represents sulphur or oxygen,
L₁ represents an unsubstituted or substituted C₁- to C₄-alkylene chain or a C₂- to C₄-alkenyl chain,
   where double bonds are not cumulated and
   where the individual carbon atoms may carry one or more substituents independently of one another selected from the list below:
   hydrogen, methyl, ethyl, hydroxyl, oxo, methoxy, OCH₂CH₃, OC(CH₃)₃, OCH(CH₃)₂, COOCH₃, COOCH₂CH₃, COOC(CH₃)₃, CH₂OH, CH₂OCH₃, CH₂CH(CH₃)₂ or phenyl,
   or
   two substituents attached to two adjacent carbon atoms of the alkylene chain together with these two carbon atoms form
   a 5- or 6-membered unsubstituted or substituted saturated carbocycle,
   where the substituents independently of one another are selected from the list below:
   hydrogen, fluorine, methyl, ethyl, propyl, methoxy, ethoxy or CF₃,
   or
   a 5- or 6-membered unsubstituted or substituted saturated heterocycle which contains an oxygen atom or a sulphur atom,
   where the substituents independently of one another are selected from the list below:
   hydrogen, fluorine, methyl, ethyl, tert-butyl, methoxy, ethoxy or CF₃,
   or
   an unsubstituted or substituted phenyl ring,
   where the substituents independently of one another are selected from the list below:
   hydrogen, chlorine, fluorine, CN, methyl, ethyl, tert-butyl, methoxy, CF₃ or CO—CH₃,
L₂ represents an unsubstituted or substituted C₁- to C₄-alkylene chain or a C₂- to C₄-alkenyl chain or a 1,3-linked cyclopentyl (3-oxo-2-azabicyclo[2.2.1]hept-2-yl) or a C₁- to C₄-alkylene chain which is interrupted by a heteroatom selected from the group consisting of oxygen, sulphur and nitrogen,
   where double bonds are not cumulated and
   where the individual carbon atoms may carry one or more substituents independently of one another selected from the list below:
   hydrogen, fluorine, chlorine, methyl, ethyl, propyl, isopropyl, tert-butyl, cyano, CF₃, hydroxyl, methoxy, O-propyl, COOCH₃, COOCH₂CH₃, COOC(CH₃)₃, COOCH(CH₃)₂, OCH₂CH₃, CH₂OH, CH₂CH(CH₃)₂, phenyl, —CH₂OCH₂CH₂—, —CHC(OCH₃)CH₂—, —C(CH₂CH₃)C(CH₃)CH₂— or —CH═CH═CH═CH—,
R⁶ represents hydrogen, Me, COMe, CHO, COCH₂OCH₃, CH₂OCH₃, COOMe, COOEt, COCF₃, CH₂CH═CH₂, CH₂C≡CH, SOCH₃ or SO₂CH₃,
R⁷ represents hydrogen, cyano, methyl, CF₃ or CFH₂,
R⁸ represents chlorine, bromine, fluorine, iodine, cyano, CF₃, CFH₂, CF₂H, CCl₃, methyl, SMe, SOMe or SO₂Me,
R⁹ represents hydrogen, methyl, ethyl, propyl, propan-2-yl, 2-methoxyethan-1-yl, prop-2-en-1-yl, CH₂OCH₃, COMe, COOMe, COOEt, COOtertBu, COCF₃ or benzyl,
R¹⁰ represents methyl, ethyl, propyl, cyclopropyl, cyclopropylmethyl, 1-cyclopropyleth-1-yl, 2-methylcyclopropyl, 2,2-dimethylcyclopropyl, 2,2-dimethylprop-1-yl, tert-butyl, cyclobutyl, 2-methylcyclobut-1-yl, 3-methylcyclobut-1-yl, butyl, 3-methylbut-1-yl, 2-methylbut-1-yl, 2-methylprop-1-yl, 1-fluoroprop-2-yl, cyclopentyl, propan-2-yl, pentan-3-yl, pentan-2-yl, pentyl, prop-2-en-1-yl, prop-2-yn-1-yl, butan-2-yl, 2,2,2-trifluoroethyl, 2,2-difluoroethyl, 2-methoxyethan-1-yl, 2-methylmercaptoethan-1-yl, 2-fluoroethan-1-yl, 2-chloroethan-1-yl, 2-cyanoethan-1-yl, 1-methoxypropan-2-yl, 3-methoxypropan-1-yl, 2-hydroxyethan-1-yl, 1-hydroxypropan-2-yl, 3-hydroxypropan-1-yl, 1-methylmercaptopropan-2-yl, 2-methyl-1-(methylsulphanyl)propan-2-yl, oxetan-3-yl, 1,1,1-trifluoropropan-2-yl, 2,2,3,3,3-pentafluoropropyl, 1,1,1-trifluoropropan-3-yl, 1,1,1-trifluorobutan-2-yl, 1,1,1-trifluorobutan-3-yl, 2-methylprop-2-en-1-yl or 1-fluoropropan-2-yl
   or
R⁹ and R¹⁰ together with the nitrogen atom to which they are attached represent an azetidinyl, pyrrolidinyl, piperidinyl, morpholinyl, azepanyl, 4-methylpiperazin-1-yl, 2-methylpiperidin-1-yl, 2-methylpyrrolidin-1-yl, 2-methylazetidin-1-yl or thiomorpholinyl ring,
   and also agrochemically active salts thereof.

Very particular preference is given to compounds of the formula (I) in which one or more of the symbols have one of the meanings below:

R¹ to R⁵ independently of one another represent hydrogen, OH, Cl, F, CH₃ or OCF₃,
where exactly one of the radicals R² and R³ represents a group of the formula E1, E2 or E3

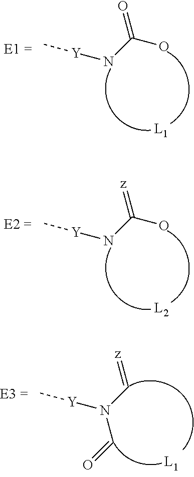

in which one or more of the symbols have one of the meanings below:
Y represents a direct bond or —CH₂—, —CH₂CH₂—, —CHMe-, —CHCF₃— or C═O,
Z represents sulphur or oxygen,
L₁ represents an unsubstituted or substituted C₂- to C₃-alkylene chain or a C₂- to C₃-alkenyl chain,
   where double bonds are not cumulated and
   where the individual carbon atoms may carry one or more substituents independently of one another selected from the list below:
   hydrogen, methyl, ethyl, hydroxyl, oxo, methoxy, OCH₂CH₃, COOCH₃, COOCH₂CH₃, CH₂OH, CH₂CH(CH₃)₂ or phenyl,
   or
   two substituents attached to two adjacent carbon atoms of the alkylene chain together with these two carbon atoms form
   a 5- or 6-membered unsubstituted saturated carbocycle,
   or
   a 5- or 6-membered unsubstituted saturated heterocycle which contains one oxygen atom,
   or
   an unsubstituted phenyl ring,
L₂ represents an unsubstituted or substituted C₂- to C₃-alkylene chain or a C₂- to C₃-alkenyl chain or a 1,3-linked cyclopentyl (3-oxo-2-azabicyclo[2.2.1]hept-2-yl) or a C₁- to C₂-alkylene chain which is interrupted by a heteroatom selected from the group consisting of oxygen, sulphur and nitrogen,
   where double bonds are not cumulated and
   where the individual carbon atoms may carry one or more substituents independently of one another selected from the list below:
   hydrogen, methyl, ethyl, cyano, CF₃, isopropyl, hydroxyl, methoxy, COOCH₃, COOCH₂CH₃, OCH₂CH₃, CH₂OH, CH₂CH(CH₃)₂, phenyl, —CH₂OCH₂CH₂—, —CHC(OCH₃)CH₂—, —C(CH₂CH₃)C(CH₃)CH₂— or —CH═CH═CH═CH—,
R⁶ represents hydrogen, Me, COMe, CHO or COCH₂OCH₃,
R⁷ represents hydrogen,
R⁸ represents chlorine, bromine, fluorine, iodine, cyano, CF₃, SMe, SOMe or SO₂Me,
R⁹ represents hydrogen, methyl, ethyl, propyl, propan-2-yl, 2-methoxyethan-1-yl or prop-2-en-1-yl,
R¹⁰ represents methyl, ethyl, propyl, cyclopropyl, cyclopropylmethyl, 1-cyclopropyleth-1-yl, 2-methylcyclopropyl, 2,2-dimethylcyclopropyl, 2,2-dimethylprop-1-yl, tert-butyl, cyclobutyl, 2-butyl, 3-methylbut-1-yl, 2-methylbut-1-yl, 2-methylprop-1-yl, 1-fluoroprop-2-yl, cyclopentyl, propan-2-yl, pentan-3-yl, pentan-2-yl, pentyl, prop-2-en-1-yl, prop-2-yn-1-yl, butan-2-yl, 2,2,2-trifluoroethyl, 2,2-difluoroethyl, 2-methoxyethan-1-yl, 2-methylmercaptoethan-1-yl, 2-fluoroethan-1-yl, 2-chloroethan-1-yl, 2-cyanoethan-1-yl, 1-methoxypropan-2-yl, 3-methoxypropan-1-yl, 2-hydroxyethan-1-yl, 1-hydroxypropan-2-yl, 3-hydroxypropan-1-yl, 1-methylmercaptopropan-2-yl, 2-methyl-1-(methylsulphanyl)propan-2-yl, oxetan-3-yl, 1,1,1-trifluoropropan-2-yl, 2,2,3,3,3-pentafluoropropyl, 1,1,1-trifluoropropan-3-yl, 1,1,1-trifluorobutan-2-yl, 1,1,1-trifluorobutan-3-yl, 2-methylprop-2-en-1-yl or 1-luoropropan-2-yl
   or
R⁹ and R¹⁰ together with the nitrogen atom to which they are attached form an azetidinyl, pyrrolidinyl, piperidinyl, morpholinyl, azepanyl, 4-methylpiperazin-1-yl, 2-methylpiperidin-1-yl, 2-methylpyrrolidin-1-yl, 2-methylazetidin-1-yl or thiomorpholinyl ring,
   and also agrochemically active salts thereof.

Special preference is given to compounds of the formula (I) in which one or more of the symbols have one of the meanings below:
R¹ represents hydrogen or OH,
R² represents hydrogen, (2,5-dioxopyrrolidin-1-yl)methyl, (2-oxopyrrolidin-1-yl)carbonyl, (3-methyl-2-oxopyrrolidin-1-yl)methyl, 1-(2,5-dioxopyrrolidin-1-yl)-2,2,2-trifluoroethyl, (2R)-2-(ethoxycarbonyl)-5-oxopyrrolidin-1-yl, (2S)-2-(ethoxycarbonyl)-5-oxopyrrolidin-1-yl, 2-(ethoxycarbonyl)-5-oxopyrrolidin-1-yl, (2R)-2-(hydroxymethyl)-5-oxopyrrolidin-1-yl, (2S)-2-(hydroxymethyl)-5-oxopyrrolidin-1-yl, 2-(hydroxymethyl)-5-oxopyrrolidin-1-yl, 2,5-dioxopyrrolidin-1-yl, 2-ethoxy-5-oxopyrrolidin-1-yl, (2R)-2-methyl-5-oxopyrrolidin-1-yl, (2S)-2-methyl-5-oxopyrrolidin-1-yl, 2-methyl-5-oxopyrrolidin-1-yl, 2-oxo-1,3-oxazolidin-3-yl, (4R)-2-oxo-4-(propan-2-yl)-1,3-oxazolidin-3-yl, (4S)-2-oxo-4-(propan-2-yl)-1,3-oxazolidin-3-yl, 2-oxo-4-(propan-2-yl)-1,3-oxazolidin-3-yl, 2-oxo-4-phenyl-1,3-oxazolidin-3-yl, 2-oxo-5-phenylpyrrolidin-1-yl, 2-oxoazepan-1-yl, 2-oxopiperidin-1-yl, 2-oxopyridin-1(2H)-yl, 2-oxopyrrolidin-1-yl, 2-thioxopyrrolidin-1-yl, 3,3-dimethyl-2,5-dioxopyrrolidin-1-yl, 3,3-dimethyl-2-oxoazetidin-1-yl, 3,5-dimethyl-2-oxopyrrolidin-1-yl, (3R,5R)-3,5-dimethyl-2-oxopyrrolidin-1-yl, (3R,5S)-3,5-dimethyl-2-oxopyrrolidin-1-yl, 3-ethyl-4-methyl-2-oxo-2,5-dihydro-1H-pyrrol-1-yl, (3R)-3-hydroxy-2-oxopyrrolidin-1-yl, (3S)-3-hydroxy-2-oxopyrrolidin-1-yl, 3-hydroxy-2-oxopyrrolidin-1-yl, 3-methyl-2,5-dioxopyrrolidin-1-yl, (3R)-3-methyl-2-oxopyrrolidin-1-yl, (3S)-3-methyl-2-oxopyrrolidin-1-yl, 3-methyl-2-oxopyrrolidin-1-yl, 3-oxomorpholin-4-yl, 4-(2-methylpropyl)-2-oxo-1,3-oxazolidin-3-yl, (4R)-4-(methoxycarbonyl)-2-oxopyrrolidin-1-yl, 4-ethyl-2-oxo-1,3-oxazolidin-3-yl, (4R)-4-hydroxy-2-oxopyrrolidin-1-yl, (4S)-4-hydroxy-2-oxopyrrolidin-1-yl, 4-hydroxy-2-oxopyrrolidin-1-yl, 4-methoxy-2-oxo-2,5-dihydro-1H-pyrrol-1-yl, (4R)-4-methyl-2-oxo-1,3-oxazolidin-3-yl, (4S)-4-methyl-2-oxo-1,3-oxazolidin-3-yl, 4-methyl-2-oxo-1,3-oxazolidin-3-yl, 4-methyl-2-oxopyrrolidin-1-yl, 5-ethyl-3-methyl-2-oxopyrrolidin-1-yl, 4,4-dimethyl-2-oxo-1,3-oxazolidin-3-yl, 5-methyl-2-oxo-1,3-oxazolidin-3-yl, 5,5-dimethyl-2-oxo-1,3-oxazolidin-3-yl, 2-ethyl-5-oxopyrrolidin-1-yl, 2-oxo-5-(propan-2-yl)pyrrolidin-1-yl, 2-oxo-3-(trifluoromethyl)pyrrolidin-1-yl, 3,3-dimethyl-2-oxopyrrolidin-1-yl, 3,3-dimethyl-2,5-dioxopyrrolidin-1-yl, 3,3-dimethyl-2-oxo-5-thioxopyrrolidin-1-yl, 3-cyano-2-oxopyrrolidin-1-yl, 3-oxo-2-azabicyclo[2.2.1]hept-2-yl, 1,3-dioxooctahydro-2H-isoindol-2-yl, 1,3-dioxo-1,3-dihydro-2H-isoindol-2-yl, 2,5-dioxo-2,5-dihydro-1H-pyrrol-1-yl,
R³ represents hydrogen, 2-oxo-1,3-oxazolidin-3-yl, 2-oxopyrrolidin-1-yl, 4-methyl-2-oxo-1,3-oxazolidin-3-yl, OCF₃, fluorine, methyl or chlorine,
   where R² and R³ are not both hydrogen,
   with the proviso that, if R² is not hydrogen,
R³ can only have one of the meanings below:
   hydrogen, OCF₃ fluorine, methyl or chlorine,
R⁴ represents hydrogen or CH₃,
R⁵ represents hydrogen,
R⁶ represents hydrogen, Me, COMe, CHO or COCH₂OCH₃,
R⁷ represents hydrogen,
R⁸ represents chlorine, bromine, fluorine, iodine, cyano, CF₃, SMe, SOMe or SO₂Me,
R⁹ represents hydrogen, methyl, ethyl, propyl, propan-2-yl, 2-methoxyethan-1-yl or prop-2-en-1-yl,
R¹⁰ represents methyl, ethyl, propyl, cyclopropyl, cyclopropylmethyl, 1-cyclopropyleth-1-yl, 2-methylcyclopropyl, 2,2-dimethylcyclopropyl, 2,2-dimethylprop-1-yl, tert-butyl, cyclobutyl, 2-butyl, 3-methylbut-1-yl, 2-methylbut-1-yl, 2-methylprop-1-yl, 1-fluoroprop-2-yl, cyclopentyl, propan-2-yl, pentan-3-yl, pentan-2-yl, pentyl, prop-2-en-1-yl, prop-2-yn-1-yl, butan-2-yl, 2,2,2-trifluoroethyl, 2,2-difluoroethyl, 2-methoxyethan-1-yl, 2-methylmercaptoethan-1-yl, 2-fluoroethan-1-yl, 2-chloroethan-1-yl, 2-cyanoethan-1-yl, 1-methoxypropan-2-yl, 3-methoxypropan-1-yl, 2-hydroxyethan-1-yl, 1-hydroxypropan-2-yl, 3-hydroxypropan-1-yl, 1-methylmercaptopropan-2-yl, 2-methyl-1-(methylsulphanyl)propan-2-yl, oxetan-3-yl, 1,1,1-trifluoropropan-2-yl, 2,2,3,3,3-pentafluoropropyl, 1,1,1-trifluoropropan-3-yl, 1,1,1-trifluorobutan-2-yl, 1,1,1-trifluorobutan-3-yl, 2-methylprop-2-en-1-yl or 1-fluoropropan-2-yl,
   or
R⁹ and R¹⁰ together with the nitrogen atom to which they are attached represent an azetidinyl, pyrrolidinyl, piperidinyl, morpholinyl, azepanyl, 4-methylpiperazin-1-yl, 2-methylpiperidin-1-yl, 2-methylpyrrolidin-1-yl, 2-methylazetidin-1-yl or thiomorpholinyl ring,
   and also agrochemically active salts thereof.

Especial preference is furthermore given to compounds of the formula (I) in which one or more of the symbols have one of the meanings below:
R¹ represents hydrogen,
R² represents hydrogen, (2,5-dioxopyrrolidin-1-yl)methyl, (2-oxopyrrolidin-1-yl)carbonyl, (3-methyl-2-oxopyrrolidin-1-yl)methyl, 1-(2,5-dioxopyrrolidin-1-yl)-2,2,2-trifluoroethyl, (2R)-2-(ethoxycarbonyl)-5-oxopyrrolidin-1-yl, (2S)-2-(ethoxycarbonyl)-5-oxopyrrolidin-1-yl, 2-(ethoxycarbonyl)-5-oxopyrrolidin-1-yl, (2R)-2-(hydroxymethyl)-5-oxopyrrolidin-1-yl, (2S)-2-(hydroxymethyl)-5-oxopyrrolidin-1-yl, 2-(hydroxymethyl)-5-oxopyrrolidin-1-yl, 2,5-dioxopyrrolidin-1-yl, 2-ethoxy-5-oxopyrrolidin-1-yl, (2R)-2-methyl-5-oxopyrrolidin-1-yl, (2S)-2-methyl-5-oxopyrrolidin-1-yl, 2-methyl-5-oxopyrrolidin-1-yl, 2-oxo-1,3-oxazolidin-3-yl, (4R)-2-oxo-4-(propan-2-yl)-1,3-oxazolidin-3-yl, (4S)-2-oxo-4-(propan-2-yl)-1,3-oxazolidin-3-yl, 2-oxo-4-(propan-2-yl)-1,3-oxazolidin-3-yl, 2-oxo-4-phenyl-1,3-oxazolidin-3-yl, 2-oxo-5-phenylpyrrolidin-1-yl, 2-oxoazepan-1-yl, 2-oxopiperidin-1-yl, 2-oxopyridin-1(2H)-yl, 2-oxopyrrolidin-1-yl, 2-thioxopyrrolidin-1-yl, 3,3-dimethyl-2,5-dioxopyrrolidin-1-yl, 3,3-dimethyl-2-oxoazetidin-1-yl, 3,5-dimethyl-2-oxopyrrolidin-1-yl, (3R,5R)-3,5-dimethyl-2-oxopyrrolidin-1-yl, (3R,5S)-3,5-dimethyl-2-oxopyrrolidin-1-yl, 3-ethyl-4-methyl-2-oxo-2,5-dihydro-1H-pyrrol-1-yl, (3R)-3-hydroxy-2-oxopyrrolidin-1-yl, (3S)-3-hydroxy-2-oxopyrrolidin-1-yl, 3-hydroxy-2-oxopyrrolidin-1-yl, 3-methyl-2,5-dioxopyrrolidin-1-yl, (3R)-3-methyl-2-oxopyrrolidin-1-yl, (3S)-3-methyl-2-oxopyrrolidin-1-yl, 3-methyl-2-oxopyrrolidin-1-yl, 3-oxomorpholin-4-yl, 4-(2-methylpropyl)-2-oxo-1,3-oxazolidin-3-yl, 4-(methoxycarbonyl)-2-oxopyrrolidin-1-yl, 4-ethyl-2-oxo-1,3-oxazolidin-3-yl, (4R)-4-hydroxy-2-oxopyrrolidin-1-yl, (4S)-4-hydroxy-2-oxopyrrolidin-1-yl, 4-hydroxy-2-oxopyrrolidin-1-yl, 4-methoxy-2-oxo-2,5-dihydro-1H-pyrrol-1-yl, (4R)-4-methyl-2-oxo-1,3-oxazolidin-3-yl, (4S)-4-methyl-2-oxo-1,3-oxazolidin-3-yl, 4-methyl-2-oxo-1,3-oxazolidin-3-yl, 4-methyl-2-oxopyrrolidin-1-yl, 5-ethyl-3-methyl-2-oxopyrrolidin-1-yl, 4,4-dimethyl-2-oxo-1,3-oxazolidin-3-yl, 5-methyl-2-oxo-1,3-oxazolidin-3-yl, 5,5-dimethyl-2-oxo-1,3-oxazolidin-3-yl, 2-ethyl-5-oxopyrrolidin-1-yl, 2-oxo-5-(propan-2-yl)pyrrolidin-1-yl, 2-oxo-3-(trifluoromethyl)pyrrolidin-1-yl, 3,3-dimethyl-2-oxopyrrolidin-1-yl, 3,3-dimethyl-2,5-dioxopyrrolidin-1-yl, 3,3-dimethyl-2-oxo-5-thioxopyrrolidin-1-yl, 3-cyano-2-oxopyrrolidin-1-yl, 3-oxo-2-azabicyclo[2.2.1]hept-2-yl, 1,3-dioxooctahydro-2H-isoindol-2-yl, 1,3-dioxo-1,3-dihydro-2H-isoindol-2-yl, 2,5-dioxo-2,5-dihydro-1H-pyrrol-1-yl,
R³ represents hydrogen, 2-oxo-1,3-oxazolidin-3-yl, 2-oxopyrrolidin-1-yl, 4-methyl-2-oxo-1,3-oxazolidin-3-yl, OCF₃, fluorine, methyl or chlorine,
   where R² and R³ do not both represent hydrogen,
   with the proviso that, if R² does not represent hydrogen,
R³ can only have one of the meanings below:
   hydrogen, OCF₃, fluorine, methyl or chlorine,
R⁴ represents hydrogen,
R⁵ represents hydrogen,
R⁶ represents hydrogen, Me, COMe, CHO or COCH₂OCH₃,
R⁷ represents hydrogen,
R⁸ represents chlorine, bromine, fluorine, iodine, cyano or CF₃,
R⁹ represents hydrogen or methyl,
R¹⁰ represents methyl, ethyl, cyclopropyl, cyclopropylmethyl, 2,2-dimethylcyclopropyl, cyclobutyl, cyclopentyl, propan-2-yl, prop-2-en-1-yl, butan-2-yl, 2,2,2-trifluoroethyl, 2,2-difluoroethyl, 1-methoxypropan-2-yl, 2-methyl-1-(methylsulphanyl)propan-2-yl, oxetan-3-yl or 2,2,3,3,3-pentafluoropropyl,
   or
R⁹ and R¹⁰ together with the nitrogen atom to which they are attached form an azetidinyl, pyrrolidinyl, piperidinyl, morpholinyl, azepanyl or thiomorpholinyl ring,
   and also agrochemically active salts thereof.

Preference is furthermore given to compounds of the formula (I) in which

the radical R² represents a group of the formula E1, E2 or E3

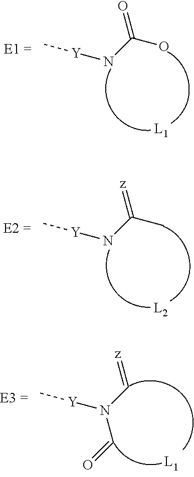

in which one or more of the symbols have one of the meanings below:
Y represents a direct bond, C═O or a C₁-C₃-alkyl chain substituted by straight-chain or branched C₁-C₄-alkyl, C₁-C₄-haloalkyl or C₁-C₄-alkoxyalkyl,
Z represents sulphur or oxygen,
L₁ represents an unsubstituted or substituted C₁- to C₄-alkylene chain or a C₂- to C₄-alkenyl chain,
   where double bonds are not cumulated and
   where the individual carbon atoms may carry one or more substituents independently of one another selected from the list below:
   hydrogen, hydroxyl, straight-chain or branched C₁-C₄-alkyl, straight-chain or branched C₁-C₄-alkoxyalkyl, straight-chain or branched C₁-C₄-alkoxycarbonyl, straight-chain or branched C₁-C₄-alkoxy-C₁-C₄-alkyl, unsubstituted or substituted phenyl or benzyl,
   or
   two substituents attached to two adjacent carbon atoms of the alkylene chain together with these two carbon atoms form
   a 5- or 6-membered unsubstituted or substituted saturated carbocycle,
   where the substituents independently of one another are selected from the list below:
   hydrogen, fluorine, optionally branched C₁-C₄-alkyl, optionally branched C₁-C₄-alkoxy or optionally branched C₁-C₄-haloalkyl,
   or
   a 5- or 6-membered unsubstituted or substituted saturated heterocycle which contains an oxygen atom or a sulphur atom,
   where the substituents independently of one another are selected from the list below:
   hydrogen, fluorine, optionally branched C₁-C₄-alkyl, optionally branched C₁-C₄-alkoxy or optionally branched C₁-C₄-haloalkyl,
   or
   an unsubstituted or substituted phenyl ring,
   where the substituents independently of one another are selected from the list below:
   hydrogen, halogen, CN, SCH₃, NO₂, optionally branched C₁-C₄-alkyl, optionally branched C₁-C₄-alkoxy, optionally branched C₁-C₄-alkylcarbonyl, optionally branched C₁-C₄-haloalkyl or optionally branched C₁-C₄-haloalkoxy,
L₂ represents an unsubstituted or substituted C₁- to C₄-alkylene chain or a C₂- to C₄-alkenyl chain or a 1,3-linked cyclopentyl (3-oxo-2-azabicyclo[2.2.1]hept-2-yl) or a C₁- to C₄-alkylene chain which is interrupted by a heteroatom selected from the group consisting of oxygen, sulphur and nitrogen,
   where double bonds are not cumulated and
   where the individual carbon atoms may carry one or more substituents independently of one another selected from the list below:
   hydrogen, hydroxyl, CH₂OH, cyano, halogen, straight-chain or branched C₁-C₄-alkyl, straight-chain or branched C₁-C₄-haloalkyl, straight-chain or branched C₁-C₄-alkoxyalkyl, straight-chain or branched C₁-C₄-alkoxycarbonyl, straight-chain or branched phenyl or benzyl, an optionally alkyl-substituted C₂-C₅-alkyl chain which may contain up to one oxygen or an optionally alkyl-substituted C₃-C₅-alkenyl chain,
   where the other substituents have one or more of the meanings mentioned above,
   and also the agrochemically active salts thereof.

Preference is furthermore given to compounds of the formula (I) in which

the radical R³ represents a group of the formula E1, E2 or E3

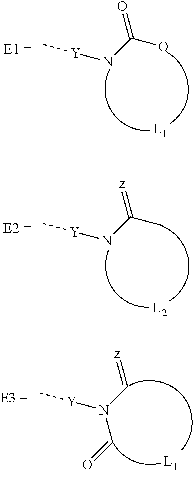

in which one or more of the symbols have one of the meanings below:
Y represents a direct bond, C═O or a C₁-C₃-alkyl chain substituted by straight-chain or branched C₁-C₄-alkyl, C₁-C₄-haloalkyl or C₁-C₄-alkoxyalkyl,
Z represents sulphur or oxygen,
L₁ represents an unsubstituted or substituted C₁- to C₄-alkylene chain or a C₂- to C₄-alkenyl chain,
   where double bonds are not cumulated and
   where the individual carbon atoms may carry one or more substituents independently of one another selected from the list below:
   hydrogen, hydroxyl, straight-chain or branched C₁-C₄-alkyl, straight-chain or branched C₁-C₄-alkoxyalkyl, straight-chain or branched C₁-C₄-alkoxycarbonyl, straight-chain or branched C₁-C₄-alkoxy-C₁-C₄-alkyl, unsubstituted or substituted phenyl or benzyl,
   or
   two substituents attached to two adjacent carbon atoms of the alkylene chain together with these two carbon atoms form
   a 5- or 6-membered unsubstituted or substituted saturated carbocycle,
   where the substituents independently of one another are selected from the list below:
   hydrogen, fluorine, optionally branched C₁-C₄-alkyl, optionally branched C₁-C₄-alkoxy or optionally branched C₁-C₄-haloalkyl,
   or
   a 5- or 6-membered unsubstituted or substituted saturated heterocycle which contains an oxygen atom or a sulphur atom,
   where the substituents independently of one another are selected from the list below:
   hydrogen, fluorine, optionally branched C₁-C₄-alkyl, optionally branched C₁-C₄-alkoxy or optionally branched C₁-C₄-haloalkyl,
   or
   an unsubstituted or substituted phenyl ring,
   where the substituents independently of one another are selected from the list below:
   hydrogen, halogen, CN, SCH₃, NO₂, optionally branched C₁-C₄-alkyl, optionally branched C₁-C₄-alkoxy, optionally branched C₁-C₄-alkylcarbonyl, optionally branched C₁-C₄-haloalkyl or optionally branched C₁-C₄-haloalkoxy,
L₂ represents an unsubstituted or substituted C₁- to C₄-alkylene chain or a C₂- to C₄-alkenyl chain or a 1,3-linked cyclopentyl (3-oxo-2-azabicyclo[2.2.1]hept-2-yl) or a C₁- to C₄-alkylene chain which is interrupted by a heteroatom selected from the group consisting of oxygen, sulphur and nitrogen,
   where double bonds are not cumulated and
   where the individual carbon atoms may carry one or more substituents independently of one another selected from the list below:
   hydrogen, hydroxyl, CH₂OH, cyano, halogen, straight-chain or branched C₁-C₄-alkyl, straight-chain or branched C₁-C₄-haloalkyl, straight-chain or branched C₁-C₄-alkoxyalkyl, straight-chain or branched C₁-C₄-alkoxycarbonyl, straight-chain or branched phenyl or benzyl, an optionally alkyl-substituted C₂-C₅-alkyl chain which may contain up to one oxygen or an optionally alkyl-substituted C₃-C₅-alkenyl chain,
   where the other substituents have one or more of the meanings mentioned above,
   and also the agrochemically active salts thereof.

Preference is furthermore given to compounds of the formula (I) in which

exactly one of the radicals R² and R³ represents a group of the formula E1

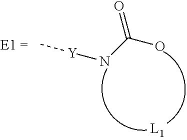

in which one or more of the symbols have one of the meanings below:
Y represents a direct bond or —CH₂—, —CH₂CH₂—, —CHMe-, —CHCF₃— or C═O,
L₁ represents an unsubstituted or substituted C₁- to C₄-alkylene chain or a C₂- to C₄-alkenyl chain,
   where double bonds are not cumulated and
   where the individual carbon atoms may carry one or more substituents independently of one another selected from the list below:
   hydrogen, methyl, ethyl, hydroxyl, methoxy, OCH₂CH₃, COOCH₃, COOCH₂CH₃, CH₂OH, CH₂CH(CH₃)₂ or phenyl,
   or
   two substituents attached to two adjacent carbon atoms of the alkylene chain together with these two carbon atoms form
   a 5- or 6-membered unsubstituted or substituted saturated carbocycle,
   where the substituents independently of one another are selected from the list below:
   hydrogen, fluorine, optionally branched C₁-C₄-alkyl, optionally branched C₁-C₄-alkoxy or optionally branched C₁-C₄-haloalkyl,
   or
   a 5- or 6-membered unsubstituted or substituted saturated heterocycle which contains an oxygen atom or a sulphur atom,
   where the substituents independently of one another are selected from the list below:
   hydrogen, fluorine, optionally branched C₁-C₄-alkyl, optionally branched C₁-C₄-alkoxy or optionally branched C₁-C₄-haloalkyl,
   or
   an unsubstituted or substituted phenyl ring,
   where the substituents independently of one another are selected from the list below:
   hydrogen, halogen, CN, SCH₃, NO₂, optionally branched C₁-C₄-alkyl, optionally branched C₁-C₄-alkoxy, optionally branched C₁-C₄-alkylcarbonyl, optionally branched C₁-C₄-haloalkyl or optionally branched C₁-C₄-haloalkoxy,
   where the other substituents have one or more of the meanings mentioned above,
   and also the agrochemically active salts thereof.

Preference is furthermore given to compounds of the formula (I) in which

exactly one of the radicals R² and R³ represents a group of the formula E2

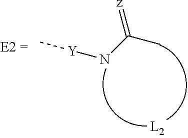

in which one or more of the symbols have one of the meanings below:
Y represents a direct bond or —CH₂—, —CH₂CH₂—, —CHMe-, —CHCF₃— or C═O,
Z represents sulphur or oxygen,
L₂ represents an unsubstituted or substituted C₁- to C₄-alkylene chain, a C₂- to C₄-alkenyl chain or a 1,3-linked cyclopentyl (3-oxo-2-azabicyclo[2.2.1]hept-2-yl) or a C₁- to C₄-alkylene chain interrupted by a heteroatom selected from the group consisting of oxygen, sulphur and nitrogen,
   where double bonds are not cumulated and
   where the individual carbon atoms may carry one or more substituents independently of one another selected from the list below:
   hydrogen, methyl, ethyl, cyano, CF₃, isopropyl, hydroxyl, methoxy, COOCH₃, COOCH₂CH₃, OCH₂CH₃, CH₂OH, CH₂CH(CH₃)₂, phenyl, —CH₂OCH₂CH₂—, —CHC(OCH₃)CH₂—C(CH₂CH₃)C(CH₃)CH₂— or —CH═CH═CH═CH—,
   where the other substituents have one or more of the meanings mentioned above,
   and also agrochemically active salts thereof.

Preference is furthermore given to compounds of the formula (I) in which

exactly one of the radicals R² and R³ represents a group of the formula E3

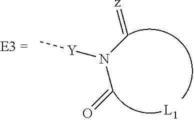

in which one or more of the symbols have one of the meanings below:
Y represents a direct bond or —CH₂—, —CH₂CH₂—, —CHMe-, —CHCF₃— or C═O,
Z represents sulphur or oxygen,
L₁ represents an unsubstituted or substituted C₁- to C₄-alkylene chain or a C₂- to C₄-alkenyl chain,
   where double bonds are not cumulated and
   where the individual carbon atoms may carry one or more substituents independently of one another selected from the list below:
   hydrogen, methyl, ethyl, hydroxyl, methoxy, OCH₂CH₃, COOCH₃, COOCH₂CH₃, CH₂OH, CH₂CH(CH₃)₂ or phenyl,
   or
   two substituents attached to two adjacent carbon atoms of the alkylene chain together with these two carbon atoms form
   a 5- or 6-membered unsubstituted or substituted saturated carbocycle,
   where the substituents independently of one another are selected from the list below:
   hydrogen, fluorine, optionally branched C₁-C₄-alkyl, optionally branched C₁-C₄-alkoxy or optionally branched C₁-C₄-haloalkyl,
   or
   a 5- or 6-membered unsubstituted or substituted saturated heterocycle which contains an oxygen atom or a sulphur atom,
   where the substituents independently of one another are selected from the list below:
   hydrogen, fluorine, optionally branched C₁-C₄-alkyl, optionally branched C₁-C₄-alkoxy or optionally branched C₁-C₄-haloalkyl,
   or
   an unsubstituted or substituted phenyl ring,
   where the substituents independently of one another are selected from the list below:
   hydrogen, halogen, CN, SCH₃, NO₂, optionally branched C₁-C₄-alkyl, optionally branched C₁-C₄-alkoxy, optionally branched C₁-C₄-alkylcarbonyl, optionally branched C₁-C₄-haloalkyl or optionally branched C₁-C₄-haloalkoxy,
   where the other substituents have one or more of the meanings mentioned above,
   and also the agrochemically active salts thereof.

Preference is furthermore given to compounds of the formula (I) in which

R² represents one of the radicals below:
(2,5-dioxopyrrolidin-1-yl)methyl, (2-oxopyrrolidin-1-yl)carbonyl, (3-methyl-2-oxopyrrolidin-1-yl)methyl, 1-(2,5-dioxopyrrolidin-1-yl)-2,2,2-trifluoroethyl, (2R)-2-(ethoxycarbonyl)-5-oxopyrrolidin-1-yl, (2S)-2-(ethoxycarbonyl)-5-oxopyrrolidin-1-yl, 2-(ethoxycarbonyl)-5-oxopyrrolidin-1-yl, (2R)-2-(hydroxymethyl)-5-oxopyrrolidin-1-yl, (2S)-2-(hydroxymethyl)-5-oxopyrrolidin-1-yl, 2-(hydroxymethyl)-5-oxopyrrolidin-1-yl, 2,5-dioxopyrrolidin-1-yl, 2-ethoxy-5-oxopyrrolidin-1-yl, (2R)-2-methyl-5-oxopyrrolidin-1-yl, (2S)-2-methyl-5-oxopyrrolidin-1-yl, 2-methyl-5-oxopyrrolidin-1-yl, 2-oxo-1,3-oxazolidin-3-yl, (4R)-2-oxo-4-(propan-2-yl)-1,3-oxazolidin-3-yl, (4S)-2-oxo-4-(propan-2-yl)-1,3-oxazolidin-3-yl, 2-oxo-4-(propan-2-yl)-1,3-oxazolidin-3-yl, 2-oxo-4-phenyl-1,3-oxazolidin-3-yl, 2-oxo-5-phenylpyrrolidin-1-yl, 2-oxoazepan-1-yl, 2-oxopiperidin-1-yl, 2-oxopyridin-1(2H)-yl, 2-oxopyrrolidin-1-yl, 2-thioxopyrrolidin-1-yl, 3,3-dimethyl-2,5-dioxopyrrolidin-1-yl, 3,3-dimethyl-2-oxoazetidin-1-yl, 3,5-dimethyl-2-oxopyrrolidin-1-yl, (3R,5R)-3,5-dimethyl-2-oxopyrrolidin-1-yl, (3R,5S)-3,5-dimethyl-2-oxopyrrolidin-1-yl, 3-ethyl-4-methyl-2-oxo-2,5-dihydro-1H-pyrrol-1-yl, (3R)-3-hydroxy-2-oxopyrrolidin-1-yl, (3S)-3-hydroxy-2-oxopyrrolidin-1-yl, 3-hydroxy-2-oxopyrrolidin-1-yl, 3-methyl-2,5-dioxopyrrolidin-1-yl, (3R)-3-methyl-2-oxopyrrolidin-1-yl, (3S)-3-methyl-2-oxopyrrolidin-1-yl, 3-methyl-2-oxopyrrolidin-1-yl, 3-oxomorpholin-4-yl, 4-(2-methylpropyl)-2-oxo-1,3-oxazolidin-3-yl, 4-(methoxycarbonyl)-2-oxopyrrolidin-1-yl, 4-ethyl-2-oxo-1,3-oxazolidin-3-yl, (4R)-4-hydroxy-2-oxopyrrolidin-1-yl, (4S)-4-hydroxy-2-oxopyrrolidin-1-yl, 4-hydroxy-2-oxopyrrolidin-1-yl, 4-methoxy-2-oxo-2,5-dihydro-1H-pyrrol-1-yl, (4R)-4-methyl-2-oxo-1,3-oxazolidin-3-yl, (4S)-4-methyl-2-oxo-1,3-oxazolidin-3-yl, 4-methyl-2-oxo-1,3-oxazolidin-3-yl, 4-methyl-2-oxopyrrolidin-1-yl, 5-ethyl-3-methyl-2-oxopyrrolidin-1-yl, 4,4-dimethyl-2-oxo-1,3-oxazolidin-3-yl, 5-methyl-2-oxo-1,3-oxazolidin-3-yl, 5,5-dimethyl-2-oxo-1,3-oxazolidin-3-yl, 2-ethyl-5-oxopyrrolidin-1-yl, 2-oxo-5-(propan-2-yl)pyrrolidin-1-yl, 2-oxo-3-(trifluoromethyl)pyrrolidin-1-yl, 3,3-dimethyl-2-oxopyrrolidin-1-yl, 3,3-dimethyl-2,5-dioxopyrrolidin-1-yl, 3,3-dimethyl-2-oxo-5-thioxopyrrolidin-1-yl, 3-cyano-2-oxopyrrolidin-1-yl or 3-oxo-2-azabicyclo[2.2.1]hept-2-yl,
   where the other substituents have one or more of the meanings mentioned above,
   and also the agrochemically active salts thereof.

Preference is furthermore given to compounds of the formula (I) in which

R³ represents methoxy,
where the other substituents have one or more of the meanings mentioned above,
and also the agrochemically active salts thereof.

Preference is furthermore given to compounds of the formula (I) in which

R⁴ represents methoxy,
where the other substituents have one or more of the meanings mentioned above,
and also the agrochemically active salts thereof.

Preference is furthermore given to compounds of the formula (I) in which

R⁶ represents one of the radicals below:
(propan-2-yloxy)carbonyl, COOCH₃, COOCH₂CH₃,
   where the other substituents have one or more of the meanings mentioned above,
   and also the agrochemically active salts thereof.

Preference is furthermore given to compounds of the formula (I) in which

R¹⁰ represents one of the radicals below:
2-ethylcyclopropyl, 1-fluoropropan-2-yl, 1-methylcyclopropyl, 2-fluoroethyl, 2-methylcyclopropyl, 3-methylcyclobutyl,
   where the other substituents have one or more of the meanings mentioned above,
   and also the agrochemically active salts thereof.

Preference is furthermore given to compounds of the formula (I) in which

R³ represents one of the radicals below:
2-oxo-1,3-oxazolidin-3-yl, 2-oxopyrrolidin-1-yl or 4-methyl-2-oxo-1,3-oxazolidin-3-yl,
   where the other substituents have one or more of the meanings mentioned above,
   and also the agrochemically active salts thereof.

Preference is furthermore given to compounds of the formula (I) in which

R¹ and R⁵ both represent hydrogen,
where the other substituents have one or more of the meanings mentioned above,
and also the agrochemically active salts thereof.

Preference is furthermore given to compounds of the formula (I) in which

R⁶ represents hydrogen,
where the other substituents have one or more of the meanings mentioned above,
and also the agrochemically active salts thereof.

Preference is furthermore given to compounds of the formula (I) in which

R⁷ represents hydrogen,
where the other substituents have one or more of the meanings mentioned above,
and also the agrochemically active salts thereof.

Preference is furthermore given to compounds of the formula (I) in which

R⁸ represents chlorine, bromine, fluorine, iodine, cyano or CF₃,
where the other substituents have one or more of the meanings mentioned above,
and also the agrochemically active salts thereof.

Preference is furthermore given to compounds of the formula (I) in which

R⁹ represents H or Me,
where the other substituents have one or more of the meanings mentioned above,
and also the agrochemically active salts thereof.

Preference is furthermore given to compounds of the formula (I) in which

R¹, R⁵, R⁶ and R⁷ represent hydrogen,
where the other substituents have one or more of the meanings mentioned above,
and also the agrochemically active salts thereof.

Preference is furthermore given to compounds of the formula (I) in which
R¹⁰ represents methyl, ethyl, propyl, cyclopropyl, cyclopropylmethyl, 1-cyclopropyleth-1-yl, 2-methylcyclopropyl, 2,2-dimethylcyclopropyl, 2,2-dimethylprop-1-yl, tert-butyl, cyclobutyl, butyl, 3-methylbut-1-yl, 2-methylbut-1-yl, 2-methylprop-1-yl, 1-fluoroprop-2-yl, cyclopentyl, propan-2-yl, pentan-3-yl, pentan-2-yl, pentyl, prop-2-en-1-yl, prop-2-yn-1-yl, butan-2-yl, 2,2,2-trifluoroethyl, 2,2-difluoroethyl, 2-methoxyethan-1-yl, 2-methylmercaptoethan-1-yl, 2-fluoroethan-1-yl, 2-chlorioethan-1-yl, 2-cyanoethan-1-yl, 1-methoxypropan-2-yl, 3-methoxypropan-1-yl, 1-methylmercaptopropan-2-yl, 2-methyl-1-(methylsulphanyl)propan-2-yl, oxetan-3-yl, 1,1,1-trifluoropropan-2-yl, 2,2,3,3,3-pentafluoropropyl, 1,1,1-trifluoropropan-3-yl, 1,1,1-trifluorobutan-2-yl, 1,1,1-trifluorobutan-3-yl, 2-methylprop-2-en-1-yl or 1-fluoropropan-2-yl,
   where the other substituents have one or more of the meanings mentioned above,
   and also the agrochemically active salts thereof.

The radical definitions and preferred ranges mentioned above can be combined with one another as desired. Moreover, individual definitions may not apply.

Examples of inorganic acids are hydrohalic acids, such as hydrogen fluoride, hydrogen chloride, hydrogen bromide and hydrogen iodide, sulphuric acid, phosphoric acid and nitric acid, and acidic salts, such as NaHSO₄ and KHSO₄. Suitable organic acids are, for example, formic acid, carbonic acid and alkanoic acids, such as acetic acid, trifluoroacetic acid, trichloroacetic acid and propionic acid, and also glycolic acid, thiocyanic acid, lactic acid, succinic acid, citric acid, benzoic acid, cinnamic acid, oxalic acid, alkylsulphonic acids (sulphonic acids having straight-chain or branched alkyl radicals of 1 to 20 carbon atoms), arylsulphonic acids or aryldisulphonic acids (aromatic radicals, such as phenyl and naphthyl, which carry one or two sulphonic acid groups), alkylphosphonic acids (phosphonic acids having straight-chain or branched alkyl radicals of 1 to 20 carbon atoms), arylphosphonic acids or aryldiphosphonic acids (aromatic radicals, such as phenyl and naphthyl, which carry one or two phosphonic acid radicals), where the alkyl and aryl radicals may carry further substituents, for example p-toluenesulphonic acid, salicylic acid, p-aminosalicylic acid, 2-phenoxybenzoic acid, 2-acetoxybenzoic acid, etc.

Suitable metal ions are in particular the ions of the elements of the second main group, in particular calcium and magnesium, of the third and fourth main group, in particular aluminium, tin and lead, and also of the first to eighth transition group, in particular chromium, manganese, iron, cobalt, nickel, copper, zinc and others. Particular preference is given to the metal ions of the elements of the fourth period. Here, the metals can be present in various valencies that they can assume.

Substituted groups may be mono- or polysubstituted, where in the case of polysubstitution the substituents may be identical or different.

In the definitions of the symbols given in the formulae above, collective terms were used which are generally representative for the following substituents:

halogen: fluorine, chlorine, bromine and iodine;
aryl: an unsubstituted or unbranched or substituted 5- to 15-membered partially or fully unsaturated mono-, bi- or tricyclic ring system having up to 3 ring members selected from the groups C(═O), (C═S), where at least one of the rings of the ring system is fully unsaturated, such as, for example (but not limited thereto) benzene, naphthalene, tetrahydronaphthalene, anthracene, indane, phenanthrene, azulene;
alkyl: saturated straight-chain or branched hydrocarbon radicals having 1 to 10 carbon atoms, such as, for example (but not limited thereto) methyl, ethyl, propyl, 1-methylethyl, butyl, 1-methyl-propyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl, heptyl, 1-methylhexyl, octyl, 1,1-dimethylhexyl, 2-ethylhexyl, 1-ethylhexyl, nonyl, 1,2,2-trimethylhexyl, decyl;
haloalkyl: straight-chain or branched alkyl groups having 1 to 4 carbon atoms (as mentioned above), where in these groups some or all of the hydrogen atoms may be replaced by halogen atoms as mentioned above, such as, for example (but not limited thereto), C₁-C₂-haloalkyl, such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2-difluoroethyl, 2,2-dichloro-2-fluoroethyl, 2,2,2-trichloroethyl, pentafluoroethyl and 1,1,1-trifluoroprop-2-yl;
alkenyl: unsaturated straight-chain or branched hydrocarbon radicals having 2 to 16 carbon atoms and at least one double bond in any position, such as, for example (but not limited thereto), C₂-C₆-alkenyl, such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl;
alkynyl: straight-chain or branched hydrocarbon groups having 2 to 16 carbon atoms and at least one triple bond in any position, such as, for example (but not limited thereto), C₂-C₆-alkynyl, such as ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl and 1-ethyl-1-methyl-2-propynyl;
alkoxy: saturated straight-chain or branched alkoxy radicals having 1 to 4 carbon atoms, such as, for example (but not limited thereto), C₁-C₄-alkoxy, such as methoxy, ethoxy, propoxy, 1-methylethoxy, butoxy, 1-methylpropoxy, 2-methylpropoxy, 1,1-dimethylethoxy;
haloalkoxy: straight-chain or branched alkoxy groups having 1 to 4 carbon atoms (as mentioned above), where some or all of the hydrogen atoms in these groups may be replaced by halogen atoms as mentioned above, such as, for example (but not limited thereto), C₁-C₂-haloalkoxy, such as chloromethoxy, bromomethoxy, dichloromethoxy, trichloromethoxy, fluoromethoxy, difluoromethoxy, trifluoromethoxy, chlorofluoromethoxy, dichlorofluoromethoxy, chlorodifluoromethoxy, 1-chloroethoxy, 1-bromoethoxy, 1-fluoroethoxy, 2-fluoroethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy, pentafluoro-ethoxy and 1,1,1-trifluoroprop-2-oxy;
thioalkyl: saturated straight-chain or branched alkylthio radicals having 1 to 6 carbon atoms, such as, for example (but not limited thereto), C₁-C₆-alkylthio, such as methylthio, ethylthio, propylthio, 1-methylethylthio, butylthio, 1-methylpropylthio, 2-methylpropylthio, 1,1-dimethylethylthio, pentylthio, 1-methylbutylthio, 2-methylbutylthio, 3-methylbutylthio, 2,2-dimethylpropylthio, 1-ethylpropylthio, hexylthio, 1,1-dimethylpropylthio, 1,2-dimethylpropylthio, 1-methylpentylthio, 2-methylpentylthio, 3-methylpentylthio, 4-methylpentylthio, 1,1-dimethylbutylthio, 1,2-dimethylbutylthio, 1,3-dimethylbutylthio, 2,2-dimethylbutylthio, 2,3-dimethylbutylthio, 3,3-dimethylbutylthio, 1-ethylbutylthio, 2-ethylbutylthio, 1,1,2-trimethylpropylthio, 1,2,2-trimethylpropylthio, 1-ethyl-1-methylpropylthio and 1-ethyl-2-methylpropylthio;
thiohaloalkyl: straight-chain or branched alkylthio groups having 1 to 6 carbon atoms (as mentioned above), where some or all of the hydrogen atoms in these groups may be replaced by halogen atoms as mentioned above, such as, for example (but not limited thereto) C₁-C₂-haloalkylthio, such as chloromethylthio, bromomethylthio, dichloromethylthio, trichloromethylthio, fluoromethylthio, difluoromethylthio, trifluoromethylthio, chlorofluoromethylthio, dichlorofluoromethylthio, chlorodifluoromethylthio, 1-chloroethylthio, 1-bromoethylthio, 1-fluoroethylthio, 2-fluoroethylthio, 2,2-difluoroethylthio, 2,2,2-trifluoroethylthio, 2-chloro-2-fluoroethylthio, 2-chloro-2,2-difluoroethylthio, 2,2-dichloro-2-fluoroethylthio, 2,2,2-trichloroethylthio, pentafluoroethylthio and 1,1,1-trifluoroprop-2-ylthio;
cycloalkyl: mono-, bi- or tricyclic saturated hydrocarbon groups having 3 to 12 carbon ring members, such as, for example (but not limited thereto), cyclopropyl, cyclobutyl, cyclopentyl and cyclohexyl, bicyclo[1.0.1]butane, decalinyl, norbornyl;
cycloalkenyl: mono-, bi- or tricyclic non-aromatic hydrocarbon groups having 5 to 15 carbon ring members and at least one double bond, such as, for example (but not limited thereto) cyclopenten-1-yl, cyclohexen-1-yl, cyclohepta-1,3-dien-1-yl, norbornen-1-yl;
(alkoxy)carbonyl: an alkoxy group having 1 to 4 carbon atoms (as mentioned above) which is attached to the skeleton via a carbonyl group (—CO—);
heterocyclyl: a three- to fifteen-membered saturated or partially unsaturated heterocycle which contains one to four heteroatoms from the group consisting of oxygen, nitrogen and sulphur: mono-, bi- or tricyclic heterocycles containing, in addition to carbon ring members, one to three nitrogen atoms and/or one oxygen or sulphur atom or one or two oxygen and/or sulphur atoms; if the ring contains a plurality of oxygen atoms, these are not directly adjacent; such as, for example (but not limited thereto), oxiranyl, aziridinyl, 2-tetrahydrofuranyl, 3-tetrahydrofuranyl, 2-tetrahydrothienyl, 3-tetrahydrothienyl, 2-pyrrolidinyl, 3-pyrrolidinyl, 3-isoxazolidinyl, 4-isoxazolidinyl, 5-isoxazolidinyl, 3-isothiazolidinyl, 4-isothiazolidinyl, 5-isothiazolidinyl, 3-pyrazolidinyl, 4-pyrazolidinyl, 5-pyrazolidinyl, 2-oxazolidinyl, 4-oxazolidinyl, 5-oxazolidinyl, 2-thiazolidinyl, 4-thiazolidinyl, 5-thiazolidinyl, 2-imidazolidinyl, 4-imidazolidinyl, 1,2,4-oxadiazolidin-3-yl, 1,2,4-oxadiazolidin-5-yl, 1,2,4-thiadiazolidin-3-yl, 1,2,4-thiadiazolidin-5-yl, 1,2,4-triazolidin-3-yl, 1,3,4-oxadiazolidin-2-yl, 1,3,4-thiadiazolidin-2-yl, 1,3,4-triazolidin-2-yl, 2,3-dihydrofur-2-yl, 2,3-dihydrofur-3-yl, 2,4-dihydrofur-2-yl, 2,4-dihydrofur-3-yl, 2,3-dihydrothien-2-yl, 2,3-dihydrothien-3-yl, 2,4-dihydrothien-2-yl, 2,4-dihydrothien-3-yl, 2-pyrrolin-2-yl, 2-pyrrolin-3-yl, 3-pyrrolin-2-yl, 3-pyrrolin-3-yl, 2-isoxazolin-3-yl, 3-isoxazolin-3-yl, 4-isoxazolin-3-yl, 2-isoxazolin-4-yl, 3-isoxazolin-4-yl, 4-isoxazolin-4-yl, 2-isoxazolin-5-yl, 3-isoxazolin-5-yl, 4-isoxazolin-5-yl, 2-isothiazolin-3-yl, 3-isothiazolin-3-yl, 4-isothiazolin-3-yl, 2-isothiazolin-4-yl, 3-isothiazolin-4-yl, 4-isothiazolin-4-yl, 2-isothiazolin-5-yl, 3-isothiazolin-5-yl, 4-isothiazolin-5-yl, 2,3-dihydropyrazol-1-yl, 2,3-dihydropyrazol-2-yl, 2,3-dihydropyrazol-3-yl, 2,3-dihydropyrazol-4-yl, 2,3-dihydropyrazol-5-yl, 3,4-dihydropyrazol-1-yl, 3,4-dihydropyrazol-3-yl, 3,4-dihydropyrazol-4-yl, 3,4-dihydropyrazol-5-yl, 4,5-dihydropyrazol-1-yl, 4,5-dihydropyrazol-3-yl, 4,5-dihydropyrazol-4-yl, 4,5-dihydropyrazol-5-yl, 2,3-dihydrooxazol-2-yl, 2,3-dihydrooxazol-3-yl, 2,3-dihydrooxazol-4-yl, 2,3-dihydrooxazol-5-yl, 3,4-dihydrooxazol-2-yl, 3,4-dihydrooxazol-3-yl, 3,4-dihydrooxazol-4-yl, 3,4-dihydrooxazol-5-yl, 3,4-dihydrooxazol-2-yl, 3,4-dihydrooxazol-3-yl, 3,4-dihydrooxazol-4-yl, 2-piperidinyl, 3-piperidinyl, 4-piperidinyl, 1,3-dioxan-5-yl, 2-tetrahydropyranyl, 4-tetrahydropyranyl, 2-tetrahydrothienyl, 3-hexahydropyridazinyl, 4-hexahydropyridazinyl, 2-hexahydropyrimidinyl, 4-hexahydropyrimidinyl, 5-hexahydropyrimidinyl, 2-piperazinyl, 1,3,5-hexahydrotriazin-2-yl und 1,2,4-hexahydrotriazin-3-yl;
hetaryl: unsubstituted or optionally substituted, 5- to 15-membered partially or fully unsaturated mono-, bi- or tricyclic ring system where at least one of the rings of the ring system is fully unsaturated, comprising one to four heteroatoms from the group consisting of oxygen, nitrogen and sulphur, if the ring contains a plurality of oxygen atoms, these are not directly adjacent;
such as, for example (but not limited thereto),
5-membered heteroaryl which contains one to four nitrogen atoms or one to three nitrogen atoms and one sulphur or oxygen atom: 5-membered heteroaryl groups which, in addition to carbon atoms, may contain one to four nitrogen atom or one to three nitrogen atoms and one sulphur or oxygen atom as ring members, for example 2-furyl, 3-furyl, 2-thienyl, 3-thienyl, 2-pyrrolyl, 3-pyrrolyl, 3-isoxazolyl, 4-isoxazolyl, 5-isoxazolyl, 3-isothiazolyl, 4-isothiazolyl, 5-isothiazolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 2-imidazolyl, 4-imidazolyl, 1,2,4-oxadiazol-3-yl, 1,2,4-oxadiazol-5-yl, 1,2,4-thiadiazol-3-yl, 1,2,4-thiadiazol-5-yl, 1,2,4-triazol-3-yl, 1,3,4-oxadiazol-2-yl, 1,3,4-thiadiazol-2-yl and 1,3,4-triazol-2-yl;
benzo-fused 5-membered heteroaryl which contains one to three nitrogen atoms or one nitrogen atom and one oxygen or sulphur atom: 5-membered heteroaryl groups which, in addition to carbon atoms, may contain one to four nitrogen atoms or one to three nitrogen atoms and one sulphur or oxygen atom as ring members, and in which two adjacent carbon ring members or one nitrogen and one adjacent carbon ring member may be bridged by a buta-1,3-diene-1,4-diyl group in which one or two carbon atoms may be replaced by nitrogen atoms; for example benzindolyl, benzimidazolyl, benzothiazolyl, benzopyrazolyl, benzofuryl;
5-membered heteroaryl which contains one to four nitrogen atoms and is attached via nitrogen or benzo-fused 5-membered heteroaryl which contains one to three nitrogen atoms and is attached via nitrogen: 5-membered heteroaryl groups which, in addition to carbon atoms, may contain one to four nitrogen atoms or one to three nitrogen atoms as ring members and in which two adjacent carbon ring members or one nitrogen and one adjacent carbon ring member may be bridged by a buta-1,3-diene-1,4-diyl group in which one or two carbon atoms may be replaced by nitrogen atoms, where these rings are attached to the skeleton via one of the nitrogen ring members, for example 1-pyrrolyl, 1-pyrazolyl, 1,2,4-triazol-1-yl, 1-imidazolyl, 1,2,3-triazol-1-yl, 1,3,4-triazol-1-yl;
6-membered heteroaryl which contains one to three or one to four nitrogen atoms: 6-membered heteroaryl groups which, in addition to carbon atoms, may contain one to three or one to four nitrogen atoms as ring members, for example 2-pyridinyl, 3-pyridinyl, 4-pyridinyl, 3-pyridazinyl, 4-pyridazinyl, 2-pyrimidinyl, 4-pyrimidinyl, 5-pyrimidinyl, 2-pyrazinyl, 1,3,5-triazin-2-yl and 1,2,4-triazin-3-yl.

Not included are combinations which contradict natural laws and which the person skilled in the art would therefore have excluded based on his expert knowledge. Excluded are, for example, ring structures having three or more adjacent oxygen atoms.

The present invention furthermore relates to a process for preparing the diaminopyrimidines of the formula (I) according to the invention

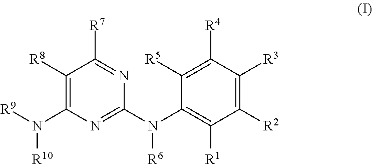

hereinbelow, depending on the appropriate process, also referred to by formula (Ia), (Ib) or (Ic),

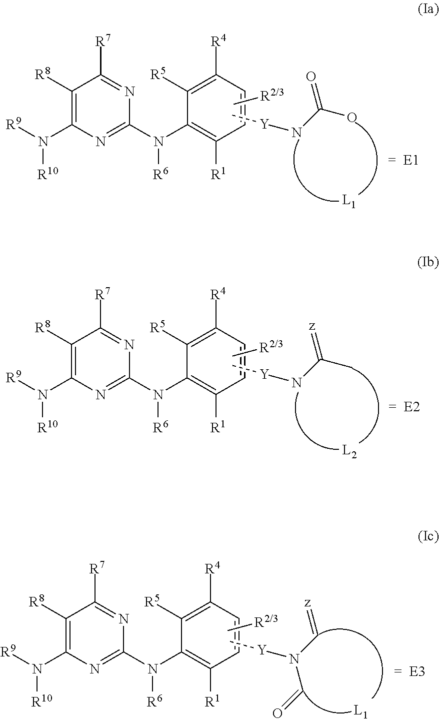

having in each case a heterocyclic side chain in the R² or R³ position (meta or para),
comprising at least one of steps (a) to (d) below:
(a) the reaction of 2,4-dihalopyrimidines of the formula (III) with amines of the formula (II) in the presence of a base, if appropriate in the presence of a solvent, if appropriate in the presence of a catalyst, to give compounds of the formula (V), according to the reaction scheme below (Scheme 1):

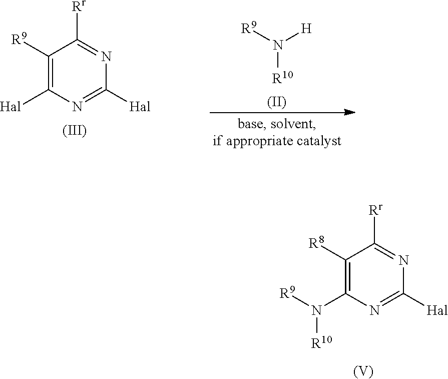

where Hal=F, Cl, Br, I
(b) the reaction of compounds of the formula (V) with aromatic amines of the formula (IV), if appropriate in the presence of an acid, if appropriate in the presence of a solvent, according to the reaction scheme below (Scheme 2):

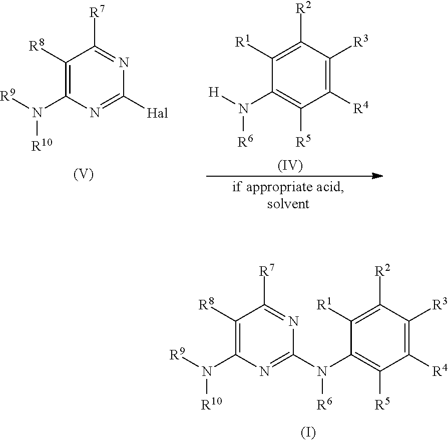

where Hal=F, Cl, Br, I
(c) the reaction of compounds of the formula (III) with anilines of the formula (IV) in the presence of a solvent and a catalyst, to give compounds of the formula (VI), according to the reaction scheme below (Scheme 3):

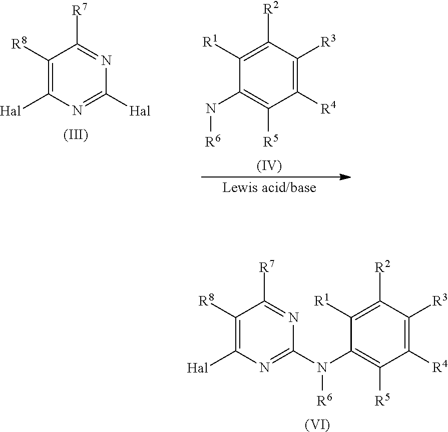

(d) the reaction of compounds of the formula (VI) with amines of the formula (II) in the presence of a base, if appropriate in the presence of a solvent, if appropriate in the presence of a catalyst, to give compounds of the formula (I), according to the reaction scheme below (Scheme 4):

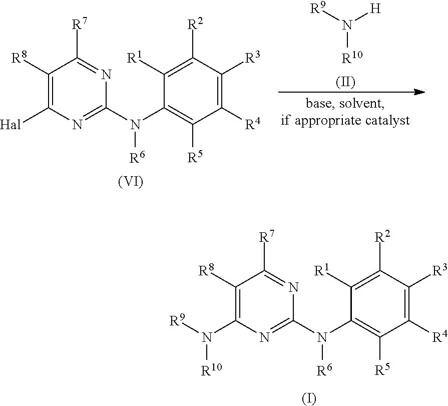

where the definitions of the radicals R¹ to R¹⁰ in the above schemes correspond to the definitions given above and Hal represents F, Cl, Br, I.

Scheme 1 shows one way for synthesizing the intermediates of the formula (V).

The alkylamino compounds of the formula (II) are either commercially available or can be prepared by literature procedures. One method for preparing suitable cyclopropylamino compounds of type (II) is, for example, the rearrangement of suitable carboxylic acid derivatives to the corresponding amino compounds (described, for example, in J. Am. Chem. Soc. 1961, 83, 3671-3678). Other methods, for example for preparing cyclobutylamino compounds of type (II), comprise the hydroboration of suitable cyclobutenes and subsequent treatment with NH₂SO₃H (for example Tetrahedron 1970, 26, 5033-5039), the reductive amination of cyclobutanones (described, for example, in J. Org. Chem. 1964, 29, 2588-2592) and also the reduction of nitro- or nitrosocyclobutanes (see, for example, J. Am. Chem. Soc. 1953, 75, 4044; Can. J. Chem. 1963, 41, 863-875) or azidocyclobutanes (described, for example, in Chem. Pharm. Bull. 1990, 38, 2719-2725; J. Org. Chem. 1962, 27, 1647-1650). The halogen-substituted amino compounds of the formula (II) are either commercially available or can be prepared by literature procedures. One method for preparing suitable halogen-substituted amino compounds (II) is, for example, the reduction of corresponding carboximides (described, for example, in EP30092) or corresponding oximes or azides (described, for example, in Chem. Ber. 1988, 119, 2233) or nitro compounds (described, for example, in J. Am. Chem Soc, 1953, 75, 5006). A further alternative consists in the treatment of corresponding aminocarboxylic acids with SF₄ in HF (described, for example, in J. Org. Chem. 1962, 27, 1406). The ring-opening of substituted aziridines with HF as described in J. Org. Chem. 1981, 46, 4938. Further methods for preparing halogen-substituted amino compounds (II) comprise the cleavage of corresponding phthalimides according to Gabriel (described, for example, in DE 3429048), the aminolysis of suitable haloalkyl halides (described, for example, in U.S. Pat. No. 2,539,406) or the degradation of corresponding carboxylic acid azides (described, for example, in DE3611195). Using suitable fluorinating agents (for example DAST), aminoaldehydes or -ketones can be converted into the corresponding difluoroalkylamines (WO2008008022), whereas amino alcohols form the corresponding monofluoroalkylamines (for example WO2006029115).

Analogously, using suitable chlorinating and brominating agents, chloro- and bromoalkylamines, respectively, can be obtained from amino alcohols (J. Org. Chem. 2005, 70, 7364, or Org. Lett., 2004, 6, 1935).

Suitable substituted 2,4-dihalopyrimidines (III) are either commercially available or can be prepared according to literature procedures, for example from commercially available substituted uracils (for example R⁸═CN: J. Org. Chem. 1962, 27, 2264; J. Chem. Soc. 1955, 1834; Chem. Ber. 1909, 42, 734; R⁸═CF₃: J. Fluorine Chem. 1996, 77, 93; see also WO 2000/047539). One way of preparing the compound (V) is shown in Scheme 1.

Using a suitable base at a temperature of from −30° C. to +80° C. in a suitable solvent, such as, for example, dioxane, THF, dimethylformamide or acetonitrile, initially an amine (II) is reacted with a 2,4-dihalopyrimidine (III) over a period of 1-24 h. Suitable for the use as base are, for example, inorganic salts, such as NaHCO₃, Na₂CO₃ or K₂CO₃, organometallic compounds, such as LDA or NaHMDS, or amine bases, such as ethyldiisopropylamine, DBU, DBN or tri-n-butylamine. Alternatively, the reaction can also be carried out as described, for example, in Org. Lett. 2006, 8, 395 with the aid of a suitable transition metal catalyst, such as, for example, palladium, together with a suitable ligand, such as, for example, triphenylphosphine or xanthphos.

One way of preparing the compound (I) is shown in Scheme 2.

The substituted aromatic amines (IV) are either commercially available or can be prepared from commercially available precursors by methods known from the literature. Aromatic amines carrying one or more identical or different substituents in the aromatic moiety can be prepared by a large number of methods described in the relevant literature. By way of example, some of the methods are mentioned below.

Cyclic radicals R¹ to R⁵ attached via nitrogen can be prepared, for example, by condensation of nitroaminoaromatics with haloalkylcarbonyl halides or diesters or diester equivalents or lactones; the subsequent reduction of the nitro group affords the desired aromatic amine.

The aromatic amines of the formula (IV) are divided into:

amines of the formula (IVa) (preparation see Scheme 10) for preparing compounds of the formula (I) in which exactly one of the radicals R² and R³ represents a group E1 (compounds of the formula (Ia)),
(IVb) (preparation see Schemes 16 and 17) for preparing compounds of the formula (I) in which exactly one of the radicals R² and R³ represents a group E2 (compounds of the formula (Ib))
and
(IVc) (preparation see Scheme 22) for preparing compounds of the formula (I) in which exactly one of the radicals R² and R³ represents a group E3 (compounds of the formula (Ic)).

The intermediate (V) is reacted in the presence of Brønsted acids, such as, for example, anhydrous hydrochloric acid, camphorsulphonic acid or p-toluenesulphonic acid, in a suitable solvent, such as, for example, dioxane, THF, DMSO, DME, 2-methoxyethanol, n-butanol or acetonitrile, at a temperature of 0° C.-140° C. over a period of 1-48 h with an aromatic amine (IV). Analogously described, for example, in Bioorg. Med. Chem. Lett. 2006, 16, 2689; GB2002 A1-2369359, Org. Lett. 2005, 7, 4113.

Alternatively, the reaction of (V) and (IV) to give (I) can also be carried out with base catalysis, i.e. using, for example, carbonates, such as potassium carbonate, alkoxides, such as potassium tert-butoxide, or hydrides, such as sodium hydride, where the catalytic use of a transition metal, such as, for example, palladium, together with a suitable ligand, such as, for example, xanthphos, may also be useful.

Finally, it is possible to carry out the reaction of (V) and (IV) to give (I) in the absence of solvents and/or Brønsted acids and also under MW conditions (described, for example, in Bioorg. Med. Chem. Lett. 2006, 16, 108; Bioorg. Med. Chem. Lett. 2005, 15, 3881).

One way of preparing the compound (VI) is shown in Scheme 3.

Initially, using a suitable Lewis acid or a suitable base at a temperature of from −15° C. to 100° C. in a suitable inert solvent, such as, for example, 1,4-dioxane, diethyl ether, THF, n-butanol, tert-butanol, dichloroethane or dichloromethane, an aniline (IV) is reacted with a 2,4-dihalopyrimidine (III) for a period of 1-24 h. Suitable for use as base are, for example, inorganic salts, such as NaHCO₃, Na₂CO₃ or K₂CO₃, organometallic compounds, such as LDA or NaHMDS, or amine bases, such as ethyldiisopropylamine, DBU, DBN or tri-n-butylamine. Suitable for use as Lewis acid are, for example (but not limited thereto) halides of the metals zinc (for example ZnCl₂), magnesium, copper, tin or titanium (see, for example, US 2005/0256145 or WO 2005/023780 and the literature cited therein).

One way of preparing the compound (I) is shown in Scheme 4.

For preparing compounds of the formula (I), the intermediate (VI) is then reacted in the presence of bases, such as, for example, carbonates, such as potassium carbonate, alkoxides, such as potassium tert-butoxide, or hydrides, such as sodium hydride, in a suitable solvent, such as, for example, dioxane, THF, DMSO, DME, 2-methoxyethanol, n-butanol or acetonitrile, at a temperature of 0° C.-140° C. over a period of 1-48 h with amines of the formula (II) where the catalytic use of a transition metal, such as, for example, palladium, together with a suitable ligand, such as, for example, triphenylphosphine or xanthphos, may also be useful.

Syntheses of compounds of the type (Ia) substituted by cyclic carbamates comprising at least one of steps (e) to (j) below:
(e) the reaction of compounds of the formula (V) with anilines of the formula (IVa) substituted by cyclic carbamates, if appropriate in the presence of an acid, if appropriate in the presence of a solvent, according to the reaction scheme below (Scheme 5):

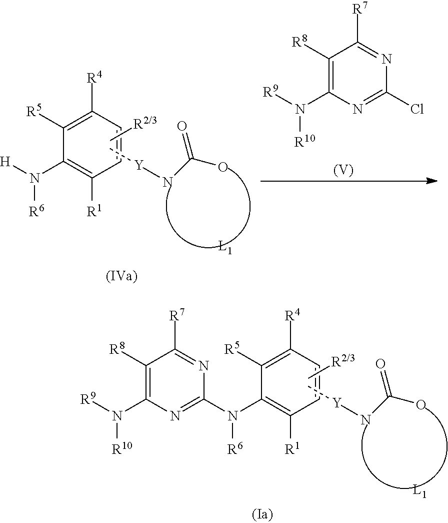

(f) the reaction of halogen-substituted compounds of the formula (VII) with cyclic carbamates of the formula (VIII) in the presence of a solvent and in the presence of a catalyst, according to the reaction scheme below (Scheme 6):

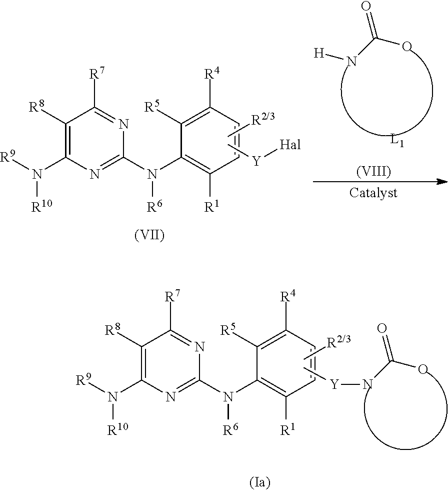

(g) the reaction of dihalo-substituted compounds of the formula (III) with anilines of the formula (IVa) substituted by cyclic carbamates, if appropriate in the presence of a Lewis acid, if appropriate in the presence of a solvent, according to the reaction scheme below (Scheme 7):

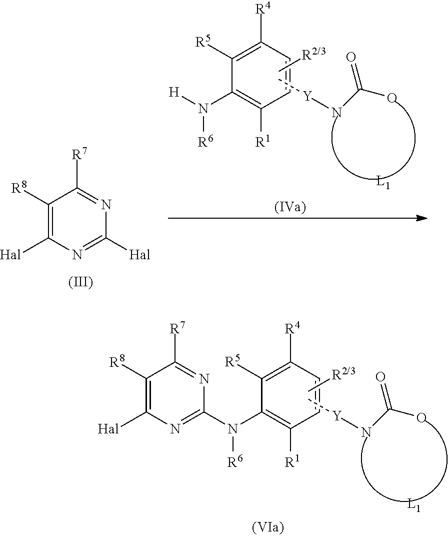

(h) the reaction of halo-substituted compounds of the formula (VIa) with amines of the formula (II) in the presence of a base, if appropriate in the presence of a solvent, if appropriate in the presence of a catalyst, to give compounds of the formula (Ia), according to the reaction scheme below (Scheme 8):

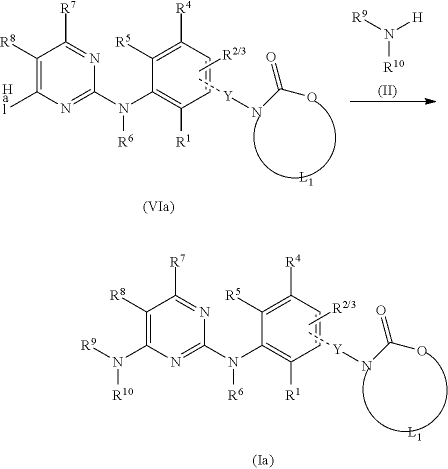

(i) the reaction of open-chain carbamate-substituted compounds of the formula (IX) with compounds of the formula (V) in the presence of a base, if appropriate in the presence of a solvent, if appropriate in the presence of a catalyst, to give compounds of the formula (Ia), according to the reaction scheme below (Scheme 9):

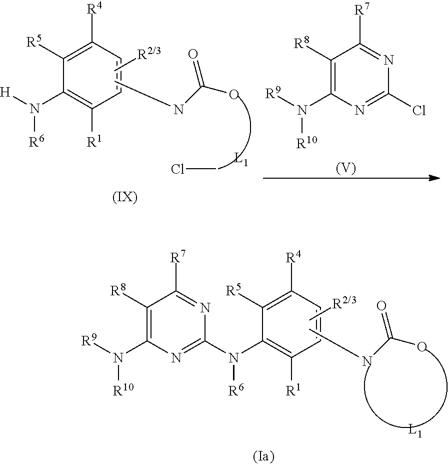

The aromatic amines (IVa) substituted by cyclic carbamates are either commercially available, they can be prepared by methods known from the literature from commercially available precursors or they are prepared as described below:
(j) the reaction of halogen-substituted anilines of the formula (X) with cyclic carbamates of the formula (VIII) in the presence of a solvent and in the presence of a catalyst, according to the reaction scheme below (Scheme 10):

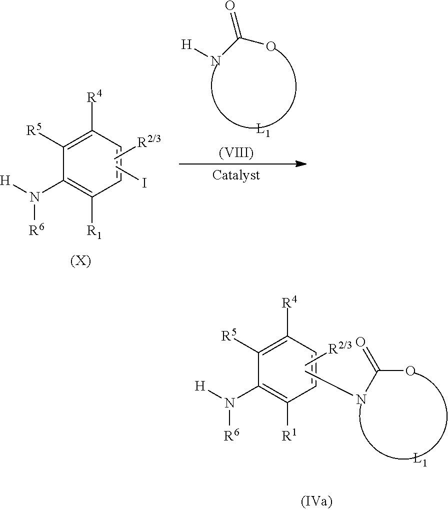

One way of preparing the compound (Ia) is shown in Scheme 5.

The intermediate (V) is reacted in the presence of Brønsted acids, such as, for example, anhydrous hydrochloric acid, camphorsulphonic acid or p-toluenesulphonic acid, in a suitable solvent, such as, for example, dioxane, THF, DMSO, DME, 2-methoxyethanol, n-butanol or acetonitrile, at a temperature of 0° C.-140° C. over a period of 1-48 h with an aromatic amine (IVa). Analogously described, for example, in Bioorg. Med. Chem. Lett. 2006, 16, 2689; GB2002 A1-2369359, Org. Lett. 2005, 7, 4113.

Alternatively, the reaction of (V) and (IVa) to give (Ia) can also be carried out with base catalysis, i.e. using, for example, carbonates, such as potassium carbonate, alkoxides, such as potassium tert-butoxide, or hydrides, such as sodium hydride, where the catalytic use of a transition metal such as, for example, palladium, together with a suitable ligand, such as, for example, xanthphos, may also be useful.

Finally, it is possible to carry out the reaction of (V) and (IVa) to give (Ia) in the absence of solvents and/or Brønsted acids (described, for example, in Bioorg. Med. Chem. Lett. 2006, 16, 108; Bioorg. Med. Chem. Lett. 2005, 15, 3881).

A further way of preparing the compound (Ia) is shown in Scheme 6.

The products (Ia) can be prepared by a copper-catalysed cross coupling between oxazolidinones (VIII) and aryl halides (VII) in the presence of a source of copper, a ligand and a base, in various solvents and at various temperatures. Various copper sources can be used, usually CuI, CuSO₄, Cu powder. Numerous ligands, such as, for example, 1,2-diaminocyclohexane or MeNHCH₂CH₂NHMMe may be employed. Suitable bases are, for example, K₂CO₃, K₃PO₄, Cs₂CO₃. These reactions can also be carried out under microwave conditions. For general reviews, see: Chem. Rev. 2006, 106, 2651; Synlett 2003, 2428 and the references cited. For specific examples, see: Org. Lett. 2003, 5, 963; J. Am. Chem. Soc. 2007, 129, 3490; Org. Lett., 2006, 8, 5609; Bioorg. Med. Chem. Lett. 2004, 14, 1221; Tetrahedron Lett. 2004, 45, 2311; J. Am. Chem. Soc. 2001, 123, 7727.

These amination reactions can also be carried out using other catalyst systems based, for example, on palladium or iron. (For general reviews, see: Chem. Rev. 2006, 106, 2651; for specific examples, see: Angew. Chem. Int. Ed. 2007, 46, 8862; Angew. Chem. Int. Ed. 2007, 46, 934; J. Am. Chem. Soc. 2002, 124, 6043).

One way of preparing the compound (VIa) is shown in Scheme 7.

Initially, using a suitable Lewis acid or a suitable base at a temperature of from −15° C. to 100° C. in a suitable inert solvent, such as, for example, 1,4-dioxane, diethyl ether, THF, n-butanol, tert-butanol, dichloroethane or dichloromethane, an aniline (IVa) is reacted with a 2,4-dihalopyrimidine (III) over a period of 1-24 h. Suitable for use as base are, for example, inorganic salts, such as NaHCO₃, Na₂CO₃ or K₂CO₃, organometallic compounds, such as LDA or NaHMDS, or amine bases, such as ethyldiisopropylamine, DBU, DBN or tri-n-butylamine. Suitable for use as Lewis acid are, for example (but not limited thereto) halides of the metals zinc (for example ZnCl₂), magnesium, copper, tin or titanium (see, for example, US 2005/0256145 or WO 2005/023780 and the literature cited therein).

One way of preparing the compound (Ia) is shown in Scheme 8.

For preparing compounds of the formula (Ia), the intermediate (VIa) is then reacted in the presence of bases, such as, for example, carbonates, such as potassium carbonate, alkoxides, such as potassium tert-butoxide, or hydrides, such as sodium hydride, in a suitable solvent, such as, for example dioxane, THF, DMSO, DME, 2-methoxyethanol, n-butanol or acetonitrile, at a temperature of 0° C.-140° C. over a period of 1-48 h with amines of the formula (II), where the catalytic use of a transition metal, such as, for example, palladium, together with a suitable ligand, such as, for example, triphenylphosphine or xanthphos, may also be useful.

A further way of preparing the compound (Ia) is shown in Scheme 9.

The intermediate (V) is reacted in the presence of Brønsted acids, such as, for example, anhydrous hydrochloric acids, camphorsulphonic acid or p-toluenesulphonic acid, in a suitable solvent, such as, for example, dioxane, THF, DMSO, DME, 2-methoxyethanol, n-butanol or acetonitrile, at a temperature of 0° C.-140° C. over a period of 1-48 h with an aromatic amine (IX).

Alternatively, the reaction of (V) with (IX) to give (Ia) can also be carried out under base catalysis, using, for example, carbonates, such as potassium carbonate, alkoxides, such as potassium tert-butoxide, or hydrides, such as, for example, sodium hydride. The synthesis of compounds of the formula (IX) and the ring closure under basic conditions to give the oxazolidinone are described, for example, in Tetrahedron Lett. 1988, 29, 5095, or DE3704632.

One way of preparing the compound (IVa) is shown in Scheme 10.

The intermediates of the formula (IVa) can be prepared via copper-catalysed cross coupling between oxazolidinones (VIII) and aryl halides (X) in the presence of a source of copper, a ligand and a base, in various solvents and at various temperatures. Various sources of copper can be used, usually CuI, CuSO₄, Cu powder. Numerous ligands, such as, for example, 1,2-diaminocyclohexane or MeNHCH₂CH₂NHMMe, may be employed. Suitable bases are, for example, K₂CO₃, K₃PO₄, Cs₂CO₃. These reactions can also be carried out under microwave conditions. For general reviews, see: Chem. Rev. 2006, 106, 2651; Synlett 2003, 2428 and references cited. For specific examples, see: Org. Lett. 2003, 5, 963; J. Am. Chem. Soc. 2007, 129, 3490; Org. Lett., 2006, 8, 5609; Bioorg. Med. Chem. Lett. 2004, 14, 1221; Tetrahedron Lett. 2004, 45, 2311; J. Am. Chem. Soc. 2001, 123, 7727.

Cyclic carbamates (oxazolidinones) of the formula (VIII) are either commercially available or can be prepared by methods known from the literature from commercially available precursors. Oxazolidinones of the formula (VIII) can be prepared, for example, from amino alcohol derivatives, open-chain carbamates, epoxides or aziridines (Review: Chem. Rev. 1996, 96, 835; for individual examples see also: Synthesis 2007, 3111; J. Org. Chem. 2006, 71, 5023; WO 2005/033095 A1, J. An. Chem. 1989, 111, 2211 and the references cited therein).

Synthesis of lactams and thiolactams of type (Ib):
(l) the reaction of compounds of the formula (V) with lactam- or thiolactam-substituted anilines of the formula (IVb), if appropriate in the presence of an acid, if appropriate in the presence of a solvent, according to the reaction scheme below (Scheme 11):

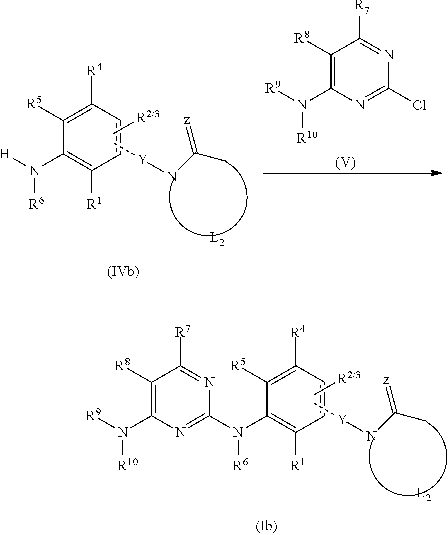

(m) the reaction of compounds of the formula (Ib-I) in the presence of suitable reagents, if appropriate in the presence of a solvent, to give the corresponding thioderivatives of type (Ib-II), according to the reaction scheme below (Scheme 12):

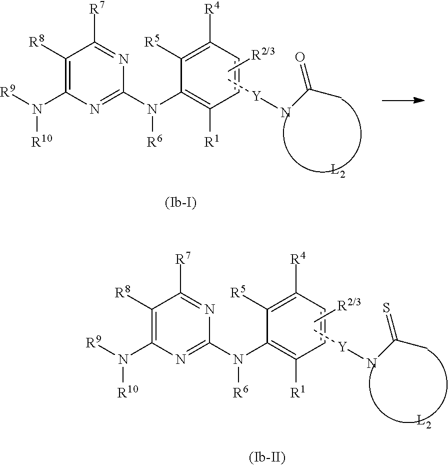

(n) the reaction of halogen-substituted compounds of the formula (VII) with lactams of the formula (XI) in the presence of a solvent and in the presence of a catalyst, according to the reaction scheme below (Scheme 13):

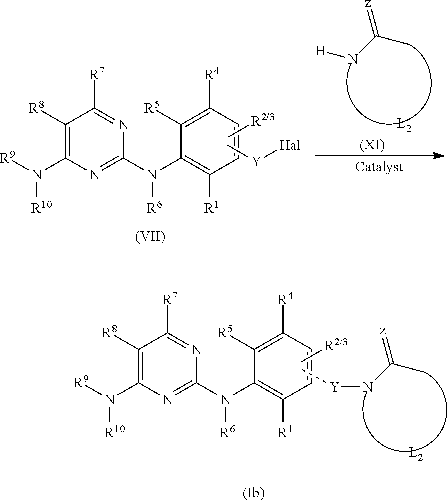

(o) the reaction of compounds of the formula (Ib-III) in the presence of suitable reagents, if appropriate in the presence of a solvent, to give the corresponding derivatives of type (Ib), according to the reaction scheme below (Scheme 14):

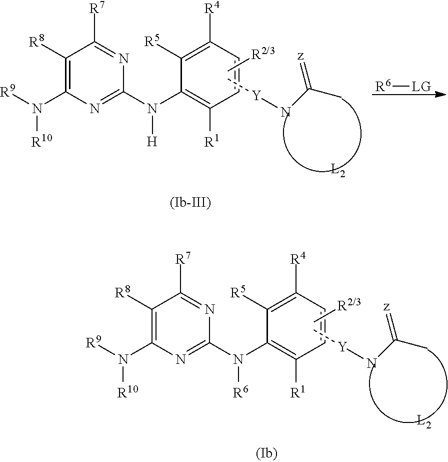

(p) the reaction of iodo- or bromo-substituted compounds (VII) with lactam-substituted compounds of the formula (XI) in the presence of suitable reagents, if appropriate in the presence of a solvent, to give the corresponding carbonyl derivatives of type (Ib-IV), according to the reaction scheme below (Scheme 15):

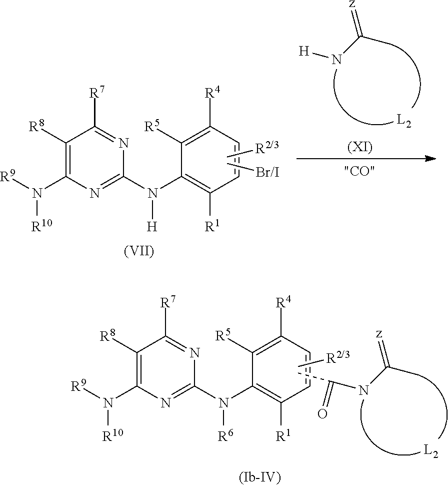

The lactam-substituted aromatic amines (IVb) are either commercially available, can be prepared by methods known from the literature from commercially available precursors or are described below:
(q) the reduction of lactam-substituted nitro compounds of the formula (XII) in the presence of suitable reagents, if appropriate in the presence of a solvent, according to the reaction scheme below (Scheme 16):

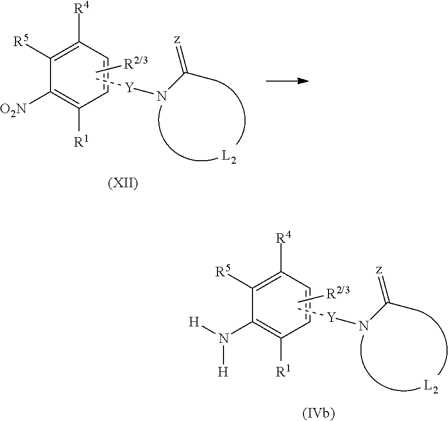

(r) the reaction of iodo- or bromo-substituted anilines (X) with lactams of the formula (XI) in the presence of suitable reagents, if appropriate in the presence of a solvent, according to the reaction scheme below (Scheme 17):

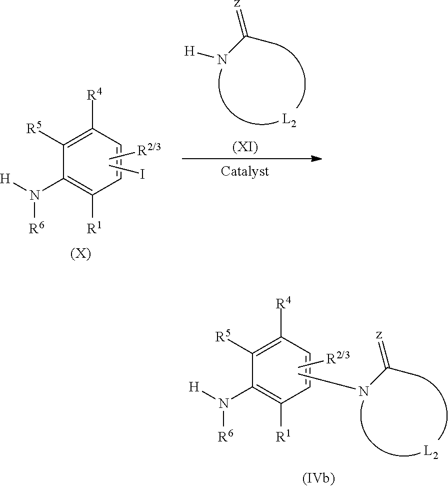

(s) the reaction of nitro compounds of the formula (XIII) with lactams of the formula (XI) in the presence of suitable reagents, if appropriate in the presence of a solvent, and according to the reaction scheme below (Scheme 18). The nitro compounds of the formula (XII) can then be reduced as already mentioned above (see Scheme 16).

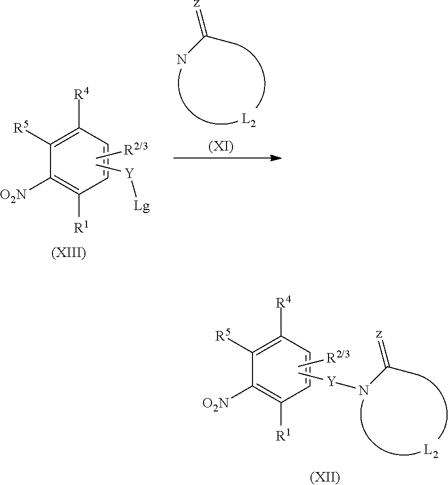

(t) the reaction of compounds of the formula (XIV) with nitro compounds of the formula (XV) in the presence of suitable reagents, if appropriate in the presence of a solvent, to give nitro esters of type (XVI), and subsequent reductive cyclization to lactams of the formula (XIa) (Scheme 19). The definitions of the R-groups in (XIV) and (XVI) correspond to the respective definitions for L₂ in (XI).

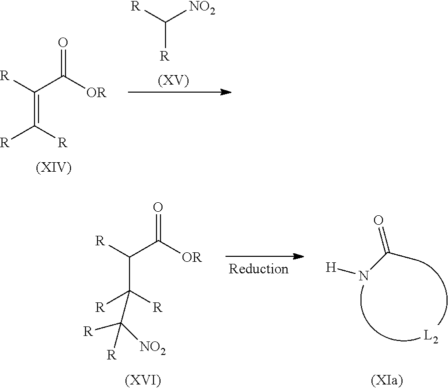

One way of preparing the compound (Ib) is shown in Scheme 11.

The intermediate (V) is reacted in the presence of Brønsted acids, such as, for example, anhydrous hydrochloric acid, camphorsulphonic acid or p-toluenesulphonic acid, in a suitable solvent, such as, for example, dioxane, THF, DMSO, DME, 2-methoxyethanol, n-butanol or acetonitrile, at a temperature of 0° C.-140° C. over a period of 1-48 h with an aromatic amine (IVb). Analogously described, for example, in Bioorg. Med. Chem. Lett. 2006, 16, 2689; GB2002 A1-2369359, Org. Lett. 2005, 7, 4113.

Alternatively, the reaction of (V) and (IVb) to give (Ib) can also be carried out with base catalysis, i.e. using, for example, carbonates, such as potassium carbonates, alkoxides, such as potassium tert-butoxide, or hydrides, such as sodium hydride, where the catalytic use of a transition metal, such as, for example, palladium, together with a suitable ligand, such as, for example, xanthphos, may also be useful.

Finally, it is possible to carry out the reaction of (V) and (IVb) to give (Ib) in the absence of solvents and/or Brønsted acids (described, for example, in Bioorg. Med. Chem. Lett. 2006, 16, 108; Bioorg. Med. Chem. Lett. 2005, 15, 3881).

A further way of preparing the compound (Ib) is shown in Scheme 12.

Thiolactams of the formula (Ib-II) can be prepared, for example, by sulphurization of lactams of the formula (Ib-I) in the presence of suitable reagents, such as, for example, Lawesson reagent. This reaction can be carried out in various solvents, for example toluene, xylene, THF or pyridine, and at various temperatures, also under microwave conditions (see Synthesis 2006, 2327; Eur. J. Org. Chem 2005, 505; Synthesis 1994, 993).

There are many descriptions of the synthesis of thiolactames from the corresponding lactams in the literature, and it is possible to use P₄S₁₀, boron sulphide, ethylaluminium sulphide or similar reagents (see J. Org. Chem 2003, 68, 5792; Tetrahedron Lett. 2001, 57, 9635).

A further way of preparing the compound (Ib) is shown in Scheme 13.

The products of the formula (Ib) can be prepared by a copper-catalysed cross coupling between oxazolidinones (XI) and aryl halides (VII) in the presence of a source of copper, a ligand and a base in various solvents and at various temperatures. Various sources of copper can be used, usually CuI, CuSO₄, Cu powder. Numerous ligands, such as, for example, 1,2-diaminocyclohexane or MeNHCH₂CH₂NHMMe can be employed. Suitable bases are, for example, K₂CO₃, K₃PO₄, Cs₂CO₃. These reactions can also be carried out under microwave conditions. For general reviews, see: Chem. Rev. 2006, 106, 2651; Synlett 2003, 2428 and the references cited. For specific examples, see: Org. Lett. 2003, 5, 963; J. Am. Chem. Soc. 2007, 129, 3490; Org. Lett., 2006, 8, 5609; Bioorg. Med. Chem. Lett. 2004, 14, 1221; Tetrahedron Lett. 2004, 45, 2311; J. Am. Chem. Soc. 2001, 123, 7727.

Aminations of this type can also be carried out using other catalyst systems based on Pd or iron (Review, see: Chem. Rev. 2006, 106, 2651; for individual examples, see: Angew. Chem. Int. Ed. 2007, 46, 8862; Angew. Chem. Int. Ed. 2007, 46, 934; J. Am. Chem. Soc. 2002, 124, 6043).

A further way of preparing the compound (Ib) is shown in Scheme 14.

If R⁶ represents hydrogen, the anilinopyrimidines of type (Ib-III) can be protected at the anile-NH using suitable reagents. Thus, for example, it is possible to benzylate with various substituted benzyl halides in the presence of a base in various solvents and at various temperatures (see WO 07/073,117). The methylation in this location succeeds, for example, with methyl iodide and sodium hydride as base, as described, for example, in WO 05/005438; Chem. Pharm. Bull. 2000, 48, 1504; or J. Med. Chem. 1993, 36, 1993, in various solvents and at a various temperatures. Carbamate protections in such systems are usually carried out using BOC₂O, if appropriate a suitable catalyst, such as DMAP, if appropriate a base, in various solvents and at various temperatures (see, for example, WO 04/087698).

A further way of preparing the compound (Ib) is shown in Scheme 15.

The anilinopyrimidines of type (Ib-IV) can be prepared under microwave conditions, by Pd-catalysed aminocarbonylation of the halogen-substituted compounds of type (VII) and the corresponding cyclic lactams (XI) in various solvents, such as, for example, THF or water, at various temperatures (60-200° C.). This reaction can be carried out using Mo(CO)₆ as a source of carbon monoxide, a base, such as DBU, and a source of Pd, such as, for example, Pd(OAc)₂, in the presence or absence of suitable ligands such as, for example, dppf or PPh₃ (see: Tetrahedron Lett. 2007, 48, 2339; Tetrahedron 2006, 62, 4671; Organometallics 2006, 25, 1434).

However, the aminocarbonylation can also be carried out under classic thermal conditions, without microwave support. It is also possible to use carbon monoxide and other sources of carbon monoxide, such as DMF. Instead of Pd, it is also possible to use nickel. (See: J. Org. Chem. 2002, 67, 6232; Angew. Chem. Int. Ed. 2007, 46, 8460; Org. Lett. 2007, 9, 4615).

One way of preparing the compound (IVb) is shown in Scheme 16.

The nitro compounds of type (XII) can be reduced by various methods to the corresponding anilines of type (IVb). The reduction can be carried out, for example, by catalytic hydrogenation using Pd/C, PtO₂, Raney Ni and hydrogen, or using Pd/C and NH₄HCO₂, in various solvents, such as, for example, MeOH, EtOH, THF or dioxane (see: Bioorg. Med. Chem. Lett. 2006, 16, 3430; US 2005/0049286 A1; J. Med. Chem. 1991, 34, 2954; J. Org. Chem. 1990, 55, 3195).

The reduction can also be carried out using metals, such as Zn, Sn or iron, in the presence of acids, such as AcOH, HCl. Moreover, it is also possible to use other reducing agents, such as SnCl₂ or TiCl₃ (see: J. Am. Chem. Soc. 2006, 128, 1162, Bioorg. Med. Chem. Lett. 2004, 14, 2905; J. Med. Chem. 1989, 32, 1612).

One way of preparing the compound (IVb) is shown in Scheme 17.

The products of the formula (IVb) can be prepared via a copper-catalysed cross coupling between lactams (XI) and halogenated anilines of type (X) in the presence of a source of copper, a ligand and a base, in various solvents and at various temperatures. Various sources of copper can be used, usually CuI, CuSO₄, Cu powder. Numerous ligands, such as, for example, 1,2-diaminocyclohexane or MeNHCH₂CH₂NHMMe, can be employed. Suitable for use as bases are, for example, K₂CO₃, K₃PO₄, Cs₂CO₃. These reactions can also be carried out under microwave conditions. For general reviews, see: Chem. Rev. 2006, 106, 2651; Synlett 2003, 2428 and the references cited. For specific examples, see: Org. Lett. 2003, 5, 963; J. Am. Chem. Soc. 2007, 129, 3490; Org. Lett., 2006, 8, 5609; Bioorg. Med. Chem. Lett. 2004, 14, 1221; Tetrahedron Lett. 2004, 45, 2311; J. Am. Chem. Soc. 2001, 123, 7727.

The halogenated anilines of type (X) are either commercially available or can be obtained by processes known from the literature from commercially available precursors.

One way of preparing the compound (XII) is shown in Scheme 18.

Compounds having a suitable leaving group of type (XIII) and lactams of type (XI) can be reacted in the presence of a base, such as NaH, to give compounds of type (XII) (see Eur. J. Med. Chem. 1991, 26, 415; Bioorg. Med. Chem. Lett. 2006, 16, 3430).

The cyclic lactams of type (XI) are either commercially available or can be prepared by methods known from the literature, such as, for example, Beckmann rearrangement of aldoximes or ketoximes, intramolecular cyclization of amino acids or amino esters (see Tetrahedron Lett. 1980, 21, 243; J. Med. Chem. 1996, 39, 1898), intramolecular cyclization catalysed by metals, such as, for example, Pd (J. Org. Chem. 2000, 65, 6249), intramolecular free-radical cyclization (J. Org. Chem. 1998, 63, 804), aminolysis of cyclic esters (WO 2007/127688 A2; WO 2005/113504 A1; J. Org. Chem. 1991, 56, 5982). One way of synthesizing lactam-substituted nitro compounds of type (XIIa) is the reaction of corresponding nitroanilines of type (XXI) with lactones of type (XIa), for example in hydrochloric acid (see Indian J. Chem Section B 1986, 25B, 395):

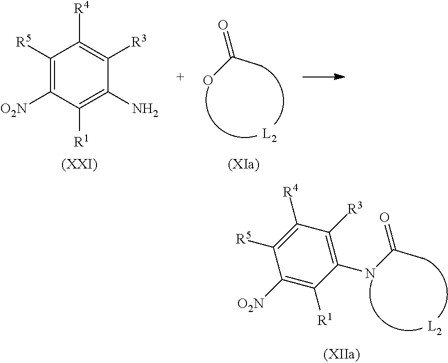

A further way for preparing the cyclic lactams of type (XIa) is shown in Scheme 19.

Nitro compounds of type (XV) can be reacted in the presence of a base, such as, for example, K₂CO₃ or NaH, by a Michael addition to α,β-unsaturated carbonyl compounds of type (XIV), to give the corresponding adducts of type (XVI). The nitro esters of type (XVI) can be reduced by numerous methods (see Scheme 16), resulting in their spontaneous cyclization to the corresponding lactams (XI).

The nitro compounds of type (XV) are either commercially available or can be prepacked by methods known from the literature (see: Org. React. 1962, 12, 101; J. Org. Chem. 2006, 71, 4585; J. Org. Chem 1989, 54, 5783).

Synthesis of oxo-substituted lactams and thiolactams of type (Ic):
(u) the reaction of compounds of the formula (V) with oxo-substituted lactams or thiolactams of the formula (IVc), if appropriate in the presence of an acid, if appropriate in the presence of a solvent, according to the reaction scheme below (Scheme 20):

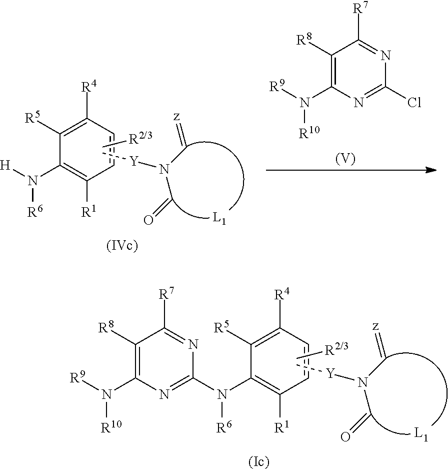

The amines (IVc) substituted by oxo-substituted cyclic carbamates are either commercially available, can be prepared by methods known from the literature from commercially available precursors or are described below.
(v) the reaction of nitrophenyl-substituted amines of the formula (XVII) with cyclic anhydrides of the formula (XVIII) in the presence of suitable reagents, if appropriate in the presence of a solvent, according to the reaction scheme below (Scheme 21):

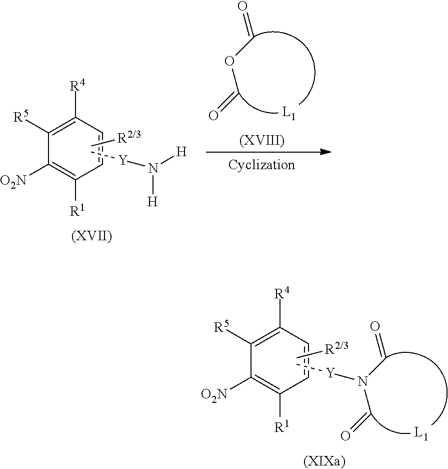

(w) the reduction of nitrophenyl-substituted oxo-substituted cyclic carbamates of the formula (XIX) in the presence of suitable reagents, if appropriate in the presence of a solvent, according to the reaction scheme below (Scheme 22):

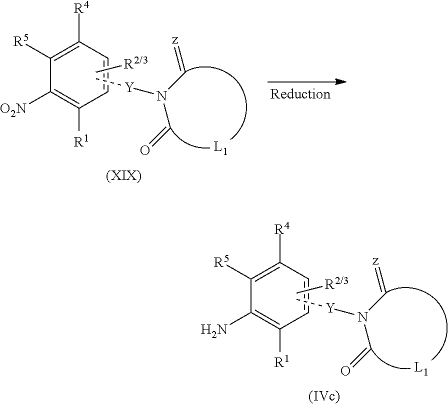

(x) the preparation of the thio derivatives of the formula (XIX) from the corresponding succinimide precursors (XIX), according to the reaction scheme below (Scheme 23):

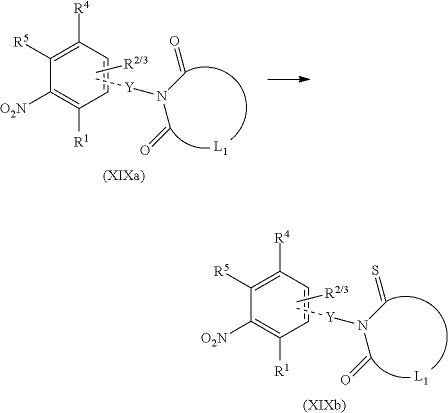

One way of preparing the compound (Ic) is shown in Scheme 20.

The intermediate (V) is reacted in the presence of Brønsted acids, such as, for example, anhydrous hydrochloric acid, camphorsulphonic acid or p-toluenesulphonic acid, in a suitable solvent, such as, for example, dioxane, THF, DMSO, DME, 2-methoxyethanol, n-butanol or acetonitrile, at a temperature of 0° C.-140° C. over a period of 1-48 h with an aromatic amine (IVc). Analogously described, for example, in Bioorg. Med. Chem. Lett. 2006, 16, 2689; GB2002 A1-2369359, Org. Lett. 2005, 7, 4113.

Alternatively, the reaction of (V) and (IVc) to give (Ic) can also be carried out with base catalysis, i.e. using, for example, carbonates, such as potassium carbonates, alkoxides, such as potassium tert-butoxide, or hydrides, such as sodium hydride, where the catalytic use of a transition metal, such as, for example, palladium, together with a suitable ligand, such as, for example, xanthphos, may also be useful.

Finally, it is possible to carry out the reaction of (V) and (IVc) to give (Ic) in the absence of solvents and/or Brønsted acids (described, for example, in Bioorg. Med. Chem. Lett. 2006, 16, 108; Bioorg. Med. Chem. Lett. 2005, 15, 3881).

One way of preparing the compound (XIXa) is shown in Scheme 21.

The reaction of appropriate nitroanilines of type (XVII) with cyclic anhydrides (XVIII) leads to open-chain amides. These open-chain amides cyclize spontaneously or in the presence of a weak base, such as, for example, NaOAc, to give the desired succinimides of type (XIXa). This reaction can be carried out in various solvents, such as toluene or dioxane (see: Eur. J. Med. Chem. 2007, 42, 10; Synth. Com. 2005, 35, 2017).

One way of preparing the compound (IVc) is shown in Scheme 22.

By numerous methods (see also Scheme 16), the nitro compounds of type (XIX) can be reduced to the corresponding anilines of type (IVc).

Alternatively, the nitro compounds of type (XIX) can also be prepared by Cu-catalysed couplings between nitroanilines and the appropriate succinimides (see Synthesis 2006, 1868).

One way of preparing the compound (XIXb) is shown in Scheme 23.

Oxo-substituted thiolactams of the formula (XIXb) can be prepared, for example, by sulphurization of oxo-substituted lactams of the formula (XIXa) in the presence of suitable reagents, such as, for example, Lawesson reagent. This reaction can be carried out in various solvents, for example toluene, xylene, THF or pyridine, and at various temperatures, including under microwave conditions (see Synthesis 1996, 1485).

In general, it also possible to choose other routes for preparing the compounds (I) according to the invention. Some are shown in an exemplary manner in Scheme 24:

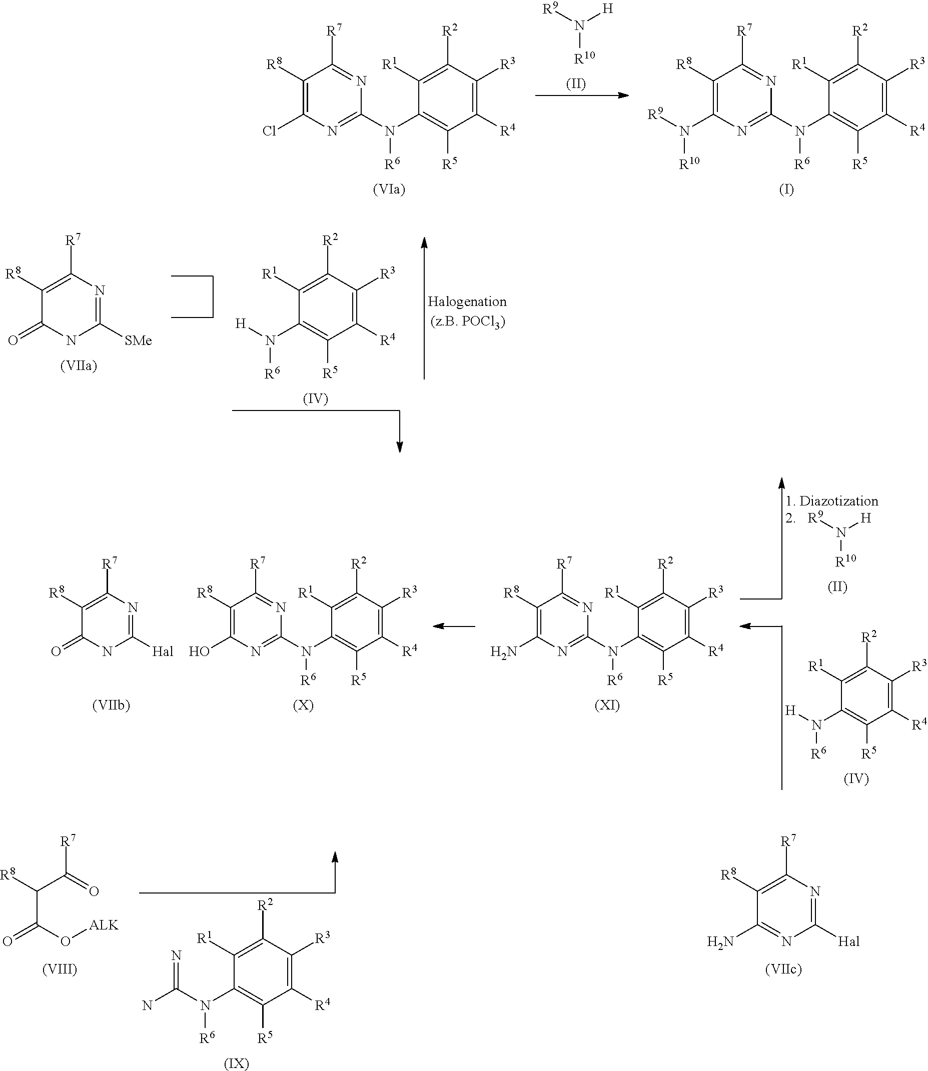

In general, compounds of the formula (I) can also be prepared, for example, by sequential nucleophilic addition of an aliphatic amine (II) and a (hetero)aromatic amine (IV) to a suitable substituted pyrimidine (III), as outlined below in Scheme 25:

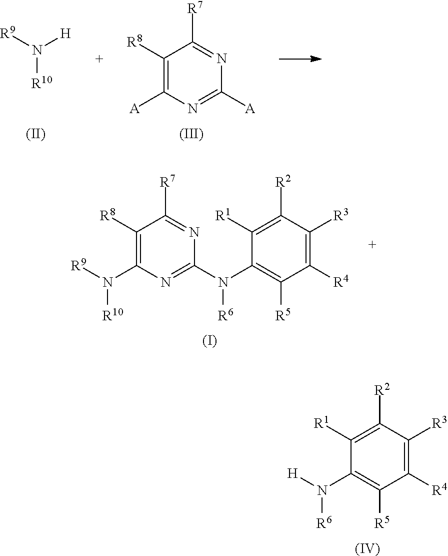

Here, A, in each case independently of one another, represent suitable leaving groups, for example a halogen atom (F, Cl, Br, I), SMe, SO₂Me, SOMe or else triflate (CF₃SO₂O: for pyrimidines known from WO2005095386).

The synthesis of diaminopyrimidines of the formula (I) according to Scheme 25 or else by other routes has been described in the literature in many different instances (see, for example, also WO 07/140,957, WO 06/021544, WO 07/072,158, WO 07/003,596, WO 05/016893, WO 05/013996, WO 04/056807, WO 04/014382, WO 03/030909).

Another important method, many instances of which have been described, for synthesizing lactam-substituted aromatics which may also be mentioned is the route depicted in Scheme 26 below (see, for example, WO 05/079791 A1):

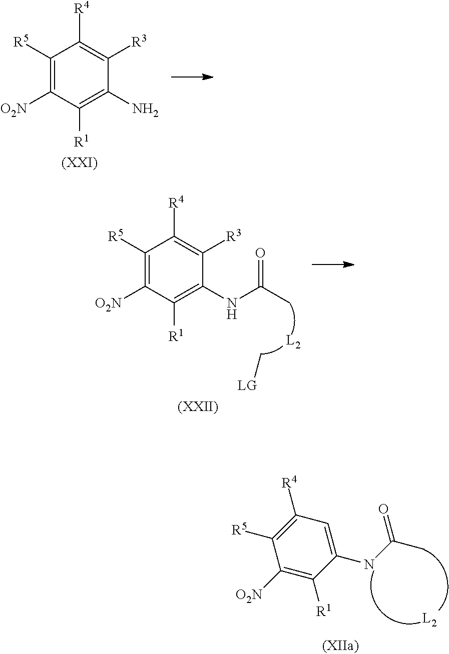

The processes according to the invention for preparing the compounds of the formulae (I), (Ia), (Ib) and (Ic) are preferably carried out using one or more reaction auxiliaries.

Suitable reaction auxiliaries are, if appropriate, the customary inorganic or organic bases or acid acceptors. These preferably include alkali metal and alkaline earth metal acetates, amides, carbonates, bicarbonates, hydrides, hydroxides, and alkoxides, such as, for example, sodium acetate, potassium acetate or calcium acetate, lithium amide, sodium amide, potassium amide or calcium amide, sodium carbonate, potassium carbonate or calcium carbonate, sodium bicarbonate, potassium bicarbonate, or calcium bicarbonate, lithium hydride, sodium hydride, potassium hydride or calcium hydride, lithium hydroxide, sodium hydroxide, potassium hydroxide or calcium hydroxide, sodium methoxide, ethoxide, n- or i-propoxide, n-, s- or t-butoxide or potassium methoxide, ethoxide, n- or i-propoxide, n-, s- or t-butoxide; furthermore also basic organic nitrogen compounds, such as, for example, trimethylamine, triethylamine, tripropylamine, tributylamine, ethyldiisopropylamine, N,N-dimethylcyclohexylamine, dicyclohexylamine, ethyldicyclohexylamine, N,N-dimethylaniline, N,N-dimethylbenzylamine, pyridine, 2-methyl-, 3-methyl-, 4-methyl-, 2,4-dimethyl-, 2,6-dimethyl-, 3,4-dimethyl- and 3,5-dimethylpyridine, 5-ethyl-2-methylpyridine, 4-dimethylaminopyridine, N-methylpiperidine, 1,4-diazabicyclo[2.2.2]-octane (DABCO), 1,5-diazabicyclo[4.3.0]non-5-ene (DBN) or 1,8-diazabicyclo[5.4.0]undec-7-ene (DBU).

The processes according to the invention are preferably carried out using one or more diluents. Suitable diluents are virtually all inert organic solvents. These preferably include aliphatic and aromatic, unbranched or halogenated hydrocarbons, such as pentane, hexane, heptane, cyclohexane, petroleum ether, benzine, ligroin, benzene, toluene, xylene, methylene chloride, ethylene chloride, chloroform, carbon tetrachloride, chlorobenzene and o-dichlorobenzene, ethers, such as diethyl ether and dibutyl ether, glycol dimethyl ether and diglycol dimethyl ether, tetrahydrofuran and dioxane, ketones, such as acetone, methyl ethyl ketone, methyl isopropyl ketone or methyl isobutyl ketone, esters, such as methyl acetate or ethyl acetate, nitriles, such as, for example, acetonitrile or propionitrile, amides, such as, for example, dimethylformamide, dimethylacetamide and N-methylpyrrolidone, and also dimethyl sulphoxide, tetramethylene sulphone and hexamethylphosphoric triamide and DMPU.

In the processes according to the invention, the reaction temperatures can be varied within a relatively wide range. In general, the processes are carried out at temperatures between 0° C. and 250° C., preferably at temperatures between 10° C. and 185° C.

The processes according to the invention are generally carried out under atmospheric pressure. However, it is also possible to operate under elevated or reduced pressure.

For carrying out the processes according to the invention, the starting materials required in each case are generally employed in approximately equimolar amounts. However, it is also possible to use in each case one of the components employed in a relatively large excess. Work-up in the processes according to the invention is in each case carried out by customary methods (cf. the Preparation Examples).

Some of the compounds of the formula (V) are novel and thus also form part of the subject-matter of the present invention.

Novel are compounds of the formula (V)

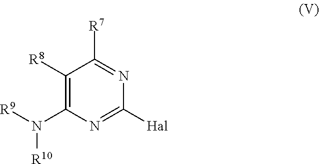

in which
R⁷ represents hydrogen,
   and, if
R⁸ represents CF₃, CFH₂ or CF₂H,
Hal represents F, Cl, Br or I,
R⁹ represents hydrogen, ethyl, propyl, propan-2-yl, 2-methoxyethan-1-yl, prop-2-en-1-yl, CH₂OCH₃, COMe, COOMe, COOEt, COOtertBu, COCF₃ or benzyl,
R¹⁰ represents ethyl, propyl, cyclopropyl, cyclopropylmethyl, 1-cyclopropyleth-1-yl, 2-methylcyclopropyl, 2,2-dimethylcyclopropyl, 2,2-dimethylprop-1-yl, tert-butyl, cyclobutyl, 2-methylcyclobut-1-yl, 3-methylcyclobut-1-yl, butyl, 3-methylbut-1-yl, 2-methylbut-1-yl, 2-methylprop-1-yl, 1-fluoroprop-2-yl, cyclopentyl, propan-2-yl, pentan-3-yl, pentan-2-yl, pentyl, prop-2-en-1-yl, butan-2-yl, 2,2,2-trifluoroethyl, 2,2-difluoroethyl, 2-methoxyethan-1-yl, 2-methylmercaptoethan-1-yl, 2-fluoroethan-1-yl, 2-chloroethan-1-yl, 2-cyanoethan-1-yl, 1-methoxypropan-2-yl, 3-methoxypropan-1-yl, 1-methylmercaptopropan-2-yl, 2-methyl-1-(methylsulphanyl)propan-2-yl, oxetan-3-yl, 1,1,1-trifluoropropan-2-yl, 2,2,3,3,3-pentafluoropropyl, 1,1,1-trifluoropropan-3-yl, 1,1,1-trifluorobutan-2-yl, 1,1,1-trifluorobutan-3-yl, 2-methylprop-2-en-1-yl or 1-fluoropropan-2-yl,
   or
R⁹ and R¹⁰ together with the nitrogen atom to which they are attached form an unsubstituted or substituted 3- to 7-membered saturated cycle which may contain up to one further heteroatom,
   where the substituents independently of one another are selected from the group consisting of methyl, fluorine, chlorine and/or bromine atoms, cyano, hydroxyl, methoxy, CF₃,
   and where the heteroatoms are selected from the group consisting of oxygen, sulphur and nitrogen.

Novel are compounds of the formula (V)

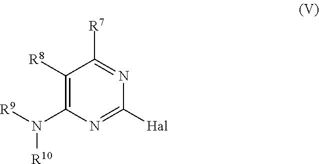

in which
R⁷ represents hydrogen,
   and, if
R⁸ represents iodine,
Hal represents F, Cl, Br or I,
R⁹ represents hydrogen, methyl, ethyl, propyl, propan-2-yl, 2-methoxyethan-1-yl, prop-2-en-1-yl, CH₂OCH₃, COMe, COOMe, COOEt, COOtertBu, COCF₃ or benzyl,
R¹⁰ represents methyl, ethyl, propyl, cyclopropyl, cyclopropylmethyl, 1-cyclopropyleth-1-yl, 2-methylcyclopropyl, 2,2-dimethylcyclopropyl, 2,2-dimethylprop-1-yl, tert-butyl, cyclobutyl, 2-methylcyclobut-1-yl, 3-methylcyclobut-1-yl, butyl, 3-methylbut-1-yl, 2-methylbut-1-yl, 2-methylprop-1-yl, 1-fluoroprop-2-yl, cyclopentyl, propan-2-yl, pentan-3-yl, pentan-2-yl, pentyl, prop-2-en-1-yl, butan-2-yl, 2,2,2-trifluoroethyl, 2,2-difluoroethyl, 2-methoxyethan-1-yl, 2-methylmercaptoethan-1-yl, 2-fluoroethan-1-yl, 2-chloroethan-1-yl, 2-cyanoethan-1-yl, 1-methoxypropan-2-yl, 3-methoxypropan-1-yl, 1-methylmercaptopropan-2-yl, 2-methyl-1-(methylsulphanyl)propan-2-yl, oxetan-3-yl, 1,1,1-trifluoropropan-2-yl, 2,2,3,3,3-pentafluoropropyl, 1,1,1-trifluoropropan-3-yl, 1,1,1-trifluorobutan-2-yl, 1,1,1-trifluorobutan-3-yl, 2-methylprop-2-en-1-yl or 1-fluoropropan-2-yl,
   or
R⁹ and R¹⁰ together with the nitrogen atom to which they are attached form an unsubstituted or substituted 3- to 7-membered saturated cycle which may contain up to one further heteroatom,
   where the substituents independently of one another are selected from the group consisting of methyl, fluorine, chlorine and/or bromine atoms, cyano, hydroxyl, methoxy, CF₃,
   and where the heteroatoms are selected from the group consisting of oxygen, sulphur and nitrogen.

Novel are compounds of the formula (V)

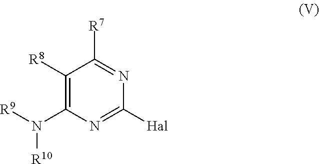

in which
R⁷ represents hydrogen,
   and, if
R⁸ represents SMe, SOMe or SO₂Me,
Hal represents F, Cl, Br or I,
R⁹ represents hydrogen, methyl, ethyl, propyl, propan-2-yl, 2-methoxyethan-1-yl, prop-2-en-1-yl, CH₂OCH₃, COMe, COOMe, COOEt, COOtertBu, COCF₃ or benzyl,
R¹⁰ represents ethyl, propyl, cyclopropyl, cyclopropylmethyl, 1-cyclopropyleth-1-yl, 2-methylcyclopropyl, 2,2-dimethylcyclopropyl, 2,2-dimethylprop-1-yl, tert-butyl, cyclobutyl, 2-methylcyclobut-1-yl, 3-methylcyclobut-1-yl, butyl, 3-methylbut-1-yl, 2-methylbut-1-yl, 2-methylprop-1-yl, 1-fluoroprop-2-yl, cyclopentyl, propan-2-yl, pentan-3-yl, pentan-2-yl, pentyl, prop-2-en-1-yl, butan-2-yl, 2,2,2-trifluoroethyl, 2,2-difluoroethyl, 2-methoxyethan-1-yl, 2-methylmercaptoethan-1-yl, 2-fluoroethan-1-yl, 2-chloroethan-1-yl, 2-cyanoethan-1-yl, 1-methoxypropan-2-yl, 3-methoxypropan-1-yl, 1-methylmercaptopropan-2-yl, 2-methyl-1-(methylsulphanyl)propan-2-yl, oxetan-3-yl, 1,1,1-trifluoropropan-2-yl, 2,2,3,3,3-pentafluoropropyl, 1,1,1-trifluoropropan-3-yl, 1,1,1-trifluorobutan-2-yl, 1,1,1-trifluorobutan-3-yl, 2-methylprop-2-en-1-yl or 1-fluoropropan-2-yl,
   or
R⁹ and R¹⁰ together with the nitrogen atom to which they are attached form an unsubstituted or substituted 3- to 7-membered saturated cycle which may contain up to one further heteroatom,
   where the substituents independently of one another are selected from the group consisting of methyl, fluorine, chlorine and/or bromine atoms, cyano, hydroxyl, methoxy, CF₃,
   and where the heteroatoms are selected from the group consisting of oxygen, sulphur and nitrogen.

Novel are compounds of the formula (V)

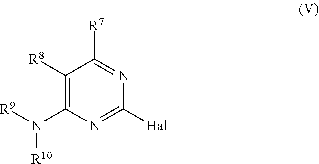

in which
R⁷ represents hydrogen,
   and, if
R⁸ represents cyano,
Hal represents F, Cl, Br or I,
R⁹ represents hydrogen, methyl, propyl, propan-2-yl, 2-methoxyethan-1-yl, prop-2-en-1-yl, CH₂OCH₃, COMe, COOMe, COOEt, COOtertBu, COCF₃, or benzyl,
R¹⁰ represents propyl, cyclopropylmethyl, 1-cyclopropyleth-1-yl, 2-methylcyclopropyl, 2,2-dimethylcyclopropyl, 2,2-dimethylprop-1-yl, tert-butyl, cyclobutyl, 2-methylcyclobut-1-yl, 3-methylcyclobut-1-yl, 3-methylbut-1-yl, 2-methylbut-1-yl, 1-fluoroprop-2-yl, cyclopentyl, propan-2-yl, pentan-3-yl, pentan-2-yl, pentyl, butan-2-yl, 2,2-difluoroethyl, 2-methylmercaptoethan-1-yl, 2-chloroethan-1-yl, 1-methoxypropan-2-yl, 3-methoxypropan-1-yl, 1-methylmercaptopropan-2-yl, 2-methyl-1-(methylsulphanyl)propan-2-yl, oxetan-3-yl, 1,1,1-trifluoropropan-2-yl, 2,2,3,3,3-pentafluoropropyl, 1,1,1-trifluoropropan-3-yl, 1,1,1-trifluorobutan-2-yl, 1,1,1-trifluorobutan-3-yl, 2-methylprop-2-en-1 or 1-fluoropropan-2-yl.

Novel are compounds of the formula (V)

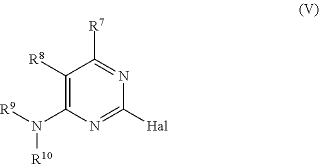

in which
R⁷ represents hydrogen,
   and, if
R⁸ represents Br, Cl or CF₃,
Hal represents Cl,
R⁹ represents hydrogen,
R¹⁰ represents propyl, 2-methylcyclopropyl, 3-methylcyclobutyl, 2-ethylcyclopropyl, such as, for example:

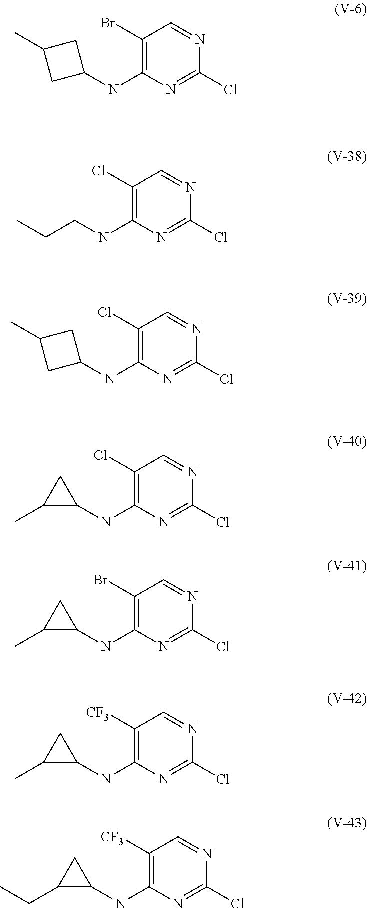

Some of the compounds of the formula (VI) are novel and thus also form part of the subject-matter of the present invention.

Novel are compounds of the formula (VI)

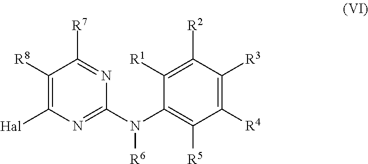

in which the symbols are as defined below:
R¹ to R⁸ have the general, preferred, particularly preferred, very particularly preferred and especially preferred meanings given above and
   Hal represents fluorine, chlorine, bromine or iodine.

Some of the compounds of the formula (VII) are novel and thus also form part of the subject-matter of the present invention.

Novel are compounds of the formula (VIIa),

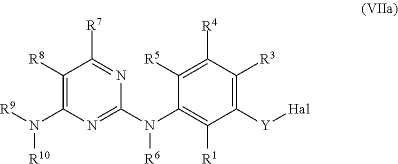

in which the symbols are as defined below:
Y represents a direct bond,
Hal represents bromine or iodine,
R¹ and R⁵ represent hydrogen,
R³ and R⁶ to R¹⁰ have the general, preferred, particularly preferred, very particularly preferred and especially preferred meanings given above.

Novel are compounds of the formula (VIIb)

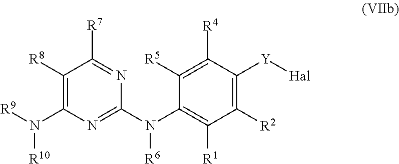

in which the symbols are as defined below:
Y is a direct bond,
Hal represents bromine or iodine,
R¹ and R⁵ represent hydrogen,
R² and R⁶ to R¹⁰ have the general, preferred, particularly preferred, very particularly preferred and especially preferred meanings given above.

The invention furthermore provides the non-medicinal use of the diaminopyrimidines according to the invention or of mixtures of these for controlling unwanted microorganisms.

The invention furthermore provides a composition for controlling unwanted microorganisms, comprising at least one diaminopyrimidine according to the present invention.

Moreover, the invention relates to a method for controlling unwanted microorganisms, characterized in that the diaminopyrimidines according to the invention are applied to the microorganisms and/or their habitat.

The compounds according to the invention have strong microbicidal action and can be used for controlling unwanted microorganisms, such as fungi and bacteria, in crop protection and in the protection of materials.

The diaminopyrimidines of the formula (I) according to the invention have very good fungicidal properties and can be used in crop protection, for example for controlling Plasmodiophoromycetes, Oomycetes, Chytridiomycetes, Zygomycetes, Ascomycetes, Basidiomycetes and Deuteromycetes.

In crop protection, bactericides can be used for controlling Pseudomonadaceae, Rhizobiaceae, Enterobacteriaceae, Corynebacteriaceae and Streptomycetaceae.

The fungicidal compositions according to the invention can be used for the curative or protective control of phytopathogenic fungi. Accordingly, the invention also relates to curative and protective methods for controlling phytopathogenic fungi using the active compounds or compositions according to the invention, which are applied to the seed, the plant or plant parts, the fruit or the soil on which the plants grow.

The compositions according to the invention for controlling phytopathogenic fungi in crop protection comprise an effective, but non-phytotoxic amount of the active compounds according to the invention. “Effective, but non-phytotoxic amount” means an amount of the composition according to the invention which is sufficient to control the fungal disease of the plant in a satisfactory manner or to eradicate the fungal disease completely, and which, at the same time, does not cause any significant symptoms of phytotoxicity. In general, this application rate may vary within a relatively wide range. It depends on a plurality of factors, for example on the fungus to be controlled, the plant, the climatic conditions and the ingredients of the compositions according to the invention.

According to the invention, it is possible to treat all plants and parts of plants. Plants are to be understood here as meaning all plants and plant populations, such as wanted and unwanted wild plants or crop plants (including naturally occurring crop plants). Crop plants can be plants which can be obtained by conventional breeding and optimization methods or by biotechnological and genetic engineering methods or combinations of these methods, including the transgenic plants and including plant cultivars which can or cannot be protected by varietal property rights. Parts of plants are to be understood as meaning all above-ground and below-ground parts and organs of the plants, such as shoot, leaf, flower and root, examples which may be mentioned being leaves, needles, stems, trunks, flowers, fruit bodies, fruits and seeds and also roots, tubers and rhizomes. Plant parts also include harvested material and vegetative and generative propagation material, for example seedlings, tubers, rhizomes, cuttings and seeds.

The following plants may be mentioned as plants which can be treated according to the invention: cotton, flax, grapevines, fruit, vegetables, such as Rosaceae sp. (for example pomaceous fruit, such as apples and pears, but also stone fruit, such as apricots, cherries, almonds and peaches and soft fruit such as strawberries), Ribesioidae sp., Juglandaceae sp., Betulaceae sp., Anacardiaceae sp., Fagaceae sp., Moraceae sp., Oleaceae sp., Actinidaceae sp., Lauraceae sp., Musaceae sp. (for example banana trees and plantations), Rubiaceae sp. (for example coffee), Theaceae sp., Sterculiceae sp., Rutaceae sp. (for example lemons, oranges and grapefruit), Solanaceae sp. (for example tomatoes), Liliaceae sp., Asteraceae sp. (for example lettuce), Umbelliferae sp., Cruciferae sp., Chenopodiaceae sp., Cucurbitaceae sp. (for example cucumbers), Alliaceae sp. (for example leek, onions), Papilionaceae sp. (for example peas); major crop plants, such Gramineae sp. (for example maize, lawn, cereals such as wheat, rye, rice, barley, oats, millet and triticale), Asteraceae sp. (for example sunflowers), Brassicaceae sp. (for example white cabbage, red cabbage, broccoli, cauliflowers, brussel sprouts, pak choi, kohlrabi, garden radish, and also oilseed rape, mustard, horseradish and cress), Fabacae sp. (for example beans, peas), Papilionaceae sp. (for example soya beans), Solanaceae sp. (for example potatoes), Chenopodiaceae sp. (for example sugarbeet, fodderbeet, swiss chard, beetroot); crop plants and ornamental plants in garden and forest; and also in each case genetically modified varieties of these plants. Preferably, cereal plants are treated according to the invention.

Some pathogens of fungal diseases which can be treated according to the invention may be mentioned by way of example, but not by way of limitation:

Diseases caused by powdery mildew pathogens, such as, for example, Blumeria species, such as, for example, Blumeria graminis; Podosphaera species, such as, for example, Podosphaera leucotricha; Sphaerotheca species, such as, for example, Sphaerotheca fuliginea; Uncinula species, such as, for example, Uncinula necator;

Diseases caused by rust disease pathogens, such as, for example, Gymnosporangium species, such as, for example, Gymnosporangium sabinae; Hemileia species, such as, for example, Hemileia vastatrix; Phakopsora species, such as, for example, Phakopsora pachyrhizi and Phakopsora meibomiae; Puccinia species, such as, for example, Puccinia recondita or Puccinia triticina; Uromyces species, such as, for example, Uromyces appendiculatus;

Diseases caused by pathogens from the group of the Oomycetes, such as, for example, Bremia species, such as, for example, Bremia lactucae; Peronospora species, such as, for example, Peronospora pisi or P. brassicae; Phytophthora species, such as, for example Phytophthora infestans; Plasmopara species, such as, for example, Plasmopara viticola; Pseudoperonospora species, such as, for example, Pseudoperonospora humuli or Pseudoperonospora cubensis; Pythium species, such as, for example, Pythium ultimum;

Leaf blotch diseases and leaf wilt diseases caused, for example, by Alternaria species, such as, for example, Alternaria solani; Cercospora species, such as, for example, Cercospora beticola; Cladiosporium species, such as, for example, Cladiosporium cucumerinum; Cochliobolus species, such as, for example, Cochliobolus sativus (conidia form: Drechslera, Syn: Helminthosporium); Colletotrichum species, such as, for example, Colletotrichum lindemuthanium; Cycloconium species, such as, for example, Cycloconium oleaginum; Diaporthe species, such as, for example, Diaporthe citri; Elsinoe species, such as, for example, Elsinoe fawcettii; Gloeosporium species, such as, for example, Gloeosporium laeticolor; Glomerella species, such as, for example, Glomerella cingulata; Guignardia species, such as, for example, Guignardia bidwelli; Leptosphaeria species, such as, for example, Leptosphaeria maculans; Magnaporthe species, such as, for example, Magnaporthe grisea; Microdochium species, such as, for example, Microdochium nivale; Mycosphaerella species, such as, for example, Mycosphaerella graminicola and M. fijiensis; Phaeosphaeria species, such as, for example, Phaeosphaeria nodorum; Pyrenophora species, such as, for example, Pyrenophora teres; Ramularia species, such as, for example, Ramularia collocygni; Rhynchosporium species, such as, for example, Rhynchosporium secalis; Septoria species, such as, for example, Septoria apii; Typhula species, such as, for example, Typhula incarnata; Venturia species, such as, for example, Venturia inaequalis;

Root and stem diseases caused, for example, by Corticium species, such as, for example, Corticium graminearum; Fusarium species, such as, for example, Fusarium oxysporum; Gaeumannomyces species, such as, for example, Gaeumannomyces graminis; Rhizoctonia species, such as, for example Rhizoctonia solani; Tapesia species, such as, for example, Tapesia acuformis; Thielaviopsis species, such as, for example, Thielaviopsis basicola;

Ear and panicle diseases (including maize cobs) caused, for example, by Alternaria species, such as, for example, Alternaria spp.; Aspergillus species, such as, for example, Aspergillus flavus; Cladosporium species, such as, for example, Cladosporium cladosporioides; Claviceps species, such as, for example, Claviceps purpurea; Fusarium species, such as, for example, Fusarium culmorum; Gibberella species, such as, for example, Gibberella zeae; Monographella species, such as, for example, Monographella nivalis; Septoria species, such as for example, Septoria nodorum;

Diseases caused by smut fungi, such as, for example, Sphacelotheca species, such as, for example, Sphacelotheca reiliana; Tilletia species, such as, for example, Tilletia caries; T. controversa; Urocystis species, such as, for example, Urocystis occulta; Ustilago species, such as, for example, Ustilago nuda; U. nuda tritici;

Fruit rot caused, for example, by Aspergillus species, such as, for example, Aspergillus flavus; Botrytis species, such as, for example, Botrytis cinerea; Penicillium species, such as, for example, Penicillium expansum and P. purpurogenum; Sclerotinia species, such as, for example, Sclerotinia sclerotiorum;

Verticilium species, such as, for example, Verticilium alboatrum;

Seed- and soil-borne rot and wilt diseases, and also diseases of seedlings, caused, for example, by Fusarium species, such as, for example, Fusarium culmorum; Phytophthora species, such as, for example, Phytophthora cactorum; Pythium species, such as, for example, Pythium ultimum; Rhizoctonia species, such as, for example, Rhizoctonia solani; Sclerotium species, such as, for example, Sclerotium rolfsii;

Cancerous diseases, galls and witches' broom caused, for example, by Nectria species, such as, for example, Nectria galligena;

Wilt diseases caused, for example, by Monilinia species, such as, for example, Monilinia laxa;

Deformations of leaves, flowers and fruits caused, for example, by Taphrina species, such as, for example, Taphrina deformans;

Degenerative diseases of woody plants caused, for example, by Esca species, such as, for example, Phaemoniella clamydospora and Phaeoacremonium aleophilum and Fomitiporia mediterranea;

Diseases of flowers and seeds caused, for example, by Botrytis species, such as, for example, Botrytis cinerea;

Diseases of plant tubers caused, for example, by Rhizoctonia species, such as, for example, Rhizoctonia solani; Helminthosporium species, such as, for example, Helminthosporium solani;

Diseases caused by bacterial pathogens, such as, for example, Xanthomonas species, such as, for example, Xanthomonas campestris pv. oryzae; Pseudomonas species, such as, for example, Pseudomonas syringae pv. lachrymans; Erwinia species, such as, for example, Erwinia amylovora.

Preference is given to controlling the following diseases of soya beans:

Fungal diseases on leaves, stems, pods and seeds caused, for example, by alternaria leaf spot (Alternaria spec. atrans tenuissima), anthracnose (Colletotrichum gloeosporoides dematium var. truncatum), brown spot (Septoria glycines), cercospora leaf spot and blight (Cercospora kikuchii), choanephora leaf blight (Choanephora infundibulifera trispora (Syn.)), dactuliophora leaf spot (Dactuliophora glycines), downy mildew (Peronospora manshurica), drechslera blight (Drechslera glycini), frogeye leaf spot (Cercospora sojina), leptosphaerulina leaf spot (Leptosphaerulina trifolii), phyllostica leaf spot (Phyllosticta sojaecola), pod and stem blight (Phomopsis sojae), powdery mildew (Microsphaera diffusa), pyrenochaeta leaf spot (Pyrenochaeta glycines), rhizoctonia aerial, foliage, and web blight (Rhizoctonia solani), rust (Phakopsora pachyrhizi Phakopsora meibomiae), scab (Sphaceloma glycines), stemphylium leaf blight (Stemphylium botryosum), target spot (Corynespora cassiicola).

Fungal diseases on roots and the stem base caused, for example, by black root rot (Calonectria crotalariae), charcoal rot (Macrophomina phaseolina), fusarium blight or wilt, root rot, and pod and collar rot (Fusarium oxysporum, Fusarium orthoceras, Fusarium semitectum, Fusarium equiseti), mycoleptodiscus root rot (Mycoleptodiscus terrestris), neocosmospora (Neocosmospora vasinfecta), pod and stem blight (Diaporthe phaseolorum), stem canker (Diaporthe phaseolorum var. caulivora), phytophthora rot (Phytophthora megasperma), brown stem rot (Phialophora gregata), pythium rot (Pythium aphanidermatum, Pythium irregulare, Pythium debaryanum, Pythium myriotylum, Pythium ultimum), rhizoctonia root rot, stem decay, and damping-off (Rhizoctonia solani), sclerotinia stem decay (Sclerotinia sclerotiorum), sclerotinia southern blight (Sclerotinia rolfsii), thielaviopsis root rot (Thielaviopsis basicola).

In the present case, undesired microorganisms are understood as meaning phytopathogenic fungi and bacteria. Thus, the substances according to the invention can be employed for protecting plants against attack by the abovementioned pathogens within a certain period of time after the treatment. The period of time within which their protection is effected is generally extended from 1 to 10 days, preferably 1 to 7 days, after the plants have been treated with the active compounds.

The fact that the active compounds, at the concentrations required for the controlling of plant diseases, are well tolerated by plants permits the treatment of above-ground plant parts, of vegetative propagation material and seed, and of the soil.

In this context, the active compounds according to the invention can be employed particularly successfully for controlling cereal diseases such as, for example, against Erysiphe species, against Puccinia and against Fusaria species, rice diseases such as, for example against Pyricularia and Rhizoctonia and diseases in viticulture, fruit production and vegetable production such as, for example, against Botrytis, Venturia, Sphaerotheca and Podosphaera species.

The active compounds according to the invention are also suitable for increasing the yield. Moreover, they display a low degree of toxicity and are well tolerated by plants.

If appropriate, the compounds according to the invention can, at certain concentrations or application rates, also be used as herbicides, safeners, growth regulators or agents to improve plant properties, or as microbicides, for example as fungicides, antimycotics, bactericides, viricides (including agents against viroids) or as agents against MLO (Mycoplasma-like organisms) and RLO (Rickettsia-like organisms). If appropriate, they can also be employed as intermediates or precursors for the synthesis of other active compounds.

At certain concentrations and application rates, the active compounds according to the invention can also be used as herbicides, for influencing plant growth and also for controlling animal pests as insecticide. If appropriate, they can also be employed as intermediates and precursors for the synthesis of further active compounds.

The active compounds according to the invention, in combination with good plant tolerance and favourable toxicity to warm-blooded animals and being tolerated well by the environment, are suitable for protecting plants and plant organs, for increasing harvest yields and for improving the quality of harvested material in agriculture, in horticulture, in animal husbandry, in forests, in gardens and leisure facilities, in the protection of stored products and of materials, and in the hygiene sector. They are preferably employed as crop protection agents. They are active against normally sensitive and resistant species and against all or some stages of development.

The treatment according to the invention of the plants and plant parts with the active compounds or compositions is carried out directly or by action on their surroundings, habitat or storage space using customary treatment methods, for example by dipping, spraying, atomizing, irrigating, evaporating, dusting, fogging, broadcasting, foaming, painting, spreading-on, watering (drenching), drip irrigating and, in the case of propagation material, in particular in the case of seeds, furthermore as a powder for dry seed treatment, a solution for wet seed treatment, a water-soluble powder for slurry treatment, by encrusting, by coating with one or more coats, etc. It is furthermore possible to apply the active compounds by the ultra-low-volume method or to inject the active compound preparation or the active compound itself into the soil.

In addition, by the treatment according to the invention it is possible to reduce the mycotoxin content in the harvested material and the foodstuffs and feedstuffs prepared therefrom. Particular, but not exclusive, mention may be made here of the following mycotoxins: deoxynivalenol (DON), nivalenol, 15-Ac-DON, 3-Ac-DON, T2- and HT2-toxin, fumonisins, zearalenone, moniliformin, fusarin, diacetoxyscirpenol (DAS), beauvericin, enniatin, fusaroproliferin, fusarenol, ochratoxins, patulin, ergot alkaloids and aflatoxins produced, for example, by the following fungi: Fusarium spec., such as Fusarium acuminatum, F. avenaceum, F. crookwellense, F. culmorum, F. graminearum (Gibberella zeae), F. equiseti, F. fujikoroi, F. musarum, F. oxysporum, F. proliferatum, F. poae, F. pseudograminearum, F. sambucinum, F. scirpi, F. semitectum, F. solani, F. sporotrichoides, F. langsethiae, F. subglutinans, F. tricinctum, F. verticillioides, inter alia, and also by Aspergillus spec., Penicillium spec., Claviceps purpurea, Stachybotrys spec., inter alia.

In the protection of materials, the compositions or active compounds according to the invention can furthermore be employed for protecting industrial materials against attack and destruction by unwanted microorganisms, such as, for example, fungi.

In the present context, industrial materials are understood as meaning nonliving materials which have been made for use in technology. For example, industrial materials which are to be protected by active compounds according to the invention from microbial modification or destruction can be glues, sizes, paper and board, textiles, leather, timber, paints and plastic articles, cooling lubricants and other materials which are capable of being attacked or destroyed by microorganisms. Parts of production plants, for example cooling-water circuits, which can be adversely affected by the multiplication of microorganisms may also be mentioned within the materials to be protected. Industrial materials which may be mentioned with preference for the purposes of the present invention are glues, sizes, paper and board, leather, timber, paints, cooling lubricants and heat-transfer fluids, especially preferably wood. The compositions or active compounds according to the invention can prevent disadvantageous effects such as rotting, decay, discoloration, decoloration or the formation of mould.

The method according to the invention for controlling unwanted fungi can also be employed for protecting storage goods. Here, storage goods are to be understood as meaning natural substances of vegetable or animal origin or process products thereof of natural origin, for which long-term protection is desired. Storage goods of vegetable origin, such as, for example, plants or plant parts, such as stems, leaves, tubers, seeds, fruits, grains, can be protected freshly harvested or after processing by (pre)drying, moistening, comminuting, grinding, pressing or roasting. Storage goods also include timber, both unprocessed, such as construction timber, electricity poles and barriers, or in the form of finished products, such as furniture. Storage goods of animal origin are, for example, hides, leather, furs and hairs. The active compound combinations according to the invention can prevent disadvantageous effects, such as rotting, decay, discoloration, decoloration or the formation of mould.

Microorganisms capable of degrading or changing the industrial materials which may be mentioned are, for example, bacteria, fungi, yeasts, algae and slime organisms. The active compounds according to the invention preferably act against fungi, in particular moulds, wood-discoloring and wood-destroying fungi (Basidiomycetes) and against slime organisms and algae. Microorganisms of the following genera may be mentioned as examples: Alternaria, such as Alternaria tenuis; Aspergillus, such as Aspergillus niger; Chaetomium, such as Chaetomium globosum; Coniophora, such as Coniophora puetana; Lentinus, such as Lentinus tigrinus; Penicillium, such as Penicillium glaucum; Polyporus, such as Polyporus versicolor; Aureobasidium, such as Aureobasidium pullulans; Sclerophoma, such as Sclerophoma pityophila; Trichoderma, such as Trichoderma viride; Escherichia, such as Escherichia coli; Pseudomonas, such as Pseudomonas aeruginosa; Staphylococcus, such as Staphylococcus aureus.

The present invention furthermore relates to a composition for controlling unwanted microorganisms comprising at least one of the diaminopyrimidines according to the invention. These are preferably fungicidal compositions comprising auxiliaries, solvents, carriers, surfactants or extenders suitable for use in agriculture.

According to the invention, a carrier is a natural or synthetic, organic or inorganic substance with which the active compounds are mixed or bonded for better applicability, in particular for application to plants or parts of plants or seed. The carrier, which may be solid or liquid, is generally inert and should be suitable for use in agriculture.

Suitable solid carriers are: for example ammonium salts and ground natural minerals, such as kaolins, clays, talc, chalk, quartz, attapulgite, montmorillonite or diatomaceous earth, and ground synthetic minerals, such as finely divided silica, alumina and silicates; suitable solid carriers for granules are: for example crushed and fractionated natural rocks, such as calcite, marble, pumice, sepiolite and dolomite, and also synthetic granules of inorganic and organic meals, and granules of organic material, such as paper, sawdust, coconut shells, maize cobs and tobacco stalks; suitable emulsifiers and/or foam-formers are: for example nonionic and anionic emulsifiers, such as polyoxyethylene fatty acid esters, polyoxyethylene fatty alcohol ethers, for example alkylaryl polyglycol ethers, alkylsulphonates, alkyl sulphates, arylsulphonates and also protein hydrolysates; suitable dispersants are nonionic and/or ionic substances, for example from the classes of the alcohol/POE and/or POP ethers, acid and/or POP/POE esters, alkylaryl and/or POP/POE ethers, fat and/or POP/POE adducts, POE and/or POP polyols derivatives, POE and/or POP/sorbitan or sugar adducts, alkyl or aryl sulphates, sulphonates and phosphates, or the corresponding PO ether adducts. Furthermore suitable oligo- or polymers, for example those derived from vinylic monomers, from acrylic acid, from EO and/or PO alone or in combination with, for example, (poly)alcohols or (poly)amines. It is also possible to employ lignin and its sulphonic acid derivatives, unmodified and modified celluloses, aromatic and/or aliphatic sulphonic acids and their adducts with formaldehyde.

The active compounds can be converted to the customary formulations, such as solutions, emulsions, wettable powders, water- and oil-based suspensions, powders, dusts, pastes, soluble powders, soluble granules, granules for broadcasting, suspension-emulsion concentrates, natural materials impregnated with active compound, synthetic materials impregnated with active compound, fertilizers and also microencapsulations in polymeric substances.

The active compounds can be used as such, in the form of their formulations or the use forms prepared therefrom, such as ready-to-use solutions, emulsions, water- or oil-based suspensions, powders, wettable powders, pastes, soluble powders, dusts, soluble granules, granules for broadcasting, suspension-emulsion concentrates, natural materials impregnated with active compound, synthetic materials impregnated with active compound, fertilizers and also microencapsulations in polymeric substances. Application is carried out in a customary manner, for example by pouring, spraying, atomizing, broadcasting, dusting, foaming, painting-on, etc. It is furthermore possible to apply the active compounds by the ultra-low-volume method or to inject the preparation of active compound or the active compound itself into the soil. It is also possible to treat the seed of the plants.

The formulations mentioned can be prepared in a manner known per se, for example by mixing the active compounds with at least one customary extender, solvent or diluent, emulsifier, dispersant and/or binder or fixative, wetting agent, water repellant, if appropriate siccatives and UV stabilizers and if appropriate colorants and pigments, antifoams, preservatives, secondary thickeners, glues, gibberellins and other processing auxiliaries.

The compositions according to the invention include not only formulations which are already ready to use and can be applied to the plant or the seed using a suitable apparatus, but also commercial concentrates which have to be diluted with water prior to use.

The active compounds according to the invention can be present as such or in their (commercial) formulations and also in the use forms prepared from these formulations as a mixture with other (known) active compounds, such as insecticides, attractants, sterilants, bactericides, acaricides, nematicides, fungicides, growth regulators, herbicides, fertilizers, safeners and/or semiochemicals.

Suitable for use as auxiliaries are substances which are suitable for imparting to the composition itself and/or to preparations derived therefrom (for example spray liquors, seed dressings) particular properties such as certain technical properties and/or also particular biological properties. Typical suitable auxiliaries are: extenders, solvents and carriers.

Suitable extenders are, for example, water, polar and nonpolar organic chemical liquids, for example from the classes of the aromatic and non-aromatic hydrocarbons (such as paraffins, alkylbenzenes, alkylnaphthalenes, chlorobenzenes), the alcohols and polyols (which, if appropriate, may also be substituted, etherified and/or esterified), the ketones (such as acetone, cyclohexanone), esters (including fats and oils) and (poly)ethers, the unsubstituted and substituted amines, amides, lactams (such as N-alkylpyrrolidones) and lactones, the sulphones and sulphoxides (such as dimethyl sulphoxide).

Liquefied gaseous extenders or carriers are liquids which are gaseous at ambient temperature and under atmospheric pressure, for example aerosol propellants, such as halogenated hydrocarbons, and also butane, propane, nitrogen and carbon dioxide.

Tackifiers, such as carboxymethylcellulose and natural and synthetic polymers in the form of powders, granules and latices, such as gum arabic, polyvinyl alcohol, polyvinyl acetate, or else natural phospholipids, such as cephalins and lecithins and synthetic phospholipids can be used in the formulations. Other possible additives are mineral and vegetable oils.

If the extender used is water, it is also possible to use, for example, organic solvents as auxiliary solvents. Suitable liquid solvents are essentially: aromatic compounds, such as xylene, toluene or alkylnaphthalenes, chlorinated aromatic compounds or chlorinated aliphatic hydrocarbons, such as chlorobenzenes, chloroethylenes or methylene chloride, aliphatic hydrocarbons, such as cyclohexane or paraffins, for example mineral oil fractions, alcohols, such as butanol or glycol, and also ethers and esters thereof, ketones, such as acetone, methyl ethyl ketone, methyl isobutyl ketone or cyclohexanone, strongly polar solvents, such as dimethylformamide and dimethyl sulphoxide, and also water.

The compositions according to the invention may additionally comprise further components, such as, for example, surfactants. Suitable surfactants are emulsifiers and/or foam-formers, dispersants or wetting agents having ionic or nonionic properties, or mixtures of these surfactants. Examples of these are salts of polyacrylic acid, salts of lignosulphonic acid, salts of phenolsulphonic acid or naphthalenesulphonic acid, polycondensates of ethylene oxide with fatty alcohols or with fatty acids or with fatty amines, substituted phenols (preferably alkylphenols or arylphenols), salts of sulphosuccinic esters, taurine derivatives (preferably alkyl taurates), phosphoric esters of polyethoxylated alcohols or phenols, fatty esters of polyols, and derivatives of the compounds containing sulphates, sulphonates and phosphates, for example alkylaryl polyglycol ethers, alkylsulphonates, alkyl sulphates, arylsulphonates, protein hydrolysates, lignosulphite waste liquors and methylcellulose. The presence of a surfactant is required if one of the active compounds and/or one of the inert carriers is insoluble in water and the application is carried out in water. The proportion of surfactants is between 5 and 40 percent by weight of the compositions according to the invention.

It is possible to use colorants such as inorganic pigments, for example iron oxide, titanium oxide, Prussian blue, and organic dyes, such as alizarin dyes, azo dyes and metal phthalocyanine dyes, and trace nutrients, such as salts of iron, manganese, boron, copper, cobalt, molybdenum and zinc.

Other possible additives are perfumes, mineral or vegetable oils, if appropriate modified, waxes and nutrients (including trace nutrients), such as salts of iron, manganese, boron, copper, cobalt, molybdenum and zinc.

Stabilizers, such as low-temperature stabilizers, preservatives, antioxidants, light stabilizers or other agents which improve chemical and/or physical stability may also be present.

If appropriate, it is also possible for other additional components to be present, for example protective colloids, binders, glues, thickeners, thixotropic agents, penetrants, stabilizers, sequestrants, complex fomers. In general, the active compounds can be combined with any solid or liquid additive customarily used for formulation purposes.

The formulations generally comprise between 0.05 and 99% by weight, 0.01 and 98% by weight, preferably between 0.1 and 95% by weight, particularly preferably between 0.5 and 90% by weight, of active compound, very particularly preferably between 10 and 70 percent by weight.

The formulations described above can be employed in a method according to the invention for controlling unwanted microorganisms where the diaminopyrimidines according to the invention are applied to the microorganisms and/or their habitat.

The active compounds according to the invention, as such or in their formulations, can also be used in a mixture with known fungicides, bactericides, acaricides, nematicides or insecticides, for example to broaden the activity spectrum or to prevent the development of resistance.

Suitable mixing partners are, for example, known fungicides, insecticides, acaricides, nematicides or else bactericides (see also Pesticide Manual, 13th ed.).

A mixture with other known active compounds, such as herbicides, or with fertilizers and growth regulators, safeners and/or semiochemicals is also possible.

Application is carried out in a manner adapted to the use forms.

The control of phytopathogenic harmful fungi which damage plants post-emergence is primarily by treating the soil and the above-ground parts of the plants with crop protection compositions. Owing to concerns with a view to a possible impact of the crop protection compositions on the environment and human and animal health, there are efforts to reduce the amount of active compounds applied.

The active compounds can be applied as such, in the form of their formulations and the use forms prepared therefrom, such as ready-to-use solutions, suspensions, wettable powders, pastes, soluble powders, dusts and granules. Application is carried out in a customary manner, for example by watering, spraying, atomizing, broadcasting, dusting, foaming, painting-on, etc. It is also possible to apply the active compounds by the ultra-low-volume method or to inject the preparation of active compound or the active compound itself into the soil. It is also possible to treat the seed of the plants.

When using the active compounds according to the invention as fungicides, the application rates can be varied within a relatively wide range, depending on the type of application. The application rate of the active compounds according to the invention is
in the treatment of parts of plants, for example leaves: from 0.1 to 10 000 g/ha, preferably from 10 to 1000 g/ha, particularly preferably from 50 to 300 g/ha (when the application is by watering or dripping, it is possible to reduce the application rate even more, in particular when inert substrates such as rock wool or perlite are used);
in the treatment of seed: from 2 to 200 g per 100 kg of seed, preferably from 3 to 150 g per 100 kg of seed, particularly preferably from 2.5 to 25 g per 100 kg of seed, very particularly preferably from 2.5 to 12.5 g per 100 kg of seed;
in soil treatment: from 0.1 to 10 000 g/ha, preferably from 1 to 5000 g/ha.

These application rates are mentioned only in an exemplary manner and not limiting for the purpose of the invention.

The compounds according to the invention can also be used for protecting objects which come into contact with salt water or brackish water, such as hulls, screens, nets, buildings, moorings and signalling systems, against colonization.

The active compounds according to the invention, alone or in combination with other active compounds, can further more be employed as antifouling agents.

The treatment method according to the invention can be used for treating genetically modified organisms (GMOs), for example plants or seeds. Genetically modified plants (or transgenic plants) are plants in which a heterologous gene has been stably integrated into the genome. The expression “heterologous gene” essentially means a gene which is provided or assembled outside the plant and when introduced in the nuclear, chloroplastic or mitochondrial genome gives the transformed plant new or improved agronomic or other properties by expressing a protein or polypeptide of interest or by downregulating or silencing other gene(s) which is/are present in the plant (using for example, antisense technology, cosuppression technology or RNA interference RNAi-technology). A heterologous gene that is located in the genome is also called a transgene. A transgene that is defined by its particular location in the plant genome is called a transformation or transgenic event.

Depending on the plant species or plant cultivars, their location and growth conditions (soils, climate, vegetation period, diet), the treatment according to the invention may also result in superadditive (“synergistic”) effects. Thus, for example, the following effects, which exceed the effects which were actually to be expected, are possible: reduced application rates and/or a widening of the activity spectrum and/or an increase in the activity of the active compounds and compositions which can be used according to the invention, better plant growth, increased tolerance to high or low temperatures, increased tolerance to drought or to water or soil salt content, increased flowering performance, easier harvesting, accelerated maturation, higher harvest yields, bigger fruits, larger plant height, greener leaf colour, earlier flowering, higher quality and/or a higher nutritional value of the harvested products, higher sugar concentration within the fruits, better storage stability and/or processability of the harvested products.

In the present case, unwanted phytopathogenic fungi and/or microorganisms and/or viruses are to be understood as meaning phytopathogenic fungi, bacteria and viruses. Thus, the substances according to the invention can be employed for protecting plants against attack by the abovementioned pathogens within a certain period of time after the treatment. The period of time within which protection is effected generally extends from 1 to 10 days, preferably 1 to 7 days, after the treatment of the plants with the active compounds.

Plants and plant cultivars which are preferably treated according to the invention include all plants with genetic material which bestows upon these plants particularly advantageous useful properties (whether this was achieved by breeding and/or biotechnology is immaterial).

Plants and plant cultivars which are also preferably treated according to the invention are resistant against one or more biotic stress factors, i.e. said plants have a better defense against animal and microbial pests, such as against nematodes, insects, mites, phytopathogenic fungi, bacteria, viruses and/or viroids.

Plants and plant cultivars which may also be treated according to the invention are those plants which are resistant to one or more abiotic stress factors. Abiotic stress conditions may include, for example, drought, cold temperature exposure, heat exposure, osmotic stress, flooding, increased soil salinity, increased mineral exposure, ozone exposure, high light exposure, limited availability of nitrogen nutrients, limited availability of phosphorus nutrients or shade avoidance.

Plants and plant cultivars which may also be treated according to the invention are those plants characterized by enhanced yield characteristics. Increased yield in said plants can be the result of, for example, improved plant physiology, growth and development, such as water use efficiency, water retention efficiency, improved nitrogen use, enhanced carbon assimilation, improved photosynthesis, increased germination efficiency and accelerated maturation. Yield can furthermore by affected by improved plant architecture (under stress and non-stress conditions), including early flowering, flowering control for hybrid seed production, seedling vigour, plant size, internode number and distance, root growth, seed size, fruit size, pod size, pod or ear number, seed number per pod or ear, seed mass, enhanced seed filling, reduced seed dispersal, reduced pod dehiscence and lodging resistance. Further yield traits include seed composition, such as carbohydrate content, protein content, oil content and composition, nutritional value, reduction in anti-nutritional compounds, improved processability and better storage stability.

Plants that may be treated according to the invention are hybrid plants that already express the characteristic of heterosis or the hybrid effect which results in generally higher yield, vigour, health and resistance towards biotic and abiotic stress factors. Such plants are typically made by crossing an inbred male sterile parent line (the female parent) with another inbred male fertile parent line (the male parent). Hybrid seed is typically harvested from the male sterile plants and sold to growers. Male sterile plants can sometimes (e.g. in corn) be produced by detasseling, (i.e. the mechanical removal of the male reproductive organs or male flowers) but, more typically, male sterility is the result of genetic determinants in the plant genome. In that case, and especially when seed is the desired product to be harvested from the hybrid plants, it is typically useful to ensure that male fertility in the hybrid plants, which contain the genetic determinants responsible for male sterility, is fully restored. This can be accomplished by ensuring that the male parents have appropriate fertility restorer genes which are capable of restoring the male fertility in hybrid plants that contain the genetic determinants responsible for male sterility. Genetic determinants for male sterility may be located in the cytoplasm. Examples of cytoplasmic male sterility (CMS) were for instance described in Brassica species. However, genetic determinants for male sterility can also be located in the nuclear genome. Male sterile plants can also be obtained by plant biotechnology methods such as genetic engineering. A particularly useful means of obtaining male sterile plants is described in WO 89/10396 in which, for example, a ribonuclease such as a barnase is selectively expressed in the tapetum cells in the stamens. Fertility can then be restored by expression in the tapetum cells of a ribonuclease inhibitor such as barstar.

Plants or plant cultivars (obtained by plant biotechnology methods such as genetic engineering) which may be treated according to the invention are herbicide-tolerant plants, i.e. plants made tolerant to one or more given herbicides. Such plants can be obtained either by genetic transformation, or by selection of plants containing a mutation imparting such herbicide tolerance.

Herbicide-tolerant plants are for example glyphosate-tolerant plants, i.e. plants made tolerant to the herbicide glyphosate or salts thereof. For example, glyphosate-tolerant plants can be obtained by transforming the plant with a gene encoding the enzyme 5-enolpyruvylshikimate-3-phosphate synthase (EPSPS). Examples of such EPSPS genes are the AroA gene (mutant CT7) of the bacterium Salmonella typhimurium, the CP4 gene of the bacterium Agrobacterium sp., the genes encoding a petunia EPSPS, a tomato EPSPS, or an Eleusine EPSPS. It can also be a mutated EPSPS. Glyphosate-tolerant plants can also be obtained by expressing a gene that encodes a glyphosate oxidoreductase enzyme. Glyphosate-tolerant plants can also be obtained by expressing a gene that encodes a glyphosate acetyl transferase enzyme. Glyphosate-tolerant plants can also be obtained by selecting plants containing naturally-occurring mutations of the above-mentioned genes.

Other herbicide-resistant plants are for example plants that are made tolerant to herbicides inhibiting the enzyme glutamine synthase, such as bialaphos, phosphinothricin or glufosinate. Such plants can be obtained by expressing an enzyme detoxifying the herbicide or a mutant glutamine synthase enzyme that is resistant to inhibition. One such efficient detoxifying enzyme is, for example, an enzyme encoding a phosphinothricin acetyltransferase (such as the bar or pat protein from Streptomyces species). Plants expressing an exogenous phosphinothricin acetyltransferase have been described.

Further herbicide-tolerant plants are also plants that are made tolerant to the herbicides inhibiting the enzyme hydroxyphenylpyruvatedioxygenase (HPPD). Hydroxyphenylpyruvatedioxygenases are enzymes that catalyse the reaction in which para-hydroxyphenylpyruvate (HPP) is transformed into homogentisate. Plants tolerant to HPPD-inhibitors can be transformed with a gene encoding a naturally-occurring resistant HPPD enzyme, or a gene encoding a mutated HPPD enzyme. Tolerance to HPPD-inhibitors can also be obtained by transforming plants with genes encoding certain enzymes enabling the formation of homogentisate despite the inhibition of the native HPPD enzyme by the HPPD-inhibitor. Tolerance of plants to HPPD inhibitors can also be improved by transforming plants with a gene encoding an enzyme prephenate dehydrogenase in addition to a gene encoding an HPPD-tolerant enzyme.

Still further herbicide-resistant plants are plants that are made tolerant to acetolactate synthase (ALS) inhibitors. Known ALS-inhibitors include, for example, sulphonylurea, imidazolinone, triazolopyrimidines, pyrimidinyloxy(thio)benzoates, and/or sulphonylaminocarbonyltriazolinone herbicides. Different mutations in the ALS enzyme (also known as acetohydroxyacid synthase, AHAS) are known to confer tolerance to different herbicides and groups of herbicides. The production of sulphonylurea-tolerant plants and imidazolinone-tolerant plants has been described in the international publication WO 1996/033270. Further sulphonylurea- and imidazolinone-tolerant plants have also been described, for example in WO 2007/024782.

Other plants tolerant to imidazolinone and/or sulphonylurea can be obtained by induced mutagenesis, by selection in cell cultures in the presence of the herbicide or by mutation breeding.

Plants or plant cultivars (obtained by plant biotechnology methods such as genetic engineering) which may also be treated according to the invention are insect-resistant transgenic plants, i.e. plants made resistant to attack by certain target insects. Such plants can be obtained by genetic transformation, or by selection of plants containing a mutation imparting such insect resistance.

In the present context, the term “insect-resistant transgenic plant” includes any plant containing at least one transgene comprising a coding sequence encoding:
1) an insecticidal crystal protein from Bacillus thuringiensis or an insecticidal portion thereof, such as the insecticidal crystal proteins listed online at: http://www.lifesci.sussex.ac.uk/Home/Neil_Crickmore/Bt/, or insecticidal portions thereof, for example proteins of the Cry protein classes Cry1Ab, Cry1Ac, Cry1F, Cry2Ab, Cry3Ae or Cry3Bb or insecticidal portions thereof; or
2) a crystal protein from Bacillus thuringiensis or a portion thereof which is insecticidal in the presence of a second other crystal protein from Bacillus thuringiensis or a portion thereof, such as the binary toxin made up of the Cy34 and Cy35 crystal proteins; or
3) a hybrid insecticidal protein comprising parts of two different insecticidal crystal proteins from Bacillus thuringiensis, such as a hybrid of the proteins of 1) above or a hybrid of the proteins of 2) above, for example the Cry1A.105 protein produced by maize event MON98034 (WO 2007/027777); or
4) a protein of any one of 1) to 3) above wherein some, particularly 1 to 10, amino acids have been replaced by another amino acid to obtain a higher insecticidal activity to a target insect species, and/or to expand the range of target insect species affected, and/or because of changes induced in the encoding DNA during cloning or transformation, such as the Cry3Bb1 protein in maize events MON863 or MON88017, or the Cry3A protein in maize event MIR604;
5) an insecticidal secreted protein from Bacillus thuringiensis or Bacillus cereus, or an insecticidal portion thereof, such as the vegetative insecticidal proteins (VIP) listed at: http://www.lifesci.sussex.ac.uk/home/Neil_Crickmore/Bt/vip.html, for example proteins from the VIP3Aa protein class; or
6) a secreted protein from Bacillus thuringiensis or Bacillus cereus which is insecticidal in the presence of a second secreted protein from Bacillus thuringiensis or B. cereus, such as the binary toxin made up of the VIP1a and VIP2A proteins;
7) a hybrid insecticidal protein comprising parts from different secreted proteins from Bacillus thuringiensis or Bacillus cereus, such as a hybrid of the proteins in 1) above or a hybrid of the proteins in 2) above; or
8) a protein of any one of 1) to 3) above wherein some, particularly 1 to 10, amino acids have been replaced by another amino acid to obtain a higher insecticidal activity to a target insect species, and/or to expand the range of target insect species affected, and/or because of changes induced in the encoding DNA during cloning or transformation (while still encoding an insecticidal protein), such as the VIP3Aa protein in cotton event COT102.

Of course, insect-resistant transgenic plants, as used herein, also include any plant comprising a combination of genes encoding the proteins of any one of the above classes 1 to 8. In one embodiment, an insect-resistant plant contains more than one transgene encoding a protein of any one of the above classes 1 to 8, to expand the range of target insect species affected or to delay insect resistance development to the plants, by using different proteins insecticidal to the same target insect species but having a different mode of action, such as binding to different receptor binding sites in the insect.

Plants or plant cultivars (obtained by plant biotechnology methods such as genetic engineering) which may also be treated according to the invention are tolerant to abiotic stresses. Such plants can be obtained by genetic transformation, or by selection of plants containing a mutation imparting such stress resistance. Particularly useful stress tolerance plants include:
a. plants which contain a transgene capable of reducing the expression and/or the activity of the poly(ADP-ribose)polymerase (PARP) gene in the plant cells or plants.
b. plants which contain a stress tolerance-enhancing transgene capable of reducing the expression and/or the activity of the PARG encoding genes of the plants or plant cells;
c. plants which contain a stress tolerance-enhancing transgene coding for a plant-functional enzyme of the nicotinamide adenine dinucleotide salvage biosynthesis pathway, including nicotinamidase, nicotinate phosphoribosyltransferase, nicotinic acid mononucleotide adenyl transferase, nicotinamide adenine dinucleotide synthetase or nicotinamide phosphoribosyltransferase.

Plants or plant cultivars (obtained by plant biotechnology methods such as genetic engineering) which may also be treated according to the invention show altered quantity, quality and/or storage-stability of the harvested product and/or altered properties of specific ingredients of the harvested product such as, for example:
1) transgenic plants which synthesize a modified starch, which in its physical-chemical characteristics, in particular the amylose content or the amylose/amylopectin ratio, the degree of branching, the average chain length, the side chain distribution, the viscosity behaviour, the gelling strength, the starch grain size and/or the starch grain morphology, is changed in comparison with the synthesized starch in wild type plant cells or plants, so that this modified starch is better suited for special applications.
2) transgenic plants which synthesize non-starch carbohydrate polymers or which synthesize non-starch carbohydrate polymers with altered properties in comparison to wild type plants without genetic modification. Examples are plants which produce polyfructose, especially of the inulin and levan type, plants which produce alpha-1,4-glucans, plants which produce alpha-1,6 branched alpha-1,4-glucans, and plants producing alternan.
3) transgenic plants which produce hyaluronan.

Plants or plant cultivars (that can be obtained by plant biotechnology methods such as genetic engineering) which may also be treated according to the invention are plants, such as cotton plants, with altered fibre characteristics. Such plants can be obtained by genetic transformation, or by selection of plants containing a mutation imparting such altered fibre characteristics and include:
a) plants, such as cotton plants, which contain an altered form of cellulose synthase genes,
b) plants, such as cotton plants, which contain an altered form of rsw2 or rsw3 homologous nucleic acids;
c) plants, such as cotton plants, with an increased expression of sucrose phosphate synthase;
d) plants, such as cotton plants, with an increased expression of sucrose synthase;
e) plants, such as cotton plants, wherein the timing of the plasmodesmatal gating at the basis of the fibre cell is altered, for example through downregulation of fibre-selective β-1,3-glucanase;
f) plants, such as cotton plants, which have fibres with altered reactivity, for example through the expression of the N-acetylglucosaminetransferase gene including nodC and chitin synthase genes.

Plants or plant cultivars (that can be obtained by plant biotechnology methods such as genetic engineering) which may also be treated according to the invention are plants, such as oilseed rape or related Brassica plants, with altered oil profile characteristics. Such plants can be obtained by genetic transformation or by selection of plants containing a mutation imparting such altered oil characteristics and include:
a) plants, such as oilseed rape plants, which produce oil having a high oleic acid content;
b) plants, such as oilseed rape plants, which produce oil having a low linolenic acid content;
c) plants, such as oilseed rape plants, which produce oil having a low level of saturated fatty acids.

Particularly useful transgenic plants which may be treated according to the invention are plants which comprise one or more genes which encode one or more toxins, are the following which are sold under the trade names YIELD GARD® (for example maize, cotton, soya beans), KnockOut® (for example maize), BiteGard® (for example maize), Bt-Xtra® (for example maize), StarLink® (for example maize), Bollgard® (cotton), Nucotn® (cotton), Nucotn 33B® (cotton), NatureGard® (for example maize), Protecta® and NewLeaf® (potato). Examples of herbicide-tolerant plants which may be mentioned are maize varieties, cotton varieties and soya bean varieties which are sold under the trade names Roundup Ready® (tolerance to glyphosate, for example maize, cotton, soya beans), Liberty Link® (tolerance to phosphinothricin, for example oilseed rape), IMI® (tolerance to imidazolinone) and SCS® (tolerance to sulphonylurea, for example maize). Herbicide-resistant plants (plants bred in a conventional manner for herbicide tolerance) which may be mentioned include the varieties sold under the name Clearfield® (for example maize).

Particularly useful transgenic plants which may be treated according to the invention are plants containing transformation events, or a combination of transformation events, that are listed for example in the databases for various national or regional regulatory agencies (see for example http://gmoinfo.jrc.it/gmp_browse.aspx and http://www.agbios.com/dbase.php).

According to the invention, the plants listed can be treated particularly advantageously with the compounds of the general formula (I) or the active compound mixtures according to the invention. The preferred ranges indicated above for the active compounds and mixtures also apply to the treatment of these plants. Particular emphasis is given to treating the plants with the compounds and mixtures specifically indicated in the present text.

The compositions or active compounds according to the invention can also be used to protect plants for a certain period after treatment against attack by the pathogens mentioned. The period for which protection is provided generally extends over 1 to 28 days, preferably over 1 to 14 days, particularly preferably over 1 to 10 days, very particularly preferably over 1 to 7 days, after the treatment of the plants with the active compounds, or over up to 200 days after seed treatment.

Preparation and use of the active compounds of the formulae (I), (Ia), (Ib) and (Ic) according to the invention is shown in the examples below. However, the invention is not limited to these examples.

Synthesis of Intermediates of the Formula (V) (cf. Scheme 1) 2,5-Dichloro-N-(3-methoxypropan-2-yl)pyrimidin-4-amine (V-1)

At −10° C., 5.42 g (39.3 mmol) of potassium carbonate are added to a solution of 6.00 g (32.7 mmol) of 2,4,5-trichloropyrimidine in 100 ml of acetonitrile. 3.06 g (34.4 mmol) of 2-amino-1-methoxypropane are then added dropwise as a 20% strength solution in acetonitrile. With stirring, the reaction mixture is allowed to warm to room temperature overnight. The reaction mixture is then stirred into 250 ml of ice-water/dilute hydrochloric acid (1:1). The resulting precipitate is filtered off and dried. This gives 5.10 g (64%) of 2,5-dichloro-N-(3-methoxypropan-2-yl)pyrimidin-4-amine (log P (pH 2.3): 2.10); ¹H NMR (400 MHz, MeCN-d) δ=8.02 (s, 1H), 6.03 (br. s, 1H), 4.39-4.33 (m, 1H), 3.48-3.40 (m, 2H), 3.33 (s., 3H), 1.23 (d, 3H).

### 2,5-Dichloro-N-(2,2-difluoroethyl)pyrimidin-4-amine (V-2)

At −10° C., 6.14 g (44.4 mmol) of potassium carbonate are added to a solution of 5.43 g (29.6 mmol) of 2,4,5-trichloropyrimidine in 40 ml of acetonitrile. 2.40 g (29.6 mmol) of 2,2-difluoroethanamine are then added dropwise as a 30% strength solution in acetonitrile. With stirring, the reaction mixture is allowed to warm to room temperature overnight. The reaction mixture is stirred into 250 ml of ice-water/dilute hydrochloric acid (1:1). The mixture is extracted with dichloromethane (2×200 ml), the combined organic phases are then washed with water (100 ml) and dried over MgSO₄ and the solvent is removed under reduced pressure. This gives 6.10 g (90%) of 2,5-dichloro-N-(2,2-difluoroethyl)pyrimidin-4-amine (log P (pH 2.3): 1.96); ¹H NMR (400 MHz, MeCN-d) δ=8.10 (s, 1H), 6.47 (br. s, 1H), 6.02 (tt, 1H), 3.86 (m, 2H).

### 2-Chloro-N-(3-methoxypropan-2-yl)-5-trifluoromethylpyrimidin-4-amine (V-3)

At −10° C., 1.91 g (13.8 mmol) of potassium carbonate are added to a solution of 2.00 g (9.22 mmol) of 2,4-dichloro-5-trifluoropyrimidine in 80 ml of acetonitrile. 0.86 g (9.68 mmol) of 2-amino-1-methoxypropane then added dropwise as a 30% strength solution in acetonitrile. With stirring, the reaction mixture is allowed to warm to room temperature overnight. The reaction mixture is then stirred into 250 ml of ice-water and extracted with dichloromethane (3×100 ml). The combined organic phases are separated off, washed with water (2×100 ml), dried over MgSO₄ and freed from the solvent under reduced pressure. The crude product is purified by column chromatography on silica gel (cyclohexane/ethyl acetate). This gives 0.75 g (26%) of 2-chloro-N-(3-methoxypropan-2-yl)-5-trifluoromethylpyrimidin-4-amine (log P (pH 2.3): 2.75); ¹H NMR (400 MHz, DMSO-d) δ=8.28 (s, 1H), 3.56-3.52 (m, 3H), 3.33-3.32 (d, 3H), 1.24-1.22 (q, 3H).

### 2,5-Dichloro-N-(2,2-difluoroethyl)pyrimidin-4-amine (V-4)

At −10° C., 6.14 g (44.4 mmol) of potassium carbonate are added to a solution of 5.43 g (29.6 mmol) of 2,4,5-trichloropyrimidine in 40 ml of acetonitrile. 2.40 g (29.6 mmol) of 2,2-difluoroethanamine are then added dropwise as a 30% strength solution in acetonitrile. With stirring, the reaction mixture is allowed to warm to room temperature overnight. The reaction mixture is stirred into 250 ml of ice-water/dilute hydrochloric acid (1:1). The mixture is extracted with dichloromethane (2×200 ml), the combined organic phases are then washed with water (100 ml) and dried over MgSO₄ and the solvent is removed under reduced pressure. This gives 6.10 g (90%) of 2,5-dichloro-N-(2,2-difluoroethyl)pyrimidin-4-amine (log P (pH 2.3): 1.96); ¹H NMR (400 MHz, MeCN-d) δ=8.10 (s, 1H), 6.47 (br. s, 1H), 6.02 (tt, 1H), 3.86 (m, 2H).

### 2,5-Dichloro-N-(2,2,2-trifluoroethyl)pyrimidin-4-amine (V-5)

At 50° C., 18.1 g (130 mmol) of potassium carbonate are added to a solution of 16.0 g (87.2 mmol) of 2,4,5-trichloropyrimidine in 100 ml of acetonitrile. 9.07 g (91.6 mmol) of 2,2,2-trifluoroethanamine are then added dropwise as a 30% strength solution in acetonitrile. The reaction mixture is stirred at 50° C. for 16 h. After cooling, the reaction mixture is stirred into 250 ml of ice-water. The mixture is extracted with ethyl acetate (2×200 ml), the combined organic phases are then washed with water (2×100 ml) and dried over MgSO₄ and the solvent is removed under reduced pressure. The crude product is stirred into cyclohexane and the precipitated solid is, after 2 h, filtered off and dried. This gives 13.9 g (64%) of 2.5-dichloro-N-(2,2,2-trifluoroethyl)pyrimidin-4-amine (log P (pH 2.3): 2.26); ¹H NMR (400 MHz, DMSO-d) δ=8.29 (s, 1H), 8.25 (br. s, 1H), 4.24-4.15 (m, 2H).

The compounds below can be prepared in an analogous manner:

5-Bromo-2-chloro-N-(3-methylcyclobutyl)pyrimidin-4-amine (V-6) (Main isomer: log P (pH 2.3): 3.47; ¹H NMR (400 MHz, DMSO-d6) δ=8.19 (s, 1H), 7.46 (s, 1H), 4.25-4.30 (m, 1H), 2.31-2.35 (m, 3H), 1.93-1.99 (m, 2H), 1.05 (d, 3H).

2,5-Dichloro-N-cyclopropylpyrimidin-4-amine (V-7) (log P (pH 2.3): 1.79); ¹NMR (400 MHz, DMSO-d) δ=8.11 (s, 1H), 7.71 (br. s, 1H), 2.89-2.84 (m, 1H), 0.79-0.64 (m, 4H).

5-Bromo-2-chloro-N-cyclopropylpyrimidin-4-amine (V-8) (log P (pH 2.3): 1.97); ¹H NMR (400 MHz, MeCN-d) δ=8.12 (s, 1H), 6.17 (br. s, 1H), 2.87-2.80 (m, 1H), 0.85-0.79 (m, 2H) 0.66-0.62 (m, 2H).

2-Chloro-N-cyclopropyl-5-iodopyrimidin-4-amine (V-9) (log P (pH 2.3): 2.19); ¹H NMR (400 MHz, MeCN-d) δ=8.28 (s, 1H), 5.96 (br. s, 1H), 2.85-2.80 (m, 1H), 0.84-0.79 (m, 2H) 0.64-0.61 (m, 2H).

2,5-Dichloro-N-(cyclopropylmethyl)pyrimidin-4-amine (V-10) (log P (pH 2.3): 2.51); ¹H NMR (400 MHz, MeCN-d) δ=8.01 (s, 1H), 6.34 (br. s, 1H), 3.33-3.29 (m, 2H), 1.16-1.06 (m, 1H), 0.54-0.45 (m, 2H) 0.33-0.24 (m, 2H).

2,5-Dichloro-N-(1-cyclopropylethyl)pyrimidin-4-amine (V-11) (log P (pH 2.3): 2.97); ¹H NMR (400 MHz, DIMETHYL SULPHOXIDE-d) δ=8.10 (s, 1H), 7.47.7.46 (br. s, 1H), 1.27-1.26 (d, 3H), 1.16-1.11 (m, 1H), 0.49-0.43 (m, 2H), 0.41-0.39 (m, 2H).

5-Bromo-2-chloro-N-(cyclopropylmethyl)pyrimidin-4-amine (V-12) (log P (pH 2.3): 2.69); ¹H NMR (400 MHz, DIMETHYL SULPHOXIDE-d) δ=8.20 (s, 1H), 7.58 (br. s, 1H), 3.25 (tr, 2 H), 1.14 (br. m, 1H), 0.44 (m, 2H), 0.26 (m, 2H).

2-Chloro-N-cyclopropyl-5-trifluoromethylpyrimidin-4-amine (V-13) (log P (pH 2.3): 2.39); ¹H NMR (400 MHz, MeCN-d) δ=8.28 (s, 1H), 6.34 (br. s, 1H), 2.91-2.86 (m, 1H), 0.85-0.80 (m, 2H), 0.66-0.62 (m, 2H).

2-Chloro-N-(cyclopropylmethyl)-5-trifluoromethylpyrimidin-4-amine (V-14) (log P (pH 2.3): 3.40); ¹H NMR (400 MHz, DMSO-d6) δ=8.05 (s, 1H), 7.51 (br. s., 1H), 3.02 (t, 2H), 0.79-0.89 (m, 1H), 0.11-0.17 (m, 2H), −0.03-0.03 (m, 2H); M+H=252.0.

2,5-Dichloro-N-cyclobutylpyrimidin-4-amine (V-15) (log P (pH 2.3): 2.62); ¹H NMR (400 MHz, MeCN-d) δ=8.00 (s, 1H), 6.31 (br. s, 1H), 4.54-4.46 (m, 1H), 2.39-2.31 (m, 2H), 2.15-2.04 (m, 2H), 1.83-1.77 (m, 2H).

5-Bromo-2-chloro-N-cyclobutylpyrimidin-4-amine (V-16) (log P (pH 2.3): 2.87); ¹H NMR (400 MHz, DIMETHYL SULPHOXIDE-d) δ=8.20 (s, 1H), 7.52 (br. s, 1H), 4.45 (br. m, 1H), 2.24 (m, 2H), 2.17 (m, 2H), 1.69 (m, 2H).

2-Chloro-N-cyclobutyl-5-trifluoromethylpyrimidin-4-amine (V-17) (log P (pH 2.3): 3.20); ¹H NMR (400 MHz, MeCN-d) δ=8.27 (s, 1H), 6.19 (br. s, 1H), 4.64-4.56 (m, 1H), 2.40-2.32 (m, 2H), 2.14-2.04 (m, 2H), 1.82-1.74 (m, 2H).

5-Bromo-2-chloro-N-(3-methoxypropan-2-yl)pyrimidin-4-amine (V-18) (log P (pH 2.3): 2.26); ¹H NMR (400 MHz, DIMETHYL SULPHOXIDE-d) δ=8.22 (s, 1H), 6.98 (br. d, 1H), 4.36 (br. m, 1H), 3.48 (dd, 1H), 3.36 (dd, 1H), 3.28 (s, 1H), 1.17 (d, 3H).

2,5-Dichloro-N-(propan-2-yl)pyrimidin-4-amine (V-19) (log P (pH 2.3): 2.46); ¹H NMR (400 MHz, MeCN-d) δ=7.99 (s, 1H), 5.92 (br. s, 1H), 4.31-4.23 (m, 1H), 1.25 (d, 6H)

2,5-Dichloro-N-methyl-N-(propan-2-yl)pyrimidin-4-amine (V-20) (log P (pH 2.3): 3.16); ¹H NMR (400 MHz, MeCN-d) δ=8.04 (s, 1H), 4.82-4.76 (m, 1H), 3.03 (s., 3H), 1.22 (d, 6H)

2,5-Dichloro-N-(cyclopentyl)pyrimidin-4-amine (V-21) (log P (pH 2.3): 3.16); ¹H NMR (400 MHz, DMSO-d) δ=8.11-8.09 (d, 1H), 7.36 (d, 1H), 4.36-4.28 (m, 1H), 1.98-1.93 (m, 2H), 1.73-1.67 (m, 2H), 1.64-1.53 (m, 4H)

2,5-Dichloro-N-(prop-2-en-1-yl)pyrimidin-4-amine (V-22) (log P (pH 2.3): 2.12); ¹H NMR (400 MHz, MeCN-d) δ=8.03 (s, 1H), 6.40 (br. s, 1H), 5.98-5.88 (m, 1H), 5.23-5.12 (m, 2H), 4.09-4.06 (m, 2H).

2,5-Dichloro-N-(butan-2-yl)pyrimidin-4-amine (V-23) (log P (pH 2.3): 2.94); ¹H NMR (400 MHz, MeCN-d) δ=7.99 (s, 1H), 5.90 (br. s, 1H), 4.15-4.08 (m, 1H), 1.67-1.56 (m, 2H), 1.21 (d, 3H), 0.91 (t, 3H).

2,5-Dichloro-N-ethyl-N-methylpyrimidin-4-amine (V-24) (log P (pH 2.3): 2.68); ¹H NMR (400 MHz, DMSO-d) δ=8.14 (s, 1H), 3.67 (q, 2H), 3.18 (s, 3H), 1.19 (t, 3H).

2,5-Dichloro-N-ethylpyrimidin-4-amine (V-25) (log P (pH 2.3): 1.93); ¹H NMR (400 MHz, MeCN-d) δ=7.99 (s, 1H), 6.23 (br. s, 1H), 3.48 (q, 2H), 1.20 (t, 3H).

2,5-Dichloro-N-methyl-N-cyclopropylpyrimidin-4-amine (V-26) (log P (pH 2.3): 2.82.); ¹H NMR (400 MHz, MeCN-d) δ=8.09 (s, 1H), 3.15-3.12 (m, 1H), 3.11 (s, 3H), 0.87-0.82 (m, 2H), 0.72-0.70 (m, 2H).

2,5-Dichloro-N-(2,2-dimethylcyclopropyl)pyrimidin-4-amine (V-27) (log P (pH 2.3): 3.04); ¹H NMR (400 MHz, DMSO-d) δ=8.12 (s, 1H), 7.63 (s(br), 1H), 2.53 (m, 1H), 1.12 (s, 3H), 0.93 (s, 3H), 0.73 (m, 2H).

5-Fluoro-2-chloro-N-cyclobutylpyrimidin-4-amine (V-28) (log P (pH 2.3): 2.17); ¹H NMR (400 MHz, MeCN-d) δ=7.84 (d, 1H), 6.37 (br. s, 1H), 4.54-4.43 (m, 1H), 2.40-2.30 (m, 2H), 2.12-2.04 (m, 2H), 1.91-1.71 (m, 2H).

2,5-Dichloro-N-(oxetan-3-yl)pyrimidin-4-amine (V-29) (log P (pH 2.3): 1.31); ¹H NMR (400 MHz, MeCN-d) δ=8.07 (s, 1H), 6.72 (br. s, 1H), 5.09-5.03 (m, 1H), 4.85-4.83 (m, 2H), 4.66-4.62 (m, 2H).

2-Chloro-4-[(1-methoxypropan-2-yl)-amino]pyrimidine-5-carbonitrile (V-30) (log P (pH 2.3): 1.83); ¹H NMR (400 MHz, DMSO-d6) δ=8.50 (s, 1H), 8.10 (br. s., 1H), 4.47-4.40 (m, 1H), 3.42-3.29 (m, 2H), 3.27 (s, 3H), 1.14-1.13 (d, 2H); M+H=227.0.

2,5-Dichloro-N-[2-methyl-1-(methylsulphanyl)propan-2-yl]pyrimidin-4-amine (V-31) (log P (pH 2.3): 3.47); ¹H NMR (400 MHz, MeCN-d) δ=8.03 (s, 1H), 5.89 (br. s, 1H), 3.09 (s, 2H), 2.09 (s, 3H), 1.53 (s, 6H).

4-(2,5-Dichloropyrimidin-4-yl)thiomorpholine (V-32) (log P (pH 2.3): 2.84); ¹H NMR (400 MHz, DMSO-d) δ=8.27 (s, 1H), 3.99-3.96 (m, 4H), 2.76-2.73 (m, 4H).

4-(2,5-Dichloropyrimidin-4-yl)morpholine (V-33) (log P (pH 2.3): 1.99); ¹H NMR (400 MHz, DMSO-d) δ 8.27 (s, 1H), 3.76-3.69 (m, 8H).

2,5-Dichloro-4-(pyrrolidin-1-yl)pyrimidine (V-34) (log P (pH 2.3): 2.78); ¹H NMR (400 MHz, DMSO-d) δ=8.09 (s, 1H), 3.75-3.71 (m, 4H), 1.92-1.86 (m, 4H).

4-(Azetidin-1-yl)-2,5-dichloropyrimidine (V-35) (log P (pH 2.3): 2.11); ¹H NMR (400 MHz, acetonitrile-d) δ=7.91 (s, 1H), 4.28 (t, 4H), 2.35 (quint, 2H)

2,5-Dichloro-4-(piperidin-1-yl)pyrimidine (V-36) (log P (pH 2.3): 3.52); ¹H NMR (400 MHz, DMSO-d) δ=8.20 (s, 1H), 3.71-3.69 (m, 4H), 1.67-1.59 (m, 6H).

2,5-Dichloro-N-(1,1,1-trifluoropropan-2-yl)pyrimidin-4-amine (V-37) (log P (pH 2.3): 2.66); ¹H NMR (400 MHz, MeCN-d) δ=8.15 (s, 1H), 6.27 (br. s, 1H), 5.11-5.02 (m, 1H), 1.45 (d, 3H).

2,5-Dichloro-N-propylpyrimidin-4-amine (V-38) MATA2888-1-1: log P (pH 2.3): 2.42; ¹H NMR (400 MHz, DMSO-d) δ=8.14 (s, 1H), 7.94 (br. s, 1H), 3.30 (t, 2H), 1.58-1.53 (m, 2H), 0.87 (t, 3H).

2,5-Dichloro-N-(3-methylcyclobutyl)pyrimidin-4-amine (V-39) (Main isomer: log P (pH 2.3): 3.20; ¹H NMR (400 MHz, DMSO-d6) δ=8.10 (s, 1H), 7.72 (s, 1H), 4.25-4.31 (m, 1H), 2.29-2.35 (m, 3H), 1.92-1.99 (m, 2H), 1.06 (d, 3H).

2,5-Dichloro-N-(2-methylcyclopropyl)pyrimidin-4-amine (V-40) (log P (pH 2.3): 2.53; ¹H NMR (400 MHz, DMSO-d6, Main isomer) δ=8.10 (s, 1H), 7.49 (s, 1H), 2.48-2.49 (m, 1H), 1.09 (d, 3 H), 0.96-1.02 (m, 1H), 0.81-0.85 (m, 1H), 0.53-0.58 (m, 1H).

5-Bromo-2-chloro-N-(2-methylcyclopropyl)pyrimidin-4-amine (V-41) (log P (pH 2.3): 2.68; ¹H NMR (400 MHz, DMSO-d6, Main isomer) δ=8.19 (s, 1H), 7.71 (s, 1H), 1.09 (d, 3H), 0.90-1.06 (m, 2H), 0.81-0.86 (m, 1H), 0.53-0.58 (m, 1H).

2-Chloro-N-(2-methylcyclopropyl)-5-(trifluoromethyl)pyrimidin-4-amine (V-42) (logP (pH 2.3): 3.02; ¹H NMR (600 MHz, DMSO-d6, Main isomer) δ=8.39 (s, 1H), 8.00 (s, 1H), 1.10 (d, 3H), 0.84-1.08 (m, 3H), 0.57-0.66 (m, 1H).

2,5-Dichloro-N-(2-ethylcyclopropyl)pyrimidin-4-amine (V-43) (log P (pH 2.3): 3.10; ¹H NMR (400 MHz, DMSO-d6, Main isomer) δ=8.10 (s, 1H), 7.70 (s, 1H), 2.48-2.56 (m, 1H), 1.25-1.40 (m, 2H), 1.00-1.04 (q, 2H), 0.85-0.77 (m, 1H), 0.82-0.84 (m, 1H), 0.56-0.60 (m, 1H).

Synthesis of Intermediates of the Formula (VI) (cf. Scheme 3)

### 1-(3-{[4-Chloro-5-(trifluoromethyl)pyrimidin-2-yl]amino}phenyl)pyrrolidin-2-one (VI-1)

15.7 g (230 ml, 115 mmol) of a 0.5 molar solution of ZnCl₂ in THF are added to a solution of 25.0 g (115 mmol) of 2,4-dichloro-5-trifluoropyrimidine in 150 ml of dichloroethane/tert-butanol (1:1), and the mixture is stirred at room temperature for 30 min. 18.4 g (105 mmol) of 1-(3-aminophenyl)pyrrolidin-2-one (source: MATRIX, ASINEX.) and 16.6 ml (115 mmol) of triethylamine are then added, and the mixture is stirred at room temperature overnight. The crystals formed are filtered off with suction, washed with dichloromethane and dried. This gives 11.5 g (30%) of the desired product. The mother liquor is concentrated and the residue is stirred with 100 ml of isopropanol for 3 h, and the solid is filtered off and dried. This gives 16.0 g (41%) of 1-(3-{[4-chloro-5-(trifluoromethyl)pyrimidin-2-yl]amino}phenyl)pyrrolidin-2-one (log P (pH 2.3): 2.98). ¹H NMR (400 MHz, DMSO-d) δ=10.50 (br. s, 1H) 8.75 (s, 1H), 7.98 (s, 1H), 7.47-7.42 (m 2H), 7.32 (dd, 1H), 3.84-3.81 (m, 2H), 2.53-2.50 (m, 2H), 2.12-2.05 (m, 2H).

The compounds of the formula (VI) below can be prepared in an analogous manner:

1-(5-{[4-Chloro-5-(trifluoromethyl)pyrimidin-2-yl]amino}-2-fluorophenyl)pyrrolidin-2-one (VI-2)

### (log P (pH 2.3): 2.84); ¹H NMR (400 MHz, DMSO-d) δ=10.52 (br.s, 1H), 8.75 (s, 1H), 7.72 (dd, 1H), 7.61-7.57 (m 1H), 7.27 (dd, 1H), 3.77-3.74 (m, 2H), 2.45-2.41 (m, 2H), 2.17-2.04 (m, 2H).

1-(3-{[5-(Difluoromethyl)-4-fluoropyrimidin-2-yl]amino}phenyl)pyrrolidin-2-one (VI-3) (log P (pH 2.3): 2.28); ¹H NMR (400 MHz, DMSO-d6): δ=10.22 (s, 1H), 8.72-8.68 (d, 1H), 7.96 (s, 1H), 7.47-7.28 (m, 3H), 7.05 (t, 1H, J=54 Hz), 3.87-3.80 (m, 3H), 2.12-1.97 (m, 2 H), 1.20-1.06 (m, 1H); M+H=323.1

The compound of the formula (IVa) below can be prepared in an analogous manner:

3-(4-{[4-Chloro-5-(trifluoromethyl)pyrimidin-2-yl]amino}phenyl)-1,3-oxazolidin-2-one (VI-4) (log P (pH 2.3): 2.74); ¹H NMR (400 MHz, DMSO-d6): δ=10.46 (s, 1H), 8.73 (s, 1H), 7.65-7.68 (d, 2H), 7.53-7.55 (d, 2H), 4.44 (t, 2H), 4.05 (t, 2H), M+H=359.0 [Cl].

Synthesis of Compounds of the Formula (I) (cf. Scheme 4)

### Method B:

### 1-[3-({5-(Trifluoromethyl)-4-[(1,1,1-trifluoropropan-2-yl)amino]pyrimidin-2-yl}amino)phenyl]-pyrrolidin-2-one (Example 220)

475 mg (4.2 mmol) of 1,1,1-trifluoropropan-2-amine are added to a solution of 500 mg (1.4 mmol) of 1-(3-{[4-chloro-5-(trifluoromethyl)pyrimidin-2-yl]amino}phenyl)pyrrolidin-2-one in 10 ml of acetonitrile, and the mixture is stirred at 80° C. overnight. After cooling, the reaction mixture is stirred into ice-water and then extracted with ethyl acetate. The organic phase is washed with water, separated off, dried over MgSO₄ and then concentrated on a rotary evaporator. This gives 430 mg (66%) of 1-[3-({5-(trifluoromethyl)-4-[(1,1,1-trifluoropropan-2-yl)amino]pyrimidin-2-yl}amino)phenyl]pyrrolidin-2-one (log P (pH 2.3): 3.08). ¹H NMR (400 MHz, DMSO-d) δ=9.58 (s, 1H), 8.29 (s, 1H), 8.06 (s, 1H), 7.38-7.32 (m 1H), 7.27-7.21 (m, 2H), 6.76 (d, 1H), 5.43-5.35 (m, 1H), 3.82-3.79 (m, 2H), 2.51-2.43 (m, 2H), 2.10-2.03 (m, 2H), 1.42 (d, 3H).

Synthesis of Compounds of the Formula (Ia) (cf. Scheme 5)

### Method A:

### 3-(3-{[5-Chloro-4-(cyclobutylamino)pyrimidin-2-yl]amino}phenyl)-1,3-oxazolidin-2-one (Example 59)

A mixture of 0.21 g (1.0 mmol) of 2,5-dichloro-N-cyclobutylpyrimidin-4-amine, 0.23 g (1.30 mmol) of 3-(3-aminophenyl)-1,3-oxazolidin-2-one and 0.15 g (0.80 mmol) of 4-toluenesulphonic acid in 5 ml of dioxane is stirred at 100° C. for 40 h. After cooling, the reaction mixture is concentrated under reduced pressure and the residue is taken up in 50 ml of ethyl acetate. The organic phase is washed with 10 ml of aq. NaHCO₃ and then with 10 ml of water, dried over MgSO₄ and freed from the solvent under reduced pressure. The crude product is purified by column chromatography on silica gel (cyclohexane/ethyl acetate). This gives 0.11 g of the desired product (log P (pH 2.3): 1.64). ¹H NMR (400 MHz, DIMETHYL SULPHOXIDE-d) δ=9.01 (s, 1H), 7.96 (s, 1H), 7.91 (s, 1H), 7.47 (d, 1H), 7.23 (t, 1H), 7.14 (m, 1H), 6.96 (m, 1 H), 4.63 (br. m, 1H), 4.43 (dd, 2H), 4.05 (dd, 2H), 2.28 (br. m, 2H), 2.13 (br. m, 2H), 1.67 (br. m, 2H).

Synthesis of Intermediates for Method C (cf. Scheme 6)

### 5-Chloro-N⁴-cyclopropyl-N²-(4-iodophenyl)pyrimidine-2,4-diamine

Under an atmosphere of argon, a mixture of 0.20 g (0.98 mmol) of 2,5-dichloro-N-cyclopropylpyrimidin-4-amine, 0.27 g (1.22 mmol) of 4-iodoaniline and 0.14 g (0.83 mmol) of 4-toluenesulphonic acid in 5 ml of dioxane is stirred at 105° C. for 18 hours. After cooling, the reaction mixture is concentrated under reduced pressure and the residue is taken up in 50 ml of water, neutralized with saturated aqueous NaHCO₃ solution and extracted with ethyl acetate. The organic phase is dried over MgSO₄ and freed from the solvent under reduced pressure. The crude product is purified by column chromatography on silica gel (cyclohexane/ethyl acetate). This gives 0.65 g of the desired product (log P (pH 2.3): 2.73). ¹H NMR (400 MHz, DIMETHYL SULPHOXIDE-d) δ=9.17 (s, 1H), 7.95 (s, 1H), 7.91 (m, 1H), 7.69-7.60 (m, 2H), 7.55-7.53 (m, 2H), 7.04 (s, 1H), 2.86-2.81 (m, 1H), 0.81-0.76 (m, 2H), 0.66-0.62 (m, 2H),

Synthesis of Intermediates of the Formula (VIa) (cf. Scheme 7) 3-(4-{[4-Chloro-5-(trifluoromethyl)pyrimidin-2-yl]amino}phenyl)-1,3-oxazolidin-2-one

At 0° C., 18 ml (18 mmol) of a 1M solution of zinc chloride in ether are added dropwise to a solution of 3.26 g (15 mmol) of 2,4-dichloro-5-trifluoropyrimidine in a mixture of 40 ml of dichlorethane and 40 ml or tert-butanol, and the mixture is stirred at the same temperature for 1 hour. 2.67 g (15 mmol) of 3-(4-aminophenyl)-1,3-oxazolidin-2-one are then added, and 2.3 ml of triethylamine in a mixture of 5 ml of dichloroethane and 5 ml of tert-butanol are then added dropwise. The mixture is stirred at 20° C. for 40 hours. The mixture is then freed from the solvent under reduced pressure and stirred with a mixture of 100 ml of water and 100 ml of ethyl acetate. The organic phase is then separated off, dried over MgSO₄ and freed from the solvent under reduced pressure. The crude product is then saturated with 100 ml of ethyl acetate. This gives 4.7 g of the desired product (log P (pH 2.3): 2.76). ¹H NMR (400 MHz, DMSO-d6): δ=10.46 (s, 1H), 8.73 (s, 1H), 7.65-7.68 (d, 2H), 7.53-7.55 (d, 2H), 4.44 (t, 2H), 4.05 (t, 2H), M+H=359.0 [Cl].

Synthesis of Intermediates of the Formula (IVa) (cf. Scheme 10)

### 3-(3-Aminophenyl)-1,3-oxazolidin-2-one

Under an atmosphere of argon, 0.26 g (2.3 mmol) of 1,2-diaminocyclohexane is added at 20° C. to a solution of 5.0 g (22.8 mmol) of 3-iodoaniline, 3.0 g (34.2 mmol) of 1,3-oxazolidin-2-one, 6.3 g (45.7 mmol) of potassium carbonate and 0.17 g (0.91 mmol) of copper(I) iodide in 40 ml of dioxane. The reaction mixture is stirred at 100° C. for 18 hours. After cooling, the reaction mixture is filtered off through kieselguhr and freed from the solvent under reduced pressure. The residue is then stirred in 50 ml of dichloromethane and 50 ml of water. To remove the oxazolidinone, the residue is then once more taken up in ethyl acetate and again freed from the solvent under reduced pressure. This gives 23 mg of the desired product (log P (pH 2.3): −0.18). ¹H NMR (400 MHz, DIMETHYL SULPHOXIDE-d) δ=6.98 (t, 1H), 6.84 (s, 1H), 6.66 (m, 1H), 6.35 (m 1H), 4.98 (br. s, 2H), 4.37 (dd, 2H), 3.96 (dd, 2H).

### (4R)-3-(3-Aminophenyl)-4-methyl-1,3-oxazolidin-2-one

At 20° C., 0.32 g (3.7 mmol) of 1,2-diaminocyclohexane is added to a solution of 2.0 g (9.1 mmol) of 3-iodoaniline, 1.6 g (16.0 mmol) of (4R)-4-methyl-1,3-oxazolidin-2-one, 6.0 g (18.3 mmol) of caesium carbonate and 0.7 g (3.7 mmol) of copper(I) iodide in 20 ml of dioxane. The reaction mixture is stirred in a microwave at 160° C. for one hour. After cooling, the reaction mixture is filtered off through kieselguhr and freed from the solvent under reduced pressure. This gives 0.91 g of the desired product (log P (pH 2.3): 0.27). ¹H NMR (400 MHz, acetonitrile-d) δ=7.09 (t, 1H), 6.78 (t, 1H), 6.70-6.64 (m, 1H), 6.49-6.44 (m, 1H), 4.53-4.43 (m, 2H), 4.22 (s, 2H), 3.99-3.90 (m, 1H), 1.22 (d, 3H)

The compounds of the formula (IVa) below can be prepared in an analogous manner:

(4R)-3-(3-Aminophenyl)-4-isopropyl-1,3-oxazolidin-2-one (log P (pH 2.3): 1.18); ¹H NMR (400 MHz, DMSO-d) δ=7.00 (t, 1H), 6.74 (t, 1H), 6.62-6.46 (m, 1H), 6.42-6.36 (m, 1H), 4.99 (s, 2H), 4.45-4.15 (m, 3H), 2.05-1.94 (m, 1H), 0.79 (dd, 6H)

3-(3-Aminophenyl)-5-methyl-1,3-oxazolidin-2-one (log P (pH 2.3): 0.33); ¹H NMR (400 MHz, DMSO-d) δ=6.98 (t, 1H), 6.84 (t, 1H), 6.66-6.61 (m, 1H), 6.36-6.31 (m, 1H), 4.96 (s, 2H), 4.78-4.68 (m, 1H), 4.06 (dd, 1H), 3.54 (dd, 1H), 1.39 (d, 3H).

Synthesis of Compounds of the Formula (Ib) (cf. Scheme 11)

### Method A (Microwave):

### 1-(3-{[5-Chloro-4-(cyclopropylamino)pyrimidin-2-yl]amino}phenyl)-5-ethyl-3-methylpyrrolidin-2-one (Example 206)

A mixture of 67 mg (0.33 mmol) of 2,5-dichloro-N-cyclopropylpyrimidin-4-amine, 90 mg (0.41 mmol) of 1-(3-aminophenyl)-5-ethyl-3-methylpyrrolidin-2-one and 53 g (0.28 mmol) of 4-toluenesulphonic acid in 2 ml of dioxane is reacted at 160° C. in a microwave for 30 minutes. After cooling, the reaction mixture is concentrated under reduced pressure and the residue is taken up in 50 ml of ethyl acetate. The organic phase is washed with 10 ml of saturated aqueous NaHCO₃, dried over MgSO₄ and freed from the solvent under reduced pressure. This gives 74 mg of the desired product (log P (pH 2.3): 2.19). (two diastereoisomers) ¹HNMR (400 MHz, DMSO-d) δ=9.09 (s, 1H minor), 9.06 (s, 1H major), 7.90 (s, 1H major); MM+1=386.1

### Method A

### Ethyl 1-(3-{[5-chloro-4-(cyclopropylamino)pyrimidin-2-yl]amino}phenyl)-5-oxo-L-prolinate (Example 190)

A mixture of 250 mg (1.23 mmol) of 2,5-dichloro-N-cyclopropylpyrimidin-4-amine, 380 mg (1.53 mmol) of ethyl 1-(3-aminophenyl)-5-oxo-L-prolinate and 170 mg (0.98 mmol) of 4-toluenesulphonic acid in 12 ml of dioxane is stirred at 105° C. for 32 h. After cooling, the reaction mixture is poured into ice-water, ethyl acetate and NaHCO₃ solution are added and the organic phase is separated off, washed once with water, dried over MgSO₄ and freed from the solvent under reduced pressure. This gives 350 mg of the desired product (log P (pH 2.3): 1.69). ¹H NMR (400 MHz, DMSO-d) δ=9.08 (s, 1H), 7.98 (dd, 1H), 7.89 (s, 1H), 7.62 (dd 1H), 7.19 (dd, 1H), 6.97-6.94 (m, 2H), 4.79-4.77 (m, 1H), 4.14-4.08 (m, 2H), 2.97-2.92 (m, 1H), 2.54-2.42 (m, 2H), 2.07-2.02 (m, 2H), 1.13 (t, 3H), 0.76-0.74 (m, 2H), 0.65-0.64 (m, 2H).

### Synthesis of the Intermediate:

### Ethyl 1-(3-aminophenyl)-5-oxo-L-prolinate

A mixture of 3.23 g (20.5 mmol) of ethyl 5-oxo-L-prolinate, 3.0 g (13.7 mmol) of 3-iodoaniline, 0.52 g (2.73 mmol) of copper(I) iodide, 0.24 g (2.74 mmol) of N,N′-dimethylenethyldiamine and 8.9 g (27 mmol) of caesium carbonate is taken up in 28 ml of dioxane and stirred at 100° C. for 24 hours. After cooling, the reaction solution is filtered through a silica gel cartridge, the cartridge is washed with ethyl acetate and the filtrate is concentrated under reduced pressure. This gives 3.5 g of the desired product (log P (pH 2.3): 0.84). ¹H NMR (400 MHz, MeCN-d) δ=7.04 (dd, 1 H), 6.85 (dd, 1H), 6.65 (dd 1H), 6.44 (dd, 1H), 4.68-4.65 (m, 1H), 4.14 (q, 2H), 4.07 (br.s, 2 H), 2.60-2.53 (m, 1H), 2.45-2.39 (m, 2H), 2.11-1.96 (m, 1H), 1.21 (t, 3H).

Method C: (cf. Scheme 13)

### 1-(3-{[5-Chloro-4-(cyclopropylamino)pyrimidin-2-yl]amino}phenyl)-5-ethoxypyrrolidin-2-one (Example 106)

Under an atmosphere of argon, 14 mg (0.16 mmol) of 1,2-diaminocyclohexane are added at 20° C. to a mixture of 0.30 g (0.78 mmol) of 5-chloro-N⁴-cyclopropyl-N²-(3-iodophenyl)pyrimidine-2,4-diamine, 0.15 g (1.16 mmol) of 5-ethoxypyrrolidin-2-one, 0.51 g (1.55 mmol) of caesium carbonate and 30 g (0.15 mmol) of copper(I) iodide in 15 ml of dioxane. The mixture is stirred at 110° C. for 18 hours. After cooling, the reaction mixture is filtered off through kieselguhr and freed from the solvent under reduced pressure. The residue is then taken up in 15 ml of ethyl acetate and 25 ml of water. The organic phase is separated off and again freed from the solvent under reduced pressure. The residue is then saturated with tert-butyl methyl ether and again filtered off. The residue obtained is 0.31 g of the desired product (log P (pH 2.3): 1.65). ¹H NMR (400 MHz, DMSO-d) δ=9.10 (s, 1H), 8.02 (s, 1H), 7.94-7.92 (m, 1H), 7.64 (d 1H), 7.22 (d, 1H), 7.02-6.97 (m, 2H), 5.42 (d, 1H), 3.44 (q, 2H), 2.97-2.92 (m, 1H), 2.36-2.19 (m, 2H), 2.02-1.97 (m, 2H), 1.05 (t, 3H), 0.74-0.72 (m, 2H), 0.65-0.63 (m, 2H).

Method F: (cf. Scheme 14)

### N-{5-Chloro-4-[cyclopropylmethyl)amino]pyrimidin-2-yl}-N-[3-(2-oxopyrrolidin-1-yl)phenyl]acetamide (Example 184)

104 mg (0.29 mmol) of 1-[3-({5-chloro-4-[cyclopropyl(methyl)amino]pyrimidin-2-yl}amino)phenyl]-pyrrolidin-2-one are taken up in 1.08 g (10.6 mmol) of acetic anhydride, 4.45 g (14.3 mmol) of triethylamine and 30 mg (0.25 mmol) of DMAP added and the mixture is stirred under microwave conditions at 150° C. for 1 h. After cooling, the reaction mixture is freed from the solvent under reduced pressure. Ethyl acetate and NaHCO₃ solution are added to the residue, and the organic phase is separated off, washed twice with water, dried over MgSO₄ and again freed from the solvent under reduced pressure. The crude product is purified by column chromatography on silica gel (cyclohexane/ethyl acetate). This gives 95 mg of the desired product (log P (pH 2.3): 2.35). ¹H NMR (400 MHz, DMSO-d) δ=8.20 (s, 1H), 7.59 (dd, 1H), 7.47 (s, 1H), 7.35 (dd 1H), 6.93 (dd, 1H), 3.79 (t, 2H), 3.03 (s, 3H), 2.29 (s, 3H), 2.07-1.97 (m, 2H), 0.79-0.74 (m, 2H), 0.64-0.62 (m, 2H).

### N-[5-Chloro-4-(cyclopropylamino)pyrimidin-2-yl]-N-[3-(2-oxopyrrolidin-1-yl)phenyl]-formamide (Example 217)

350 mg (1.0 mmol) of 1-(3-{[5-chloro-4-(cyclopropylamino)pyrimidin-2-yl]amino}phenyl)pyrrolidin-2-one, 603 mg (4.07 mmol) of triethyl orthoformate and 17.5 mg (0.1 mmol) of p-toluenesulphonic acid are taken up in 4 ml of toluene and, under microwave conditions, stirred at 180° C. for 4 h. After cooling, the reaction mixture is poured into ice-water, ethyl acetate and NaHCO₃ solution are added and the organic phase is separated off, washed once with water, dried over MgSO₄ and again freed from the solvent under reduced pressure. The crude product is purified by column chromatography on RP18 (water/CH₃CN). This gives 75 mg of the desired product (log P (pH 2.3): 1.22). ¹H NMR (400 MHz, DMSO-d) δ=9.81 (s, 1H), 8.02 (s, 1H), 7.72 (d, 1H), 7.62-7.59 (m, 2H), 7.43 (dd 1H), 6.98 (d, 1H), 3.81 (t, 2H), 3.04-3.00 (m, 1H), 2.09-2.02 (m, 2H), 0.75-0.62 (m, 4H).

### N-[5-Chloro-4-(cyclopropylamino)pyrimidin-2-yl]-2-methoxy-N-[3-(2-oxopyrrolidin-1-yl)phenyl]acetamide (Example 231)

500 mg (1.54 mmol) of 1-(3-{[5-chloro-4-(cyclopropylamino)pyrimidin-2-yl]amino}phenyl)pyrrolidin-2-one and 10 mg (0.07 mmol) of DMAP are taken up in 1 ml of acetonitrile. 632 mg (5.8 mmol) of methoxyacetyl chloride are then added. After 2 h of stirring at room temperature, 301 mg (2.18 mmol) of potassium carbonate are added, and the mixture is stirred at room temperature for 16 h and under reflux for a further 16 h. After cooling, the reaction mixture is poured into ice-water, ethyl acetate and NaHCO₃ solution are added and the organic phase is separated off, washed twice with water, dried over MgSO₄ and again freed from the solvent under reduced pressure. The crude product is purified by column chromatography on silica gel (cyclohexane/ethyl acetate). This gives 23 mg of the desired product (log P (pH 2.3): 1.87). ¹H NMR (400 MHz, DMSO-d) δ=9.28 (s, 1H), 7.99 (s, 1H), 7.93 (s, 1H) 7.69-7.65 (m, 1H), 7.30-7.19 (m, 2H), 4.34 (s, 2H), 3.78-3.75 (m, 2H), 2.93-2.90 (m, 1H), 2.08-2.02 (m, 2H), 0.76-0.63 (m, 4H).

### Ethyl [5-chloro-4-(cyclopropylamino)pyrimidin-2-yl][3-(2-oxopyrrolidin-1-yl)phenyl]-carbamate (Example 330)

Under argon, 250 mg (0.72 mmol) of 1-(3-{[5-chloro-4-(cyclopropylamino)pyrimidin-2-yl]amino}phenyl)pyrrolidin-2-one are dissolved in 10 ml of DMF, and 43 mg (1.1 mmol) of sodium hydride (60%) are then added at 0° C. After 30 min of stirring, 118 mg (1.1 mmol) of ethyl chloroformate are added dropwise. After 12 h of stirring at room temperature, the reaction mixture is poured into ice-water, ethyl acetate and NaHCO₃ solution are added and the organic phase is separated off, washed twice with water, dried over MgSO₄ and again freed from the solvent under reduced pressure. The crude product is purified by column chromatography on silica gel (cyclohexane/ethyl acetate). This gives 310 mg of the desired product (log P (pH 2.3): 2.04); ¹H NMR (400 MHz, DMSO-d) δ=8.11 (s, 1H), 7.65 (dd, 1H), 7.41 (dd, 1H), 7.39 (br. s, 1H), 7.32 (dd, 1H), 6.96 (dd, 1H), 4.16 (q, 2H), 3.79 (t, 2H), 2.78-2.73 (m, 1H), 2.52-2.44 (m, 2H), 2.07-2.03 (m, 2H), 1.18 (t, 3H), 0.65-0.62 (m, 4H).

The compound below can be prepared in an analogous manner:

Isopropyl [5-chloro-4-(cyclopropylamino)pyrimidin-2-yl][3-(2-oxopyrrolidin-1-yl)phenyl]carbamate (Example 329) (log P (pH 2.3): 2.24); ¹H NMR (400 MHz, DMSO-d) δ=8.11 (s, 1H), 7.64 (dd, 1H), 7.40 (dd, 1H), 7.36 (br. s, 1H), 7.31 (dd, 1H), 6.94 (dd, 1H), 4.92 (h, 1H), 3.79 (t, 2H), 2.81-2.77 (m, 1H), 2.53-2.44 (m, 2H), 2.09-2.01 (m, 2H), 1.19 (d, 6 H), 0.67-0.63 (m, 4H).

Synthesis of Compounds of the Formula (Ib-II) (cf. Scheme 12)

### Method E:

### 1-(3-{[5-Chloro-4-(cyclopropylamino)pyrimidin-2-yl]amino}phenyl)pyrrolidine-2-thione (Example 33)

3.0 g (8.7 mmol) of 1-(3-{[5-chloro-4-(cyclopropylamino)pyrimidin-2-yl]amino}phenyl)pyrrolidin-2-one are dissolved in 30 ml of pyridine and, at 100° C., reacted with 3.5 g (8.7 mmol) of 4-methoxyphenyldithiophosphonic anhydride (Lawesson reagent) for 5 hours. Another 0.35 g (0.87 mmol) of 4-methoxyphenyldithiophosphonic anhydride (Lawesson reagent) is then added, and the mixture is stirred at 100° C. for another 5 hours. After cooling, the reaction solution is poured into 500 ml of ice-water and 50 ml of dilute hydrochloric acid, and the mixture is stirred for 30 minutes. The precipitate is then filtered off, washed thoroughly three times with 300 ml of water and triturated with 50 ml of tert-butyl methyl ether. The solid is once more filtered off with suction and stirred with 80 ml of water. The mixture is neutralized with saturated NaHCO₃ solution and stirred at 20° C. for one hour, and the solid is filtered off. This solid is then triturated with 80 ml of isopropanol and again filtered off with suction. This gives 2.1 g of the desired product (log P (pH 7): 2.54). ¹H NMR (400 MHz, DMSO-d) δ=9.43 (s, 1H), 8.19 (dd, 1H), 7.95 (s, 1H), 7.68 (dd, 1H), 7.33-7.28 (m, 2H), 6.99 (dd, 1H), 4.07 (t, 2H), 3.03 (t, 2H), 2.88-2.84 (m, 1H), 2.15-2.07 (m, 2H), 0.75-0.62 (m, 4H).

### Synthesis of Compounds of the Formula (Ib-IV)

### Method G:

### 1-(3-{[4-(Cyclopropylamino)-5-(trifluoromethyl)pyrimidin-2-yl]amino}phenyl)pyrrolidin-2-one (Example 236) (cf. Scheme 15)

250 mg (0.647 mmol) of 5-chloro-N⁴-cyclopropyl-N²-(3-iodophenyl)pyrimidine-2,4-diamine, 165 mg (1.94 mmol) of 2-pyrrolidone, 29 mg (0.129 mmol) of Pd(OAc)₂ are initially charged in 1.5 ml of tetrahydrofuran in a 2.5 ml microwave vial. 170 mg (0.647 mmol) of Mo(CO)₆ and 295 mg (1.94 mmol) of DBU are then added, and the mixture is stirred in the closed vial at 100° C. with microwave irradiation for 10 minutes. After the reaction has ended, the mixture is cooled and filtered through Celite, the Celite is washed with ethyl acetate and the filtrate is evaporated to dryness on a rotary evaporator. The crude product obtained is separated chromatographically on reversed-phase material (Analogix SF25-100) using water/acetonitrile. This gives 62 mg of 1-(3-{[4-(cyclopropylamino)-5-(trifluoromethyl)pyrimidin-2-yl]amino}phenyl)pyrrolidin-2-one (log P (pH 2.3): 1.49. ¹H NMR (400 MHz, DMSO-d) δ=9.17 (s, 1H), 8.12 (t, 1H), 7.94-7.86 (m, 2H), 7.25 (t, 1H), 7.08-6.98 (m, 2H), 3.80 (t, 2H), 2.86-2.77 (m, 1H), 2.08-1.97 (m, 2H), 0.80-0.74 (m, 2H), 0.66-0.60 (m, 2H).

### Synthesis of the Intermediate:

### 5-Chloro-N⁴-cyclopropyl-N²-(3-iodophenyl)pyrimidine-2,4-diamine (VII-1)

13.4 g (61 mmol) of 3-iodoaniline and 6.75 g (39 mmol) of p-toluenesulphonic acid are added to a solution of 10 g (49 mmol) of 2,5-dichloro-N-cyclopropylpyrimidin-4-amine in 250 ml of dioxane, and the mixture is stirred at 105° C. for 16 h. After cooling, the reaction mixture is filtered off with suction, suspended in water, stirred for 30 min and again filtered off with suction. The mixture is then again suspended in water and neutralized with 1N NaOH, and the residue formed is again filtered off with suction. The residue is washed with water. This gives 18.5 g (96.7%) of 5-chloro-N⁴-cyclopropyl-N²-(3-iodophenyl)pyrimidine-2,4-diamine (log P (pH 2.3): 3.08. ¹H NMR (400 MHz, DMSO-d) δ=9.94 (s, 1H), 8.45 (dd, 1H), 8.07 (s, 1H), 7.86 (br. s, 1H), 7.61 (dd 1 H), 7.34 (dd, 1H), 7.08 (dd, 1H), 2.90-2.86 (m, 1H), 0.93-0.88 (m, 2H), 0.76-0.72 (m, 2H).

The compounds of the formula (VII) below can be prepared in an analogous manner:

5-Bromo-N⁴-cyclopropyl-N²-(3-iodophenyl)pyrimidine-2,4-diamine (VII-2), (log P (pH 2.3): 3.34 ¹H NMR (400 MHz, DMSO-d) δ=9.44 (br. s, 1H), 8.50 (dd, 1H), 8.05 (s, 1H), 7.63 (dd 1 H), 7.27 (dd, 1H), 7.13 (br. s, 1H), 7.04 (dd, 1H), 2.87-2.83 (m, 1H), 0.91-0.86 (m, 2H), 0.72-0.68 (m, 2H).

5-Chloro-N²-[3-bromo-4-(trifluoromethoxy)phenyl]-N⁴-cyclopropylpyrimidine-2,4-diamine (VII-3)

(log P (pH 2.3): 3.99)

5-Chloro-N²-[3-bromo-4-(methyl)phenyl]-N⁴-cyclopropylpyrimidine-2,4-diamine (VII-4) (log P (pH 2.3): 2.91)

N²-(3-Bromophenyl)-5-chloro-N⁴-cyclopropylpyrimidine-2,4-diamine (VII-5) (log P (pH 2.3): 2.91)

N²-(3-Bromo-4-chlorophenyl)-5-chloro-N⁴-cyclopropylpyrimidine-2,4-diamine (VII-6) (log P (pH 2.3): 3.55)

N²-[3-Bromo-5-(trifluoromethyl)phenyl]-5-chloro-N⁴-cyclopropylpyrimidine-2,4-diamine (VII-7) (log P (pH 2.3): 4.6); ¹H NMR (400 MHz, DMSO-d6) δ=9.65 (s, 1H), 8.48 (s, 1H), 8.23 (s, 1H), 7.99 (s, 1H), 7.35 (s, 1H), 7.25 (s, 1H), 2.81-2.86 (m, 1H), 0.81-0.85 (m, 2H), 0.68-0.71 (m, 2H).

N²-(4-Bromophenyl)-5-chloro-N⁴-cyclopropylpyrimidine-2,4-diamine (VII-8) (log P (pH 2.3): 2.55)

5-Bromo-N²-(4-bromophenyl)-N⁴-(cyclopropylmethyl)pyrimidine-2,4-diamine (VII-9) (log P (pH 2.3): 2.84)

5-Bromo-N²-(4-bromophenyl)-N⁴-cyclobutylpyrimidine-2,4-diamine (VII-10) (log P (pH 2.3): 3.18)

5-Bromo-N²-(4-bromophenyl)-N⁴-cyclopropylpyrimidine-2,4-diamine (VII-11) (log P (pH 2.3): 2.7)

5-Chloro-N⁴-cyclobutyl-N²-(4-iodophenyl)pyrimidine-2,4-diamine (VII-12) (log P (pH 2.3): 3.49); ¹H NMR (400 MHz, DMSO-d) δ=9.09 (s, 1H), 7.91 (s, 1H), 7.60-7.52 (m, 4H), 6.99 (d, 1H), 4.58-4.46 (m, 1H), 2.35-2.25 (m, 2H), 2.20-2.07 (m, 2H), 1.79-1.65 (m, 2H)

### Synthesis of Intermediates of the Formula (IVb)

### 1-(5-Amino-2-fluorophenyl)pyrrolidin-2-one (cf. Scheme 16)

950 mg (78.8 mmol) of 1-(2-fluoro-5-nitrophenyl)pyrrolidin-2-one are dissolved in 150 ml of methanol, 2 g of Pd/C (10%) are added and the mixture is stirred in an autoclave at a hydrogen pressure of 5 bar at 30° C. for 10 h. The catalyst is filtered off with suction and the filtrate is then evaporated to dryness on a rotary evaporator, giving 14.5 g (96%) of 1-(5-amino-2-fluorophenyl)pyrrolidin-2-one (log P (pH 2.3): 0.31. ¹H NMR (400 MHz, MeCN-d) δ=6.88 (dd, 1 H), 6.63 (dd 1H), 6.52 (ddd, 1H), 3.71 (t, 2H), 2.43-2.37 (m, 2H), 2.16-2.09 (m, 2H).

### 1-(3-Aminophenyl)-5-methylpyrrolidin-2-one (cf. Scheme 17)

A mixture of 10.1 g (100 mmol) of 5-methylpyrrolidin-2-one, 15 g (67 mmol) of 3-iodoaniline, 2.56 g (13.4 mmol) of copper(I) iodide, 0.25 g (26 mmol) of N,N′-dimethylenethyldiamine and 43.7 g (134 mmol) of caesium carbonate is taken up in 180 ml of dioxane and stirred at 100° C. overnight. After cooling, the reaction mixture is filtered through a silica gel cartridge, the cartridge is washed with ethyl acetate and the filtrate is concentrated under reduced pressure. The crude product is purified by column chromatography on silica gel (cyclohexane/ethyl acetate). This gives 7.5 g of the desired product (log P (pH 2.3): 0.52). ¹H NMR (400 MHz, DMSO-d) δ=6.99 (t, 1H), 6.67 (t, 1H), 6.56-6.48 (m, 1H), 6.43-6.37 (m, 1H), 4.94 (s, 2H), 4.24-4.15 (m, 1H), 2.50-2.00 (m, 3H), 1.70-1.55 (m, 1H), 1.12 (d, 3H)

### 1-(3-Aminophenyl)-5-ethyl-3-methylpyrrolidin-2-one (cf. Scheme 17)

A mixture of 515 mg (2.3 mmol) of 3-iodoaniline, 500 mg (3.46 mmol) of 5-ethyl-3-methylpyrrolidin-2-one and 88 g (0.46 mmol) of copper(I) iodide, 86 mg (0.9 mmol) of N,N′-dimethylenethyldiamine and 1.5 g (4.6 mmol) of caesium carbonate is taken up in 10 ml of dioxane and reacted at 160° C. in a microwave for 45 minutes. After cooling, the reaction mixture is concentrated under reduced pressure and purified by column chromatography on silica gel (water/CH₃CN). This gives 190 mg of the desired product (log P (pH 2.3): 1.31). (Two diastereoisomers) ¹H NMR (400 MHz, DMSO-d) δ=7.03-6.95 (m, 1H), 6.74-6.70 (m, 1H), 6.61-6.53 (m, 1H), 6.45-6.35 (m, 1H), 4.91 (s, 2H), 4.06-3.90 (m, 1H), 2.70-1.20 (5H), 1.15 (t, 3H minor), 1.11 (t, 3H major), 0.82 (t, 3H major), 0.76 (t, 3H minor). MM+1=219.2

The compounds of type (IVb) below can be prepared in an analogous manner:

1-(3-Aminophenyl)-5-ethylpyrrolidin-2-one (log P (pH 2.3): 0.94); ¹H NMR (400 MHz, DMSO-d) δ=6.98 (t, 1H), 6.65 (t, 1H), 6.54-6.48 (m, 1H), 6.43-6.37 (m, 1H), 4.91 (s, 2H), 4.12-4.05 (m, 1H), 2.50-2.29 (m, 2H), 2.25-2.15 (m, 1H), 1.78-1.68 (m, 1H), 1.63-1.53 (m, 1H), 1.43-1.30 (m, 1H), 0.79 (t, 3H)

1-(3-Aminophenyl)-5-(trifluoromethyl)pyrrolidin-2-one (log P (pH 2.3): 1.07); ¹H NMR (400 MHz, DMSO-d) δ=7.01 (t, 1H), 6.63 (t, 1H), 6.54-6.49 (m, 1H), 6.48-6.43 (m, 1H), 5.05-4.90 (m, 3H), 2.60-1.95 (m, 4H)

### 1-(3-Amino-5-methoxyphenyl)pyrrolidin-2-one

3.2 g (13.5 mmol) of 1-(3-methoxy-5-nitrophenyl)pyrrolidin-2-one are dissolved in 60 ml of methanol and, in an autoclave under a hydrogen pressure of 3 bar, stirred over 500 mg of Pd/C 10% at 30° C. The catalyst is filtered off with suction and the filtrate is evaporated to dryness on a rotary evaporator, giving 2.10 g of the desired product (log P (pH 2.3): 0.60); ¹H NMR (400 MHz, DMSO-d) δ=6.48-6.46 (m, 2H), 5.97 (dd, 1H), 4.97 (br.s, 2H), 3.72 (t, 2H), 3.65 (s, 3H), 2.43 (t, 2 H), 2.05-1.97 (m, 2H).

### Synthesis of Intermediates of the Formula (XII)

### 3-Methyl-1-(3-nitrobenzyl)pyrrolidin-2-one (cf. Scheme 18)

Under argon, 4.0 g (18.5 mmol) of 3-methylpyrrolidin-2-one are initially charged at 0° C. in THF. 0.963 g (24 mmol) of NaH (60% in paraffin) is then added, and the mixture is stirred 0° C. for 30 min. A solution of 4.0 g (18.5 mmol) of 3-nitrobenzyl bromide in 10 ml of THF is then added dropwise, and the mixture is stirred at room temperature for 3 h. The reaction solution is then concentrated under reduced pressure and taken up in ice-water/1N hydrochloric acid (1:1), and the organic phase is separated off, washed and dried over Na₂SO₄. After removal of the solvent under reduced pressure, the crude product is purified by column chromatography on silica gel (cyclohexane/ethyl acetate 2:1 to 0:1). This gives 3.9 g of the desired product (log P (pH 2.3): 1.82). ¹H NMR (400 MHz, DMSO-d) δ=8.13-8.10 (m, 1H), 8.05-8.04 (m, 1H), 7.68-7.62 (m, 2 H), 4.51 (s, 2H), 3.26-3.17 (m, 2H), 2.45-2.39 (m, 1H), 2.25-2.17 (m, 1H), 1.61-1.52 (m, 1H), 1.11 (d, 3H).

### 1-(3-Methoxy-5-nitrophenyl)pyrrolidin-2-one

5.98 g (43.3 mmol) of potassium carbonate are added to a solution of 5.90 g (21.6 mmol) of 4-chloro-N-(3-methoxy-5-nitrophenyl)butanamide in 120 ml of acetonitrile, and the reaction mixture is heated at 80° C. After 4 h, the reaction mixture is stirred into ice-water/dilute hydrochloric acid. The precipitate formed is filtered off, washed with water and dried. This gives 4.50 g (log P (2.3): 1.99); ¹H NMR (400 MHz, DMSO-d) δ=8.23 (dd, 1H), 7.58 (dd, 1H), 7.47 (dd, 1H), 3.90 (t, 2 H), 3.82 (s, 3H), 2.55 (t, 2H), 2.13-2.05 (m, 2H).

Synthesis of Intermediates of the Formula (XIIa) (cf. Scheme 19)

### 1-(2-Fluoro-5-nitrophenyl)pyrrolidin-2-one

A mixture of 30 g (192 mmol) of 2-fluoro-5-nitroaniline and 15.4 g (175 mmol) of butyrolactone in 15 ml of hydrochloric acid is stirred at 160° C. for 8 h. After cooling, the reaction mixture is taken up in 200 ml of ethyl acetate, 15 g of kieselguhr are added to the solution and the mixture stirred at 40° C. for a further 30 min. The mixture is concentrated under reduced pressure and the residue is taken up in 50 ml of ethyl acetate. The mixture is filtered off, and the solvent is then removed under reduced pressure. The crude product is purified by column chromatography on silica gel (cyclohexane/ethyl acetate 1:1 to 0:1). This gives 22 g of the desired product as a crude material. The product is then triturated with MTBE, filtered off and reconcentrated, giving 17.4 g (43%) of the desired product in a 97% pure form (log P (pH 2.3): 1.47). ¹H NMR (400 MHz, MeCN-d) δ=8.37 (dd, 1H), 8.12 (ddd, 1H), 7.43-7.33 (m, 1H), 3.86-3.83 (m, 2H), 2.49-2.44 (m, 2H), 2.22-2.17 (m, 2H).

### 4-Chloro-N-(3-methoxy-5-nitrophenyl)butanamide

4.00 g (23.8 mmol) of 3-methoxy-5-nitroaniline are heated to boiling point in 160 ml of toluene, and 3.35 g (23.8 mmol) of 4-chlorobuteryl chloride are then added. After 6 h of stirring under reflux, the reaction mixture is allowed to cool to room temperature and the precipitate is filtered off with suction. This gives 6.10 g (log P (2.3): 2.46); ¹H NMR (400 MHz, DMSO-d) δ=8.13 (dd, 1 H), 7.59 (dd, 1H), 7.39 (dd, 1H), 3.85 (s, 3H), 3.69 (t, 2H), 2.52 (t, 2H), 2.10-2.03 (m, 2H).

### Synthesis of Intermediates of the Formula (XIa)

### Methyl 2-methyl-4-nitrohexanoate [cf. Scheme 19 and Paragraph (t)]

At 55-60° C., 88 g (1 mol) of nitropropane and 5.52 g (40 mmol) of potassium carbonate are initially charged in 32 g of methanol. 20 g (0.2 mol) of methyl acrylate are slowly added dropwise, and stirring at 55-60° C. is continued overnight. After cooling, the insolubles are filtered off and the solvent is removed under reduced pressure. Subsequent distillation under high vacuum affords 4.9 g of the desired product (log P (pH 2.3): 2.23). (two diastereoisomers) ¹H NMR (400 MHz, acetonitrile-d) δ=4.58-4.45 (m, 1H), 3.65 (s, 3H major), 3.60 (s, 3H minor), 2.49-2.30 (m, 1H), 2.09-1.74 (m, 4H), 1.18-1.12 (m, 3H), 0.96 (t, 3H)

### 5-Ethyl-3-methylpyrrolidin-2-one [cf. Scheme 19 and Paragraph (t)]

23 g (121 mmol) of methyl 2-methyl-4-nitrohexanoate are dissolved in 227 ml of ethanol, 1.4 g of Raney-Ni are added and the mixture is stirred at 50° C. in an autoclave at a hydrogen pressure of 70 bar. The catalyst is filtered off with suction and the filtrate is evaporated to dryness on a rotary evaporator, giving 19.7 g of the desired product as a mixture of isomers (log P (pH 2.3): 1.02 and 0.96); ¹H NMR (400 MHz, DMSO-d) δ=7.45 (s, 1H), 3.45-3.25 (m, 1H), 2.40-2.30 (m, 1H), 1.93-1.67 (m, 1H), 1.52-1.28 (m, 2H), 1.20-1.0 (m, 4H), 0.90-0.80 (m, 3H). MM+1=128.2

Synthesis of Compounds of the Formula (Ic) (cf. Scheme 20)

### Method A:

### 1-(2-Chloro-5-{[4-(cyclopropylamino)-5-(trifluoromethyl)pyrimidin-2-yl]amino}phenyl)pyrrolidine-2,5-dione (Example 119)

A mixture of 150 mg (0.63 mmol) of 2-chloro-5-trifluoromethyl-N-cyclopropylpyrimidin-4-amine, 170 mg (0.76 mmol) of 1-(5-amino-2-chlorophenyl)pyrrolidine-2,5-dione and 92 mg (0.54 mmol) of 4-toluenesulphonic acid in 6 ml of dioxane is stirred at 105° C. for 18 h. After cooling, the reaction mixture is concentrated under reduced pressure, ethyl acetate and NaHCO₃ solution are added and the organic phase is separated off, washed once with water, dried over MgSO₄ and again freed from the solvent under reduced pressure. The crude product is purified by column chromatography on silica gel (cyclohexane/ethyl acetate 1:1). This gives 250 mg of the desired product (log P (pH 2.3): 2.49). ¹H NMR (400 MHz, DMSO-d) δ=9.81 (m, 1H), 8.20-8.19 (d, 1 H), 7.96 (m, 1H), 7.91-7.88 (m, 1H), 7.52-7.49 (m, 1H), 6.90 (m, 1H), 2.88-2.86 (m, 4H), 0.73-0.79 (m, 2H), 0.66-0.64 (m, 2H),

Synthesis of Intermediates of the Formula (XIXa) (cf. Scheme 21)

### 3,3-Dimethyl-1-(3-nitrophenyl)pyrrolidine-2,5-dione

In a mixture of 50 ml of toluene and 25 ml of dioxane, 8.0 g (58 mmol) of 3-aminonitrobenzene and 7.4 g (58 mmol) of 2,2-dimethylsuccinic anhydride are heated under reflux for 2 h. Filtration with suction gives 14.0 g of crude 3,3-dimethyl-4-[(3-nitrophenyl)amino]-4-oxobutanoic acid which is directly reacted further. The acid is dissolved in 40 ml of acetic anhydride, 0.67 g (8.17 mmol) of sodium acetate is added and the mixture is stirred at 60° C. for 2 h. The reaction mixture is then poured into ice-water with stirring, and the solid formed is filtered off with suction. Washing with water and drying on a clay plate gives 14.5 g of the desired product (log P (pH 2.3): 2.01). ¹H NMR (400 MHz, ACETONITRILE-d) δ=8.24 (m, 2H), 7.76 (m, 2H), 2.74 (s, 2H), 1.39 (s, 2H).

Synthesis of Intermediates of the Formula (IVc) (cf. Scheme 22)

### 1-(3-Amino-4-chlorophenyl)pyrrolidine-2,5-dione

1.0 g (3.96 mmol) of 1-(4-chloro-3-nitrophenyl)-1H-pyrrole-2,5-dione is dissolved in 20 ml of THF, 300 mg of Pd/C (10%) are added and the mixture is stirred at room temperature in an autoclave at a hydrogen pressure of 10 bar for 10 h. The catalyst is filtered off with suction and the filtrate is then evaporated to dryness on a rotary evaporator, giving 820 mg (76%) of 1-(3-amino-4-chlorophenyl)pyrrolidine-2,5-dione (log P (pH 2.3): 0.79); ¹H NMR (400 MHz, DMSO-d) δ=7.19-7.15 (m, 1H), 6.65-6.63 (m, 1H), 6.48-6.47 (d, 1H), 5.38-5.33 (br, s, 2H), 2.91-2.84 (m, 4H)

### 1-(3-Aminophenyl)-3,3-dimethylpyrrolidine-2,5-dione

8.3 g (33.3 mmol) of 3,3-dimethyl-1-(3-nitrophenyl)pyrrolidine-2,5-dione are dissolved in 150 ml of ethyl acetate, 400 mg of Pd/C (10%) and 9.45 g (150 mmol) of ammonium formate are added and the mixture is stirred at room temperature overnight. The catalyst is filtered off with suction and the filtrate is then evaporated to dryness on a rotary evaporator, the residue is taken up in 250 ml of ethyl acetate/dioxane 2:1 and the mixture is washed once with water. The organic phase is removed, then dried over MgSO₄ and again freed from the solvent under reduced pressure. This gives 6.8 g of the desired product (log P (pH 2.3): 0.86); ¹H NMR (400 MHz, ACETONITRILE-d) δ=7.16 (t, 1H), 6.67 (dd, 1H), 6.48 m, 2H), 4.31 (br. s, 2H), 2.66 (s, 2H), 1.34 (s, 6H)

### EXAMPLES

The compounds of the formula (I) listed in Table I below are also obtained by the methods given above.

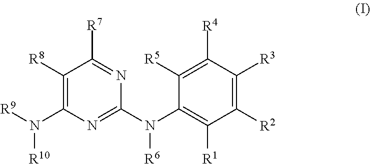

TABLE I

| Ex. | R¹ | R² | R³ | R⁴ | R⁵ | R⁶ | R⁷ | R⁸ | R⁹ | R¹⁰ | log p |
|---|---|---|---|---|---|---|---|---|---|---|---|
| 1 | H | H | 2-Oxopyrrolidin-1-yl | H | H | H | H | Cl | H | 1-Methoxypropan-2-yl | 1.51[a]; |
| | | | | | | | | | | | 1.51[b] |
| 2 | H | 2-Oxopyrrolidin-1-yl | H | H | H | H | H | Cl | H | 1-Methoxypropan-2-yl | 1.53[a] |
| 3 | H | H | 4-Methyl-2-oxo-1,3- | H | H | H | H | Cl | H | 1-Methoxypropan-2-yl | 1.54[b] |
| | | | oxazolidin-3-yl | | | | | | | | |
| 4 | H | H | 4-Methyl-2-oxo-1,3- | H | H | H | H | Br | H | 1-Methoxypropan-2-yl | 1.61[b] |
| | | | oxazolidin-3-yl | | | | | | | | |
| 5 | H | 2-Oxopyrrolidin-1-yl | H | H | H | H | H | Br | H | 1-Methoxypropan-2-yl | 1.6[b] |
| 6 | H | 2-Oxopyrrolidin-1-yl | H | H | H | H | H | CF₃ | H | 1-Methoxypropan-2-yl | 2.4[a]; |
| | | | | | | | | | | | 2.79[c] |
| 7 | H | H | 2-Oxopyrrolidin-1-yl | H | H | H | H | Cl | H | Butan-2-yl | 1.65[a] |
| 8 | H | 2-Oxopyrrolidin-1-yl | H | H | H | H | H | Cl | H | Butan-2-yl | 1.84[a]; |
| | | | | | | | | | | | 2.92[c] |
| 9 | H | H | 2-Oxopyrrolidin-1-yl | H | H | H | H | Cl | H | 2,2,2-Trifluoroethyl | 1.96[a]; |
| | | | | | | | | | | | 2.34[c] |
| 10 | H | 2-Oxopyrrolidin-1-yl | H | H | H | H | H | Cl | H | 2,2,2-Trifluoroethyl | 2.08[a]; |
| | | | | | | | | | | | 2.42[c] |
| 11 | H | H | 2-Oxo-1,3-oxazolidin-3-yl | H | H | H | H | Iodine | H | Cyclopropyl | 1.41[a]; |
| | | | | | | | | | | | 2.48[c] |
| 12 | H | H | 2-Oxo-1,3-oxazolidin-3-yl | H | H | H | H | Cl | H | 1-Methoxypropan-2-yl | 1.46[b] |
| 13 | H | H | 2-Oxopyrrolidin-1-yl | H | H | H | H | Cl | H | Prop-2-en-1-yl | 1.38[a]; |
| | | | | | | | | | | | 2.29[c] |
| 14 | H | 2-Oxopyrrolidin-1-yl | H | H | H | H | H | Cl | H | Prop-2-en-1-yl | 1.45[a]; |
| | | | | | | | | | | | 2.37[c] |
| 15 | H | H | 2-Oxo-1,3-oxazolidin-3-yl | H | H | H | H | Cl | H | Prop-2-en-1-yl | 1.25[a]; |
| | | | | | | | | | | | 2.25[c] |
| 16 | H | 2-Oxopyridin-1(2H)-yl | H | H | H | H | H | Cl | H | Cyclobutyl | 2.17[b] |
| 17 | H | H | 2-Oxo-1,3-oxazolidin-3-yl | H | H | H | H | Cl | H | Oxetan-3-yl | 0.26[a]; |
| | | | | | | | | | | | 0.76[c] |
| 18 | H | H | 2-Oxopyrrolidin-1-yl | H | H | H | H | Cl | H | Oxetan-3-yl | 0.41[a]; |
| | | | | | | | | | | | 0.9[c] |
| 19 | H | 4-Methyl-2-oxo-1,3-oxazolidin-3-yl | H | H | H | H | H | CF₃ | H | Cyclopropyl | 2.4[b] |
| 20 | H | 2-Oxopyrrolidin-1-yl | Trifluoromethoxy | H | H | H | H | Cl | H | Cyclopropyl | 2.34[a]; |
| | | | | | | | | | | | 2.91[c] |
| 21 | H | H | 2-Oxopyrrolidin-1-yl | H | H | H | H | Cl | H | Propan-2-yl | 1.44[a]; |
| | | | | | | | | | | | 2.56[c] |
| 22 | H | 2-Oxopyrrolidin-1-yl | H | H | H | H | H | Cl | H | Propan-2-yl | 1.52[a]; |
| | | | | | | | | | | | 2.64[c] |
| 23 | H | H | 2-Oxo-1,3-oxazolidin-3-yl | H | H | H | H | Cl | H | 2,2,2-Trifluorethyl | 1.83[b] |
| 24 | H | 2-Oxopyrrolidin-1-yl | H | H | H | H | H | Cyano | H | 1-Methoxypropan-2-yl | 1.99[b] |
| 25 | H | 3,3-Dimethyl-2,5- | H | H | H | H | H | Cl | H | Cyclopropyl | 1.83[a] |
| | | dioxopyrrolidin-1-yl | | | | | | | | | |
| 26 | H | 3-Methyl-2-oxopyrrolidin-1- | H | H | H | H | H | Cl | H | Cyclopropyl | 1.76[a] |
| | | yl | | | | | | | | | |
| 27 | H | 3-Methyl-2-oxopyrrolidin-1- | H | H | H | H | H | Br | H | Cyclobutyl | 2.24[a]; |
| | | yl | | | | | | | | | 2.09[b] |
| 28 | H | 3,3-Dimethyl-2,5- | H | H | H | H | H | Br | H | Cyclopropyl | 1.99[b] |
| | | dioxopyrrolidin-1-yl | | | | | | | | | |
| 29 | H | 3,3-Dimethyl-2,5- | H | H | H | H | H | Cl | H | Cyclobutyl | 2.07[b] |
| | | dioxopyrrolidin-1-yl | | | | | | | | | |
| 30 | H | 2-Methyl-5-oxopyrrolidin-1- | H | H | H | H | H | Cl | H | Cyclopropyl | 1.6[a]; |
| | | yl | | | | | | | | | 1.6[b] |
| 31 | H | 4-Ethyl-2-oxo-1,3- | H | H | H | H | H | Cl | H | Cyclopropyl | 1.86[a] |
| | | oxazolidin-3-yl | | | | | | | | | |
| 32 | H | 4-Ethyl-2-oxo-1,3- | H | H | H | H | H | Br | H | Cyclopropyl | 2.01[a] |
| | | oxazolidin-3-yl | | | | | | | | | |
| 33 | H | 2-Thioxopyrrolidin-1-yl | H | H | H | H | H | Cl | H | Cyclopropyl | 1.69[a]; |
| | | | | | | | | | | | 2.54[c] |
| 34 | H | 4-Ethyl-2-oxo-1,3- | H | H | H | H | H | Br | H | Cyclobutyl | 2.24[b] |
| | | oxazolidin-3-yl | | | | | | | | | |
| 35 | H | 4-Ethyl-2-oxo-1,3- | H | H | H | H | H | Cl | H | Cyclobutyl | 2.09[b] |
| | | oxazolidin-3-yl | | | | | | | | | |
| 36 | H | 2-Methyl-5-oxopyrrolidin-1- | H | H | H | H | H | Br | H | Cyclobutyl | 1.97[b] |
| | | yl | | | | | | | | | |
| 37 | H | 2-Methyl-5-oxopyrrolidin-1- | H | H | H | H | H | Cl | H | Cyclobutyl | 1.83[b] |
| | | yl | | | | | | | | | |
| 38 | H | 2-Oxopyrrolidin-1-yl | H | H | H | H | H | Cl | H | CH₃ | 1.05[a] |
| 39 | H | 2-Oxo-4-(propan-2-yl)-1,3- | H | H | H | H | H | Cl | H | Cyclopropyl | 2.13[a] |
| | | oxazolidin-3-yl | | | | | | | | | |
| 40 | H | H | 2-Oxo-1,3-oxazolidin-3-yl | H | H | H | H | Cl | H | 2-Methyl-1- | 1.95[a]; |
| | | | | | | | | | | (methylsulphanyl)propan- | 3.09[c] |
| | | | | | | | | | | 2-yl | |
| 41 | H | 2-Oxopyrrolidin-1-yl | H | H | H | H | H | Cl | H | 2-Methyl-1- | 2.17[a]; |
| | | | | | | | | | | (methylsulphanyl)propan- | 3.26[c] |
| | | | | | | | | | | 2-yl | |
| 42 | H | 2-Oxopyrrolidin-1-yl | H | H | H | H | H | Cl | H | Cyclopentyl | 1.92[b] |
| 43 | H | 2-Oxopyrrolidin-1-yl | H | H | H | H | H | Fluorine | H | Cyclobutyl | 1.31[a]; |
| | | | | | | | | | | | 2.32[c] |
| 44 | H | 2-Oxopyrrolidin-1-yl | H | H | H | H | H | Cl | H | 2,2-Difluorethyl | 1.64[a]; |
| | | | | | | | | | | | 2.19[c] |
| 45 | H | 2-Oxo-4-(propan-2-yl)-1,3- | H | H | H | H | H | Br | H | Cyclopropyl | 2.24[a] |
| | | oxazolidin-3-yl | | | | | | | | | |
| 46 | H | 2-Oxo-4-(propan-2-yl)-1,3- | H | H | H | H | H | Cl | H | Cyclobutyl | 2.41[a] |
| | | oxazolidin-3-yl | | | | | | | | | |
| 47 | H | 4-(2-Methylpropyl)-2-oxo- | H | H | H | H | H | Cl | H | Cyclopropyl | 2.54[a] |
| | | 1,3-oxazolidin-3-yl | | | | | | | | | |
| 48 | H | 4-(2-Methylpropyl)-2-oxo- | H | H | H | H | H | Br | H | Cyclopropyl | 2.69[a] |
| | | 1,3-oxazolidin-3-yl | | | | | | | | | |
| 49 | H | 4-(2-Methylpropyl)-2-oxo- | H | H | H | H | H | Cl | H | Cyclobutyl | 2.83[a] |
| | | 1,3-oxazolidin-3-yl | | | | | | | | | |
| 50 | H | 4-(2-Methylpropyl)-2-oxo- | H | H | H | H | H | Br | H | Cyclobutyl | 3.05[a] |
| | | 1,3-oxazolidin-3-yl | | | | | | | | | |
| 51 | H | 2-Oxopyrrolidin-1-yl | Fluorine | H | H | H | H | Cl | H | 2,2-Difluoroethyl | 1.72[a]; |
| | | | | | | | | | | | 2.15[c] |
| 52 | H | 3,3-Dimethyl-2-oxoazetidin-1-yl | H | H | H | H | H | Br | H | Cyclopropyl | 2.18[b] |
| 53 | H | 3,3-Dimethyl-2-oxoazetidin- | H | H | H | H | H | Cl | H | Cyclopropyl | 2.07[b] |
| | | 1-yl | | | | | | | | | |
| 54 | H | 2-Oxoazepan-1-yl | H | H | H | H | H | Br | H | Cyclopropyl | 1.82[a] |
| 55 | H | 2-Oxoazepan-1-yl | H | H | H | H | H | Cl | H | Cyclopropyl | 1.7[a] |
| 56 | H | 4-Methyl-2-oxo-1,3- | H | H | H | H | H | Br | H | Cyclobutyl | 1.97[a] |
| | | oxazolidin-3-yl | | | | | | | | | |
| 57 | H | 4-Methyl-2-oxo-1,3- | H | H | H | H | H | Cl | H | Cyclobutyl | 1.81[a] |
| | | oxazolidin-3-yl | | | | | | | | | |
| 58 | H | 2-Oxopiperidin-1-yl | H | H | H | H | H | Fluorine | H | Cyclobutyl | 1.29[a] |
| 59 | H | 2-Oxo-1,3-oxazolidin-3-yl | H | H | H | H | H | Cl | H | Cyclobutyl | 1.64[a]; |
| | | | | | | | | | | | 1.64[b] |
| 60 | H | 2-Methyl-5-oxopyrrolidin-1- | H | H | H | H | H | Br | H | Cyclopropyl | 1.72[a]; |
| | | yl | | | | | | | | | 1.72[b] |
| 61 | H | H | 2-Oxo-1,3-oxazolidin-3-yl | H | H | H | H | Cl | H | CH₃ | 0.73[a]; |
| | | | | | | | | | | | 1.82[c] |
| 62 | H | 3,3-Dimethyl-2,5- | H | H | H | H | H | CF₃ | H | Cyclopropyl | 2.63[a] |
| | | dioxopyrrolidin-1-yl | | | | | | | | | |
| 63 | H | 3,3-Dimethyl-2,5- | H | H | H | H | H | Br | H | Cyclobutyl | 2.19[a] |
| | | dioxopyrrolidin-1-yl | | | | | | | | | |
| 64 | H | H | 2-Oxo-1,3-oxazolidin-3-yl | H | H | H | H | Cl | H | Propan-2-yl | 1.49[b] |
| 65 | H | 3-Methyl-2,5- | H | H | H | H | H | Cl | H | Cyclopropyl | 1.59[b] |
| | | dioxopyrrolidin-1-yl | | | | | | | | | |
| 66 | H | 3-Methyl-2,5- | H | H | H | H | H | Br | H | Cyclopropyl | 1.78[b] |
| | | dioxopyrrolidin-1-yl | | | | | | | | | |
| 67 | H | 2-Oxo-1,3-oxazolidin-3-yl | H | H | H | H | H | Cl | H | 1-Methoxypropan-2-yl | 1.54[b] |
| 68 | H | 3-Methyl-2,5- | H | H | H | H | H | Cl | H | 1-Methoxypropan-2-yl | 1.59[b] |
| | | dioxopyrrolidin-1-yl | | | | | | | | | |
| 69 | H | 2-Oxo-1,3-oxazolidin-3-yl | H | H | H | H | H | Br | H | 1-Methoxypropan-2-yl | 1.61[b] |
| 70 | H | 3-Methyl-2,5- | H | H | H | H | H | Br | H | 1-Methoxypropan-2-yl | 1.68[b] |
| | | dioxopyrrolidin-1-yl | | | | | | | | | |
| 71 | H | 3-Methyl-2,5- | H | H | H | H | H | Cl | H | Cyclopropylmethyl | 1.68[b] |
| | | dioxopyrrolidin-1-yl | | | | | | | | | |
| 72 | H | 2-Oxo-1,3-oxazolidin-3-yl | H | H | H | H | H | Br | H | Cyclobutyl | 1.8[b] |
| 73 | H | 3-Methyl-2,5- | H | H | H | H | H | Br | H | Cyclobutyl | 1.9[b] |
| | | dioxopyrrolidin-1-yl | | | | | | | | | |
| 74 | H | 3-Methyl-2,5- | H | H | H | H | H | Br | H | Cyclopropylmethyl | 1.78[b] |
| | | dioxopyrrolidin-1-yl | | | | | | | | | |
| 75 | H | (4R)-2-Oxo-4-(propan-2-yl)- | H | H | H | H | H | Cl | H | Cyclopropyl | 2.39[b] |
| | | 1,3-oxazolidin-3-yl | | | | | | | | | |
| 76 | H | (4S)-2-Oxo-4-(propan-2-yl)- | H | H | H | H | H | Cl | H | Cyclopropyl | 2.47[b] |
| | | 1,3-oxazolidin-3-yl | | | | | | | | | |
| 77 | H | 2-Oxopyrrolidin-1-yl | H | H | H | H | H | Cl | CH₃ | Propan-2-yl | 2.1[a]; |
| | | | | | | | | | | | 3.18[c] |
| 78 | H | (3S)-3-Hydroxyl-2- | H | H | H | H | H | Cl | H | Cyclopropyl | 1.14[a]; |
| | | oxopyrrolidin-1-yl | | | | | | | | | 1.84[c] |
| 79 | H | (2R)-2-(Hydroxylmethyl)-5- | H | H | H | H | H | Br | H | Cyclopropyl | 1.04[a]; |
| | | oxopyrrolidin-1-yl | | | | | | | | | 1.65[c] |
| 80 | H | 3,5-Dimethyl-2- | H | H | H | H | H | Cl | H | Cyclobutyl | 2.2[b] |
| | | oxopyrrolidin-1-yl | | | | | | | | | |
| 81 | H | 3,5-Dimethyl-2- | H | H | H | H | H | Cl | H | Cyclopropyl | 1.95[b] |
| | | oxopyrrolidin-1-yl | | | | | | | | | |
| 82 | H | (2R)-2-(Hydroxylmethyl)-5- | H | H | H | H | H | Cl | CH₃ | Cyclopropyl | 1.54[a]; |
| | | oxopyrrolidin-1-yl | | | | | | | | | 2.06[c] |
| 83 | H | 2-Oxopyrrolidin-1-yl | H | H | H | CH₃ | H | Cl | H | Cyclobutyl | 1.63[b] |
| 84 | H | 3,5-Dimethyl-2- | H | H | H | H | H | Br | H | Cyclopropyl | 2.91[c]; |
| | | oxopyrrolidin-1-yl | | | | | | | | | 2.01[b] |
| 85 | H | 3,5-Dimethyl-2- | H | H | H | H | H | Br | H | Cyclobutyl | 2.3[b] |
| | | oxopyrrolidin-1-yl | | | | | | | | | |
| 86 | H | (2R)-2-(Hydroxylmethyl)-5- | H | H | H | H | H | Cl | H | Cyclopropyl | 0.98[a] |
| | | oxopyrrolidin-1-yl | | | | | | | | | |
| 87 | H | 2-Oxo-5-phenylpyrrolidin-1- | H | H | H | H | H | Cl | H | Cyclopropyl | 2.1[a] |
| | | yl | | | | | | | | | |
| 88 | H | 2-Oxo-4-phenyl-1,3- | H | H | H | H | H | Cl | H | Cyclopropyl | 2.27[b] |
| | | oxazolidin-3-yl | | | | | | | | | |
| 89 | H | 2-Oxo-4-phenyl-1,3- | H | H | H | H | H | Cl | H | Cyclobutyl | 2.65[b] |
| | | oxazolidin-3-yl | | | | | | | | | |
| 90 | H | 2-Oxo-4-phenyl-1,3- | H | H | H | H | H | Br | H | Cyclobutyl | 2.8[b] |
| | | oxazolidin-3-yl | | | | | | | | | |
| 91 | H | 2-Oxo-4-phenyl-1,3- | H | H | H | H | H | Br | H | Cyclopropyl | 2.3[b] |
| | | oxazolidin-3-yl | | | | | | | | | |
| 92 | H | (4S)-4-Methyl-2-oxo-1,3- | H | H | H | H | H | Cl | H | Cyclopropyl | 2.42[c]; |
| | | oxazolidin-3-yl | | | | | | | | | 1.54[b] |
| 93 | H | (4S)-4-Methyl-2-oxo-1,3- | H | H | H | H | H | Cl | H | Cyclobutyl | 1.82[b] |
| | | oxazolidin-3-yl | | | | | | | | | |
| 94 | H | (4S)-4-Methyl-2-oxo-1,3- | H | H | H | H | H | Br | H | Cyclobutyl | 1.95[b] |
| | | oxazolidin-3-yl | | | | | | | | | |
| 95 | H | (4S)-4-Methyl-2-oxo-1,3- | H | H | H | H | H | Br | H | Cyclopropyl | 1.65[b] |
| | | oxazolidin-3-yl | | | | | | | | | |
| 96 | H | 4-Methyl-2-oxopyrrolidin-1- | H | H | H | H | H | Cl | H | Cyclopropyl | 1.75[b] |
| | | yl | | | | | | | | | |
| 97 | H | 4-Methyl-2-oxopyrrolidin-1- | H | H | H | H | H | Cl | H | Cyclobutyl | 2.02[b] |
| | | yl | | | | | | | | | |
| 98 | H | 4-Methyl-2-oxopyrrolidin-1- | H | H | H | H | H | Br | H | Cyclobutyl | 2.16[b] |
| | | yl | | | | | | | | | |
| 99 | H | 4-Methyl-2-oxopyrrolidin-1- | H | H | H | H | H | Br | H | Cyclopropyl | 2.79[c]; |
| | | yl | | | | | | | | | 1.85[b] |
| 100 | H | (4S)-2-Oxo-4-(propan-2-yl)- | H | H | H | H | H | Cl | H | Cyclobutyl | 2.38[b] |
| | | 1,3-oxazolidin-3-yl | | | | | | | | | |
| 101 | H | (4R)-2-Oxo-4-(propan-2-yl)- | H | H | H | H | H | Cl | H | Cyclobutyl | 2.38[b] |
| | | 1,3-oxazolidin-3-yl | | | | | | | | | |
| 102 | H | (4R)-4-Methyl-2-oxo-1,3- | H | H | H | H | H | Cl | H | Cyclobutyl | 1.56[a] |
| | | oxazolidin-3-yl | | | | | | | | | |
| 103 | H | (4R)-4-Methyl-2-oxo-1,3- | H | H | H | H | H | Cl | H | Cyclopropyl | 1.8[a] |
| | | oxazolidin-3-yl | | | | | | | | | |
| 104 | H | (4R)-4-Methyl-2-oxo-1,3- | H | H | H | H | H | Br | H | Cyclopropyl | 2.03[b] |
| | | oxazolidin-3-yl | | | | | | | | | |
| 105 | H | 2-Oxo-1,3-oxazolidin-3-yl | CH₃ | H | H | H | H | Cl | H | Cyclobutyl | 1.6[b] |
| 106 | H | 2-Ethoxy-5-oxopyrrolidin-1- | H | H | H | H | H | Cl | H | Cyclopropyl | 1.65[a] |
| | | yl | | | | | | | | | |
| 107 | H | (4R)-4-Methyl-2-oxo-1,3- | H | H | H | H | H | Br | H | Cyclobutyl | 1.94[b] |
| | | oxazolidin-3-yl | | | | | | | | | |
| 108 | H | (4R)-4-Hydroxyl-2- | H | H | H | H | H | Cl | H | Cyclopropyl | 0.98[a]; |
| | | oxopyrrolidin-1-yl | | | | | | | | | 1.68[c] |
| 109 | H | (4S)-2-Oxo-4-(propan-2-yl)- | H | H | H | H | H | Cl | H | Cyclopropylmethyl | 2.17[b] |
| | | 1,3-oxazolidin-3-yl | | | | | | | | | |
| 110 | H | (4S)-2-Oxo-4-(propan-2-yl)- | H | H | H | H | H | Cl | H | Propan-2-yl | 2.1[b] |
| | | 1,3-oxazolidin-3-yl | | | | | | | | | |
| 111 | H | (4S)-2-Oxo-4-(propan-2-yl)- | H | H | H | H | H | Cl | H | 2,2,2-Trifluoroethyl | 2.81[b] |
| | | 1,3-oxazolidin-3-yl | | | | | | | | | |
| 112 | H | (4S)-2-Oxo-4-(propan-2-yl)- | H | H | H | H | H | Cl | H | 2,2-Difluoroethyl | 2.39[b] |
| | | 1,3-oxazolidin-3-yl | | | | | | | | | |
| 113 | H | 3-Methyl-2-oxopyrrolidin-1- | H | H | H | H | H | Cl | H | Cyclobutyl | 2.01[b] |
| | | yl | | | | | | | | | |
| 114 | H | 2-Oxopiperidin-1-yl | H | H | H | H | H | Cl | H | Propan-2-yl | 1.46[b] |
| 115 | H | (4S)-2-Oxo-4-(propan-2-yl)- | H | H | H | H | H | Cl | CH₃ | Cyclopropyl | 3.09[a] |
| | | 1,3-oxazolidin-3-yl | | | | | | | | | |
| 116 | H | 4-Methyl-2-oxo-1,3- | H | H | H | H | H | Cl | H | Cyclopropylmethyl | 1.64[b] |
| | | oxazolidin-3-yl | | | | | | | | | |
| 117 | H | (4S)-2-Oxo-4-(propan-2-yl)- | H | H | H | H | H | Cl | H | 1-Methoxypropan-2-yl | 2.1[b] |
| | | 1,3-oxazolidin-3-yl | | | | | | | | | |
| 118 | H | 4-Methyl-2-oxo-1,3- | H | H | H | H | H | Cl | H | 1-Methoxypropan-2-yl | 1.6[a]; |
| | | oxazolidin-3-yl | | | | | | | | | 1.6[b] |
| 119 | H | 2,5-Dioxopyrrolidin-1-yl | Chlorine | H | H | H | H | CF₃ | H | Cyclopropyl | 2.49[b] |
| 120 | H | 4-Methyl-2-oxo-1,3- | H | H | H | H | H | Cl | H | 2,2- | 2.15[a]; |
| | | oxazolidin-3-yl | | | | | | | | Dimethylcyclopropyl | 3.12[c]; |
| | | | | | | | | | | | 2.15[b] |
| 121 | H | 3-Methyl-2,5- | H | H | H | H | H | Cl | H | 2,2- | 2.13[a]; |
| | | dioxopyrrolidin-1-yl | | | | | | | | Dimethylcyclopropyl | 2.92[c]; |
| | | | | | | | | | | | 2.13[b] |
| 122 | H | 3,3-Dimethyl-2,5- | H | H | H | H | H | Cl | H | 2,2- | 2.42[a]; |
| | | dioxopyrrolidin-1-yl | | | | | | | | Dimethylcyclopropyl | 3.28[c]; |
| | | | | | | | | | | | 2.42[b] |
| 123 | H | (3R)-3-Methyl-2- | H | H | H | H | H | Cl | H | Ccyclopropyl | 2.86[a]; |
| | | oxopyrrolidin-1-yl | | | | | | | | | 2.86[b] |
| 124 | H | (3S)-3-Methyl-2- | H | H | H | H | H | Cl | H | Cyclopropyl | 2.86[a]; |
| | | oxopyrrolidin-1-yl | | | | | | | | | 2.86[b] |
| 125 | H | (2S)-2-Methyl-5- | H | H | H | H | H | Cl | H | Cyclopropyl | 2.59[a]; |
| | | oxopyrrolidin-1-yl | | | | | | | | | 2.59[b] |
| 126 | H | (2R)-2-Methyl-5- | H | H | H | H | H | Cl | H | Cyclopropyl | 2.59[a]; |
| | | oxopyrrolidin-1-yl | | | | | | | | | 2.59[b] |
| 127 | H | 2-Oxopyrrolidin-1-yl | H | H | H | H | H | Cl | H | 2,2- | 1.98[a]; |
| | | | | | | | | | | Dimethylcyclopropyl | 2.99[c]; |
| | | | | | | | | | | | 1.98[b] |
| 128 | H | (4S)-2-Oxo-4-(propan-2-yl)- | H | H | H | H | H | Br | H | Cyclopropylmethyl | 2.28[b] |
| | | 1,3-oxazolidin-3-yl | | | | | | | | | |
| 129 | H | (4S)-2-Oxo-4-(propan-2-yl)- | H | H | H | H | H | Br | H | Propan-2-yl | 2.24[b] |
| | | 1,3-oxazolidin-3-yl | | | | | | | | | |
| 130 | H | (4S)-2-Oxo-4-(propan-2-yl)- | H | H | H | H | H | Br | H | 2,2-Difluoroethyl | 2.5[b] |
| | | 1,3-oxazolidin-3-yl | | | | | | | | | |
| 131 | H | 4-Methyl-2-oxo-1,3- | H | H | H | H | H | Br | H | Cyclopropylmethyl | 1.75[b] |
| | | oxazolidin-3-yl | | | | | | | | | |
| 132 | H | 3,3-Dimethyl-2,5- | H | H | H | H | H | Br | H | Cyclopropylmethyl | 2.02[b] |
| | | dioxopyrrolidin-1-yl | | | | | | | | | |
| 133 | H | 2-Methyl-5-oxopyrrolidin-1- | H | H | H | H | H | Br | H | Cyclopropylmethyl | 1.77[b] |
| | | yl | | | | | | | | | |
| 134 | H | 2-Oxopiperidin-1-yl | H | H | H | H | H | Br | H | Propan-2-yl | 1.57[b] |
| 135 | H | 2-Oxopiperidin-1-yl | H | H | H | H | H | Br | H | 2,2-Difluoroethyl | 1.75[b] |
| 136 | H | (2S)-2-(Hydroxylmethyl)-5- | H | H | H | H | H | Cl | H | Cyclopropyl | 0.96[a]; |
| | | oxopyrrolidin-1-yl | | | | | | | | | 1.6[c] |
| 137 | H | 4-(Methoxycarbonyl)-2- | H | H | H | H | H | Cl | H | Cyclopropyl | 1.52[b] |
| | | oxopyrrolidin-1-yl | | | | | | | | | |
| 138 | H | 2-Oxo-1,3-oxazolidin-3-yl | H | H | H | H | H | Cl | H | Propan-2-yl | 1.56[b] |
| 139 | H | 4-Methyl-2-oxo-1,3- | H | H | H | H | H | Cl | H | Propan-2-yl | 1.64[b] |
| | | oxazolidin-3-yl | | | | | | | | | |
| 140 | H | 3,3-Dimethyl-2,5- | H | H | H | H | H | Cl | H | Propan-2-yl | 1.81[b] |
| | | dioxopyrrolidin-1-yl | | | | | | | | | |
| 141 | H | 3,5-Dimethyl-2- | H | H | H | H | H | Cl | H | Propan-2-yl | 3.15[c]; |
| | | oxopyrrolidin-1-yl | | | | | | | | | 1.88[b] |
| 142 | H | H | 4-Methyl-2-oxo-1,3- | H | H | H | H | Cl | H | Propan-2-yl | 1.61[b] |
| | | | oxazolidin-3-yl | | | | | | | | |
| 143 | H | 3-Oxomorpholin-4-yl | H | H | H | H | H | Cl | H | Propan-2-yl | 1.41[b] |
| 144 | H | 2-Methyl-5-oxopyrrolidin-1- | H | H | H | H | H | Cl | H | Propan-2-yl | 1.66[b] |
| | | yl | | | | | | | | | |
| 145 | H | 3-Methyl-2,5- | H | H | H | H | H | Cl | H | Propan-2-yl | 1.59[b] |
| | | dioxopyrrolidin-1-yl | | | | | | | | | |
| 146 | H | 2-Oxo-1,3-oxazolidin-3-yl | H | H | H | H | H | Br | H | Propan-2-yl | 1.63[b] |
| 147 | H | 4-Methyl-2-oxo-1,3- | H | H | H | H | H | Br | H | Propan-2-yl | 1.73[b] |
| | | oxazolidin-3-yl | | | | | | | | | |
| 148 | H | 3,3-Dimethyl-2,5- | H | H | H | H | H | Br | H | Propan-2-yl | 1.93[b] |
| | | dioxopyrrolidin-1-yl | | | | | | | | | |
| 149 | H | (3R,5S)-3,5-Dimethyl-2- | H | H | H | H | H | Br | H | Propan-2-yl | 1.98[b] |
| | | oxopyrrolidin-1-yl | | | | | | | | | |
| 150 | H | H | 4-Methyl-2-oxo-1,3- | H | H | H | H | Br | H | Propan-2-yl | 1.68[b] |
| | | | oxazolidin-3-yl | | | | | | | | |
| 151 | H | 3-Oxomorpholin-4-yl | H | H | H | H | H | Br | H | Propan-2-yl | 1.47[b] |
| 152 | H | 2-Methyl-5-oxopyrrolidin-1- | H | H | H | H | H | Br | H | Propan-2-yl | 1.73[b] |
| | | yl | | | | | | | | | |
| 153 | H | 3-Methyl-2,5- | H | H | H | H | H | Br | H | Propan-2-yl | 1.68[b] |
| | | dioxopyrrolidin-1-yl | | | | | | | | | |
| 154 | H | 2-Oxo-1,3-oxazolidin-3-yl | H | H | H | H | H | Cl | H | 2,2-Difluoroethyl | 1.63[b] |
| 155 | H | 4-Methyl-2-oxo-1,3- | H | H | H | H | H | Cl | H | 2,2-Difluoroethyl | 1.81[b] |
| | | oxazolidin-3-yl | | | | | | | | | |
| 156 | H | 3,3-Dimethyl-2,5- | H | H | H | H | H | Cl | H | 2,2-Difluoroethyl | 2.01[b] |
| | | dioxopyrrolidin-1-yl | | | | | | | | | |
| 157 | H | (3R,5S)-3,5-Dimethyl-2- | H | H | H | H | H | Cl | H | 2,2-Difluoroethyl | 2.11[b] |
| | | oxopyrrolidin-1-yl | | | | | | | | | |
| 158 | H | H | 4-Methyl-2-oxo-1,3- | H | H | H | H | Cl | H | 2,2-Difluoroethyl | 1.68[b] |
| | | | oxazolidin-3-yl | | | | | | | | |
| 159 | H | 3-Oxomorpholin-4-yl | H | H | H | H | H | Cl | H | 2,2-Difluoroethyl | 1.46[b] |
| 160 | H | 2-Methyl-5-oxopyrrolidin-1- | H | H | H | H | H | Cl | H | 2,2-Difluoroethyl | 1.81[b] |
| | | yl | | | | | | | | | |
| 161 | H | 3-Methyl-2,5- | H | H | H | H | H | Cl | H | 2,2-Difluoroethyl | 1.68[b] |
| | | dioxopyrrolidin-1-yl | | | | | | | | | |
| 162 | H | 2-Oxo-1,3-oxazolidin-3-yl | H | H | H | H | H | Br | H | 2,2-Difluoroethyl | 1.71[b] |
| 163 | H | 4-Methyl-2-oxo-1,3- | H | H | H | H | H | Br | H | 2,2-Difluoroethyl | 1.88[b] |
| | | oxazolidin-3-yl | | | | | | | | | |
| 164 | H | 3,3-Dimethyl-2,5- | H | H | H | H | H | Br | H | 2,2-Difluorethyl | 2.08[b] |
| | | dioxopyrrolidin-1-yl | | | | | | | | | |
| 165 | H | H | 4-Methyl-2-oxo-1,3- | H | H | H | H | Br | H | 2,2-Difluoroethyl | 1.78[b] |
| | | | oxazolidin-3-yl | | | | | | | | |
| 166 | H | 3-Oxomorpholin-4-yl | H | H | H | H | H | Br | H | 2,2-Difluoroethyl | 1.51[b] |
| 167 | H | 2-Methyl-5-oxopyrrolidin-1- | H | H | H | H | H | Br | H | 2,2-Difluoroethyl | 1.91[b] |
| | | yl | | | | | | | | | |
| 168 | H | 3-Methyl-2,5- | H | H | H | H | H | Br | H | 2,2-Difluoroethyl | 1.78[b] |
| | | dioxopyrrolidin-1-yl | | | | | | | | | |
| 169 | H | 4-Methyl-2-oxo-1,3- | H | H | H | H | H | Cl | H | 2,2,2-Trifluoroethyl | 2.16[b] |
| | | oxazolidin-3-yl | | | | | | | | | |
| 170 | H | 3,3-Dimethyl-2,5- | H | H | H | H | H | Cl | H | 2,2,2-Trifluoroethyl | 2.36[b] |
| | | dioxopyrrolidin-1-yl | | | | | | | | | |
| 171 | H | (3R,5S)-3,5-Dimethyl-2- | H | H | H | H | H | Cl | H | 2,2,2-Trifluoroethyl | 2.51[b] |
| | | oxopyrrolidin-1-yl | | | | | | | | | |
| 172 | H | H | 4-Methyl-2-oxo-1,3- | H | H | H | H | Cl | H | 2,2,2-Trifluoroethyl | 2.06[b] |
| | | | oxazolidin-3-yl | | | | | | | | |
| 173 | H | 3-Oxomorpholin-4-yl | H | H | H | H | H | Cl | H | 2,2,2-Trifluoroethyl | 1.73[b] |
| 174 | H | 2-Methyl-5-oxopyrrolidin-1- | H | H | H | H | H | Cl | H | 2,2,2-Trifluoroethyl | 2.16[b] |
| | | yl | | | | | | | | | |
| 175 | H | 2-Oxo-1,3-oxazolidin-3-yl | H | H | H | H | H | Cl | H | 2,2,2-Trifluoroethyl | 2.01[b] |
| 176 | H | 4-Methyl-2-oxo-1,3- | H | H | H | H | H | Cl | CH₃ | Cyclopropyl | 2.29[b] |
| | | oxazolidin-3-yl | | | | | | | | | |
| 177 | H | 3,3-Dimethyl-2,5- | H | H | H | H | H | Cl | CH₃ | Cyclopropyl | 2.55[b] |
| | | dioxopyrrolidin-1-yl | | | | | | | | | |
| 178 | H | (3R,5S)-3,5-Dimethyl-2- | H | H | H | H | H | Cl | CH₃ | Cyclopropyl | 2.61[b] |
| | | oxopyrrolidin-1-yl | | | | | | | | | |
| 179 | H | H | 4-Methyl-2-oxo-1,3- | H | H | H | H | Cl | CH₃ | Cyclopropyl | 2.16[b] |
| | | | oxazolidin-3-yl | | | | | | | | |
| 180 | H | 2-Methyl-5-oxopyrrolidin-1- | H | H | H | H | H | Cl | CH₃ | Cyclopropyl | 2.26[b] |
| | | yl | | | | | | | | | |
| 181 | H | 3-Methyl-2,5- | H | H | H | H | H | Cl | H | 2,2,2-Trifluoroethyl | 2.08[b] |
| | | dioxopyrrolidin-1-yl | | | | | | | | | |
| 182 | H | 3-Methyl-2,5- | H | H | H | H | H | Cl | CH₃ | Cyclopropyl | 2.21[b] |
| | | dioxopyrrolidin-1-yl | | | | | | | | | |
| 183 | H | (4R)-4-Methyl-2-oxo-1,3- | H | H | H | H | H | Br | H | 1-Methoxypropan-2-yl | 1.71[b] |
| | | oxazolidin-3-yl | | | | | | | | | |
| 184 | H | 2-Oxopyrrolidin-1-yl | H | H | H | Acetyl | H | Cl | CH₃ | Cyclopropyl | 2.38[c]; |
| | | | | | | | | | | | 2.35[b] |
| 185 | H | 3-Methyl-2-oxopyrrolidin-1- | H | H | H | H | H | Cl | H | Propan-2-yl | 3.07[c]; |
| | | yl | | | | | | | | | 1.82[b] |
| 186 | H | 3-Methyl-2-oxopyrrolidin-1- | H | H | H | H | H | Cl | CH₃ | Cyclopropyl | 3.48[c]; |
| | | yl | | | | | | | | | 2.66[b] |
| 187 | H | 3-Methyl-2-oxopyrrolidin-1- | H | H | H | H | H | Br | H | Cyclopropyl | 2.83[c]; |
| | | yl | | | | | | | | | 1.87[b] |
| 188 | H | 3-Methyl-2,5- | H | H | H | H | H | Cl | H | Cyclobutyl | 2.67[c]; |
| | | dioxopyrrolidin-1-yl | | | | | | | | | 1.76[b] |
| 189 | H | (3R,5S)-3,5-Dimethyl-2- | H | H | H | H | H | Br | H | 2,2-Difluoroethyl | 3.24[b] |
| | | oxopyrrolidin-1-yl | | | | | | | | | |
| 190 | H | (2S)-2-(Ethoxycarbonyl)-5- | H | H | H | H | H | Cl | H | Cyclopropyl | 2.53[c]; |
| | | oxopyrrolidin-1-yl | | | | | | | | | 1.69[b] |
| 191 | H | (2S)-2-(Ethoxycarbonyl)-5- | H | H | H | H | H | Br | H | Cyclopropyl | 2.65[c] |
| | | oxopyrrolidin-1-yl | | | | | | | | | |
| 192 | H | 3,3-Dimethyl-2,5- | H | H | H | H | H | CF₃ | H | Cyclobutyl | 3.03[b] |
| | | dioxopyrrolidin-1-yl | | | | | | | | | |
| 193 | H | (2S)-2-(Ethoxycarbonyl)-5- | H | H | H | H | H | CF₃ | H | Cyclopropyl | 2.82[c]; |
| | | oxopyrrolidin-1-yl | | | | | | | | | 2.56[b] |
| 194 | H | 2-Oxo-1,3-oxazolidin-3-yl | H | H | H | H | H | CF₃ | H | Cyclobutyl | 2.58[b] |
| 195 | H | 2,5-Dioxopyrrolidin-1-yl | H | H | H | H | H | Cl | H | Cyclopropyl | 1.98[c]; |
| | | | | | | | | | | | 1.4[b]; |
| | | | | | | | | | | | 0.99[d] |
| 196 | H | 3,3-Dimethyl-2,5- | H | H | H | H | H | CF₃ | H | Cyclopropylmethyl | 2.81[b] |
| | | dioxopyrrolidin-1-yl | | | | | | | | | |
| 197 | H | 2-Oxopyrrolidin-1-yl | Fluorine | H | H | H | H | Cl | H | Propan-2-yl | 2.55[c]; |
| | | | | | | | | | | | 1.55[b] |
| 198 | H | 2-Oxopyrrolidin-1-yl | Fluorine | H | H | H | H | Cl | CH₃ | Cyclopropyl | 2.92[c]; |
| | | | | | | | | | | | 2.53[b] |
| 199 | H | 2-Oxopyrrolidin-1-yl | Fluorine | H | H | H | H | Cl | H | CH₃ | 1.91[c]; |
| | | | | | | | | | | | 1.28[b] |
| 200 | H | 2-Oxopyrrolidin-1-yl | H | H | H | H | H | Cl | H | 2-Methylcyclopropyl | 1.72[b] |
| 201 | H | 2-Oxopyrrolidin-1-yl | Fluorine | H | H | H | H | CF₃ | H | Cyclopropylmethyl | 2.41[b] |
| 202 | H | 2,5-Dioxopyrrolidin-1-yl | H | H | H | H | H | Cl | H | 1-Methoxypropan-2-yl | 2.13[c]; |
| | | | | | | | | | | | 1.26[b]; |
| | | | | | | | | | | | 1.02[d] |
| 203 | H | 2,5-Dioxopyrrolidin-1-yl | H | H | H | H | H | Cl | H | Cyclobutyl | 2.32[c]; |
| | | | | | | | | | | | 1.48[b]; |
| | | | | | | | | | | | 1.24[d] |
| 204 | H | 2-Oxopyrrolidin-1-yl | H | H | H | H | H | Cl | H | Ethyl | 2.24[c]; |
| | | | | | | | | | | | 1.29[b] |
| 205 | H | 2-Oxo-1,3-oxazolidin-3-yl | H | H | H | H | H | Cl | H | 2-Methylcyclopropyl | 1.67[b] |
| 206 | H | 5-Ethyl-3-methyl-2- | H | H | H | H | H | Cl | H | Cyclopropyl | 3.15[c]; |
| | | oxopyrrolidin-1-yl | | | | | | | | | 2.19[b] |
| 207 | H | 2-Oxopyrrolidin-1-yl | H | H | H | H | H | Cl | CH₃ | Ethyl | 2.91[c]; |
| | | | | | | | | | | | 1.79[b] |
| 208 | H | 5-Ethyl-3-methyl-2- | H | H | H | H | H | Br | H | Cyclopropyl | 3.31[c]; |
| | | oxopyrrolidin-1-yl | | | | | | | | | 2.32[b] |
| 209 | H | 5-Ethyl-3-methyl-2- | H | H | H | H | H | Br | H | Cyclopropylmethyl | 3.69[c]; |
| | | oxopyrrolidin-1-yl | | | | | | | | | 2.41[b] |
| 210 | H | 5-Ethyl-3-methyl-2- | H | H | H | H | H | Br | H | Propan-2-yl | 3.7[c]; |
| | | oxopyrrolidin-1-yl | | | | | | | | | 2.36[b] |
| 211 | H | 5-Ethyl-3-methyl-2- | H | H | H | H | H | Br | H | Cyclobutyl | 2.63[b]; |
| | | oxopyrrolidin-1-yl | | | | | | | | | 2.19[d] |
| 212 | H | 5-Ethyl-3-methyl-2- | H | H | H | H | H | Cl | H | Cyclopropylmethyl | 3.54[c]; |
| | | oxopyrrolidin-1-yl | | | | | | | | | 2.33[b] |
| 213 | H | 2-Oxopiperidin-1-yl | H | H | H | H | H | Cl | H | 2-Methylcyclopropyl | 2.48[c]; |
| | | | | | | | | | | | 1.7[b] |
| 214 | H | 2-Thioxopyrrolidin-1-yl | H | H | H | H | H | CF₃ | H | Cyclopropyl | 2.86[c]; |
| | | | | | | | | | | | 2.59[b] |
| 215 | H | 4-Methyl-2-oxo-1,3- | H | H | H | H | H | Cl | H | 2-Methylcyclopropyl | 2.79[c]; |
| | | oxazolidin-3-yl | | | | | | | | | 1.84[b] |
| 216 | H | (4R)-2-Oxo-4-(propan-2-yl)- | H | H | H | H | H | Cl | H | 2-Methylcyclopropyl | 3.46[c]; |
| | | 1,3-oxazolidin-3-yl | | | | | | | | | 2.43[b] |
| 217 | H | 2-Oxopyrrolidin-1-yl | H | H | H | Formyl | H | Cl | H | Cyclopropyl | 1.49[b] |
| 218 | H | 2-Oxopyrrolidin-1-yl | H | H | H | H | H | CF₃ | H | 2,2,3,3,3- | 3.07[c]; |
| | | | | | | | | | | Pentafluorpropyl | 3.1[b] |
| 219 | H | 2-Oxopyrrolidin-1-yl | H | H | H | H | H | CF₃ | H | 2-Methylcyclopropyl | 2.79[b] |
| 220 | H | 2-Oxopyrrolidin-1-yl | H | H | H | H | H | CF₃ | H | 1,1,1-Trifluoropropan-2- | 3.05[c]; |
| | | | | | | | | | | yl | 3.08[b] |
| 221 | H | 2-Oxopyridin-1(2H)-yl | H | H | H | H | H | Br | H | Cyclobutyl | 1.71[b] |
| 222 | H | 2-Oxopyrrolidin-1-yl | H | H | H | H | H | Cl | | —CH₂CH₂CH₂CH₂— | 1.67[b] |
| 223 | H | 2-Oxopiperidin-1-yl | H | H | H | H | H | Br | H | 2-Methylcyclopropyl | 2.67[c]; |
| | | | | | | | | | | | 1.81[b] |
| 224 | H | 3-Ethyl-4-methyl-2-oxo-2,5- | H | H | H | H | H | Cl | H | Cyclopropyl | 2.2[a] |
| | | dihydro-1H-pyrrol-1-yl | | | | | | | | | |
| 225 | H | 2-Oxopyrrolidin-1-yl | H | H | H | H | H | Cl | | —CH₂CH₂CH₂CH₂CH₂— | 3.53[c]; |
| | | | | | | | | | | | 2.57[b] |
| 226 | H | 2-Oxopyrrolidin-1-yl | H | H | H | H | H | Cl | | —CH₂CH₂OCH₂CH₂— | 2.29[c]; |
| | | | | | | | | | | | 1.98[b] |
| 227 | H | 2-Oxo-1,3-oxazolidin-3-yl | H | H | H | H | H | Br | H | 2-Methylcyclopropyl | 2.79[c]; |
| | | | | | | | | | | | 1.77[b] |
| 228 | H | 2-Oxopyrrolidin-1-yl | H | H | H | H | H | Cl | | —CH₂—CH₂—S—CH₂—CH₂— | 3.01[c]; |
| | | | | | | | | | | | 2.63[b] |
| 229 | H | 2-Oxopyrrolidin-1-yl | H | H | H | H | H | Br | H | 2-Methylcyclopropyl | 2.82[c]; |
| | | | | | | | | | | | 1.84[b] |
| 230 | H | (4S)-4-Methyl-2-oxo-1,3- | H | H | H | H | H | Cl | | —CH₂CH₂CH₂— | 2.75[c]; |
| | | oxazolidin-3-yl | | | | | | | | | 1.58[b] |
| 231 | H | 2-Oxopyrrolidin-1-yl | H | H | H | Methoxyacetyl | H | Cl | H | Cyclopropyl | 1.82[c]; |
| | | | | | | | | | | | 1.87[b] |
| 232 | H | 3-Ethyl-4-methyl-2-oxo-2,5- | H | H | H | H | H | Br | H | Cyclopropyl | 2.42[a] |
| | | dihydro-1H-pyrrol-1-yl | | | | | | | | | |
| 233 | H | (3-Methyl-2-oxopyrrolidin-1- | H | H | H | H | H | Cl | CH₃ | Cyclopropyl | 2.5[a]; |
| | | yl)methyl | | | | | | | | | 3.25[c] |
| 234 | H | (3-Methyl-2-oxopyrrolidin-1- | H | H | H | H | H | Cl | H | Cyclopropyl | 1.7[a]; |
| | | yl)methyl | | | | | | | | | 2.57[c] |
| 235 | H | (2,5-Dioxopyrrolidin-1- | H | H | H | H | H | Cl | H | Cyclopropyl | 2.32[c]; |
| | | yl)methyl | | | | | | | | | 1.42[b] |
| 236 | H | (2-Oxopyrrolidin-1- | H | H | H | H | H | Cl | H | Cyclopropyl | 1.49[b] |
| | | yl)carbonyl | | | | | | | | | |
| 237 | H | 4-Methoxy-2-oxo-2,5- | H | H | H | H | H | Cl | H | Cyclopropyl | 1.55[a]; |
| | | dihydro-1H-pyrrol-1-yl | | | | | | | | | 2.45[c] |
| 238 | H | 4-Methoxy-2-oxo-2,5- | H | H | H | H | H | Br | H | Cyclopropyl | 1.77[a]; |
| | | dihydro-1H-pyrrol-1-yl | | | | | | | | | 2.65[c] |
| 239 | H | 4-Methyl-2-oxo-1,3- | H | H | H | H | H | Br | H | 2-Methylcyclopropyl | 2.9[c]; |
| | | oxazolidin-3-yl | | | | | | | | | 1.83[b] |
| 240 | H | 4-Methyl-2-oxopyrrolidin-1- | H | H | H | H | H | CF₃ | H | Cyclopropyl | 3.06[c]; |
| | | yl | | | | | | | | | 2.65[b] |
| 241 | H | 3-Methyl-2-oxopyrrolidin-1- | H | H | H | H | H | Cl | H | 2-Methylcyclopropyl | 3.17[c]; |
| | | yl | | | | | | | | | 2.06[b] |
| 242 | H | 2-Oxopyrrolidin-1-yl | H | H | H | H | H | Cl | H | 2-Ethylcyclopropyl | 1.99[b] |
| 243 | H | 2-Methyl-5-oxopyrrolidin-1- | H | H | H | H | H | CF₃ | H | Cyclopropyl | 2.73[c]; |
| | | yl | | | | | | | | | 2.41[b] |
| 244 | H | 3-Methyl-2,5- | H | H | H | H | H | CF₃ | H | Cyclopropyl | 2.53[c]; |
| | | dioxopyrrolidin-1-yl | | | | | | | | | 2.29[b] |
| 245 | H | 3,5-Dimethyl-2- | H | H | H | H | H | CF₃ | H | Cyclopropyl | |
| | | oxopyrrolidin-1-yl | | | | | | | | | |
| 246 | H | 3,3-Dimethyl-2,5- | H | H | H | H | H | Cl | H | 2-Methylcyclopropyl | 2.92[c]; |
| | | dioxopyrrolidin-1-yl | | | | | | | | | 2.09[b] |
| 247 | H | 3-Methyl-2,5- | H | H | H | H | H | CF₃ | H | Cyclobutyl | 3.04[c]; |
| | | dioxopyrrolidin-1-yl | | | | | | | | | 2.67[b] |
| 248 | H | (4R)-2-Oxo-4-(propan-2-yl)- | H | H | H | H | H | Br | H | Cyclobutyl | 3.71[c]; |
| | | 1,3-oxazolidin-3-yl | | | | | | | | | 2.54[b] |
| 249 | H | (4R)-2-Oxo-4-(propan-2-yl)- | H | H | H | H | H | CF₃ | H | Cyclopropyl | 3.34[c]; |
| | | 1,3-oxazolidin-3-yl | | | | | | | | | 3.04[b] |
| 250 | H | (4R)-2-Oxo-4-(propan-2-yl)- | H | H | H | H | H | Br | H | Cyclopropyl | 3.18[c]; |
| | | 1,3-oxazolidin-3-yl | | | | | | | | | 2.22[b] |
| 251 | H | 3-Methyl-2-oxopyrrolidin-1- | H | H | H | H | H | CF₃ | H | Cyclobutyl | 3.67[c]; |
| | | yl | | | | | | | | | 3.16[b] |
| 252 | H | 2-Oxopyrrolidin-1-yl | H | H | H | H | H | CF₃ | H | 1-Methylcyclopropyl | 2.95[c]; |
| | | | | | | | | | | | 2.44[b] |
| 253 | H | 2-Methyl-5-oxopyrrolidin-1- | H | H | H | H | H | Cl | H | 3-Methylcyclobutyl | 3.16[c]; |
| | | yl | | | | | | | | | 2.15[b] |
| 254 | H | 2-Oxopyrrolidin-1-yl | H | H | H | H | H | Cl | H | 2-Methylcyclopropyl | |
| 255 | H | 2-Ethyl-5-oxopyrrolidin-1-yl | H | H | H | H | H | CF₃ | H | Cyclopropyl | 3.08[c]; |
| | | | | | | | | | | | 2.75[b] |
| 256 | H | 2-Oxo-1,3-oxazolidin-3-yl | H | H | H | H | H | Cl | H | 3-Methylcyclobutyl | 2.99[c] |
| 257 | H | 3,3-Dimethyl-2,5- | H | H | H | H | H | Br | H | 3-Methylcyclobutyl | 3.66[c]; |
| | | dioxopyrrolidin-1-yl | | | | | | | | | 2.58[b] |
| 258 | H | 2-Oxo-5- | H | H | H | H | H | Br | H | Cyclobutyl | 3.24[c]; |
| | | (trifluoromethyl)pyrrolidin-1- | | | | | | | | | 2.3[b] |
| | | yl | | | | | | | | | |
| 259 | H | 3,3-Dimethyl-2,5- | H | H | H | H | H | Cl | H | 3-Methylcyclobutyl | 3.22[c]; |
| | | dioxopyrrolidin-1-yl | | | | | | | | | 2.43[b] |
| 260 | H | 2-Oxo-5- | H | H | H | H | H | Cl | H | Cyclopropyl | 2.67[c]; |
| | | (trifluoromethyl)pyrrolidin-1- | | | | | | | | | 1.89[b] |
| | | yl | | | | | | | | | |
| 261 | H | (4R)-4-Methyl-2-oxo-1,3- | H | H | H | H | H | CF₃ | H | Cyclopropyl | 2.7[c]; |
| | | oxazolidin-3-yl | | | | | | | | | 2.4[b] |
| 262 | H | (4R)-4-Methyl-2-oxo-1,3- | H | H | H | H | H | Br | H | Propan-2-yl | 2.83[c]; |
| | | oxazolidin-3-yl | | | | | | | | | 1.73[b] |
| 263 | H | (4R)-2-Oxo-4-(propan-2-yl)- | H | H | H | H | H | CF₃ | H | Cyclobutyl | 3.85[c]; |
| | | 1,3-oxazolidin-3-yl | | | | | | | | | 3.47[b] |
| 264 | H | 5-Methyl-2-oxo-1,3- | H | H | H | H | H | Cl | H | Cyclopropyl | 2.56[c]; |
| | | oxazolidin-3-yl | | | | | | | | | 1.65[b] |
| 281 | H | 3-Methyl-2,5- | H | H | H | H | H | Br | H | 3-Methylcyclobutyl | 2.98[c]; |
| | | dioxopyrrolidin-1-yl | | | | | | | | | 2.23[b] |
| 282 | H | 5-Ethyl-3-methyl-2- | H | H | H | H | H | Cl | H | Cyclobutyl | 3.77[c]; |
| | | oxopyrrolidin-1-yl | | | | | | | | | 2.49[b] |
| 283 | H | 2-Oxopyrrolidin-1-yl | H | H | H | H | H | Cl | H | 2-Methylcyclopropyl | |
| 284 | H | 4-Methyl-2-oxopyrrolidin-1- | H | H | H | H | H | CF₃ | H | Cyclobutyl | 3.57[c]; |
| | | yl | | | | | | | | | 3.06[b] |
| 285 | H | 3-(2-Chloroethyl)-2- | H | H | H | H | H | Br | H | Cyclopropyl | 1.92[b] |
| | | oxoimidazolidin-1-yl | | | | | | | | | |
| 286 | H | 3-(2-Chloroethyl)-2- | H | H | H | H | H | Cl | H | Cyclopropyl | 1.82[b] |
| | | oxoimidazolidin-1-yl | | | | | | | | | |
| 287 | H | 5-Methyl-2-oxo-1,3- | H | H | H | H | H | Br | H | Cyclobutyl | 3.17[c]; |
| | | oxazolidin-3-yl | | | | | | | | | 2.04[b] |
| 288 | H | 2,5-Dioxopyrrolidin-1-yl | H | H | H | H | H | Br | H | 3-Methylcyclobutyl | 1.86[b] |
| 289 | H | 3,5-Dimethyl-2- | H | H | H | H | H | CF₃ | H | Cyclobutyl | 3.74[c]; |
| | | oxopyrrolidin-1-yl | | | | | | | | | 3.27[b] |
| 290 | H | 3-Methyl-2-oxoimidazolidin- | H | H | H | H | H | Br | H | 3-Methylcyclobutyl | 2[b] |
| | | 1-yl | | | | | | | | | |
| 291 | H | 3,5-Dimethyl-2- | H | H | H | H | H | Br | H | 3-Methylcyclobutyl | 2.66[b] |
| | | oxopyrrolidin-1-yl | | | | | | | | | |
| 292 | H | 3-Methyl-2-oxopyrrolidin-1- | H | H | H | H | H | CF₃ | H | Cyclopropyl | 3.05[c]; |
| | | yl | | | | | | | | | 2.65[b] |
| 293 | H | 3-Methyl-2-oxoimidazolidin- | H | H | H | H | H | CF₃ | H | Cyclopropyl | 2.22[b] |
| | | 1-yl | | | | | | | | | |
| 294 | H | 2-Ethyl-5-oxopyrrolidin-1-yl | H | H | H | H | H | Cl | H | Cyclopropyl | 2.74[c]; |
| | | | | | | | | | | | 1.87[b] |
| 295 | H | 3,3-Dimethyl-2,5- | H | H | H | H | H | Br | H | 2-Methylcyclopropyl | 3.11[c]; |
| | | dioxopyrrolidin-1-yl | | | | | | | | | 2.2[b] |
| 296 | H | 3-Methyl-2-oxoimidazolidin- | H | H | H | H | H | Iodine | H | Cyclopropyl | 1.57[b] |
| | | 1-yl | | | | | | | | | |
| 297 | H | 2-Oxopyrrolidin-1-yl | H | H | H | H | H | CF₃ | H | Propan-2-yl | 3[c]; |
| | | | | | | | | | | | 2.35[b] |
| 298 | H | 2-Oxopyrrolidin-1-yl | H | H | H | H | H | CF₃ | H | 1-Fluoropropan-2-yl | 2.48[b] |
| 299 | H | 2-Oxopyrrolidin-1-yl | H | H | H | H | H | Cl | H | 3-Methylcyclobutyl | 3.26[c]; |
| | | | | | | | | | | | 3.25[b] |
| 300 | H | H | 2-Oxo-1,3-oxazolidin-3-yl | H | H | H | H | Cl | H | 2-Methylcyclopropyl | |
| 301 | H | 2-Oxopyrrolidin-1-yl | H | H | H | H | H | CF₃ | —CH₂CH₂CH₂CH₂— | | 3.24[c]; |
| | | | | | | | | | | | 2.45[b] |
| 302 | H | 2-Oxopyrrolidin-1-yl | H | H | H | H | H | CF₃ | H | 2-Fluoroethyl | 2.67[c]; |
| | | | | | | | | | | | 2.05[b] |
| 303 | H | 2-Oxopyrrolidin-1-yl | H | H | H | H | H | CF₃ | H | Cyclopentyl | 3.04[b] |
| 304 | H | 2-Oxopyrrolidin-1-yl | H | H | H | H | H | Cl | CH₃ | 2-Methylcyclopropyl | 3.53[c]; |
| | | | | | | | | | | | 2.66[b] |
| 305 | H | 2-Oxopyrrolidin-1-yl | CH₃ | H | H | H | H | Cl | H | Cyclopropyl | 2.32[c]; |
| | | | | | | | | | | | 1.44[b] |
| 306 | H | 2-Oxopyrrolidin-1-yl | H | H | H | H | H | CF₃ | H | 2,2,2-Trifluoroethyl | 2.66[c]; |
| | | | | | | | | | | | 2.6[b] |
| 307 | H | 2-Oxopyrrolidin-1-yl | H | H | H | H | H | Cl | H | Propyl | 1.49[b] |
| 308 | H | H | 2-Oxo-1,3-oxazolidin-3-yl | H | H | H | H | Br | H | 2-Methylcyclopropyl | 2.91[c]; |
| | | | | | | | | | | | 1.97[b] |
| 309 | H | 2-Oxopyrrolidin-1-yl | H | H | H | Methoxycarbonyl | H | CF₃ | H | Cyclopropyl | 2.37[c]; |
| | | | | | | | | | | | 2.25[b] |
| 310 | H | 2-Oxopyrrolidin-1-yl | H | CH₃ | H | H | H | CF₃ | H | Cyclopropyl | 2.98[c]; |
| | | | | | | | | | | | 2.51[b] |
| 311 | H | 2-Oxopyrrolidin-1-yl | H | H | H | H | H | CF₃ | H | 2,2-Difluoroethyl | 2.52[c]; |
| | | | | | | | | | | | 2.38[b] |
| 312 | H | 5-Methyl-2-oxo-1,3- | H | H | H | H | H | Cl | H | Cyclobutyl | 3.02[c]; |
| | | oxazolidin-3-yl | | | | | | | | | 1.91[b] |
| 313 | H | 3-Methyl-2-oxoimidazolidin- | H | H | H | H | H | Cl | H | 2,2- | 1.93[a]; |
| | | 1-yl | | | | | | | | Dimethylcyclopropyl | 3.06[c]; |
| | | | | | | | | | | | 1.93[b] |
| 314 | H | 2-Oxo-5- | H | H | H | H | H | CF₃ | H | Cyclopropyl | 2.98[c]; |
| | | (trifluoromethyl)pyrrolidin-1- | | | | | | | | | 2.74[b] |
| | | yl | | | | | | | | | |
| 315 | H | 2-Oxopyrrolidin-1-yl | H | H | H | H | H | Cl | H | 2-Methylcyclopropyl | |
| 316 | H | 2-Oxopyrrolidin-1-yl | CH₃ | H | H | H | H | CF₃ | H | Cyclopropyl | 2.62[c]; |
| | | | | | | | | | | | 2.16[b] |
| 317 | H | 2-Methyl-5-oxopyrrolidin-1- | H | H | H | H | H | Br | H | 3-Methylcyclobutyl | 2.17[b] |
| | | yl | | | | | | | | | |
| 318 | H | 2-Oxopyrrolidin-1-yl | H | methoxy | H | H | H | Cl | H | Cyclopropyl | 2.46[c]; |
| | | | | | | | | | | | 1.64[b] |
| 319 | H | 2-Oxopyrrolidin-1-yl | H | H | H | H | H | CF₃ | Cyclopentyl | CH₃ | 3.56[b] |
| 320 | H | 2-Oxopyrrolidin-1-yl | H | H | H | H | H | CF₃ | —CH₂CH₂CH₂CH₂CH₂— | | 3.29[b] |
| 321 | H | 2-Oxopyrrolidin-1-yl | H | H | H | H | H | CF₃ | H | 2,2- | 3.04[b] |
| | | | | | | | | | | Dimethylcyclopropyl | |
| 322 | H | 2-Oxopyrrolidin-1-yl | H | H | H | H | H | CF₃ | H | Oxetan-3-yl | 1.84[b] |
| 323 | H | 2-Oxopyrrolidin-1-yl | H | H | H | H | H | CF₃ | H | CH₃ | 1.63[b] |
| 324 | H | 2-Oxopyrrolidin-1-yl | Methoxy | H | H | H | H | CF₃ | H | Cyclopropyl | 2.51[c]; |
| | | | | | | | | | | | 1.98[b] |
| 325 | H | 2-Oxopyrrolidin-1-yl | H | H | H | CH₃ | H | Cl | CH₃ | Cyclopropyl | 3.66[c]; |
| | | | | | | | | | | | 1.98[b] |
| 326 | H | 2-Oxopyrrolidin-1-yl | H | methoxy | H | H | H | CF₃ | H | Cyclopropyl | 2.8[c]; |
| | | | | | | | | | | | 2.47[b] |
| 327 | H | 2-Oxopyrrolidin-1-yl | H | H | H | Methoxycarbonyl | H | Cl | H | Cyclopropyl | 1.99[c]; |
| | | | | | | | | | | | 1.77[b] |
| 328 | H | 2-Oxopyrrolidin-1-yl | Methoxy | H | H | H | H | Cl | H | Cyclopropyl | 2.18[c]; |
| | | | | | | | | | | | 1.3[b] |
| 329 | H | 2-Oxopyrrolidin-1-yl | H | H | H | (Propan- | H | Cl | H | Cyclopropyl | 2.55[c]; |
| | | | | | | 2- | | | | | 2.24[b] |
| | | | | | | yloxy)carbonyl | | | | | |
| 330 | H | 2-Oxopyrrolidin-1-yl | H | H | H | Ethoxycarbonyl | H | Cl | H | Cyclopropyl | 2.24[c]; |
| | | | | | | | | | | | 2.04[b] |
| 331 | H | 3,5-Dimethyl-2- | H | H | H | H | H | Cl | H | 2,2-Difluoroethyl | 2.11[b] |
| | | oxopyrrolidin-1-yl | | | | | | | | | |
| 332 | H | 2-Oxopyrrolidin-1-yl | H | H | H | H | H | Cl | H | 2-Methylcyclopropyl | |
| 333 | H | 3-Methyl-2,5- | H | H | H | H | H | Cl | H | 3-Methylcyclobutyl | 3.09[c]; |
| | | dioxopyrrolidin-1-yl | | | | | | | | | 2.09[b] |

Examples 254, 283, 332, 315 are the isolated stereoisomers trans1, trans2, cis2 and cis1 which can be obtained from the stereoisomer mixture of Example 200.

The log P values were measured in accordance with EEC Directive 79/831 Annex V.A8 by HPLC (High Performance Liquid Chromatography) on reversed-phase columns (C 18) using the methods below:

[a] The determination was carried out in the acidic range at pH 2.3 using the mobile phases 0.1% aqueous phosphoric acid and acetronitrile

linear gradient from 10% acetonitrile to 95% acetronitrile.

[b] The LC-MS determination in the acid range was carried out at pH 2.7 using the mobile phases 0.1% aqueous formic acid and acetonitrile (contains 0.1% formic acid)

linear gradient from 10% acetonitrile to 95% acetonitrile.

[c] The LC-MS determination in the neutral range was carried out at pH 7.8 using the mobile phases 0.001 molar aqueous ammonium bicarbonate solution and acetonitrile

linear gradient from 10% acetonitrile to 95% acetonitrile.

Calibration was carried out using unbranched alkan-2-ones (having 3 to 16 carbon atoms) with known log P values (determination of the log P values by the retention times using linear interpolation between two successive alkanones).

The lambda-maX values were determined in the maxima of the chromatographic signals using the UV spectra from 200 nm to 400 nm.

| Ex. No. | | |
|---|---|---|
| from | | |
| Table I | ¹H NMR | M + 1 |
| 1 | ¹H NMR (pyrimidine-H): δ = 8.03 | 376 |
| 2 | ¹H NMR (pyrimidine-H): δ = 8.04 | 376 |
| 3 | | 392 |
| 4 | ¹H NMR (pyrimidine-H): δ = 8.01 (s, 2 H) | 437 |
| 5 | ¹H NMR (pyrimidine-H): δ = 8.01 (s, 2 H) | 421 |
| 6 | ¹H NMR (pyrimidine-H): δ = 8.18 | 410 |
| 7 | ¹H NMR (pyrimidine-H): δ = 7.89 | 360 |
| 8 | ¹H NMR (pyrimidine-H): δ = 8.03 | 360 |
| 9 | ¹H NMR (pyrimidine-H): δ = 8.02 | 386 |
| 10 | ¹H NMR (pyrimidine-H): δ = 8.04 | 386 |
| 11 | ¹H NMR (pyrimidine-H): δ = 8.11 | 438 |
| 12 | | 377.1 |
| 13 | ¹H NMR δ = 8.95 (s, 1 H), 7.91 (s, 1 H), 7.67 (d, 2 H), 7.47 (d 2 H), | 344 |
| | 7.12 (m, 1 H), 6.00-5.91 (m, 1 H), 5.20-5.08 (m, 2 H), 4.06 (m, 2 H), 3.81 (t, | |
| | 2 H), 2.53-2.43 (m, 2 H), 2.09-2.02 (m, 2 H). | |
| 14 | ¹H NMR (pyrimidine-H): δ = 7.92 | 344 |
| 15 | ¹H NMR (pyrimidine-H): δ = 7.91 | 346 |
| 16 | ¹H NMR (pyrimidine-H): δ = 7.93 (s, 1 H) | 368 |
| 17 | ¹H NMR (pyrimidine-H): δ = 7.97 | 362 |
| 18 | ¹H NMR (pyrimidine-H): δ = 7.9 | 360 |
| 19 | ¹H NMR: δ = 9.54 (s, 1 H), 8.18 (s, 1 H), 7.95 (s, 1 H), 7.74 (dd, 1 H); | 394 |
| | 7.29 (t, 1 H), 7.06 (dd, 1 H), 6.84 (br. s, 1 H), 4.52 (m 2 H), 3.98 (t, 1 H), | |
| | 2.94 (m, 1 H), 1.21 (d, 3 H), 0.74 (m, 2 H), 0.65 (m, 2 H). | |
| 20 | ¹H NMR (pyrimidine-H): δ = 7.94 | 428 |
| 21 | ¹H NMR δ = 8.94 (s, 1 H), 7.98 (s, 1 H), 7.69 (d, 2 H), 7.49 (d 2 H), | 346 |
| | 6.44 (m, 1 H), 4.33 (m, 1 H), 3.79 (t, 2 H), 2.53-2.43 (m, 2 H), | |
| | 2.09-2.02 (m, 2 H), 1.24 (d, 6 H). | |
| 22 | ¹H NMR δ = 9.49 (s, 1 H), 7.99 (s, 1 H), 7.96 (s, 1 H), 7.37 (d, 1 H), | 346 |
| | 7.28 (m, 2 H), 7.17 (br.s, 1 H), 4.43 (m, 1 H), 3.81 (t, 2 H), 2.09 (m, 2 H), | |
| | 1.23 (d, 6 H). | |
| 23 | | 388 |
| 24 | ¹H NMR (pyrimidine-H): δ = 8.3 | 367.2 |
| 25 | ¹H NMR (pyrimidine-H): δ = 7.88 (s, 1 H) | 386 |
| 26 | ¹H NMR δ = 9.02 (s, 1 H), 8.06 (s, 1 H), 7.90 (s, 1 H), 7.59 (m, 1 H), | 358 |
| | 7.20 (m, 2 H), 6.95 (br. s, 1 H), 3.74 (m, 2 H), 2.96 (m, 1 H), 2.59 (m, 1 H), | |
| | 2.32 (m, 1 H), 1.67 (m, 1 H), 1.15 (d, 3 H), 0.71 (m, 2 H), 0.64 (m, 2 H). | |
| 27 | ¹H NMR (pyrimidine-H): δ = 8.02 (s, 1 H) | 417 |
| 28 | ¹H NMR (pyrimidine-H): δ = 8.12 (s, 1 H) | 431 |
| 29 | ¹H NMR (pyrimidine-H): δ = 8.03 (s, 1 H) | 400 |
| 30 | ¹H NMR (pyrimidine-H): δ = 7.90 (s, 1 H) | 358 |
| 31 | ¹H NMR δ = 9.10 (s, 1 H), 7.92 (s, 1 H), 7.91 (s, 1 H), 7.67 (dd, 1 H), | 374 |
| | 7.24 (t, 1 H), 7.03 (dd, 1 H), 6.98 (br. s, 1 H), 4.50 (m, 1 H), 4.48 (m, 1 | |
| | H), 4.11 (m, 1 H), 2.92 (m, 1 H), 0.85 (m, 5 H), 0.75 (m, 2 H), 0.65 (m, 2 | |
| | H). | |
| 32 | ¹H NMR (pyrimidine-H): δ = 7.98 (s, 1 H) | 419 |
| 33 | ¹H NMR δ = 9.43 (s, 1 H), 8.19 (dd, 1 H), 7.95 (s, 1 H), 7.68 (dd, 1 H), | 360 |
| | 7.33-7.28 (m, 2 H), 6.99 (dd, 1 H), 4.07 (t, 2 H), 3.03 (t, 2 H), | |
| | 2.88-2.84 (m, 1 H), 2.15-2.07 (m, 2 H), 0.75-0.62 (m, 4 H). | |
| 34 | ¹H NMR δ = 9.08 (s, 1 H), 8.00 (s, 1 H), 7.97 (s, 1 H), 7.46 (dd, 1 H), | 433 |
| | 7.25 (t, 1 H), 7.01 (dd, 1 H), 6.69 (d, 1 H), 4.52 (m, 1 H), 4.47 (m, 2 H), | |
| | 4.14 (m, 1 H), 2.28 (m, 2 H), 2.12 (m, 2 H), 1.69 (m, 4 H), 0.82 (t, 3 H). | |
| 35 | ¹H NMR (pyrimidine-H): δ = 7.91 (s, 1 H) | 388 |
| 36 | ¹H NMR δ = 8.00 (s, 1H) | 417.1 |
| 37 | ¹H NMR δ = 9.03 (s, 1 H), 7.93 (s, 1 H), 7.91 (s, 1 H), 7.44 (dd, 1 H), | 372 |
| | 7.23 (t, 1 H), 6.96 (m, 2 H), 4.56 (m, 1 H), 4.29 (m, 1 H), 2.41 (m, 2 H), | |
| | 2.27 (m, 3 H), 2.12 (m, 2 H), 1.69 (m, 3 H), 1.16 (d, 3 H). | |
| 38 | ¹H NMR δ = 8.97 (s, 1 H), 8.00 (s, 1 H), 7.88 (s, 1 H), 7.47 (d, 1 H), | 318 |
| | 7.27 (d, 1 H), 7.19 (dd, 1 H), 6.94 (br.s, 1 H), 3.80 (t, 2 H), 2.94 (d, 3 H), | |
| | 2.08 (m, 2 H). | |
| 39 | ¹H NMR δ = 9.09 (br. s, 1 H), 7.95 (s, 1 H), 7.91 (s, 1 H), 7.67 (dd, 1 H), | 388 |
| | 7.24 (t, 1 H), 7.04 (dd, 1 H), 6.97 (br. s, 1 H), 4.42 (m, 2 H), 4.21 (m, 1 | |
| | H), 2.93 (m, 1 H), 2.02 (m, 1 H), 0.85 (d, 3 H), 0.76 (d, 3 H), 0.74 (m, 2 | |
| | H), 0.66 (m, 2 H). | |
| 40 | ¹H NMR (pyrimidine-H): δ = 7.98 | 408 |
| 41 | ¹H NMR (pyrimidine-H): δ = 7.94 | 406 |
| 42 | ¹H NMR δ = 8.96 (s, 1 H), 7.99 (s, 1 H), 7.89 (s, 1 H), 7.45-7.42 (m, 1 H), | 372.1 |
| | 7.23-7.19 (m, 2 H), 6.52-6.49 (m, 1 H), 5.69 (s, 1 H), 4.47-4.42 (m, 1 H), | |
| | 3.82-3.78 (m, 2 H), 2.45-2.41 (m, 2 H), 2.10-2.03 (m, 2 H), 2.02-1.95 (m, | |
| | 2 H), 1.74-1.70 (m, 2 H), 1.59-1.53 (m, 4 H) | |
| 43 | ¹H NMR δ = 8.79 (s, 1 H), 8.00 (m, 1 H), 7.82 (d, 1 H), 7.42 (m, 2 H), | 342 |
| | 7.20 (m 2 H), 4.58 (m, 1 H), 3.82 (t, 2 H), 2.53-2.43 (m, 2 H), 2.29 (m, | |
| | 2 H), 2.09 (m, 4H), 1.65 (m, 2 H). | |
| 44 | ¹H NMR (pyrimidine-H): δ = 9.01 | 368 |
| 45 | | 432 |
| 46 | ¹H NMR (pyrimidine-H): δ = 7.91 (s, 1 H) | 402 |
| 47 | | 402 |
| 48 | ¹H NMR (pyrimidine-H): δ = 7.99 (s, 1 H) | 446 |
| 49 | | 416 |
| 50 | ¹H NMR (pyrimidine-H): δ = 7.99 (s, 1 H) | 461 |
| 51 | ¹H NMR (pyrimidine-H): δ = 7.98 | 386 |
| 52 | ¹H NMR (pyrimidine-H): δ = 7.99 (s, 1H) | 402.1 |
| 53 | ¹H NMR (pyrimidine-H): δ = 7.90 (s, 1H) | 358.1 |
| 54 | ¹H NMR (pyrimidine-H): δ = 7.99 (s, 1 H) | 417 |
| 55 | ¹H NMR (pyrimidine-H): δ = 7.90 (s, 1 H) | 372 |
| 56 | ¹H NMR δ = 9.07 (s, 1 H), 8.00 (s, 1 H), 7.94 (s, 1 H), 7.48 (dd, 1 H), | 419 |
| | 7.26 (t, 1 H), 6.99 (dd, 1 H), 6.69 (8d, 1 H), 4.55 (m, 3 H), 3.99 (m, 1 H), | |
| | 2.27 (m, 2 H), 2.12 (m, 2 H), 1.68 (m, 2 H), 1.24 (d, 3 H). | |
| 57 | ¹H NMR (pyrimidine-H): δ = 7.91 (s, 1 H) | 374 |
| 58 | ¹H NMR (pyrimidine-H): δ = 7.83 (s, 1 H) | 356 |
| 59 | ¹H NMR (pyrimidine-H): δ = 7.91 (s, 1 H) | 360 |
| 60 | ¹H NMR (pyrimidine-H): δ = 7.99 (s, 1 H) | 401 |
| 61 | ¹H NMR δ = 9.00 (s, 1 H), 7.88 (s, 1 H), 7.73 (d, 2 H), 7.41 (d 2 H), | 320 |
| | 7.00 (d, 1 H), 4.40 (t, 2 H), 4.02 (t, 2 H), 2.93 (d, 3 H). | |
| 62 | ¹H NMR δ = 9.65 (s, 1 H), 8.18 (s, 1 H), 8.00 (s, 1 H), 7.72 (m, 1 H), | 420 |
| | 7.36 (t, 1 H), 6.87 (m, 1 H), 6.85 (s, 1 H), 2.87 (m, 1 H), 2.72 (s, 2 H), 1.30 (s, | |
| | 6 H), 0.71 (m, 2 H), 0.64 (m, 2 H). | |
| 63 | ¹H NMR (pyrimidine-H): δ = 8.01 (s, 1 H) | 445 |
| 64 | | 348 |
| 65 | | 371.1 |
| 67 | | 377.1 |
| 68 | | 403.1 |
| 69 | | 421.1 |
| 70 | | 447.1 |
| 71 | | 385.1 |
| 72 | | 403.0 |
| 73 | | 429.1 |
| 74 | | 429.1 |
| 75 | ¹H NMR (pyrimidine-H): δ = 7.95 (s, 1 H) | 388 |
| 76 | ¹H NMR δ = 9.10 (br. s, 1 H), 7.95 (s, 1 H), 7.91 (s, 1 H), 7.67 (dd, 1 H), | 388 |
| | 7.24 (t, 1 H), 7.04 (dd, 1 H), 6.97 (br. s, 1 H), 4.40 (m, 2 H), 4.21 (m, 1 | |
| | H), 2.93 (m, 1 H), 2.02 (m, 1 H), 0.85 (d, 3 H), 0.76 (d, 3 H), 0.74 (m, 2 | |
| | H), 0.66 (m, 2 H). | |
| 77 | ¹H NMR (pyrimidine-H): δ = 7.98 | 360 |
| 78 | ¹H NMR (pyrimidine-H): δ = 7.9 | 360 |
| 79 | ¹H NMR (pyrimidine-H): δ = 7.99 | 418 |
| 80 | ¹H NMR (pyrimidine-H): δ = 7.91 (s, 1 H) | 386 |
| 81 | ¹H NMR (pyrimidine-H): δ = 7.96 (s, 1 H) | 372 |
| 82 | ¹H NMR (pyrimidine-H): δ = 8 | 388 |
| 83 | ¹H NMR (pyrimidine-H): δ = 7.81 (s, 1H) | 372.1 |
| 84 | ¹H NMR (pyrimidine-H): δ = 7.96 (s, 1H) | 418.1 |
| 85 | ¹H NMR (pyrimidine-H): δ = 8.00 (s, 1 H) | 431 |
| 86 | ¹H NMR (pyrimidine-H): δ = 7.9 | 374 |
| 87 | | 420 |
| 88 | ¹H NMR (pyrimidine-H): δ = 7.93 (s, 1 H) | 422 |
| 89 | ¹H NMR (pyrimidine-H): δ = 8.02 (s, 1 H) | 436 |
| 90 | ¹H NMR (pyrimidine-H): δ = 8.02 (s, 1 H) | 481 |
| 91 | ¹H NMR (pyrimidine-H): δ = 7.93 (s, 1 H) | 467 |
| 92 | ¹H NMR δ = 9.11 (s, 1H), 7.91 (s, 2H), 7.72-7.65 (m, 1H), 7.25 (t, 1H), | 360.1 |
| | 7.03-6.95 (m, 2H), 4.60-4.42 (m, 2H), 3.97 (dd, 1H), 2.95-2.87 (m, 1H), | |
| | 1.22 (d, 3H), 0.8-0.6 (m, 4H) | |
| 93 | ¹H NMR δ = 9.05 (s, 1 H), 7.95 (s, 1 H), 7.91 (s, 1 H), 7.46 (dd, 1 H), | 374 |
| | 7.24 (t, 1 H), 6.98 (dd, 1 H), 6.96 (s, 1 H), 4.55 (m, 2 H), 4.00 (m, 1 H), 3.57 (s, | |
| | 1 H), 2.27 (m, 2 H), 2.14 (m, 2 H), 1.72 (m, 2 H), 1.24 (d, 3 H). | |
| 94 | ¹H NMR (pyrimidine-H): δ = 8.00 (s, 1 H) | 419 |
| 95 | ¹H NMR δ = 9.13 (s, 1 H); 8.00 (s, 1 H), 7.90 (s, 1 H), 7.67 (dd, 1 H), | 405 |
| | 7.25 (t, 1 H), 7.01 (dd, 1 H), 6.73 (s. 1 H), 4.54 (m, 2 H), 3.96 (m, 1 H), | |
| | 2.88 (m, 1 H), 1.20 (m, 3 H), 0.75 (m, 2 H), 0.66 (m, 2 H). | |
| 96 | ¹H NMR (pyrimidine-H): δ = 7.90 (s, 1 H) | 358 |
| 97 | ¹H NMR (pyrimidine-H): δ = 7.91 (s, 1 H) | 372 |
| 98 | ¹H NMR (pyrimidine-H): δ = 7.99 (s, 1 H) | 417 |
| 99 | ¹H NMR δ = 9.04 (s, 1H), 7.98 (s, 2H), 7.62-7.57 (m, 1H), 7.23-7.17 (m, | 402.1 |
| | 2H), 6.71 (s, 1H), 3.89 (dd, 1H), 3.36 (dd, 1H), 2.98-2.89 (m, 1H), | |
| | 2.61 (dd, 1H), 2.12 (dd, 1H), 1.11 (d, 3H), 0.77-0.60 (m, 4H) | |
| 100 | ¹H NMR (pyrimidine-H): δ = 7.91 (s, 1 H) | 402 |
| 101 | ¹H NMR (pyrimidine-H): δ = 7.91 (s, 1 H) | 402 |
| 102 | ¹H NMR (pyrimidine-H): δ = 7.95 (s, 1 H) | 374 |
| 103 | ¹H NMR (pyrimidine-H): δ = 7.90 (s, 1 H) | 360 |
| 104 | ¹H NMR (pyrimidine-H): δ = 8.00 (s, 1 H) | 405 |
| 105 | ¹H NMR (pyrimidine-H): δ = 7.91 (s, 1 H) | 374 |
| 106 | ¹H NMR δ = 9.10 (s, 1 H), 8.02 (s, 1 H), 7.94-7.92 (m, 1 H), 7.64 (d 1 | 388 |
| | H), 7.22 (d, 1 H), 7.02-6.97 (m, 2 H), 5.42 (d, 1 H), 3.44 (q, 2 H), | |
| | 2.97-2.92 (m, 1 H), 2.36-2.19 (m, 2 H), 2.02-1.97 (m, 2 H), 1.05 (t, 3 H), | |
| | 0.74-0.72 (m, 2 H), 0.65-0.63 (m, 2 H). | |
| 107 | ¹H NMR (pyrimidine-H): δ = 8.00 (s, 1 H) | 419 |
| 108 | ¹H NMR (pyrimidine-H): δ = 7.9 | 360 |
| 109 | ¹H NMR (pyrimidine-H): δ = 7.95 (s, 1 H) | 402 |
| 110 | ¹H NMR (pyrimidine-H): δ = 7.94 (s, 1 H) | 390 |
| 111 | ¹H NMR (pyrimidine-H): δ = 7.94 (s, 1 H) | 430 |
| 112 | ¹H NMR (pyrimidine-H): δ = 8.00 (s, 1 H) | 412 |
| 113 | ¹H NMR (pyrimidine-H): δ = 7.91 (s, 1 H) | 372 |
| 114 | ¹H NMR (pyrimidine-H): δ = 7.91 (s, 1 H) | 360 |
| 115 | ¹H NMR (pyrimidine-H): δ = 7.87 (s, 1 H) | 402 |
| 116 | ¹H NMR (pyrimidine-H): δ = 7.92 (s, 1 H) | 374 |
| 117 | ¹H NMR (pyrimidine-H): δ = 7.93 (s, 1 H) | 420 |
| 118 | ¹H NMR (pyrimidine-H): δ = 7.93 (s, 1 H) | 392 |
| 119 | ¹H NMR δ = 9.81 (m, 1 H), 8.20-8.19 (d, 1 H), 7.96 (m, 1 H), | 426.0 |
| | 7.91-7.88 (m, 1 H), 7.52-7.49 (m, 1 H), 6.90 (m, 1 H), 2.88-2.86 (m, 4 H), | |
| | 0.73-0.79 (m, 2 H), 0.66-0.64 (m, 2 H), | |
| 120 | ¹H NMR (pyrimidine-H): δ = 7.97 (s, 1H) | 388.1 |
| 121 | ¹H NMR (pyrimidine-H): δ = 7.95 (s, 1H) | 400.1 |
| 122 | ¹H NMR (pyrimidine-H): δ = 7.94 (s, 1H) | 414.1 |
| 123 | ¹H NMR (pyrimidine-H): δ = 7.90 (s, 1 H) | 358 |
| 124 | ¹H NMR (pyrimidine-H): δ = 7.90 (s, 1 H) | 358 |
| 125 | ¹H NMR (pyrimidine-H): δ = 7.90 (s, 1 H) | 358 |
| 126 | ¹H NMR (pyrimidine-H): δ = 7.90 (s, 1 H) | 358 |
| 127 | ¹H NMR (pyrimidine-H): δ = 7.97 (s, 1H) | 372.1 |
| 128 | ¹H NMR (pyrimidine-H): δ = 7.99 (s, 1 H) | 447 |
| 129 | ¹H NMR (pyrimidine-H): δ = 7.99 (s, 1 H) | 435 |
| 130 | ¹H NMR (pyrimidine-H): δ = 8.07 (s, 1 H) | 457 |
| 131 | ¹H NMR (pyrimidine-H): δ = 8.00 (s, 1 H) | 419 |
| 132 | ¹H NMR (pyrimidine-H): δ = 8.01 (s, 1 H) | 445 |
| 133 | ¹H NMR (pyrimidine-H): δ = 8.00 (s, 1 H) | 417 |
| 134 | ¹H NMR (pyrimidine-H): δ = 7.97 (s, 1 H) | 405 |
| 135 | ¹H NMR (pyrimidine-H): δ = 8.07 (s, 1 H) | 427 |
| 136 | ¹H NMR (pyrimidine-H): δ = 7.91 | 374 |
| 137 | ¹H NMR (pyrimidine-H): δ = 7.90 (s, 1 H) | 402.2 |
| 138 | | 348.0 |
| 139 | | 361.1 |
| 140 | | 387.1 |
| 141 | | 373.2 |
| 142 | | 362.0 |
| 143 | | 362.0 |
| 144 | | 359.2 |
| 145 | | 373.1 |
| 146 | | 391.0 |
| 147 | | 405.1 |
| 148 | | 431.1 |
| 149 | | 417.1 |
| 150 | | 405.1 |
| 151 | | 405.1 |
| 152 | | 403.1 |
| 153 | | 417.1 |
| 154 | | 369.1 |
| 155 | | 383.1 |
| 156 | | 409.1 |
| 157 | | 395.1 |
| 158 | | 383.1 |
| 159 | | 383.1 |
| 160 | | 381.1 |
| 161 | | 395.1 |
| 162 | | 413.0 |
| 163 | | 427.0 |
| 164 | | 453.1 |
| 165 | | 427.0 |
| 166 | | 427.0 |
| 167 | | 425.1 |
| 168 | | 439.0 |
| 169 | | 401.1 |
| 170 | | 427.1 |
| 171 | | 413.1 |
| 172 | | 401.1 |
| 173 | | 401.1 |
| 174 | | 399.1 |
| 175 | | 387.1 |
| 176 | | 373.1 |
| 177 | | 399.1 |
| 178 | | 385.2 |
| 179 | | 373.1 |
| 180 | | 371.2 |
| 181 | | 359.1 |
| 182 | | 385.1 |
| 183 | ¹H NMR (pyrimidine-H): δ = 8.02 (s, 1H) | 434.1 |
| 184 | ¹H NMR δ = 8.20 (s, 1 H), 7.59 (dd, 1 H), 7.47 (s, 1 H), 7.35 (dd 1 H), | 400 |
| | 6.93 (dd, 1 H), 3.79 (t, 2 H), 3.03 (s, 3 H), 2.29 (s, 3 H), 2.07-1.97 (m, 2 | |
| | H), 0.79-0.74 (m, 2 H), 0.64-0.62 (m, 2 H). | |
| 185 | ¹H NMR (pyrimidine-H): δ = 7.9 | 360 |
| 186 | ¹H NMR (pyrimidine-H): δ = 8.01 | 372 |
| 187 | ¹H NMR δ = 9.04 (s, 1H), 8.05 (s, 1H), 7.99 (s, 1H), 7.64-7.56 (m, 1H), | 403 |
| | 7.25-7.15 (m, 2H), 6.71 (s, 1H), 3.80-3.62 (m, 2H), 2.99-2.90 (m, 1H), | |
| | 2.63-2.50 (m, 1H), 2.36-2.50 (m, 1H), 1.72-1.60 (m, 1H), 1.14 (d, 3H), | |
| | 0.78-0.58 (m, 4H) | |
| 188 | ¹H NMR (pyrimidine-H): δ = 7.92 (s, 1H) | 386.1 |
| 190 | ¹H NMR δ = 9.08 (s, 1 H), 7.98 (dd, 1 H), 7.89 (s, 1 H), 7.62 (dd 1 H), | 416 |
| | 7.19 (dd, 1 H), 6.97-6.94 (m, 2 H), 4.79-4.77 (m, 1 H), 4.14-4.08 (m, | |
| | 2 H), 2.97-2.92 (m, 1 H), 2.54-2.42 (m, 2 H), 2.07-2.02 (m, 2 H), | |
| | 1.13 (t, 3 H), 0.76-0.74 (m, 2 H), 0.65-0.64 (m, 2 H). | |
| 191 | | 460 |
| 192 | ¹H NMR (pyrimidine-H): δ = 8.18 (s, 1H) | 434.2 |
| 193 | ¹H NMR (pyrimidine-H): δ = 8.16 | 450 |
| 194 | ¹H NMR (pyrimidine-H): δ = 8.17 (s, 1H) | 394.2 |
| 195 | ¹H NMR (pyrimidine-H): δ = 7.91 | 358 |
| 196 | ¹H NMR (pyrimidine-H): δ = 8.17 (s, 1H) | 434.2 |
| 197 | ¹H NMR (pyrimidine-H): δ = 7.89 | 364 |
| 198 | ¹H NMR (pyrimidine-H): δ = 8.01 | 376 |
| 199 | ¹H NMR (pyrimidine-H): δ = 7.88 | 336 |
| 200 | ¹H NMR: δ = 8.99 (s, 1 H), 7.96 (s, 1 H), 7.62-7.66 (m, 1 H), | 358 |
| | 7.19-7.21 (m, 2 H), 6.90 (s, 1 H), 3.72-3.83 (m, 2 H), 2.61-2.66 (m, 1 H), | |
| | 2.41-2.45 (m, 2 H), 2.01-2.09 (m, 2 H), 1.07 (d, 3 H), 0.91-0.99 (m, 2 H), | |
| | 0.78-0.82 (m, 1 H), 0.50-0.55 (m, 1 H) | |
| 201 | ¹H NMR (pyrimidine-H): δ = 8.16 (s, 1 H) | 410.2 |
| 202 | ¹H NMR (pyrimidine-H): δ = 7.92 (s, 1H) | 390.1 |
| 203 | ¹H NMR (pyrimidine-H): δ = 7.92 (s, 1H) | 372.1 |
| 204 | ¹H NMR (pyrimidine-H): δ = 7.89 | 332 |
| 205 | ¹H NMR (pyrimidine-H): δ = 9.03 (s, 1 H), 7.87 (m, 2 H), 7.67 (d, 1 H), | 360 |
| | 7.19 (m, 1 H), 7.10 (m, 1 H), 6.90 (s, 1 H), 4.38-4.47 (m, 2 H), | |
| | 3.98-4.07 (m, 2 H), 2.62-2.66 (m, 1 H), 1.07 (d, 3 H), 0.93-0.95 (m 1 H), | |
| | 0.79-0.83 (m, 1 H), 0.50-0.54 (m, 1 H | |
| 206 | ¹H NMR (pyrimidine-H): δ = 7.90 (major) (2 diastereoisomers) | 386.1 |
| 207 | ¹H NMR (pyrimidine-H): δ = 7.96 | 346 |
| 209 | ¹H NMR (pyrimidine-H): δ = 7.99 (s, 1H) (2 diastereoisomers) | 444.1 |
| 210 | ¹H NMR (pyrimidine-H): δ = 7.99 (s, 1H) (2 diastereoisomers) | 432.1 |
| 211 | ¹H NMR (pyrimidine-H): δ = 7.99 (s, 1H) (2 diastereoisomers) | 444.1 |
| 212 | ¹H NMR (pyrimidine-H): δ = 7.91 (s, 1H) (2 diastereoisomers) | 400.1 |
| 213 | ¹H NMR (pyrimidine-H): δ = 7.89 (s, 1 H) | 372 |
| 214 | ¹H NMR δ = 9.62 (s, 1 H), 8.18 (s, 1 H), 7.73 (dd, 1 H), 7.33 (dd 1 H), | 394 |
| | 7.09 (dd, 1 H), 6.87 (s, 1 H), 4.07 (t, 2 H), 3.03 (t, 2 H), 2.93 (m, 1 H), | |
| | 2.12 (m, 2 H), 0.76-0.64 (m, 4 H). | |
| 215 | ¹H NMR (pyrimidine-H): δ = 7.90 (s, 1 H) | 374 |
| 216 | ¹H NMR (pyrimidine-H): δ = 7.88 (s, 1 H) | 402 |
| 217 | ¹H NMR δ = 9.81 (s, 1 H), 8.02 (s, 1 H), 7.72 (d, 1 H), 7.62-7.59 (m, 2 | 372 |
| | H), 7.43 (dd 1 H), 6.98 (d, 1 H), 3.81 (t, 2 H), 3.04-3.00 (m, 1 H), | |
| | 2.09-2.02 (m, 2 H), 0.75-0.62 (m, 4 H). | |
| 218 | ¹H NMR (pyrimidine-H): δ = 8.29 | 470 |
| 219 | ¹H NMR (pyrimidine-H): δ = 7.98 (s, 1 H) | 392 |
| 220 | ¹H NMR δ = 9.58 (s, 1 H), 8.29 (s, 1 H), 8.06 (s, 1 H), 7.38-7.32 (m 1 | 434 |
| | H), 7.27-7.21 (m, 2 H), 6.76 (d, 1H), 5.43-5.35 (m, 1 H), | |
| | 3.82-3.79 (m, 2 H), 2.51-2.43 (m, 2 H), 2.10-2.03 (m, 2 H), 1.42 (d, 3H). | |
| 221 | | 413 |
| 222 | ¹H NMR (pyrimidine-H): δ = 7.93 | 358 |
| 223 | ¹H NMR (pyrimidine-H): δ = 7.98 (s, 1 H) | 417/419 |
| 224 | ¹H NMR (pyrimidine-H): δ = 7.91 | 384 |
| 225 | ¹H NMR (pyrimidine-H): δ = 8.06 | 372 |
| 226 | ¹H NMR δ = 9.22 (s, 1 H), 8.07 (s, 1 H), 7.98 (m, 1 H), 7.38 (m, 1 H), | 374 |
| | 7.22 (m, 2 H), 3.80 (t, 2 H), 3.71 (m, 4 H), 3.64 (m, 4 H), 2.05 (m, 2 H). | |
| 227 | ¹H NMR (pyrimidine-H): δ = 7.97 (s, 1 H) | 405/407 |
| 228 | ¹H NMR (pyrimidine-H): δ = 8.07 | 390 |
| 229 | ¹H NMR (pyrimidine-H): δ = 7.97 (s, 1 H) | 403/405 |
| 230 | ¹H NMR (pyrimidine-H): δ = 7.88 (s, 1H) | 360.1 |
| 231 | ¹H NMR (pyrimidine-H): δ = 7.99 | 416 |
| 232 | ¹H NMR (pyrimidine-H): δ = 7.99 | 428 |
| 233 | ¹H NMR (pyrimidine-H): δ = 8 | 386 |
| 234 | ¹H NMR (pyrimidine-H): δ = 8 | 372 |
| 235 | ¹H NMR (pyrimidine-H): δ = 7.9 | 372 |
| 236 | ¹H NMR (pyrimidine-H): δ = 7.92 (s, 1H) | 372.1 |
| 237 | ¹H NMR (pyrimidine-H): δ = 7.9 | 372 |
| 238 | ¹H NMR (pyrimidine-H): δ = 8.11 | 416 |
| 239 | ¹H NMR (pyrimidine-H): δ = 7.91 (s, 1 H) | 419/421 |
| 240 | ¹H NMR δ = 9.44 (s, 1H), 8.16 (s, 1H), 8.03 (s, 1H), 7.69-7.62 (m, 1H), | 392.2 |
| | 7.28-7.20 (m, 2H), 6.79 (s, 1H), 3.90 (dd, 1H), 3.37 (dd, 1H), | |
| | 3.01-2.95 (m, 1H), 2.60 (dd, 1H), 2.12 (dd, 1H), 1.11 (d, 3H), 0.77-0.60 (m, 4H) | |
| 241 | ¹H NMR δ = 9.05-8.95 (m, 1H), 8.07-7.85 (m, 2H), 7.66-7.57 (m, 1H), | 372.2 |
| | 7.24-7.16 (m, 2H), 6.95-6.85 (m, 1H), 3.78-3.47 (m, 2H), 2.70-2.45 (m, | |
| | 2H), 2.37-2.27 (m, 1H), 1.74-1.60 (m, 1H), 1.20-0.75 (m, 8H), | |
| | 0.55-0.45 (m, 1H) (4 diastereoisomers) | |
| 242 | ¹H NMR (pyrimidine-H): δ = 7.88 (s, 1 H) | 372.2 |
| 243 | ¹H NMR δ = 9.49 (s, 1H), 8.17 (s, 1H), 7.95 (s, 1H), 7.71-7.66 (m, 1H), | 392.2 |
| | 7.27 (t, 1H), 7.01-6.96 (m, 1H), 6.82 (s, 1H), 4.30-4.19 (m, 1H), | |
| | 2.97-2.89 (m, 1H), 2.51-2.25 (m, 3H), 1.70-1.60 (m, 1H), 1.12 (d, 3H), | |
| | 0.80-0.58 (m, 4H) | |
| 244 | ¹H NMR (pyrimidine-H): δ = 8.18 (s, 1H) | 406.2 |
| 245 | ¹H NMR δ = 9.51 (s, 1H minor), 9.48 (s, 1H major), 8.17 (s, 1H), | 406.2 |
| | 8.05-8.00 (m, 1H), 7.69-7.65 (m, 1H major), 7.63-7.58 (m, 1H minor), | |
| | 7.30-7.23 (m, 1H), 7.11-7.06 (m, 1H major), 6.90-6.67 (m, 1H minor + 1H), | |
| | 4.28-4.07 (1 H), 2.99-2.89 (m, 1H), 2-70-2.50 (m, 1H), 2.01-1.85 (m, 1H), | |
| | 1.28-1.08 (m, 7H), 0.78-0.58 (m, 4H) (2 diastereoisomers) | |
| 246 | ¹H NMR (pyrimidine-H): δ = 7.93 (s, 1H minor), 7.90 (s, 1 H major) (2 | 400.2 |
| | diastereoisomers) | |
| 247 | ¹H NMR δ = 9.58 (s, 1H), 8.18 (s, 1H), 7.83 (s, 1H), 7.62-7.57 (m, 1H), | 420.2 |
| | 7.37 (t, 1H), 6.90-6.86 (m, 1H), 6.70 (d, 1H), 4.66-4.55 (m, 1H), | |
| | 2.30-2.05 (m, 4H), 1.76-1.59 (m, 2H), 1.32 (d, 3H) | |
| 248 | ¹H NMR (pyrimidine-H): δ = 8.00 (s, 1H) | 448.1 |
| 249 | ¹H NMR (pyrimidine-H): δ = 8.17 (s, 1H) | 422.2 |
| 250 | ¹H NMR (pyrimidine-H): δ = 8.00 (s, 1H) | 433.1 |
| 251 | ¹H NMR (pyrimidine-H): δ = 8.16 (s, 1H) | 406.2 |
| 252 | ¹H NMR (pyrimidine-H): δ = 8.15 | 392 |
| 253 | ¹H NMR (pyrimidine-H): δ = 7.90 (s, 1 H) | 386 |
| 254 | ¹H NMR (pyrimidine-H): δ = 7.93 (s, 1 H) | 358 |
| 255 | ¹H NMR (pyrimidine-H): δ = 8.17 (s, 1H) | 406.2 |
| 256 | ¹H NMR (pyrimidine-H): δ = 7.88 (s, 1 H) | 374 |
| 257 | ¹H NMR (pyrimidine-H): δ = 7.99 (s, 1 H) | 459/461 |
| 258 | ¹H NMR (pyrimidine-H): δ = 8.00 (s, 1H) | 470.1 |
| 259 | ¹H NMR (pyrimidine-H): δ = 7.96 (s, 1 H) | 414 |
| 260 | ¹H NMR δ = 9.13 (s, 1H), 8.05 (t, 1H), 7.91 (s, 1H), 7.64-7.58 (m, 1H), | 412.2 |
| | 7.25 (t, 1H), 7.00 (s, 1H), 6.95-6.89 (m, 1H), 5.04-4.95 (m, 1H), | |
| | 2.93-2.85 (m, 1H), 2.20-2.05 (m, 2H), 0.80-0.57 (m, 4H) | |
| 261 | ¹H NMR (pyrimidine-H): δ = 8.18 (s, 1H) | 394.1 |
| 262 | ¹H NMR (pyrimidine-H): δ = 8.00 (s, 1H) | 407.1 |
| 263 | ¹H NMR (pyrimidine-H): δ = 8.17 (s, 1H) | 436.2 |
| 264 | ¹H NMR (pyrimidine-H): δ = 7.91 (s, 1H) | 360.1 |
| 265 | ¹H NMR δ = 9.50 (s, 1 H), 8.36 (s, 1 H), 7.97 (m, 1 H), 7.55 (d 1 H), | 392 |
| | 7.26 (m, 2 H), 3.80 (t, 2 H), 2.99 (m, 1 H), 2.08 (m, 2 H), 0.80 (m, 2 H), | |
| | 0.61 (m, 2 H). | |
| 266 | ¹H NMR (pyrimidine-H): δ = 7.91 (s, 1H) | 362.2 |
| 267 | ¹H NMR (pyrimidine-H): δ = 7.98 (s, 1 H) | 417/419 |
| 268 | | 350 |
| 269 | ¹H NMR (pyrimidine-H): δ = 8.14 | 380 |
| 270 | ¹H NMR (pyrimidine-H): δ = 8.14 | 366 |
| 271 | ¹H NMR (pyrimidine-H): δ = 8.00 (s, 1H) | 404 |
| 272 | ¹H NMR (pyrimidine-H): δ = 8.17 (s, 1H) | 394.2 |
| 273 | ¹H NMR (pyrimidine-H): δ = 7.90 (s, 1 H) | 388.1 |
| 274 | ¹H NMR (pyrimidine-H): δ = 7.90 (s, 1H) (2 diastereoisomers) | 434.2 |
| 275 | ¹H NMR (pyrimidine-H): δ = 8.17 (s, 1H) (2 diastereoisomers) | 406.2 |
| 276 | ¹H NMR (pyrimidine-H): δ = 8.17 (s, 1H) | 420.1 |
| 277 | ¹H NMR (pyrimidine-H): δ = 8.17 (s, 1H) (2 diastereoisomers) | 400.2 |
| 278 | ¹H NMR (pyrimidine-H) δ = 9.51 (s, 1 H), 8.17 (m, 2 H), 7.71 (dd, 1 H), | 408 |
| | 7.33 (m, 1 H), 7.10 (dd, 1 H), 6.80 (s, 1 H), 4.06-4.10 (m, 2 H), | |
| | 3.02-3.06 (m, 2 H), 2.62-2.66 (m, 1 H), 2.09-2.16 (m, 2 H), 1.05 (d, 3 H), | |
| | 1.01-1.03 (m, 1 H), 0.79-0.81 (m, 1 H), 0.50-0.54 (m, 1 H) | |
| 279 | ¹H NMR (pyrimidine-H): δ = 7.97 (s, 1 H) | 419/421 |
| 280 | ¹H NMR (pyrimidine-H): δ = 8.16 (s, 1 H) | 406 |
| 281 | ¹H NMR (pyrimidine-H): δ = 8.03 (s, 1 H) | 445/447 |
| 282 | ¹H NMR (pyrimidine-H): δ = 7.91 (s, 1H) (2 diastereoisomers) | 372.2 |
| 283 | ¹H NMR (pyrimidine-H): δ = 7.93 (s, 1 H) | 358 |
| 284 | ¹H NMR (pyrimidine-H): δ = 8.17 (s, 1H) | 446.1 |
| 285 | ¹H NMR (pyrimidine-H): δ = 7.98 (s, 1 H) | 451.0 |
| 286 | ¹H NMR (pyrimidine-H): δ = 7.90 (s, 1 H) | 407.1 |
| 287 | ¹H NMR (pyrimidine-H): δ = 8.00 (s, 1H) | |
| 288 | | 431/433 |
| 289 | ¹H NMR (pyrimidine-H): δ = 8.17 (s, 1H) (2 diastereoisomers) | |
| 290 | | 432/434 |
| 291 | | 445/447 |
| 292 | ¹H NMR (pyrimidine-H): δ = 8.17 (s, 1H) | |
| 293 | ¹H NMR δ = 9.36 (s, 1 H), 8.15 (s, 1 H), 7.89-7.88 (s, 1 H), 7.60-7.57 (m, | 393.1 |
| | 1 H), 7.21-7.18 (m, 2 H), 6.74 (s, 1 H), 3.73-3.71 (m, 2 H), 3.43-3.41 (m, 2 | |
| | H), 3.02-3.00 (m, 1 H), 2.75 (s, 3 H), 0.75-0.72 (m, 2 H), 0.66-0.64 (m, 2 | |
| | H), | |
| 294 | ¹H NMR δ = 9.09 (s, 1H), 7.91 (s, 2H), 7.67-7.61 (m, 1H), 7.22 (t, 1H), | 372.2 |
| | 7.00 (s, 1H), 6.98-6.91 (m, 1H), 4.18-4.06 (m, 1H), 2.95-2.85 (m, 1H), | |
| | 2.50-2.30 (m, 2H), 2.28-2.17 (m, 1H), 1.80-1.69 (m, 1H), 1.65-1.53 (m, | |
| | 1H), 1.43-1.28 (m, 1H), 0.85-0.55 (m, 7H) | |
| 295 | ¹H NMR (pyrimidine-H): δ = 8.00 (s, 1 H) | 445/447 |
| 296 | ¹H NMR (pyrimidine-H): δ = 8.1 (s, 1H) | 451.1 |
| 297 | | 380 |
| 298 | | 398 |
| 299 | ¹H NMR (pyrimidine-H): δ = 7.89 (s, 1 H) | 372 |
| 300 | ¹H NMR (pyrimidine-H): δ = 7.88 (s, 1 H) | 360 |
| 301 | ¹H NMR δ = 9.37 (s, 1 H), 8.23 (s, 1 H), 7.98 (m, 1 H), 7.48 (d 1 H), | 392 |
| | 7.28 (m, 2 H), 3.81 (t, 2 H), 3.61 (m, 4 H), 2.05 (m, 2 H), 1.92 (m, 4H). | |
| 302 | | 384 |
| 303 | | 406 |
| 304 | ¹H NMR (pyrimidine-H): δ = 7.99 (s, 1 H) | 372 |
| 305 | ¹H NMR (pyrimidine-H): δ = 7.89 | 358 |
| 306 | | 420 |
| 307 | | 346 |
| 308 | ¹H NMR (pyrimidine-H): δ = 7.97 (s, 1 H) | 405/407 |
| 309 | ¹H NMR (pyrimidine-H): δ = 8.34 | 436 |
| 310 | ¹H NMR (pyrimidine-H): δ = 8.16 | 392 |
| 311 | | 402 |
| 312 | ¹H NMR (pyrimidine-H): δ = 7.91 (s, 1H) | |
| 313 | ¹H NMR (pyrimidine-H): δ = 7.89 (s, 1H) | 387.2 |
| 314 | ¹H NMR (pyrimidine-H): δ = 8.18 (s, 1H) | |
| 315 | ¹H NMR (pyrimidine-H): δ = 7.96 (s, 1 H) | 358 |
| 316 | ¹H NMR (pyrimidine-H): δ = 8.16 | 392 |
| 317 | | 431/433 |
| 318 | ¹H NMR (pyrimidine-H): δ = 7.91 | 374 |
| 319 | | 420 |
| 320 | | 406 |
| 321 | | 406 |
| 322 | | 394 |
| 323 | | 352 |
| 324 | ¹H NMR (pyrimidine-H): δ = 8.14 | 408 |
| 325 | ¹H NMR (pyrimidine-H): δ = 7.86 | 372 |
| 326 | ¹H NMR (pyrimidine-H): δ = 8.17 | 408 |
| 327 | ¹H NMR (pyrimidine-H): δ = 8.11 | 402 |
| 328 | ¹H NMR (pyrimidine-H): δ = 7.87 | 374 |
| 329 | ¹H NMR δ = 8.11 (s, 1 H), 7.64 (dd, 1 H), 7.40 (dd, 1 H), 7.36 (br. s, 1 H), | 430 |
| | 7.31 (dd, 1 H), 6.94 (dd, 1 H), 4.92 (h, 1 H), 3.79 (t, 2 H), 2.81-2.77 (m, | |
| | 1 H), 2.53-2.44 (m, 2 H), 2.09-2.01 (m, 2 H), 1.19 (d, 6 H), | |
| | 0.67-0.63 (m, 4 H). | |
| 330 | ¹H NMR δ = 8.11 (s, 1 H), 7.65 (dd, 1 H), 7.41 (dd, 1 H), 7.39 (br. s, 1 H), | 416 |
| | 7.32 (dd, 1 H), 6.96 (dd, 1 H), 4.16 (q, 2 H), 3.79 (t, 2 H), 2.78-2.73 (m, | |
| | 1 H), 2.52-2.44 (m, 2 H), 2.07-2.03 (m, 2 H), 1.18 (t, 3 H), | |
| | 0.65-0.62 (m, 4 H). | |
| 332 | ¹H NMR (pyrimidine-H): δ = 7.96 (s, 1 H) | 358 |
| 333 | ¹H NMR (pyrimidine-H): δ = 7.91 (s, 1 H) | 400 |

The chemical NMR shifts in ppm were measured at 400 MHz, unless indicated otherwise in the solvent DMSO-d₆ using tetramethylsilane as internal standard.

The abbreviations below describe the signal splitting:

s=singlet, d=doublet, t=triplet, q=quadruplet, m=multiplet

### Use Examples

### Example A

### Venturia Test (Apple)/Protective

Solvents: 24.5 parts by weight of acetone
24.5 parts by weight of dimethylacetamide

Emulsifier: 1 part by weight of alkylaryl polyglycol ether

To produce a suitable preparation of active compound, 1 part by weight of active compound is mixed with the stated amounts of solvents and emulsifier, and the concentrate is diluted with water to the desired concentration.

To test for protective activity, young plants are sprayed with the preparation of active compound at the stated application rate. After the spray coating has dried on, the plants are inoculated with an aqueous conidia suspension of the apple pathogen Venturia inaequalis and then remain in an incubation cabinet at about 20° C. and 100% relative atmospheric humidity for 1 day. The plants are then placed in a greenhouse at about 21° C. and a relative atmospheric humidity of about 90%.

Evaluation is carried out 10 days after the inoculation. 0% means an efficacy which corresponds to that of the control, whereas an efficacy of 100% means that no infection is observed.

In this test, the examples Nos. 4, 5, 6, 8, 19, 20, 22, 25, 26, 27, 28, 29, 30, 31, 32, 33, 34, 35, 36, 37, 39, 44, 45, 46, 47, 48, 54, 56, 57, 62, 63, 64, 66, 67, 68, 70, 71, 72, 73, 74, 75, 76, 78, 80, 81, 84, 85, 92, 93, 94, 95, 99, 100, 102, 104, 108, 112, 113, 115, 119, 121, 122, 129, 130, 132, 133, 139, 140, 141, 145, 147, 148, 149, 153, 176, 177, 178, 179, 180, 181, 182, 186, 187, 188, 190, 192, 196, 197, 198, 200, 202, 204, 206, 208, 209, 210, 211, 215, 216, 224, 233, 234, 236, 253, 254, 257, 259, 260, 269, 281, 289, 293, 294, 296, 304, 317 and 331 from Table I show, at an active compound concentration of 100 ppm, an efficacy of 70% or more.

### Example B

### Botrytis Test (Bean)/Protective

Solvents: 24.5 parts by weight of acetone
24.5 parts by weight of dimethylacetamide

Emulsifier: 1 part by weight of alkylaryl polyglycol ether

To produce a suitable preparation of active compound, 1 part by weight of active compound is mixed with the stated amounts of solvents and emulsifier, and the concentrate is diluted with water to the desired concentration.

To test for protective activity, young plants are sprayed with the preparation of active compound at the stated application rate. After the spray coating has dried on, two small pieces of agar colonized by Botrytis cinerea are placed onto each leaf. The inoculated plants are placed in a dark chamber at about 20° C. and 100% relative atmospheric humidity.

The size of the infected areas on the leaves is evaluated 2 days after the inoculation. 0% means an efficacy which corresponds to that of the control, whereas an efficacy of 100% means that no infection is observed.

In this test, the examples Nos. 26, 39, 46, 48, 52, 63, 64, 67, 70, 72, 73, 76, 80, 84, 85, 88, 92, 94, 95, 99, 100, 102, 104, 113, 121, 129, 130, 132, 139, 140, 145, 147, 148, 149, 153, 170, 176, 177, 178, 179, 180, 182, 186, 187, 188, 192, 200, 202, 204, 206, 208, 209, 210, 211, 215, 216, 224, 254, 257, 259, 260, 269, 279, 289, 293, 294, 296, 304, 317 and 331 from Table I show, at an active compound concentration of 250 ppm, an efficacy of 70% or more.

### Example C

### Alternaria Test (Tomato)/Protective

Solvent: 49 parts by weight of N,N-dimethylformamide

Emulsifier: 1 party by weight of alkylaryl polyglycol ether

To produce a suitable preparation of active compound, 1 by weight of active compound is mixed with the stated amounts of solvent and emulsifier, and the concentrate is diluted with water to the desired concentration.

To test for protective activity, young tomato plants are sprayed with the preparation of active compound at the stated application rate. One day after the treatment, the plants are inoculated with a spore suspension of Alternaria solani and then remain at 100% rel. humidity and 22° C. for 24 h. The plants remain at 96% rel. atmospheric humidity and a temperature of 20° C.

Evaluation is carried out 7 days after the inoculation. 0% means an efficacy which corresponds to that of the control, whereas an efficacy of 100% means that no infection is observed.

In this test, the examples Nos. 5, 6, 11, 19, 20, 22, 27, 29, 31, 33, 34, 35, 36, 37, 39, 45, 46, 47, 48, 57, 64, 66, 67, 68, 70, 72, 74, 75, 76, 78, 80, 81, 82, 84, 85, 88, 92, 93, 94, 96, 99, 100, 103, 104, 106, 107, 111, 112, 115, 119, 120, 121, 122, 127, 128, 129, 130, 131, 132, 133, 134, 135, 137, 138, 140, 141, 142, 143, 144, 145, 146, 147, 148, 150, 152, 153, 154, 155, 156, 157, 159, 160, 161, 163, 164, 167, 168, 169, 170, 171, 173, 174, 177, 178, 180, 182, 184, 185, 186, 187, 188, 190, 192, 196, 197, 198, 200, 204, 205, 206, 208, 209, 210, 211, 212, 213, 215, 216, 224, 232, 233, 234, 235, 238, 240, 245, 250, 253, 254, 257, 258, 259, 260, 262, 263, 264, 265, 273, 274, 275, 279, 280, 281, 282, 283, 284, 285, 286, 289, 291, 293, 294, 295, 314, 317, 318, 322 and 331 from Table I show, at an active compound concentration of 500 ppm, an efficacy of 70% or more.

### Example D

### Sphaerotheca Test (Cucumber)/Protective

Solvent: 49 parts by weight of N,N-dimethylformamide

Emulsifier: 1 part by weight of alkylaryl polyglycol ether

To produce a suitable preparation of active compound, 1 part by weight of active compound is mixed with the stated amounts of solvent and emulsifier, and the concentrate is diluted with water to the desired concentration.

To test for protective activity, young cucumber plants are sprayed with the preparation of active compound at the stated application rate. One day after the treatment, the plants are inoculated with the spore suspension of Sphaerotheca fuliginea. The plants are then placed in a greenhouse at 70% relative atmospheric humidity and a temperature of 23° C.

Evaluation is carried out 7 days after the inoculation. 0% means an efficacy which corresponds to that of the control, whereas an efficacy of 100% means that no infection is observed.

In this test, the examples Nos. 1, 5, 6, 8, 17, 18, 19, 20, 22, 25, 26, 27, 28, 29, 30, 31, 32, 33, 34, 35, 36, 37, 39, 43, 44, 45, 46, 47, 48, 49, 51, 53, 55, 56, 57, 59, 60, 63, 65, 70, 71, 72, 73, 74, 75, 76, 77, 80, 82, 83, 84, 85, 86, 92, 93, 94, 95, 99, 100, 101, 102, 103, 104, 105, 106, 107, 108, 111, 112, 113, 115, 116, 117, 119, 120, 121, 128, 129, 130, 131, 132, 133, 134, 136, 138, 139, 140, 141, 144, 145, 148, 149, 151, 152, 153, 155, 157, 161, 167, 174, 176, 177, 178, 179, 180, 181, 182, 183, 185, 186, 187, 188, 190, 192, 193, 194, 195, 196, 197, 198, 199, 200, 201, 202, 203, 206, 207, 208, 210, 211, 215, 216, 217, 219, 223, 224, 229, 231, 232, 233, 234, 239, 240, 241, 242, 243, 245, 246, 247, 248, 249, 250, 251, 253, 254, 255, 257, 259, 260, 261, 263, 265, 266, 267, 268, 272, 273, 274, 275, 276, 277, 278, 280, 281, 282, 283, 285, 286, 289, 290, 291, 292, 294, 295, 298, 299, 302, 304, 305, 306, 309, 310, 311, 314, 315, 316, 317, 319, 324, 326, 328 and 331 from Table I show, at an active compound concentration of 500 ppm, an efficacy of 70% or more.

### Example E

Puccinia triticina Test (Wheat)/Protective

Solvent: 50 parts by weight of N,N-dimethylacetamide

Emulsifier: 1 part by weight of alkylaryl polyglycol ether

To produce a suitable preparation of active compound, 1 part by weight of active compound is mixed with the stated amounts of solvent and emulsifier, and the concentrate is diluted with water to the desired concentration.

To test for protective activity, young plants are sprayed with the preparation of active compound at the stated application rate. After the spray coating has dried on, the plants are sprayed with spores of a spore suspension of Puccinia triticina. The plants remain in an incubation cabin at 20° C. and 100% relative atmospheric humidity for 48 hours. The plants are placed in a greenhouse at a temperature of about 20° C. and a relative atmospheric humidity of about 80%.

Evaluation is carried out 8 days after the inoculation. 0% means an efficacy which corresponds to that of the control, whereas an efficacy of 100% means that no infection is observed.

In this test, the examples Nos. 5, 6, 8, 19, 20, 22, 25, 26, 27, 28, 29, 30, 31, 32, 33, 35, 36, 37, 39, 44, 45, 51, 54, 55, 56, 57, 60, 62, 63, 65, 66, 68, 70, 71, 73, 74, 76, 84, 92, 94, 95, 98, 99, 100, 104, 121, 123, 124, 125, 126, 132, 140, 141, 145, 148, 153, 177, 187, 188, 190, 192, 195, 196, 197, 198, 200, 203, 206, 208, 214, 215, 217, 219, 223, 239, 240, 246, 247, 250, 254, 260, 264, 267, 268, 269, 270, 278, 283, 285, 286, 292, 293, 294, 295, 298, 299, 302, 309, 310, 311, 315, 316, 317, 322 and 324 from Table I show, at an active compound concentration of 500 ppm, an efficacy of 70% or more.

### Example F

### Pyricularia Test (Rice)/Protective

Solvent: 28.5 parts by weight of acetone

Emulsifier: 1.5 parts by weight of alkylaryl polyglycol ether

To produce a suitable preparation of active compound, 1 part by weight of active compound is mixed with the stated amount of solvent, and a concentrate is diluted with water and the stated amount of emulsifier to the desired concentration.

To test for protective activity, young rice plants are sprayed with the preparation of active compound at the stated application rate. One day after the treatment, the plants are inoculated with an aqueous spore suspension of Pyricularia oryzae. The plants are then placed in a greenhouse at 100% relative atmospheric humidity and 25° C.

Evaluation is carried out 5 days after the inoculation. 0% means an efficacy which corresponds to that of the control, whereas an efficacy of 100% means that no infection is observed.

In this test, the compounds according to the invention Nos. 25, 26, 27, 28, 30, 36, 37, 39, 48, 54, 56, 57, 59, 60, 63, 65, 66, 68, 70, 71, 73, 75, 76, 80, 81, 84, 85, 92, 93, 94, 95, 96, 97, 98, 99, 103, 112, 113, 115, 121, 122, 139, 140, 141, 144, 145, 146, 147, 148, 149, 152, 153, 156, 157, 164, 176, 177, 178, 179, 182, 188, 192, 195, 196, 200, 206, 208, 209, 214, 215, 216, 219, 229, 234, 241, 243, 245, 247, 251, 252, 257, 265, 266, 268, 270, 278, 279, 292, 293, 296, 297, 303, 310 and 315 from Table I show, at an active compound concentration of 250 ppm, an efficacy of 80% or more.

### Example G

### Rhizoctonia Test (Rice)/Protective

Solvent: 28.5 parts by weight of acetone

Emulsifier: 1.5 parts by weight of alkylaryl polyglycol ether

To produce a suitable preparation of active compound, 1 part by weight of active compound is mixed with the stated amount of solvent, and a concentrate is diluted with water and the stated amount of emulsifier to the desired concentration.

To test for protective activity, young rice plants are sprayed with the preparation of active compound at the stated application rate. One day after the treatment, the plants are inoculated with hyphae of Rhizoctonia solani. The plants are then placed in a greenhouse at 100% relative atmospheric humidity and 25° C.

Evaluation is carried out 4 days after the inoculation. 0% means an efficacy which corresponds to that of the control, whereas an efficacy of 100% means that no infection is observed.

In this test, the compounds according to the invention Nos. 19, 25, 26, 27, 28, 29, 30, 33, 36, 37, 39, 48, 54, 56, 57, 60, 63, 65, 66, 68, 70, 71, 73, 75, 76, 80, 81, 84, 85, 92, 93, 94, 95, 96, 97, 98, 99, 112, 113, 115, 121, 122, 140, 141, 142, 144, 145, 146, 147, 148, 149, 152, 153, 155, 156, 157, 164, 176, 177, 178, 181, 182, 188, 190, 192, 195, 196, 198, 200, 206, 208, 209, 214, 215, 216, 219, 229, 234, 241, 243, 245, 247, 251, 252, 257, 265, 266, 268, 270, 278, 279, 286, 292, 293, 296, 297, 303 and 310 from Table I show, at an active compound concentration of 250 ppm, an efficacy of 80% or more.

### Example H

### Cochliobolus Test (Rice)/Protective

Solvent: 28.5 parts by weight of acetone

Emulsifier: 1.5 parts by weight of alkylaryl polyglycol ether

To produce a suitable preparation of active compound, 1 part by weight of active compound is mixed with the stated amount of solvent, and the concentrate is diluted with water and the stated amount of emulsifier to the desired concentration.

To test for protective activity, young rice plants are sprayed with the preparation of active compound at the stated application rate. One day after the treatment, the plants are inoculated with an aqueous spore suspension of Cochliobolus miyabeanus. The plants are then placed in a greenhouse at 100% relative atmospheric humidity and 25° C.

Evaluation is carried out 4 days after the inoculation. 0% means an efficacy which corresponds to that of the control, whereas an efficacy of 100% means that no infection is observed.

In this test, the compounds according to the invention Nos. 19, 25, 28, 29, 33, 36, 37, 39, 48, 60, 63, 65, 66, 70, 71, 73, 75, 76, 80, 81, 84, 85, 92, 93, 94, 95, 96, 98, 99, 103, 112, 113, 115, 121, 140, 145, 147, 148, 153, 156, 164, 176, 177, 178, 179, 181, 182, 188, 192, 193, 195, 196, 208, 209, 214, 216, 219, 229, 241, 243, 245, 247, 251, 252, 257, 265, 268, 270, 278, 279, 292, 293, 296, 297, 303 and 310 from Table I show, at an active compound concentration of 250 ppm, an efficacy of 80% or more.

### Example I

### Gibberella Test (Rice)/Protective

Solvent: 28.5 parts by weight of acetone

Emulsifier: 1.5 parts by weight of alkylaryl polyglycol ether

To produce a suitable preparation of active compound, 1 part by weight of active compound is mixed with the stated amount of solvent, and the concentrate is diluted with water and the stated amount of emulsifier to the desired concentration.

To test for protective activity, young rice plants are sprayed with the preparation of active compound at the stated application rate. One day after the treatment, the plants are inoculated with an aqueous spore suspension of Gibberella zeae. The plants are then placed in a greenhouse at 100% relative atmospheric humidity and 25° C.

Evaluation is carried out 5 days after the inoculation. 0% means an efficacy which corresponds to that of the control, whereas an efficacy of 100% means that no infection is observed.

In this test, the compounds according to the invention Nos. 19, 25, 27, 28, 33, 37, 65, 190, 195, 214 and 296 from Table I show, at an active compound concentration of 250 ppm, an efficacy of 80% or more.

### Example J

Phakopsora test (Soya Beans)/Protective

Solvent: 28.5 parts by weight of acetone

Emulsifier: 1.5 parts by weight of alkylaryl polyglycol ether

To produce a suitable preparation of active compound, 1 part by weight of active compound is mixed with the stated amount of solvent, and the concentrate is diluted with water and the stated amount of emulsifier to the desired concentration.

To test for protective activity, young plants are sprayed with the preparation of active compound at the stated application rate. One day after the treatment, the plants are inoculated with an aqueous spore suspension of Phakopsora pachyrhizi. The plants are then placed in a greenhouse at 80% relative atmospheric humidity and 20° C.

Evaluation is carried out 1 day after the inoculation. 0% means an efficacy which corresponds to that of the control, whereas an efficacy of 100% means that no infection is observed.

In this test, the compound No. 214 from Table I showed, at an active compound concentration of 250 ppm, an efficacy of 80% or more.

### Example K

Production of Fumonisin FB1 by Fusarium proliferatum

The method used was adapted to microtitre plates using the method described by Lopez-Errasquin et al.: Journal of Microbiological Methods 68 (2007) 312-317.

Fumonisin-inducing liquid medium (Jiménez et al., Int. J. Food Microbiol. (2003), 89, 185-193) was inoculated with a concentrated spore suspension of Fusarium proliferatum (350 000 spores/ml, stored at −160° C.) to a final concentration of 2000 spores/ml.

The compounds were dissolved (10 mM in 100% DMSO) and diluted to 100 μM in H₂O. The compounds were tested at 7 concentrations in a range of from 50 μM to 0.01 μM (diluted, starting with the 100 μM stock solution in 10% DMSO).

From each diluted solution, 5 μl were mixed with 95 μl of inoculated medium in a well of a 96-well microarray plate. The plate was covered and incubated at 20° C. for 6 days.

At the beginning and after 6 days, an OD measurement (OD620 multiple read per well (square: 3×3)) was carried out to calculate the “pI50” growth.

After 6 days, a sample of the liquid medium was taken and diluted in 10% acetonitrile. The concentration of FB1 in the diluted samples was analysed by HPLC-MS/MS, and the results were used to calculate the “pI50 FB1” values.

HPLC-MS/MS was carried out using the parameters below:

Mass spectrometry instrument: Applied Biosystems API4000 QTrap

### HPLC: Agilent 1100

### Autosampler: CTC HTS PAL

Chromatography column: Waters Atlantis T3 (50×2 mm)
Examples of the Measured pI50 Values

Production of Fumonisin FB1 by Fusarium proliferatum

| Ex. from | | pI50 |
|---|---|---|
| Table I | pI50 Fum | growth |
| 8 | 4.6 | 4 |
| 19 | 6.2 | 5.6 |
| 25 | 6.3 | 5.6 |
| 25 | 6.3 | 5.6 |
| 27 | 6.2 | 5.6 |
| 27 | 6.2 | 5.6 |
| 28 | 6.2 | 5.6 |
| 31 | 6.3 | 5.7 |
| 36 | 6.2 | 5.3 |
| 56 | 6.2 | 5.6 |
| 56 | 6.2 | 5.6 |
| 59 | 6.2 | 5.6 |
| 59 | 6.2 | 5.6 |
| 60 | 6.3 | 5.5 |
| 62 | 6.2 | 5.7 |
| 62 | 6.2 | 5.7 |
| 63 | 6.6 | 5.7 |
| 63 | 6.6 | 5.7 |
| 65 | 6.2 | 5.7 |
| 65 | 6.2 | 5.7 |
| 73 | 6.4 | 5.8 |
| 73 | 6.4 | 5.8 |
| 76 | 6.2 | 5.6 |
| 76 | 6.2 | 5.6 |
| 84 | 6.3 | 5.6 |
| 92 | 6.6 | 5.7 |
| 131 | 6 | 5.3 |
| 137 | 5.7 | 5.1 |
| 137 | 5.7 | 5.1 |
| 138 | 5 | 4.9 |
| 139 | 5 | 5 |
| 140 | 5.7 | 5.2 |
| 140 | 5.7 | 5.2 |
| 141 | 5.3 | 5 |
| 146 | 5.6 | 5 |
| 148 | 5.9 | 5.7 |
| 153 | 5.9 | 5.7 |
| 169 | 5.5 | 5.1 |
| 177 | 6.1 | 5.7 |
| 178 | 5.7 | 4.9 |
| 180 | 5.6 | 5.2 |
| 187 | 6.3 | 5.8 |
| 187 | 6.3 | 5.8 |
| 194 | 6.1 | 5.4 |
| 194 | 6.1 | 5.4 |
| 195 | 6.2 | 5.6 |
| 196 | 5.3 | 4.8 |
| 198 | 5.5 | 4.8 |
| 200 | 4.8 | 4.7 |
| 214 | 5.1 | 5 |
| 219 | 6.3 | 5.5 |
| 224 | 6 | 5.3 |
| 236 | 5.6 | 4.9 |

### Example L

Production of DON/acetyl-DON by Fusarium graminearum

The compounds were tested in microtitre plates at 7 concentrations of from 0.07 μM to 50 μM in a DON-inducing liquid medium (1 g of (NH₄)₂HPO₄, 0.2 g of MgSO₄×7 H₂O, 3 g of KH₂PO₄, 10 g of glycerol, 5 g of NaCl and 40 g of sucrose per litre) with oat extract (10%) and DMSO (0.5%). Inoculation was carried out using a concentrated spore suspension of Fusarium graminearum at a final concentration of 2000 spores/ml.

The plate was incubated at high atmospheric humidity at 28° C. for 7 days.

At the beginning and after 3 days, an OD measurement was carried out at OD520 (repeated measurements: 3×3 measurements per well) to calculate the growth inhibition.

After 7 days, 100 μl of an 84/16 acetonitrile/water mixture were added, and samples of the liquid medium were then taken from each well and diluted 1:100 in 10% acetonitrile. The proportions of DON and acetyl-DON in the samples were analysed by HPLC-MS/MS, and the measured values were used to calculate the inhibition of the DON/AcDON production compared to an active compound-free control.

The HPLC-MS/MS measurements were carried out using the parameters below:

Ionization type: ESI negative
Ion spray voltage: −4500 V
Spray gas temperature: 500° C.
Decluster potential: −40 V
Collision energy: −22 eV

### Collision gas: N₂

NMR trace: 355.0>264.9;
HPLC column: Waters Atlantis T3 (trifunctional C18 bondung, capped)
Particle size: 3 μm
Column dimensions: 50×2 mm

### Temperature: 40° C.

Solvent A: water/2.5 mM NH₄OAc+0.05% CH₃COOH (v/v)
Solvent B: methanol/2.5 mM NH₄OAc+0.05% CH₃COOH (v/v)
Flow rate: 400 μl/minute
Injection volume: 11 μl

### Gradient:

| Time [min] | A % | B % |
|---|---|---|
| 0 | 100 | 0 |
| 0.75 | 100 | 0 |
| 1.5 | 5 | 95 |
| 4 | 5 | 95 |
| 5 | 100 | 0 |
| 10 | 100 | 0 |

### Example of DON Inhibition

The examples Nos. 28, 31, 59, 60, 92, 138, 139, 140, 141, 148, 153, 169, 177, 178, 180, 236, 195, 198 and 200 showed an activity of >80% in the inhibition of DON/AcDON at 50 μM. The inhibition of growth of Fusarium graminearum by the examples having an activity >80% varied from 84 to 100% at 50 μM.

1. A compound of formula (I), 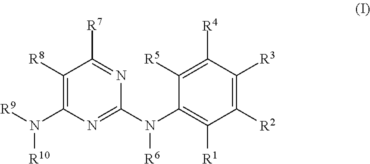
wherein:
R¹ to R⁵ independently of one another represent hydrogen, OH, halogen, cyano, C₁-C₄-alkyl, C₁-C₄-alkoxy, C₁-C₄-haloalkyl, NMe₂, SCH₃ or C₁-C₂-haloalkoxy,
where exactly one of the radicals R² and R³ represents a group of the formula E1, E2 or E3, 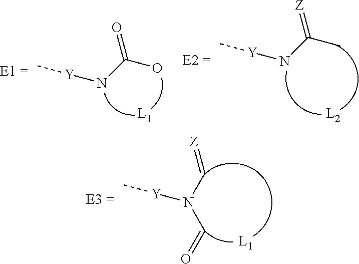
wherein:
Y represents a direct bond, C═O or a C₁-C₃-alkylene chain substituted by straight-chain or branched C₁-C₄-alkyl, C₁-C₄-haloalkyl or C₁-C₄-alkoxyalkyl,
Z represents sulphur or oxygen,
L₁ represents an unsubstituted or substituted C₁- to C₄-alkylene chain or a C₂- to C₄-alkenyl chain,
where double bonds are not cumulated and
where the individual carbon atoms may carry one or more substituents, independently of one another selected from the list below:
hydrogen, hydroxyl, oxo, straight-chain or branched C₁-C₄-alkyl, straight-chain or branched C₁-C₄-alkoxyalkyl, CH₂OH, straight-chain or branched C₁-C₄-alkoxycarbonyl, straight-chain or branched C₁-C₄-alkoxy-C₁-C₄-alkyl, straight-chain or branched phenyl or benzyl,
or
two substituents attached to two adjacent carbon atoms of the alkylene chain together with these two carbon atoms form
a 5- or 6-membered unsubstituted or substituted saturated carbocycle,
where the substituents independently of one another are selected from the list below:
hydrogen, fluorine, optionally branched C₁-C₄-alkyl, optionally branched C₁-C₄-alkoxy or optionally branched C₁-C₄-haloalkyl,
or
a 5- or 6-membered unsubstituted or substituted saturated heterocycle which contains an oxygen atom or a sulphur atom,
where the substituents independently of one another are selected from the list below:
hydrogen, fluorine, optionally branched C₁-C₄-alkyl, optionally branched C₁-C₄-alkoxy or optionally branched C₁-C₄-haloalkyl,
or
an unsubstituted or substituted phenyl ring,
where the substituents independently of one another are selected from the list below:
hydrogen, halogen, CN, SCH₃, NO₂, optionally branched C₁-C₄-alkyl, optionally branched C₁-C₄-alkoxy, optionally branched C₁-C₄-alkylcarbonyl, optionally branched C₁-C₄-haloalkyl, optionally branched C₁-C₄-haloalkoxy,
L₂ is an unsubstituted or substituted C₁- to C₄-alkylene chain, a C₂- to C₄-alkenyl chain or a 1,3-linked cyclopentyl (3-oxo-2-azabicyclo[2.2.1]hept-2-yl) or a C₁- to C₄-alkylene chain interrupted by a heteroatom selected from the group consisting of oxygen, sulphur and nitrogen,
where double bonds are not cumulated and
where the individual carbon atoms may carry one or more substituents independently of one another selected from the list below:
hydrogen, hydroxyl, CH₂OH, cyano, halogen, straight-chain or branched C₁-C₄-alkyl, straight-chain or branched C₁-C₄-haloalkyl, straight-chain or branched C₁-C₄-alkoxyalkyl, straight-chain or branched C₁-C₄-alkoxycarbonyl, straight-chain or substituted phenyl or benzyl, an optionally alkyl-substituted C₂-C₅-alkyl chain which may contain up to one oxygen, an optionally alkyl-substituted C₃-C₅-alkenyl chain,
R⁶ represents hydrogen, Me, C₁-C₄-alkylcarbonyl, CHO, C₁-C₄-alkoxy-C₁-C₄-alkylcarbonyl, C₁-C₄-alkoxy-C₁-C₄-alkyl, straight-chain or branched C₁-C₄-alkoxycarbonyl, COOBn, C₁-C₄-haloalkylcarbonyl, C₂-C₃-alkenyl, C₂-C₃-alkynyl, C₁-C₄-alkylsulphinyl, C₁-C₄-alkylsulphonyl, unsubstituted or substituted benzyl, C₁-C₄-trialkylsilyl, C₁-C₄-trialkylsilylethyl or C₁-C₄-dialkylmonophenylsilyl,
where the substituents independently of one another are selected from the group consisting of hydrogen, halogen, nitro, C₁-C₄-alkyl, C₁-C₄-alkoxy, hydroxyl, C₁-C₄-haloalkyl and cyano,
R⁷ represents hydrogen, cyano, C₁-C₃-alkyl or C₁-C₃-haloalkyl,
R⁸ represents halogen, cyano, C₁-C₂-haloalkyl, methyl, SMe, SOMe or SO₂Me,
R⁹ represents hydrogen, straight-chain or branched C₁-C₃-alkyl, 2-methoxyethan-1-yl, prop-2-en-1-yl, C₁-C₄-alkoxy(C₁-C₄)alkyl, straight-chain or branched (C₁-C₄-alkyl)carbonyl, (C₁-C₄-haloalkyl)carbonyl, unsubstituted or substituted benzyl, C₁-C₆-trialkylsilyl, C₁-C₄-trialkylsilylethyl, C₁-C₄-dialkylmonophenylsilyl, (C₁-C₄-alkoxy)carbonyl, C₁-C₆-alkylsulphinyl, C₁-C₆-alkylsulphonyl, C₁-C₆-haloalkylsulphinyl or C₁-C₆-haloalkylsulphonyl,
where the substituents independently of one another are selected from the group consisting of hydrogen, halogen, nitro, C₁-C₄-alkyl, C₁-C₄-alkoxy, hydroxyl, C₁-C₄-haloalkyl and cyano,
R¹⁰ represents straight-chain or branched unsubstituted or substituted C₁-C₇-alkyl, straight-chain or branched unsubstituted or substituted C₂-C₇-haloalkyl, unsubstituted or substituted C₃-C₇-cycloalkyl, straight-chain or branched unsubstituted or substituted C₃-C₇-cycloalkyl(C₁-C₃)alkyl, straight-chain or branched unsubstituted or substituted C₃-C₇-alkenyl, straight-chain or branched unsubstituted or substituted C₃-C₇-alkynyl, straight-chain or branched unsubstituted or substituted C₁-C₄-alkoxy(C₁-C₄)alkyl, straight-chain or branched unsubstituted or substituted C₁-C₄-haloalkoxy(C₁-C₄)alkyl, 2-methyl-1-(methylsulphanyl)propan-2-yl or oxetan-3-yl,
or
R⁹ and R¹⁰ together with the nitrogen atom to which they are attached form an unsubstituted or substituted 3- to 7-membered saturated cycle which may contain up to one further heteroatom selected from the group consisting of oxygen, sulphur and nitrogen,
where the substituents R¹⁰ independently of one another are selected from the group consisting of methyl, ethyl, isopropyl, cyclopropyl, fluorine, chlorine and/or bromine atoms, methoxy, ethoxy, methylmercapto, ethylmercapto, cyano, hydroxyl and CF₃,
and also agrochemically active salts thereof.

2. The compound of formula (I) according to claim 1 wherein:
R¹ to R⁵ independently of one another represent hydrogen, OH, Cl, F, Br, CH₃, CF₃, ethyl, OCH₃, SCH₃, OCF₂H or OCF₃,
where exactly one of the radicals R² and R³ represents a group of the formula E1, E2 or E3 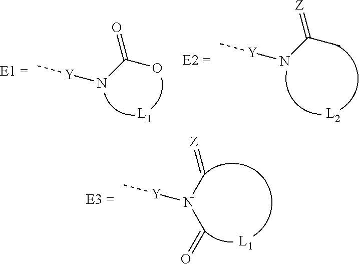
wherein:
Y represents a direct bond or —CH₂—, —CH₂CH₂—, —CH(CH₃)CH₂—, —CH₂CH(CH₃)—, —CHMe-, —CHOMe-, —CHCF₃— or C═O,
Z represents sulphur or oxygen,
L₁ represents an unsubstituted or substituted C₁- to C₄-alkylene chain or a C₂ to C₄-alkenyl chain,
where the double bonds are not cumulated and
where the individual carbon atoms may carry one or more substituents independently of one another selected from the list below:
hydrogen, methyl, ethyl, hydroxyl, oxo, methoxy, OCH₂CH₃, OC(CH₃)₃, OCH(CH₃)₂, O-propyl, O-butyl, COOCH₃, COOCH₂CH₃, COOC(CH₃)₃, COOPr, COOCH(CH₃)₂, CH₂OH, CH₂OCH₃, CH₂OCH₂CH₃, CH₂CH(CH₃)₂, CH₂C(CH₃)₃, phenyl or benzyl,
or
two substituents attached to two adjacent carbon atoms of the alkylene chain together with these two carbon atoms form
a 5- to 6-membered unsubstituted or substituted saturated carbocycle,
where the substituents independently of one another are selected from the list below:
hydrogen, fluorine, methyl, ethyl, propyl, isopropyl, tert-butyl, methoxy, ethoxy, propoxy, CF₃ or CHF₂,
or
a 5- or 6-membered unsubstituted or substituted saturated heterocycle which contains one oxygen atom or one sulphur atom,
where the substituents independently of one another are selected from the list below:
hydrogen, fluorine, methyl, ethyl, propyl, isopropyl, tert-butyl, methoxy, ethoxy, propoxy, CF₃ or CHF₂,
or
an unsubstituted or substituted phenyl ring,
where the substituents independently of one another are selected from the list below:
hydrogen, chlorine, fluorine, CN, NO₂, methyl, ethyl, propyl, isopropyl, tert-butyl, methoxy, ethoxy, propoxy, CF₃, CHF₂, OCF₃, OCHF₂, CO—CH₃ or COCH₂CH₃,
L₂ is an unsubstituted or substituted C₁- to C₄-alkylene chain, a C₂- to C₄-alkenyl chain or a 1,3-linked cyclopentyl (3-oxo-2-azabicyclo[2.2.1]hept-2-yl) or a C₁- to C₄-alkylene chain which is interrupted by a heteroatom selected from the group consisting of oxygen, sulphur and nitrogen,
where double bonds are not cumulated and
where the individual carbon atoms may carry one or more substituents independently of one another selected from the list below:
hydrogen, fluorine, chlorine, methyl, ethyl, propyl, isopropyl, tert-butyl, cyano, CF₃, hydroxyl, methoxy, O-propyl, O-isopropyl, O-butyl, O-tert-butyl, COOCH₃, COOCH₂CH₃, COOC(CH₃)₃, COOCH(CH₃)₂, COOPr, COOBu, OCH₂CH₃, CH₂OH, CH₂OMe, CH₂OEt, CH₂C(CH₃)₃, CH₂CH(CH₃)₂, phenyl, benzyl, —CH₂OCH₂CH₂—, —CH(CH₃)OCH₂CH₂—, —CH₂OCH(CH₃)CH₂—, —CH₂OCH₂CH(CH₃)—, —CHC(OCH₃)CH₂—, —C(CH₂CH₃)C(CH₃)CH₂—, —C(CH₂CH₃)C(CH₂CH₃)CH₂—, C(CH₃)C(CH₃)CH₂—, —CH═CH═CH═CH— or —C(CH₃)═CH═CH═CH—,
R⁶ represents hydrogen, Me, COMe, CHO, COCH₂OCH₃, CH₂OCH₃, COOMe, COOEt, COOtertBu, COOBn, COCF₃, CH₂CH═CH₂, CH₂C≡CH, SOCH₃, SO₂CH₃ or benzyl,
R⁷ represents hydrogen, cyano, methyl, CF₃ or CFH₂,
R⁸ represents chlorine, bromine, fluorine, iodine, cyano, CF₃, CFH₂, CF₂H, CCl₃, methyl, SMe, SOMe or SO₂Me,
R⁹ represents hydrogen, methyl, ethyl, propyl, propan-2-yl, 2-methoxyethan-1-yl, prop-2-en-1-yl, CH₂OCH₃, COMe, COOMe, COOEt, COOtertBu, COCF₃ or benzyl,
R¹⁰ represents straight-chain or branched unsubstituted or substituted C₁-C₆-alkyl, straight-chain or branched unsubstituted or substituted C₃-C₆-cycloalkyl(C₁-C₂)alkyl, unsubstituted or substituted C₃-C₆-cycloalkyl, straight-chain or branched unsubstituted or substituted C₃-C₄-alkenyl, straight-chain or branched unsubstituted or substituted C₃-C₄-alkynyl, straight-chain or branched unsubstituted or substituted C₂-C₄-haloalkyl, straight-chain or branched unsubstituted or substituted C₁-C₂-alkoxy(C₁-C₄)alkyl, straight-chain or branched unsubstituted or substituted C₁-C₂-alkylmercapto(C₁-C₄)alkyl or oxetan-3-yl,
where the substituents in R¹⁰ independently of one another are selected from the group consisting of methyl, ethyl, isopropyl, cyclopropyl, fluorine, chlorine and/or bromine atoms, methoxy, ethoxy, methylmercapto, ethylmercapto, cyano, hydroxyl and CF₃,
or
R⁹ and R¹⁰ together with the nitrogen atom to which they are attached form an azetidinyl, pyrrolidinyl, piperidinyl, morpholinyl, azepanyl, 4-methylpiperazin-1-yl, 2-methylpiperidin-1-yl, 2-methylpyrrolidin-1-yl, 2-methylazetidin-1-yl or thiomorpholinyl ring,
and also agrochemically active salts thereof.

3. The compound of formula (I) according to claim 1 wherein:
R¹ to R⁵ independently of one another represent hydrogen, OH, Cl, F, CH₃, CF₃, ethyl, OCH₃ or OCF₃,
where exactly one of the radicals R² and R³ represents a group of the formula E1, E2 or E3, 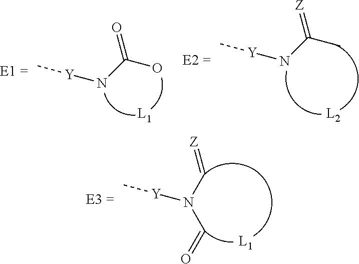
wherein:
Y represents a direct bond or —CH₂—, —CH₂CH₂—, —CHMe-, —CHEt-, —CHOMe-, —CHCF₃— or C═O,
Z represents sulphur or oxygen,
L₁ represents an unsubstituted or substituted C₁- to C₄-alkylene chain or a C₂- to C₄-alkenyl chain,
where double bonds are not cumulated and
where the individual carbon atoms may carry one or more substituents independently of one another selected from the list below:
hydrogen, methyl, ethyl, hydroxyl, oxo, methoxy, OCH₂CH₃, OC(CH₃)₃, OCH(CH₃)₂, COOCH₃, COOCH₂CH₃, COOC(CH₃)₃, CH₂OH, CH₂OCH₃, CH₂CH(CH₃)₂ or phenyl,
or
two substituents attached to two adjacent carbon atoms of the alkylene chain together with these two carbon atoms form
a 5- or 6-membered unsubstituted or substituted saturated carbocycle,
where the substituents independently of one another are selected from the list below:
hydrogen, fluorine, methyl, ethyl, propyl, methoxy, ethoxy or CF₃,
or
a 5- or 6-membered unsubstituted or substituted saturated heterocycle which contains an oxygen atom or a sulphur atom,
where the substituents independently of one another are selected from the list below:
hydrogen, fluorine, methyl, ethyl, tert-butyl, methoxy, ethoxy or CF₃,
or
an unsubstituted or substituted phenyl ring,
where the substituents independently of one another are selected from the list below:
hydrogen, chlorine, fluorine, CN, methyl, ethyl, tert-butyl, methoxy, CF₃ or CO—CH₃,
L₂ represents an unsubstituted or substituted C₁- to C₄-alkylene chain or a C₂- to C₄-alkenyl chain or a 1,3-linked cyclopentyl (3-oxo-2-azabicyclo[2.2.1]hept-2-yl) or a C₁- to C₄-alkylene chain which is interrupted by a heteroatom selected from the group consisting of oxygen, sulphur and nitrogen,
where double bonds are not cumulated and
where the individual carbon atoms may carry one or more substituents independently of one another selected from the list below:
hydrogen, fluorine, chlorine, methyl, ethyl, propyl, isopropyl, tert-butyl, cyano, CF₃, hydroxyl, methoxy, O-propyl, COOCH₃, COOCH₂CH₃, COOC(CH₃)₃, COOCH(CH₃)₂, OCH₂CH₃, CH₂OH, CH₂CH(CH₃)₂, phenyl, —CH₂OCH₂CH₂—, —CHC(OCH₃)CH₂—, —C(CH₂CH₃)C(CH₃)CH₂— or —CH═CH═CH═CH—,
R⁶ represents hydrogen, Me, COMe, CHO, COCH₂OCH₃, CH₂OCH₃, COOMe, COOEt, COCF₃, CH₂CH═CH₂, CH₂C≡CH, SOCH₃ or SO₂CH₃,
R⁷ represents hydrogen, cyano, methyl, CF₃ or CFH₂,
R⁸ represents chlorine, bromine, fluorine, iodine, cyano, CF₃, CFH₂, CF₂H, CCl₃, methyl, SMe, SOMe or SO₂Me,
R⁹ represents hydrogen, methyl, ethyl, propyl, propan-2-yl, 2-methoxyethan-1-yl, prop-2-en-1-yl, CH₂OCH₃, COMe, COOMe, COOEt, COOtertBu, COCF₃ or benzyl,
R¹⁰ represents methyl, ethyl, propyl, cyclopropyl, cyclopropylmethyl, 1-cyclopropyleth-1-yl, 2-methylcyclopropyl, 2,2-dimethylcyclopropyl, 2,2-dimethylprop-1-yl, tert-butyl, cyclobutyl, 2-methylcyclobut-1-yl, 3-methylcyclobut-1-yl, butyl, 3-methylbut-1-yl, 2-methylbut-1-yl, 2-methylprop-1-yl, 1-fluoroprop-2-yl, cyclopentyl, propan-2-yl, pentan-3-yl, pentan-2-yl, pentyl, prop-2-en-1-yl, prop-2-yn-1-yl, butan-2-yl, 2,2,2-trifluoroethyl, 2,2-difluoroethyl, 2-methoxyethan-1-yl, 2-methylmercaptoethan-1-yl, 2-fluoroethan-1-yl, 2-chloroethan-1-yl, 2-cyanoethan-1-yl, 1-methoxypropan-2-yl, 3-methoxypropan-1-yl, 2-hydroxyethan-1-yl, 1-hydroxypropan-2-yl, 3-hydroxypropan-1-yl, 1-methylmercaptopropan-2-yl, 2-methyl-1-(methylsulphanyl)propan-2-yl, oxetan-3-yl, 1,1,1-trifluoropropan-2-yl, 2,2,3,3,3-pentafluoropropyl, 1,1,1-trifluoropropan-3-yl, 1,1,1-trifluorobutan-2-yl, 1,1,1-trifluorobutan-3-yl, 2-methylprop-2-en-1-yl or 1-fluoropropan-2-yl
or
R⁹ and R¹⁰ together with the nitrogen atom to which they are attached represent an azetidinyl, pyrrolidinyl, piperidinyl, morpholinyl, azepanyl, 4-methylpiperazin-1-yl, 2-methylpiperidin-1-yl, 2-methylpyrrolidin-1-yl, 2-methylazetidin-1-yl or thiomorpholinyl ring,
and also agrochemically active salts thereof.

4. The compound of formula (I) according to claim 1 wherein:
R¹ to R⁵ independently of one another represent hydrogen, OH, Cl, F, CH₃ or OCF₃, where exactly one of the radicals R² and R³ represents a group of the formula E1, E2 or E3 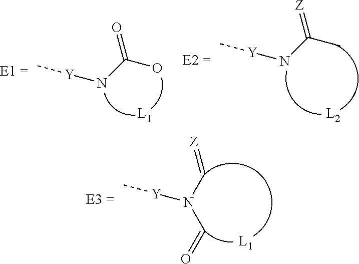
wherein:
Y represents a direct bond or —CH₂—, —CH₂CH₂—, —CHMe-, —CHCF₃— or C═O,
Z represents sulphur or oxygen,
L₁ represents an unsubstituted or substituted C₂- to C₃-alkylene chain or a C₂- to C₃-alkenyl chain,
where double bonds are not cumulated and
where the individual carbon atoms may carry one or more substituents independently of one another selected from the list below:
hydrogen, methyl, ethyl, hydroxyl, oxo, methoxy, OCH₂CH₃, COOCH₃, COOCH₂CH₃, CH₂OH, CH₂CH(CH₃)₂ or phenyl,
or
two substituents attached to two adjacent carbon atoms of the alkylene chain together with these two carbon atoms form
a 5- or 6-membered unsubstituted saturated carbocycle,
or
a 5- or 6-membered unsubstituted saturated heterocycle which contains one oxygen atom,
or
an unsubstituted phenyl ring,
L₂ represents an unsubstituted or substituted C₂- to C₃-alkylene chain or a C₂- to C₃-alkenyl chain or a 1,3-linked cyclopentyl (3-oxo-2-azabicyclo[2.2.1]hept-2-yl) or a C₁- to C₂-alkylene chain which is interrupted by a heteroatom selected from the group consisting of oxygen, sulphur and nitrogen,
where double bonds are not cumulated and
where the individual carbon atoms may carry one or more substituents independently of one another selected from the list below:
hydrogen, methyl, ethyl, cyano, CF₃, isopropyl, hydroxyl, methoxy, COOCH₃, COOCH₂CH₃, OCH₂CH₃, CH₂OH, CH₂CH(CH₃)₂, phenyl, —CH₂OCH₂CH₂—CHC(OCH₃)CH₂—, —C(CH₂CH₃)C(CH₃)CH₂— or —CH═CH═CH═CH—,
R⁶ represents hydrogen, Me, COMe, CHO or COCH₂OCH₃,
R⁷ represents hydrogen,
R⁸ represents chlorine, bromine, fluorine, iodine, cyano, CF₃, SMe, SOMe or SO₂Me,
R⁹ represents hydrogen, methyl, ethyl, propyl, propan-2-yl, 2-methoxyethan-1-yl or prop-2-en-1-yl,
R¹⁰ represents methyl, ethyl, propyl, cyclopropyl, cyclopropylmethyl, 1-cyclopropyleth-1-yl, 2-methylcyclopropyl, 2,2-dimethylcyclopropyl, 2,2-dimethylprop-1-yl, tert-butyl, cyclobutyl, 2-butyl, 3-methylbut-1-yl, 2-methylbut-1-yl, 2-methylprop-1-yl, 1-fluoroprop-2-yl, cyclopentyl, propan-2-yl, pentan-3-yl, pentan-2-yl, pentyl, prop-2-en-1-yl, prop-2-yn-1-yl, butan-2-yl, 2,2,2-trifluoroethyl, 2,2-difluoroethyl, 2-methoxyethan-1-yl, 2-methylmercaptoethan-1-yl, 2-fluoroethan-1-yl, 2-chloroethan-1-yl, 2-cyanoethan-1-yl, 1-methoxypropan-2-yl, 3-methoxypropan-1-yl, 2-hydroxyethan-1-yl, 1-hydroxypropan-2-yl, 3-hydroxypropan-1-yl, 1-methylmercaptopropan-2-yl, 2-methyl-1-(methylsulphanyl)propan-2-yl, oxetan-3-yl, 1,1,1-trifluoropropan-2-yl, 2,2,3,3,3-pentafluoropropyl, 1,1,1-trifluoropropan-3-yl, 1,1,1-trifluorobutan-2-yl, 1,1,1-trifluorobutan-3-yl, 2-methylprop-2-en-1-yl or 1-fluoropropan-2-yl
or
R⁹ and R¹⁰ together with the nitrogen atom to which they are attached form an azetidinyl, pyrrolidinyl, piperidinyl, morpholinyl, azepanyl, 4-methylpiperazin-1-yl, 2-methylpiperidin-1-yl, 2-methylpyrrolidin-1-yl, 2-methylazetidin-1-yl or thiomorpholinyl ring,
and also agrochemically active salts thereof.

5. The compound of the formula (I) according to claim 1 wherein:
R¹ represents hydrogen or OH,
R² represents hydrogen, (2,5-dioxopyrrolidin-1-yl)methyl, (2-oxopyrrolidin-1-yl)carbonyl, (3-methyl-2-oxopyrrolidin-1-yl)methyl, 1-(2,5-dioxopyrrolidin-1-yl)-2,2,2-trifluoroethyl, (2R)-2-(ethoxycarbonyl)-5-oxopyrrolidin-1-yl, (2S)-2-(ethoxycarbonyl)-5-oxopyrrolidin-1-yl, 2-(ethoxycarbonyl)-5-oxopyrrolidin-1-yl, (2R)-2-(hydroxymethyl)-5-oxopyrrolidin-1-yl, (2S)-2-(hydroxymethyl)-5-oxopyrrolidin-1-yl, 2-(hydroxymethyl)-5-oxopyrrolidin-1-yl, 2,5-dioxopyrrolidin-1-yl, 2-ethoxy-5-oxopyrrolidin-1-yl, (2R)-2-methyl-5-oxopyrrolidin-1-yl, (2S)-2-methyl-5-oxopyrrolidin-1-yl, 2-oxo-1,3-oxazolidin-3-yl, (4R)-2-oxo-4-(propan-2-yl)-1,3-oxazolidin-3-yl, (4S)-2-oxo-4-(propan-2-yl)-1,3-oxazolidin-3-yl, 2-oxo-4-(propan-2-yl)-1,3-oxazolidin-3-yl, 2-oxo-4-phenyl-1,3-oxazolidin-3-yl, 2-oxo-5-phenylpyrrolidin-1-yl, 2-oxoazepan-1-yl, 2-oxopiperidin-1-yl, 2-oxopyridin-1(2H)-yl, 2-oxopyrrolidin-1-yl, 2-thioxopyrrolidin-1-yl, 3,3-dimethyl-2,5-dioxopyrrolidin-1-yl, 3,3-dimethyl-2-oxoazetidin-1-yl, 3,5-dimethyl-2-oxopyrrolidin-1-yl, (3R,5R)-3,5-dimethyl-2-oxopyrrolidin-1-yl, (3R,5S)-3,5-dimethyl-2-oxopyrrolidin-1-yl, 3-ethyl-4-methyl-2-oxo-2,5-dihydro-1H-pyrrol-1-yl, (3R)-3-hydroxy-2-oxopyrrolidin-1-yl, (3S)-3-hydroxy-2-oxopyrrolidin-1-yl, 3-hydroxy-2-oxopyrrolidin-1-yl, 3-methyl-2,5-dioxopyrrolidin-1-yl, (3R)-3-methyl-2-oxopyrrolidin-1-yl, (3S)-3-methyl-2-oxopyrrolidin-1-yl, 3-methyl-2-oxopyrrolidin-1-yl, 3-oxomorpholin-4-yl, 4-(2-methylpropyl)-2-oxo-1,3-oxazolidin-3-yl, 4-(methoxycarbonyl)-2-oxopyrrolidin-1-yl, 4-ethyl-2-oxo-1,3-oxazolidin-3-yl, (4R)-4-hydroxy-2-oxopyrrolidin-1-yl, (4S)-4-hydroxy-2-oxopyrrolidin-1-yl, 4-hydroxy-2-oxopyrrolidin-1-yl, 4-methoxy-2-oxo-2,5-dihydro-1H-pyrrol-1-yl, (4R)-4-methyl-2-oxo-1,3-oxazolidin-3-yl, (4S)-4-methyl-2-oxo-1,3-oxazolidin-3-yl, 4-methyl-2-oxo-1,3-oxazolidin-3-yl, 4-methyl-2-oxopyrrolidin-1-yl, 5-ethyl-3-methyl-2-oxopyrrolidin-1-yl, 4,4-dimethyl-2-oxo-1,3-oxazolidin-3-yl, 5-methyl-2-oxo-1,3-oxazolidin-3-yl, 5,5-dimethyl-2-oxo-1,3-oxazolidin-3-yl, 2-ethyl-5-oxopyrrolidin-1-yl, 2-oxo-5-(propan-2-yl)pyrrolidin-1-yl, 2-oxo-3-(trifluoromethyl)pyrrolidin-1-yl, 3,3-dimethyl-2-oxopyrrolidin-1-yl, 3,3-dimethyl-2,5-dioxopyrrolidin-1-yl, 3,3-dimethyl-2-oxo-5-thioxopyrrolidin-1-yl, 3-cyano-2-oxopyrrolidin-1-yl, 3-oxo-2-azabicyclo[2.2.1]hept-2-yl, 1,3-dioxooctahydro-2H-isoindol-2-yl, 1,3-dioxo-1,3-dihydro-2H-isoindol-2-yl or 2,5-dioxo-2,5-dihydro-1H-pyrrol-1-yl,
R³ represents hydrogen, 2-oxo-1,3-oxazolidin-3-yl, 2-oxopyrrolidin-1-yl, 4-methyl-2-oxo-1,3-oxazolidin-3-yl, OCF₃, fluorine, methyl or chlorine,
where R² and R³ are not both hydrogen,
with the proviso that, if R² is not hydrogen,
R³ can only have one of the meanings below:
hydrogen, OCF₃ fluorine, methyl or chlorine,
R⁴ represents hydrogen or CH₃,
R⁵ represents hydrogen,
R⁶ represents hydrogen, Me, COMe, CHO or COCH₂OCH₃,
R⁷ represents hydrogen,
R⁸ represents chlorine, bromine, fluorine, iodine, cyano, CF₃, SMe, SOMe or SO₂Me,
R⁹ represents hydrogen, methyl, ethyl, propyl, propan-2-yl, 2-methoxyethan-1-yl or prop-2-en-1-yl,
R¹⁰ represents methyl, ethyl, propyl, cyclopropyl, cyclopropylmethyl, 1-cyclopropyleth-1-yl, 2-methylcyclopropyl, 2,2-dimethylcyclopropyl, 2,2-dimethylprop-1-yl, tert-butyl, cyclobutyl, 2-butyl, 3-methylbut-1-yl, 2-methylbut-1-yl, 2-methylprop-1-yl, 1-fluoroprop-2-yl, cyclopentyl, propan-2-yl, pentan-3-yl, pentan-2-yl, pentyl, prop-2-en-1-yl, prop-2-yn-1-yl, butan-2-yl, 2,2,2-trifluoroethyl, 2,2-difluoroethyl, 2-methoxyethan-1-yl, 2-methylmercaptoethan-1-yl, 2-fluoroethan-1-yl, 2-chloroethan-1-yl, 2-cyanoethan-1-yl, 1-methoxypropan-2-yl, 3-methoxypropan-1-yl, 2-hydroxyethan-1-yl, 1-hydroxypropan-2-yl, 3-hydroxypropan-1-yl, 1-methylmercaptopropan-2-yl, 2-methyl-1-(methylsulphanyl)propan-2-yl, oxetan-3-yl, 1,1,1-trifluoropropan-2-yl, 2,2,3,3,3-pentafluoropropyl, 1,1,1-trifluoropropan-3-yl, 1,1,1-trifluorobutan-2-yl, 1,1,1-trifluorobutan-3-yl, 2-methylprop-2-en-1-yl or 1-fluoropropan-2-yl,
or
R⁹ and R¹⁰ together with the nitrogen atom to which they are attached represent an azetidinyl, pyrrolidinyl, piperidinyl, morpholinyl, azepanyl, 4-methylpiperazin-1-yl, 2-methylpiperidin-1-yl, 2-methylpyrrolidin-1-yl, 2-methylazetidin-1-yl or thiomorpholinyl ring,
and also agrochemically active salts thereof.

6. A composition for controlling phytopathogenic harmful fungi, comprising at least one compound of formula (I) according to claim 1, and one or more extenders, surfactants or a combination thereof.

7. (canceled)

8. A method for controlling phytopathogenic harmful fungi, comprising applying the compound of formula (I) according to claim 1 to one or more phytopathogenic harmful fungi, their habitat or a combination thereof.

9. A process for preparing a composition for controlling phytopathogenic harmful fungi, comprising mixing the compound of formula (I) according to claim 1 with one or more extenders, surfactants or a combination thereof.

10. A process for preparing the compound of the formula (I) according to the invention 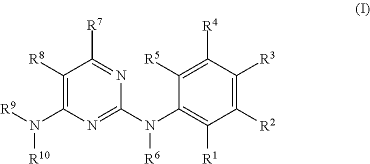
comprising at least one of steps (a) to (d) below:
(a) reacting a compound of formula (III) with a compound of formula (II) in the presence of a base, if appropriate in the presence of a solvent, if appropriate in the presence of a catalyst, to give a compound of formula (V), according to the reaction scheme below: 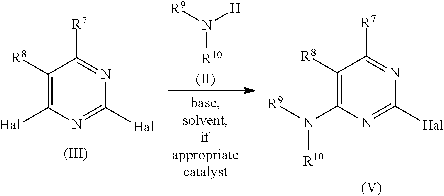
where Hal=F, Cl, Br or I;
(b) reacting the compound of formula (V) with a compound of formula (IV), if appropriate in the presence of an acid, if appropriate in the presence of a solvent, according to the reaction scheme below: 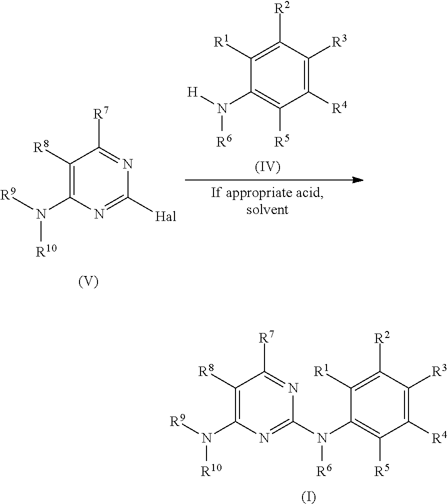
where Hal=F, Cl, Br or I;
(c) reacting the compound of formula (III) with the compound of formula (IV) in the presence of a solvent and a catalyst, to give a compound of formula (VI), according to the reaction scheme below: 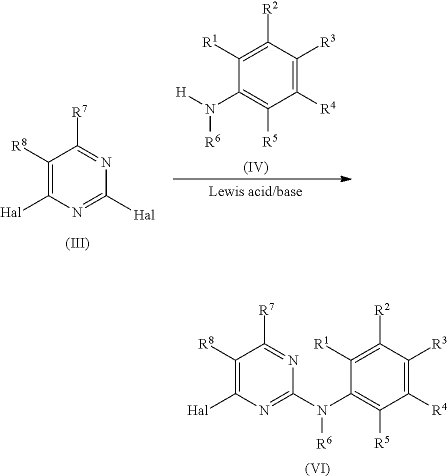
(d) reacting a compound of formula (VI) with the compound of formula (II) in the presence of a base, if appropriate in the presence of a solvent, if appropriate in the presence of a catalyst, to give the compound of formula (I), according to the reaction scheme below: 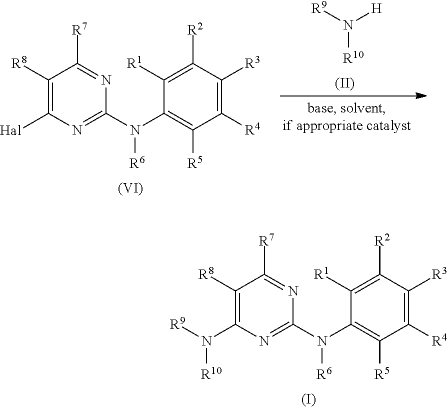
where the definitions of the radicals R¹ to R¹⁰ in the above schemes correspond to the definitions according to claim 1 and Hal represents F, Cl, Br or I.

11. The compound of formula (VI) 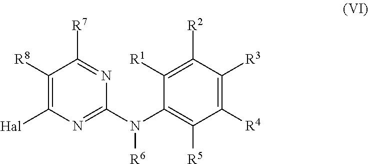
in which the symbols have the meanings below:
R¹ to R⁸ have meanings according to claim 1 and
Hal represents fluorine, chlorine, bromine or iodine.

12. A compound of formula (VIIa) 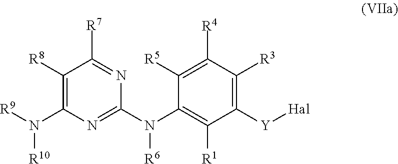
wherein:
Y represents a direct bond,
Hal represents bromine or iodine,
R¹ and R⁵ represent hydrogen,
R³ and R⁶ to R¹⁰ having meanings according to claim 1.

13. A compound of formula (VIIb) 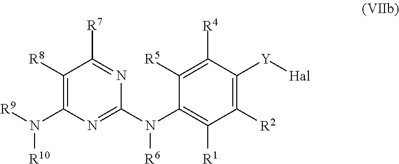
wherein:
Y represents a direct bond,
Hal represents bromine or iodine,
R¹ and R⁵ represent hydrogen,
R² and R⁶ to R¹⁰ have the meanings according to claim 1.

14. The compound of formula (V) 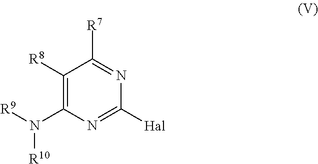
wherein:
R⁷ represents hydrogen,
and, if
R⁸ represents CF₃, CFH₂ or CF₂H,
Hal represents F, Cl, Br or I,
R⁹ represents hydrogen, ethyl, propyl, propan-2-yl, 2-methoxyethan-1-yl, prop-2-en-1-yl, CH₂OCH₃, COMe, COOMe, COOEt, COOtertBu, COCF₃ or benzyl,
R¹⁰ represents ethyl, propyl, cyclopropyl, cyclopropylmethyl, 1-cyclopropyleth-1-yl, 2-methylcyclopropyl, 2,2-dimethylcyclopropyl, 2,2-dimethylprop-1-yl, tert-butyl, cyclobutyl, 2-methylcyclobut-1-yl, 3-methylcyclobut-1-yl, butyl, 3-methylbut-1-yl, 2-methylbut-1-yl, 2-methylprop-1-yl, 1-fluoroprop-2-yl, cyclopentyl, propan-2-yl, pentan-3-yl, pentan-2-yl, pentyl, prop-2-en-1-yl, butan-2-yl, 2,2,2-trifluoroethyl, 2,2-difluoroethyl, 2-methoxyethan-1-yl, 2-methylmercaptoethan-1-yl, 2-fluoroethan-1-yl, 2-chloroethan-1-yl, 2-cyanoethan-1-yl, 1-methoxypropan-2-yl, 3-methoxypropan-1-yl, 1-methylmercaptopropan-2-yl, 2-methyl-1-(methylsulphanyl)propan-2-yl, oxetan-3-yl, 1,1,1-trifluoropropan-2-yl, 2,2,3,3,3-pentafluoropropyl, 1,1,1-trifluoropropan-3-yl, 1,1,1-trifluorobutan-2-yl, 1,1,1-trifluorobutan-3-yl, 2-methylprop-2-en-1-yl or 1-fluoropropan-2-yl,
or
R⁹ and R¹⁰ together with the nitrogen atom to which they are attached form an unsubstituted or substituted 3- to 7-membered saturated cycle which may contain up to one further heteroatom,
where the substituents independently of one another are selected from the group consisting of methyl, fluorine, chlorine and/or bromine atoms, cyano, hydroxyl, methoxy, CF₃,
and where the heteroatoms are selected from the group consisting of oxygen, sulphur and nitrogen.

15. A composition for controlling phytopathogenic harmful fungi, comprising at least one compound of formula (I) according to claim 2, and one or more extenders, surfactants or a combination thereof.

16. A composition for controlling phytopathogenic harmful fungi, comprising at least one compound of formula (I) according to claim 3, and one or more extenders, surfactants or a combination thereof.

17. A composition for controlling phytopathogenic harmful fungi, comprising at least one compound of formula (I) according to claim 4, and one or more extenders, surfactants or a combination thereof.

18. A composition for controlling phytopathogenic harmful fungi, comprising at least one compound of formula (I) according to claim 5, and one or more extenders, surfactants or a combination thereof.

19. A method for controlling phytopathogenic harmful fungi, comprising applying the one compound of formula (I) according to claim 2 to one or more phytopathogenic harmful fungi, their habitat or a combination thereof.

20. A method for controlling phytopathogenic harmful fungi, comprising applying the one compound of formula (I) according to claim 3 to one or more phytopathogenic harmful fungi, their habitat or a combination thereof.

21. A method for controlling phytopathogenic harmful fungi, comprising applying the one compound of formula (I) according to claim 4 to one or more phytopathogenic harmful fungi, their habitat or a combination thereof.

22. A method for controlling phytopathogenic harmful fungi, comprising applying the one compound of formula (I) according to claim 5 to one or more phytopathogenic harmful fungi, their habitat or a combination thereof.
